SEQUENCE LISTING (1094 sequences)

SEQ ID NO: 1 - protein, 245 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKICLTLSPST

SEQ ID NO: 2 - protein, 219 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKI

SEQ ID NO: 3 - protein, 268 aa, unidentified
  REGION 1..268: note = wild-type PD-1 polypeptide
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 4 - protein, 208 aa, Homo sapiens [sequence omitted]

SEQ ID NO: 5 - protein, 220 aa, unidentified
  REGION 1..220: note = wild-type CD28 amino acid sequence
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 6 - protein, 123 aa, unidentified
  REGION 1..123: note = wild-type CD28 amino acid sequence
MLRLLLALNLFPSIQVTGNKILVKQSPMLVAYDNAVNLSWKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 7 - protein, 101 aa, unidentified
  REGION 1..101: note = wild-type CD28 amino acid sequence
MLRLLLALNLFPSIQVTGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 8 - protein, 224 aa, Homo sapiens
APLKIQAYFNETADLPCQFANSQNQSLSELVVFWQDQENLVLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 9 - protein, 110 aa, Homo sapiens
APLKIQAYFNETADLPCQFANSQNQSLSELVVFWQDQENLVLNEVYLGKEKFDSVHSKYMNRTSFDSDSWTLRLHNLQIKDKGLYQCIIHHKKPTGMIRIHQMNSELSVL

SEQ ID NO: 10 - protein, 254 aa, unidentified
  REGION 1..254: note = wild-type 4-1BBL amino acid sequence
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 11 - protein, 174 aa, Homo sapiens
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 12 - protein, 175 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 13 - protein, 167 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPA

SEQ ID NO: 14 - protein, 255 aa, unidentified
  REGION 1..255: note = wild-type 4-1BB amino acid sequence
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 15 - protein, 133 aa, unidentified [sequence omitted]

SEQ ID NO: 16 - protein, 251 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 17 - protein, 524 aa, Homo sapiens
VNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 18 - protein, 347 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 19 - protein, 8 aa, SARS-CoV-2
NLTTRTQL

SEQ ID NO: 20 - protein, 9 aa, SARS-CoV-2
LPPAYTNSF

SEQ ID NO: 21 - protein, 9 aa, SARS-CoV-2
KVFRSSVLH

SEQ ID NO: 22 - protein, 9 aa, SARS-CoV-2
LPFFSNVTW

SEQ ID NO: 23 - protein, 9 aa, SARS-CoV-2
PFFSNVTWF

SEQ ID NO: 24 - protein, 9 aa, SARS-CoV-2
RFDNPVLPF

SEQ ID NO: 25 - protein, 9 aa, SARS-CoV-2
LPFNDGVYF

SEQ ID NO: 26 - protein, 9 aa, SARS-CoV-2
GVYFASTEK

SEQ ID NO: 27 - protein, 10 aa, SARS-CoV-2
TEKSNIIRGW

SEQ ID NO: 28 - protein, 9 aa, SARS-CoV-2
TLDSKTQSL

SEQ ID NO: 29 - protein, 9 aa, SARS-CoV-2
GVYYHKNNK

SEQ ID NO: 30 - protein, 9 aa, SARS-CoV-2
YYHKNNKSW

SEQ ID NO: 31 - protein, 10 aa, SARS-CoV-2
VYSSANNCTF

SEQ ID NO: 32 - protein, 9 aa, SARS-CoV-2
FEYVSQPFL

SEQ ID NO: 33 - protein, 9 aa, SARS-CoV-2
EYVSQPFLM

SEQ ID NO: 34 - protein, 9 aa, SARS-CoV-2
FVFKNIDGY

SEQ ID NO: 35 - protein, 9 aa, SARS-CoV-2
TPINLVRDL

SEQ ID NO: 36 - protein, 8 aa, SARS-CoV-2
LPQGFSAL

SEQ ID NO: 37 - protein, 10 aa, SARS-CoV-2
LPIGINITRF

SEQ ID NO: 38 - protein, 9 aa, SARS-CoV-2
INITRFQTL

SEQ ID NO: 39 - protein, 9 aa, SARS-CoV-2
LLALHRSYL

SEQ ID NO: 40 - protein, 9 aa, SARS-CoV-2
WTAGAAAYY

SEQ ID NO: 41 - protein, 11 aa, SARS-CoV-2
YYVGYLQPRTF

SEQ ID NO: 42 - protein, 9 aa, SARS-CoV-2
YLQPRTFLL

SEQ ID NO: 43 - protein, 8 aa, SARS-CoV-2
YLQPRTFL

SEQ ID NO: 44 - protein, 10 aa, SARS-CoV-2
SETKCTLKSF

SEQ ID NO: 45 - protein, 9 aa, SARS-CoV-2
TLKSFTVEK

SEQ ID NO: 46 - protein, 9 aa, SARS-CoV-2
QPTESIVRF

SEQ ID NO: 47 - protein, 11 aa, SARS-CoV-2
RFPNITNLCPF

SEQ ID NO: 48 - protein, 9 aa, SARS-CoV-2
GEVFNATRF

SEQ ID NO: 49 - protein, 9 aa, SARS-CoV-2
NATRFASVY

SEQ ID NO: 50 - protein, 10 aa, SARS-CoV-2
LYNSASFSTF

SEQ ID NO: 51 - protein, 9 aa, SARS-CoV-2
NSASFSTFK

SEQ ID NO: 52 - protein, 10 aa, SARS-CoV-2
RQIAPGQTGK

SEQ ID NO: 53 - protein, 9 aa, SARS-CoV-2
KIADYNYKL

SEQ ID NO: 54 - protein, 9 aa, SARS-CoV-2
NYNYLYRLF

SEQ ID NO: 55 - protein, 9 aa, SARS-CoV-2
RLFRKSNLK

SEQ ID NO: 56 - protein, 11 aa, SARS-CoV-2
KPFERDISTEI

SEQ ID NO: 57 - protein, 9 aa, SARS-CoV-2
YFPLQSYGF

SEQ ID NO: 58 - protein, 8 aa, SARS-CoV-2
QPYRVVVL

SEQ ID NO: 59 - protein, 9 aa, SARS-CoV-2
PYRVVVLSF

SEQ ID NO: 60 - protein, 9 aa, SARS-CoV-2
GPKKSTNLV

SEQ ID NO: 61 - protein, 9 aa, SARS-CoV-2
TSNQVAVLY

SEQ ID NO: 62 - protein, 9 aa, SARS-CoV-2
VYSTGSNVF

SEQ ID NO: 63 - protein, 8 aa, SARS-CoV-2
AEHVNNSY

SEQ ID NO: 64 - protein, 11 aa, SARS-CoV-2
IPIGAGICASY

SEQ ID NO: 65 - protein, 9 aa, SARS-CoV-2
SPRRARSVA

SEQ ID NO: 66 - protein, 9 aa, SARS-CoV-2
VASQSIIAY

SEQ ID NO: 67 - protein, 9 aa, SARS-CoV-2
SIIAYTMSL

SEQ ID NO: 68 - protein, 9 aa, SARS-CoV-2
LGAENSVAY

SEQ ID NO: 69 - protein, 13 aa, SARS-CoV-2
AYSNNSIAIPTNF

SEQ ID NO: 70 - protein, 9 aa, SARS-CoV-2
IPTNFTISV

SEQ ID NO: 71 - protein, 10 aa, SARS-CoV-2
TEILPVSMTK

SEQ ID NO: 72 - protein, 11 aa, SARS-CoV-2
QEVFAQVKQIY

SEQ ID NO: 73 - protein, 10 aa, SARS-CoV-2
KQIYKTPPIK

SEQ ID NO: 74 - protein, 10 aa, SARS-CoV-2
IYKTPPIKDF

SEQ ID NO: 75 - protein, 9 aa, SARS-CoV-2
LLFNKVTLA

SEQ ID NO: 76 - protein, 9 aa, SARS-CoV-2
TLADAGFIK

SEQ ID NO: 77 - protein, 10 aa, SARS-CoV-2
LADAGFIKQY

SEQ ID NO: 78 - protein, 9 aa, SARS-CoV-2
ADAGFIKQY

SEQ ID NO: 79 - protein, 14 aa, SARS-CoV-2
VLPPLLTDEMIAQY

SEQ ID NO: 80 - protein, 9 aa, SARS-CoV-2
IPFAMQMAY

SEQ ID NO: 81 - protein, 9 aa, SARS-CoV-2
SSTASALGK

SEQ ID NO: 82 - protein, 9 aa, SARS-CoV-2
VLNDILSRL

SEQ ID NO: 83 - protein, 9 aa, SARS-CoV-2
RLDKVEAEV

SEQ ID NO: 84 - protein, 10 aa, SARS-CoV-2
VEAEVQIDRL

SEQ ID NO: 85 - protein, 9 aa, SARS-CoV-2
AEVQIDRLI

SEQ ID NO: 86 - protein, 9 aa, SARS-CoV-2
LITGRLQSL

SEQ ID NO: 87 - protein, 9 aa, SARS-CoV-2
RLQSLQTYV

SEQ ID NO: 88 - protein, 9 aa, SARS-CoV-2
AEIRASANL

SEQ ID NO: 89 - protein, 9 aa, SARS-CoV-2
ASANLAATK

SEQ ID NO: 90 - protein, 9 aa, SARS-CoV-2
HLMSFPQSA

SEQ ID NO: 91 - protein, 11 aa, SARS-CoV-2
FPQSAPHGVVF

SEQ ID NO: 92 - protein, 8 aa, SARS-CoV-2
APHGVVFL

SEQ ID NO: 93 - protein, 9 aa, SARS-CoV-2
VTYVPAQEK

SEQ ID NO: 94 - protein, 10 aa, SARS-CoV-2
TYVPAQEKNF

SEQ ID NO: 95 - protein, 12 aa, SARS-CoV-2
REGVFVSNGTHW

SEQ ID NO: 96 - protein, 9 aa, SARS-CoV-2
GTHWFVTQR

SEQ ID NO: 97 - protein, 14 aa, SARS-CoV-2
TVYDPLQPELDSFK

SEQ ID NO: 98 - protein, 9 aa, SARS-CoV-2
KEIDRLNEV

SEQ ID NO: 99 - protein, 12 aa, SARS-CoV-2
QELGKYEQYIKW

SEQ ID NO: 100 - protein, 9 aa, SARS-CoV-2
YEQYIKWPW

SEQ ID NO: 101 - protein, 9 aa, SARS-CoV-2
QYIKWPWYI

SEQ ID NO: 102 - protein, 9 aa, SARS-CoV-2
FIAGLIAIV

SEQ ID NO: 103 - protein, 10 aa, SARS-CoV-2
SEPVLKGVKL

SEQ ID NO: 104 - protein, 9 aa, SARS-CoV-2
GTITVEELK

SEQ ID NO: 105 - protein, 10 aa, SARS-CoV-2
EELKKLLEQW

SEQ ID NO: 106 - protein, 9 aa, SARS-CoV-2
KLLEQWNLV

SEQ ID NO: 107 - protein, 9 aa, SARS-CoV-2
FAYANRNRF

SEQ ID NO: 108 - protein, 9 aa, SARS-CoV-2
YANRNRFLY

SEQ ID NO: 109 - protein, 10 aa, SARS-CoV-2
SYFIASFRLF

SEQ ID NO: 110 - protein, 9 aa, SARS-CoV-2
RLFARTRSM

SEQ ID NO: 111 - protein, 8 aa, SARS-CoV-2
VPLHGTIL

SEQ ID NO: 112 - protein, 10 aa, SARS-CoV-2
SELVIGAVIL

SEQ ID NO: 113 - protein, 9 aa, SARS-CoV-2
HLRIAGHHL

SEQ ID NO: 114 - protein, 9 aa, SARS-CoV-2
RIAGHHLGR

SEQ ID NO: 115 - protein, 11 aa, SARS-CoV-2
KEITVATSRTL

SEQ ID NO: 116 - protein, 10 aa, SARS-CoV-2
ATSRTLSYYK

SEQ ID NO: 117 - protein, 14 aa, SARS-CoV-2
ASQRVAGDSGFAAY

SEQ ID NO: 118 - protein, 9 aa, SARS-CoV-2
VAGDSGFAA

SEQ ID NO: 119 - protein, 9 aa, SARS-CoV-2
LPNNTASWF

SEQ ID NO: 120 - protein, 10 aa, SARS-CoV-2
KFPRGQGVPI

SEQ ID NO: 121 - protein, 13 aa, SARS-CoV-2
NTNSSPDDQIGYY

SEQ ID NO: 122 - protein, 9 aa, SARS-CoV-2
SPRWYFYYL

SEQ ID NO: 123 - protein, 9 aa, SARS-CoV-2
LLLDRLNQL

SEQ ID NO: 124 - protein, 9 aa, SARS-CoV-2
KAYNVTQAF

SEQ ID NO: 125 - protein, 13 aa, SARS-CoV-2
QELIRQGTDYKHW

SEQ ID NO: 126 - protein, 9 aa, SARS-CoV-2
ASAFFGMSR

SEQ ID NO: 127 - protein, 13 aa, SARS-CoV-2
SRIGMEVTPSGTW

SEQ ID NO: 128 - protein, 11 aa, SARS-CoV-2
GMEVTPSGTWL

SEQ ID NO: 129 - protein, 9 aa, SARS-CoV-2
TPSGTWLTY

SEQ ID NO: 130 - protein, 11 aa, SARS-CoV-2
AYKTFPPTEPK

SEQ ID NO: 131 - protein, 9 aa, SARS-CoV-2
LPAADLDDF

SEQ ID NO: 132 - protein, 5 aa, Homo sapiens
GTLRG

SEQ ID NO: 133 - protein, 5 aa, Homo sapiens
YNQSE

SEQ ID NO: 134 - protein, 5 aa, Homo sapiens
TAADM

SEQ ID NO: 135 - protein, 5 aa, Homo sapiens
AQTTK

SEQ ID NO: 136 - protein, 5 aa, Homo sapiens
VETRP

SEQ ID NO: 137 - protein, 5 aa, Homo sapiens
GDGTF

SEQ ID NO: 138 - protein, 5 aa, Homo sapiens
RNLRG

SEQ ID NO: 139 - protein, 5 aa, Homo sapiens
TAADT

SEQ ID NO: 140 - protein, 5 aa, Homo sapiens
AQITQ

SEQ ID NO: 141 - protein, 5 aa, Homo sapiens
GDRTF

SEQ ID NO: 142 - protein, 276 aa, synthetic construct
  REGION 1..276: note = synthetic sequence
  VARIANT 9: note = X is F, Y, S, or T
  VARIANT 44: note = X is K or R
  VARIANT 62: note = X is Q, G, E, or R
  VARIANT 63: note = X is N or E
  VARIANT 65: note = X is R or G
  VARIANT 66: note = X is N or K
  VARIANT 67: note = X is M or V
  VARIANT 70: note = X is H or Q
  VARIANT 73: note = X is H or Q
  VARIANT 74: note = X is D or H
  VARIANT 76: note = X is A, V, or E
  VARIANT 77: note = X is N or D
  VARIANT 79: note = X is G or R
  VARIANT 80: note = X is T or I
  VARIANT 81: note = X is L or A
  VARIANT 82: note = X is R or L
  VARIANT 83: note = X is G or R
  VARIANT 90: note = X is A or D
  VARIANT 95: note = X is I, L, or V
  VARIANT 97: note = X is I, R or M
  VARIANT 99: note = X is F or Y
  VARIANT 105: note = X is S or P
  VARIANT 107: note = X is W or G
  VARIANT 114: note = X is R, H, or Q
  VARIANT 116: note = X is D or Y
  VARIANT 127: note = X is N or K
  VARIANT 142: note = X is T or I
  VARIANT 144: note = X is K or Q
  VARIANT 145: note = X is R or H
  VARIANT 149: note = X is A or T
  VARIANT 150: note = X is A or V
  VARIANT 151: note = X is H or R
  VARIANT 156: note = X is R, L, Q, or W
  VARIANT 158: note = X is V or A
  VARIANT 161: note = X is D or E
  VARIANT 163: note = X is R or T
  VARIANT 166: note = X is D or E
  VARIANT 167: note = X is W or G
  VARIANT 184: note = X is P or A
  VARIANT 193: note = X is P or A
  VARIANT 194: note = X is P or A
  VARIANT 207: note = X is S or G
  VARIANT 246: note = X is A or S
  VARIANT 253: note = X is Q or E
  VARIANT 276: note = X is P or L
GSHSMRYFXTSVSRPGRGEPRFIAVGYVDDTQFVRFDSDAASQXMEPRAPWIEQEGPEYWDXXTXXXKAXSQXXRXXLXXXXXYYNQSEXGSHTXQXMXGCDVGXDXRFLRGYXQXAYDGKDYIALXEDLRSWTAADMAAQXTXXKWEXXXEAEQXRXYLXGXCVXXLRRYLENGKETLQRTDXPKTHMTHHXXSDHEATLRCWALXFYPAEITLTWQRDGEDQTQDTELVETRPAGDGTFQKWAXVVVPSGXEQRYTCHVQHEGLPKPLTLRWEX

SEQ ID NO: 143 - protein, 276 aa, Homo sapiens [sequence omitted]

SEQ ID NO: 144 - protein, 275 aa, Homo sapiens [sequence omitted]

SEQ ID NO: 145 - protein, 276 aa, Homo sapiens
GSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEP

SEQ ID NO: 146 - protein, 275 aa, Homo sapiens
GSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWE

SEQ ID NO: 147 - protein, 276 aa, Homo sapiens
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEP

SEQ ID NO: 148 - protein, 276 aa, Homo sapiens
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEP

SEQ ID NO: 149 - protein, 275 aa, Homo sapiens
GSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 150 - protein, 275 aa, Homo sapiens
GSHSMRYFYTSVSRPGRGEPRFIAVGYVDDTQFVRFDSDAASQRMEPRAPWIEQEGPEYWDQETRNVKAQSQTDRVDLGTLRGAYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 151 - protein, 341 aa, Homo sapiens
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEPSSQPTVPIVGIIAGLVLLGAVITGAVVAAVMWRRNSSDRKGGSYSQAASSDSAQGSDVSLTACKV

SEQ ID NO: 152 - protein, 275 aa, Homo sapiens
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 153 - protein, 275 aa, Homo sapiens
GSHSMRYFSTSVSRPGRGEPRFIAVGYVDDTQFVRFDSDAASQRMEPRAPWIEQEGPEYWDEETGKVKAHSQTDRENLRIALRAYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 154 - protein, 275 aa, Homo sapiens
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 155 - protein, 275 aa, Homo sapiens
GSHSMRYFSTSVSRPGRGEPRFIAVGYVDDTQFVRFDSDAASQRMEPRAPWIEQEGPEYWDEETGKVKAHSQTDRENLRIALRAYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 156 - protein, 275 aa, Homo sapiens
GSHSMRYFSTSVSRPGRGEPRFIAVGYVDDTQFVRFDSDAASQRMEPRAPWIEQEGPEYWDEETGKVKAHSQTDRENLRIALRCYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 157 - protein, 275 aa, Homo sapiens
GSHSMRYFSTSVSRPGRGEPRFIAVGYVDDTQFVRFDSDAASQRMEPRAPWIEQEGPEYWDEETGKVKAHSQTDRENLRIALRCYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 158 - protein, 341 aa, Homo sapiens
GSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWEPSSQPTIPIVGIIAGLVLFGAVFAGAVVAAVRWRRKSSDRKGGSYSQAASSDSAQGSDMSLTACKV

SEQ ID NO: 159 - protein, 276 aa, synthetic construct
  REGION 1..276: note = synthetic sequence
  VARIANT 9: note = X is H, Y or D
  VARIANT 11: note = X is A or S
  VARIANT 12: note = X is M or V
  VARIANT 24: note = X is A, S, or T
  VARIANT 32: note = X is Q or L
  VARIANT 41: note = X is A or T
  VARIANT 45: note = X is E, M K, or T
  VARIANT 46: note = X is A or T
  VARIANT 63: note = X is E or N
  VARIANT 66: note = X iis I or K
  VARIANT 67: note = X is Y, F, S, or C
  VARIANT 70: note = X is N or Q
  VARIANT 71: note = X is A or T
  VARIANT 74: note = X is D or Y
  VARIANT 76: note = X is E or V
  VARIANT 77: note = X is S or N
  VARIANT 80: note = X is T, N or I
  VARIANT 81: note = X is A or L
  VARIANT 82: note = X is L or R
  VARIANT 83: note = X is R or G
  VARIANT 94: note = X is T or I
  VARIANT 95: note = X is L or I
  VARIANT 97: note = X is R or S
  VARIANT 131: note = X is R or S
  VARIANT 143: note = X is S or T
  VARIANT 147: note = X is L or W
  VARIANT 152: note = X is E or V
  VARIANT 156: note = X is R, D, L or W
  VARIANT 158: note = X is A or T
  VARIANT 163: note = X is L, E or T
  VARIANT 177: note = X is E or D
  VARIANT 178: note = X is K or T
  VARIANT 180: note = X is E or Q
  VARIANT 194: note = X is I or V
GSHSMRYFXTXXSRPGRGEPRFIXVGYVDDTXFVRFDSDAXSPRXXPRAPWIEQEGPEYWDRXTQXXKTXXTQXYXXNLXXXXYYNQSEAGSHXXQXMYGCDLGPDGRLLRGHDQSAYDGKDYIALNEDLXSWTAADTAAQIXQRKXEAARXAEQXRXYLEGXCVEWLRRYLENGKXXLXRADPPKTHVTHHPXSDHEATLRCWALGFYPAEITLTWQRDGEDQTQDTELVETRPAGDRTFQKWAAVVVPSGEEQRYTCHVQHEGLPKPLTLRWEP

SEQ ID NO: 160 - protein, 276 aa, Homo sapiens [sequence omitted]

SEQ ID NO: 161 - protein, 276 aa, Homo sapiens
GSHSMRYFYTSVSRPGRGEPRFISVGYVDDTQFVRFDSDAASPREEPRAPWIEQEGPEYWDRNTQIYKAQAQTDRESLRNLRGAYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPCGDRTFQKWAAVVVPSGEEQRYTCHVQHEGLPKPLTLRWEP

SEQ ID NO: 162 - protein, 276 aa, Homo sapiens
GSHSMRYFYTSVSRPGRGEPRFISVGYVDDTQFVRFDSDAASPREEPRAPWIEQEGPEYWDRNTQIYKAQAQTDRESLRNLRGCYNQSEAGSHTLQSMYGCDVGPDGRLLRGHDQYAYDGKDYIALNEDLRSWTAADTCAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 163 - protein, 276 aa, synthetic construct
  REGION 1..276: note = synthetic sequence
  VARIANT 1: note = X is C or G
  VARIANT 6: note = X is R or K
  VARIANT 9: note = X is F, Y, S, or D
  VARIANT 14: note = X is R or W
  VARIANT 21: note = X is H or R
  VARIANT 24: note = X is A or S
  VARIANT 35: note = X is Q or R
  VARIANT 49: note = X is A or E
  VARIANT 66: note = X is N or K
  VARIANT 73: note = X is T or A
  VARIANT 77: note = X is S or N
  VARIANT 80: note = X is N or K
  VARIANT 90: note = X is A or D
  VARIANT 91: note = X is G or R
  VARIANT 94: note = X is T or I
  VARIANT 95: note = X is L or I
  VARIANT 97: note = X is W or R
  VARIANT 99: note = X is C, Y, F, or S
  VARIANT 103: note = X is L or V
  VARIANT 113: note = X is Y or H
  VARIANT 114: note = X is D or N
  VARIANT 116: note = X is Y, F, S, or L
  VARIANT 147: note = X is L or W
  VARIANT 152: note = X is E, A or T
  VARIANT 156: note = X is R, L or W
  VARIANT 163: note = X is L or T
  VARIANT 173: note = X is E or K
  VARIANT 177: note = X is E or K
  VARIANT 184: note = X is H or P
  VARIANT 194: note = X is R or V
  VARIANT 219: note = X is W or R
  VARIANT 248: note = X is V or M
  VARIANT 253: note = X is E or Q
  VARIANT 261: note = X is M or V
  VARIANT 267: note = X is P or Q
  VARIANT 273: note = X is R or S
  VARIANT 275: note = X is P or G
XSHSMXYFXTAVSXPGRGEPXFIXVGYVDDTQFVXFDSDAASPRGEPRXPWVEQEGPEYWDRETQXYKRQAQXDRVXLRXLRGYYNQSEXXSHXXQXMXGCDXGPDGRLLRGXXQXAYDGKDYIALNEDLRSWTAADTAAQITQRKXEAARXAEQXRAYLEGXCVEWLRRYLXNGKXTLQRAEXPKTHVTHHPXSDHEATLRCWALGFYPAEITLTWQXDGEDQTQDTELVETRPAGDGTFQKWAAVXVPSGXEQRYTCHXQHEGLXEPLTLXWXP

SEQ ID NO: 164 - protein, 276 aa, Homo sapiens
CSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEP

SEQ ID NO: 165 - protein, 276 aa, Homo sapiens
CSHSMRYFDTAVSRPGRGEPRFISVGYVDDTQFVRFDSDAASPRGEPRAPWVEQEGPEYWDRETQNYKRQAQADRVSLRNLRGAYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPCGDGTFQKWAAVVVPSGQEQRYTCHMQHEGLQEPLTLSWEP

SEQ ID NO: 166 - protein, 276 aa, Homo sapiens
CSHSMRYFDTAVSRPGRGEPRFISVGYVDDTQFVRFDSDAASPRGEPRAPWVEQEGPEYWDRETQNYKRQAQADRVSLRNLRGCYNQSEDGSHTLQRMYGCDLGPDGRLLRGYDQSAYDGKDYIALNEDLRSWTAADTCAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEP

SEQ ID NO: 167 - protein, 99 aa, unidentified
  REGION 1..99: note = B2M polypeptide
IQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 168 - protein, 252 aa, synthetic construct
  REGION 1..252: note = synthetic sequence
  VARIANT 79: note = X is a sequence independently selected to be 1-5 amino acid residues i) selected independently from any naturally occurring (e.g., encoded) amino acid or ii) any naturally occurring amino acid except proline or glycine
  VARIANT 81: note = X is a sequence independently selected to be 1-5 amino acid residues i) selected independently from any naturally occurring (e.g., encoded) amino acid or ii) any naturally occurring amino acid except proline or glycine
  VARIANT 126: note = X is a sequence independently selected to be 1-5 amino acid residues i) selected independently from any naturally occurring (e.g., encoded) amino acid or ii) any naturally occurring amino acid except proline or glycine
  VARIANT 128: note = X is a sequence independently selected to be 1-5 amino acid residues i) selected independently from any naturally occurring (e.g., encoded) amino acid or ii) any naturally occurring amino acid except proline or glycine
  VARIANT 215: note = X is a sequence independently selected to be 1-5 amino acid residues i) selected independently from any naturally occurring (e.g., encoded) amino acid or ii) any naturally occurring amino acid except proline or glycine
  VARIANT 217: note = X is a sequence independently selected to be 1-5 amino acid residues i) selected independently from any naturally occurring (e.g., encoded) amino acid or ii) any naturally occurring amino acid except proline or glycine
GSHSMRYFFTSVSRPGRGEPRFIAVGYVDDTQFVRFDSDAASQRMEPRAPWIEQEGPEYWDGETRKVKAHSQTHRVDLXCXAGSHTVQRMYGCDVGSDWRFLRGYHQYAYDGKDYIALKEDLRSWXCXHKWEAAHVAEQLRAYLEGTCVEWLRRYLENGKETLQRTDAPKTHMTHHAVSDHEATLRCWALSFYPAEITLTWQRDGEDQTQDTELXIXQKWAAVVVPSGQEQRYTCHVQHEGLPKPLTLRWEP

SEQ ID NO: 169 - protein, 99 aa, unidentified [sequence omitted]

SEQ ID NO: 170 - protein, 276 aa, Homo sapiens
GSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEP

SEQ ID NO: 171 - protein, 10 aa, synthetic construct
  REGION 1..10: note = synthetic sequence
  REPEAT 6..10: note = The sequence repeats n times, where n is 1, 2, 3, 4, 5, 6, 7, 8, or 9
GCGGSGGGGS

SEQ ID NO: 172 - protein, 20 aa, synthetic construct
  REGION 1..20: note = synthetic sequence
GCGGSGGGGSGGGGSGGGGS

SEQ ID NO: 173 - protein, 15 aa, synthetic construct
  REGION 1..15: note = synthetic sequence
GCGGSGGGGSGGGGS

SEQ ID NO: 174 - protein, 5 aa, synthetic construct
  REGION 1..5: note = synthetic sequence
CGGGS

SEQ ID NO: 175 - protein, 10 aa, synthetic construct
  REGION 1..10: note = synthetic sequence
  REPEAT 6..10: note = The sequence repeats n times, where n is an integer from 1 to 10
CGGGSGGGGS

SEQ ID NO: 176 - protein, 276 aa, Homo sapiens
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEP

SEQ ID NO: 177 - protein, 5 aa, synthetic construct
  REGION 1..5: note = synthetic sequence
GCGGS

SEQ ID NO: 178 - protein, 275 aa, Homo sapiens
GSHSMRYFFTSVSRPGRGEPRFIAVGYVDDTQFVRFDSDAASQKMEPRAPWIEQEGPEYWDQETRNMKAHSQTDRANLGTLRGCYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 179 - protein, 275 aa, Homo sapiens
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWE

SEQ ID NO: 180 - protein, 275 aa, Homo sapiens
GSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 181 - protein, 275 aa, Homo sapiens
GSHSMRYFSTSVSRPGRGEPRFIAVGYVDDTQFVRFDSDAASQRMEPRAPWIEQEGPEYWDEETGKVKAHSQTDRENLRIALRCYNQSEAGSHTLQMMFGCDVGSDGRFLRGYHQYAYDGKDYIALKEDLRSWTAADMAAQITQRKWEAARVAEQLRAYLEGTCVDGLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 182 - protein, 275 aa, Homo sapiens
GSHSMRYFSTSVSRPGRGEPRFIAVGYVDDTQFVRFDSDAASQRMEPRAPWIEQEGPEYWDEETGKVKAQSQTDRENLRIALRCYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 183 - protein, 275 aa, Homo sapiens
GSHSMRYFTTSVSRPGRGEPRFIAVGYVDDTQFVRFDSDAASQRMEPRAPWIEQEGPEYWDRNTRNVKAHSQIDRVDLGTLRGCYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPCGDGTFQKWASVVVPSGQEQRYTCHVQHEGLPKPLTLRWE

SEQ ID NO: 184 - protein, 275 aa, Homo sapiens
GSHSMRYFYTSVSRPGRGEPRFIAVGYVDDTQFVRFDSDAASQRMEPRAPWIEQEGPEYWDRNTRKVKAQSQTDRVDLGTLRGCYNQSEDGSHTIQRMYGCDVGPDGRFLRGYQQDAYDGKDYIALNEDLRSWTAADMAAQITQRKWETAHEAEQWRAYLEGTCVEWLRRYLENGKETLQRTDAPKTHMTHHAVSDHEATLRCWALSFYPAEITLTWQRDGEDQTQDTELVETRPCGDGTFQKWASVVVPSGQEQRYTCHVQHEGLPKPLTLRWE

SEQ ID NO: 185 - protein, 276 aa, Homo sapiens
GSHSMRYFYTSVSRPGRGEPRFISVGYVDDTQFVRFDSDAASPREEPRAPWIEQEGPEYWDRNTQIYKAQAQTDRESLRNLRGCYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPCGDRTFQKWAAVVVPSGEEQRYTCHVQHEGLPKPLTLRWEP

SEQ ID NO: 186 - protein, 275 aa, Homo sapiens
GSHSMRYFYTSVSRPGRGEPRFISVGYVDDTQFVRFDSDAASPREEPRAPWIEQEGPEYWDRNTQIYKAQAQTDRESLRNLRGCYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPCGDRTFQKWAAVVVPSGEEQRYTCHVQHEGLPKPLTLRWE

SEQ ID NO: 187 - protein, 275 aa, Homo sapiens
GSHSMRYFDTAMSRPGRGEPRFISVGYVDDTQFVRFDSDAASPREEPRAPWIEQEGPEYWDRNTQIFKTNTQTDRESLRNLRGCYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPCGDRTFQKWAAVVVPSGEEQRYTCHVQHEGLPKPLTLRWE

SEQ ID NO: 188 - protein, 275 aa, Homo sapiens
GSHSMRYFYTAMSRPGRGEPRFIAVGYVDDTQFVRFDSDAASPRMAPRAPWIEQEGPEYWDRNTQISKTNTQTYRESLRNLRGCYNQSEAGSHIIQRMYGCDVGPDGRLLRGYDQSAYDGKDYIALNEDLSSWTAADTAAQITQRKWEAAREAEQLRAYLEGLCVEWLRRYLENGKETLQRADPPKTHVTHHPISDHEATLRCWALGFYPAEITLTWQRDGEDQTQDTELVETRPCGDRTFQKWAAVVVPSGEEQRYTCHVQHEGLPKPLTLRWE

SEQ ID NO: 189 - protein, 275 aa, Homo sapiens
GSHSMRYFYTSVSRPGRGEPRFISVGYVDDTQFVRFDSDAASPREEPRAPWIEQEGPEYWDRNTQICKTNTQTYRENLRTALRCYNQSEAGSHTLQRMYGCDVGPDGRLLRGHNQFAYDGKDYIALNEDLSSWTAADTAAQITQRKWEAARVAEQLRTYLEGTCVEWLRRYLENGKETLQRADPPKTHVTHHPISDHEATLRCWALGFYPAEITLTWQRDGEDQTQDTELVETRPCGDRTFQKWAAVVVPSGEEQRYTCHVQHEGLPKPLTLRWE

SEQ ID NO: 190 - protein, 275 aa, Homo sapiens
GSHSMRYFHTAMSRPGRGEPRFITVGYVDDTLFVRFDSDATSPRKEPRAPWIEQEGPEYWDRETQISKTNTQTYRESLRNLRGCYNQSEAGSHTLQRMYGCDVGPDGRLLRGHNQYAYDGKDYIALNEDLRSWTAADTAAQISQRKLEAARVAEQLRAYLEGECVEWLRRYLENGKDKLERADPPKTHVTHHPISDHEATLRCWALGFYPAEITLTWQRDGEDQTQDTELVETRPCGDRTFQKWAAVVVPSGEEQRYTCHVQHEGLPKPLTLRWE

SEQ ID NO: 191 - protein, 275 aa, Homo sapiens
GSHSMRYFYTAMSRPGRGEPRFIAVGYVDDTQFVRFDSDAASPRMAPRAPWIEQEGPEYWDRETQKYKRQAQTDRVSLRNLRGCYNQSEAGSHTLQRMYGCDVGPDGRLLRGHDQSAYDGKDYIALNEDLSSWTAADTAAQITQRKWEAAREAEQWRAYLEGLCVEWLRRYLENGKETLQRADPPKTHVTHHPISDHEATLRCWALGFYPAEITLTWQRDGEDQTQDTELVETRPCGDRTFQKWAAVVVPSGEEQRYTCHVQHEGLPKPLTLRWE

SEQ ID NO: 192 - protein, 275 aa, Homo sapiens
GSHSMRYFYTAMSRPGRGEPRFIAVGYVDDTQFVRFDSDAASPRTEPRAPWIEQEGPEYWDRNTQIFKTNTQTYRENLRIALRCYNQSEAGSHIIQRMYGCDLGPDGRLLRGHDQSAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPCGDRTFQKWAAVVVPSGEEQRYTCHVQHEGLPKPLTLRWE

SEQ ID NO: 193 - protein, 276 aa, Homo sapiens
CSHSMRYFDTAVSRPGRGEPRFISVGYVDDTQFVRFDSDAASPRGEPRAPWVEQEGPEYWDRETQNYKRQAQADRVSLRNLRGCYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPCGDGTFQKWAAVVVPSGQEQRYTCHMQHEGLQEPLTLSWEP

SEQ ID NO: 194 - protein, 276 aa, Homo sapiens
CSHSMKYFFTSVSRPGRGEPRFISVGYVDDTQFVRFDSDAASPRGEPRAPWVEQEGPEYWDRETQKYKRQAQTDRVSLRNLRGCYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPCGDGTFQKWAAVMVPSGEEQRYTCHVQHEGLPEPLTLRWEP

SEQ ID NO: 195 - protein, 276 aa, Homo sapiens
GSHSMRYFYTAVSRPGRGEPHFIAVGYVDDTQFVRFDSDAASPRGEPRAPWVEQEGPEYWDRETQKYKRQAQTDRVSLRNLRGCYNQSEARSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPCGDGTFQKWAAVVVPSGEEQRYTCHVQHEGLPEPLTLRWEP

SEQ ID NO: 196 - protein, 276 aa, Homo sapiens
GSHSMRYFYTAVSRPGRGEPHFIAVGYVDDTQFVRFDSDAASPRGEPRAPWVEQEGPEYWDRETQKYKRQAQTDRVSLRNLRGCYNQSEAGSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPCGDGTFQKWAAVVVPSGEEQRYTCHVQHEGLPEPLTLRWEP

SEQ ID NO: 197 - protein, 276 aa, Homo sapiens
GSHSMRYFSTSVSWPGRGEPRFIAVGYVDDTQFVRFDSDAASPRGEPREPWVEQEGPEYWDRETQKYKRQAQADRVNLRKLRGCYNQSEDGSHTLQRMFGCDLGPDGRLLRGYNQFAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPCGDGTFQKWAAVVVPSGEEQRYTCHVQHEGLPEPLTLRWKP

SEQ ID NO: 198 - protein, 276 aa, Homo sapiens
CSHSMRYFDTAVSRPGRGEPRFISVGYVDDTQFVRFDSDAASPRGEPRAPWVEQEGPEYWDRETQKYKRQAQADRVNLRKLRGCYNQSEDGSHTLQWMYGCDLGPDGRLLRGYDQSAYDGKDYIALNEDLRSWTAADTAAQITQRKWEAAREAEQWRAYLEGTCVEWLRRYLENGKETLQRAEHPKTHVTHHPVSDHEATLRCWALGFYPAEITLTWQRDGEDQTQDTELVETRPCGDGTFQKWAAVVVPSGEEQRYTCHVQHEGLPEPLTLRWEP

SEQ ID NO: 199 - protein, 276 aa, Homo sapiens
CSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPCGDGTFQKWAAVVVPSGQEQRYTCHMQHEGLQEPLTLSWEP

SEQ ID NO: 200 - protein, 276 aa, Homo sapiens
CSHSMRYFYTAVSRPGRGEPRFIAVGYVDDTQFVQFDSDAASPRGEPRAPWVEQEGPEYWDRETQKYKRQAQTDRVSLRNLRGCYNQSEAGSHTLQRMYGCDLGPDGRLLRGYNQFAYDGKDYIALNEDLRSWTAADTAAQITQRKWEAARTAEQLRAYLEGTCVEWLRRYLENGKKTLQRAEHPKTHVTHHPVSDHEATLRCWALGFYPAEITLTWQRDGEDQTQDTELVETRPCGDGTFQKWAAVVVPSGEEQRYTCHVQHEGLPEPLTLRWGP

SEQ ID NO: 201 - protein, 276 aa, Homo sapiens
CSHSMRYFYTAVSRPGRGEPHFIAVGYVDDTQFVRFDSDAASPRGEPRAPWVEQEGPEYWDRETQNYKRQAQTDRVNLRKLRGCYNQSEAGSHIIQRMYGCDLGPDGRLLRGHDQLAYDGKDYIALNEDLRSWTAADTAAQITQRKWEAAREAEQLRAYLEGTCVEWLRRYLENGKETLQRAEHPKTHVTHHPVSDHEATLRCWALGFYPAEITLTWQRDGEDQTQDTELVETRPCGDGTFQKWAAVVVPSGEEQRYTCHVQHEGLPEPLTLRWEP

SEQ ID NO: 202 - protein, 219 aa, synthetic construct
  REGION 1..219: note = synthetic sequence
  VARIANT 8: note = X is any amino acid other than Asp. In some cases, X is Ala. In some cases, X is Arg
FTVTVPKXLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKI

SEQ ID NO: 203 - protein, 219 aa, synthetic construct
  REGION 1..219: note = synthetic sequence
  VARIANT 36: note = X is any amino acid other than Ile. In some cases, X is Asp
FTVTVPKDLYVVEYGSNMTIECKFPVEKQLDLAALXVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKI

SEQ ID NO: 204 - protein, 219 aa, synthetic construct
  REGION 1..219: note = synthetic sequence
  VARIANT 54: note = X is any amino acid other than Glu. In some cases, X is Arg
FTVTVPKDLYVVEYGSNMTIECKFPVEKQLDLAALIVYWEMEDKNIIQFVHGEXDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKI

SEQ ID NO: 205 - protein, 208 aa, synthetic construct
  REGION 1..208: note = synthetic sequence
  VARIANT 19: note = X is any amino acid other than Asn. In some cases, X is Ala
VIHVTKEVKEVATLSCGHXVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 206 - protein, 208 aa, synthetic construct
  REGION 1..208: note = synthetic sequence
  VARIANT 63: note = X is any amino acid other than Asn. In some cases, X is Ala
VIHVTKEVKEVATLSCGHNVSVEELAQTRIYWQKEKKMVLTMMSGDMNIWPEYKNRTIFDITXNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 207 - protein, 208 aa, synthetic construct
  REGION 1..208: note = synthetic sequence
  VARIANT 67: note = X is any amino acid other than Ile. In some cases, X is Ala
VIHVTKEVKEVATLSCGHNVSVEELAQTRIYWQKEKKMVLTMMSGDMNIWPEYKNRTIFDITNNLSXVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 208 - protein, 208 aa, synthetic construct
  REGION 1..208: note = synthetic sequence
  VARIANT 86: note = X is any amino acid other than Lys. In some cases, X is Ala
VIHVTKEVKEVATLSCGHNVSVEELAQTRIYWQKEKKMVLTMMSGDMNIWPEYKNRTIFDITNNLSIVILALRPSDEGTYECVVLXYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 209 - protein, 208 aa, synthetic construct
  REGION 1..208: note = synthetic sequence
  VARIANT 157: note = X is any amino acid other than Gln. In some cases, X is Ala
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSXDPETELYAVSSKLDFNMTTNHSFMCLIKYGHLRVNQTFNWNTTKQEHFPDN

SEQ ID NO: 210 - protein, 208 aa, synthetic construct
  REGION 1..208: note = synthetic sequence
  VARIANT 158: note = X is any amino acid other than Asp. In some cases, X is Ala
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQXPETELYAVSSKLDFNMTTNHSFMCLIKYGHLRVNQTFNWNTTKQEHFPDN

SEQ ID NO: 211 - protein, 208 aa, synthetic construct
  REGION 1..208: note = synthetic sequence
  VARIANT 25: note = X is any amino acid other than Leu. In some cases, X is Ala
VIHVTKEVKEVATLSCGHNVSVEEXAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 212 - protein, 208 aa, synthetic construct
  REGION 1..208: note = synthetic sequence
  VARIANT 31: note = X is any amino acid other than Tyr. In some cases, X is Ala
VIHVTKEVKEVATLSCGHNVSVEELAQTRIXWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 213 - protein, 208 aa, synthetic construct
  REGION 1..208: note = synthetic sequence
  VARIANT 33: note = X is any amino acid other than Gln. In some cases, X is Ala
VIHVTKEVKEVATLSCGHNVSVEELAQTRIYWXKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 214 - protein, 208 aa, synthetic construct
  REGION 1..208: note = synthetic sequence
  VARIANT 38: note = X is any amino acid other than Met. In some cases, X is Ala
VIHVTKEVKEVATLSCGHNVSVEELAQTRIYWQKEKKXVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 215 - protein, 208 aa, synthetic construct
  REGION 1..208: note = synthetic sequence
  VARIANT 39: note = X is any amino acid other than Val. In some cases, X is Ala
VIHVTKEVKEVATLSCGHNVSVEELAQTRIYWQKEKKMXLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 216 - protein, 208 aa, synthetic construct
  REGION 1..208: note = synthetic sequence
  VARIANT 49: note = X is any amino acid other than Ile. In some cases, X is Ala
VIHVTKEVKEVATLSCGHNVSVEELAQTRIYWQKEKKMVLTMMSGDMNXWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 217 - protein, 208 aa, synthetic construct
  REGION 1..208: note = synthetic sequence
  VARIANT 53: note = X is any amino acid other than Tyr. In some cases, X is Ala
VIHVTKEVKEVATLSCGHNVSVEELAQTRIYWQKEKKMVLTMMSGDMNIWPEXKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 218 - protein, 208 aa, synthetic construct
  REGION 1..208: note = synthetic sequence
  VARIANT 60: note = X is any amino acid other than Asp. In some cases, X is Ala
VIHVTKEVKEVATLSCGHNVSVEELAQTRIYWQKEKKMVLTMMSGDMNIWPEYKNRTIFXITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 219 - protein, 208 aa, synthetic construct
  REGION 1..208: note = synthetic sequence
  VARIANT 108: note = X is any amino acid other than Phe. In some cases, X is Ala
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADXPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 220 - protein, 208 aa, synthetic construct
  REGION 1..208: note = synthetic sequence
  VARIANT 156: note = X is any amino acid other than Ser. In some cases, X is Ala
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVXQDPETELYAVSSKLDFNMTTNHSFMCLIKYGHLRVNQTFNWNTTKQEHFPDN

SEQ ID NO: 221 - protein, 208 aa, synthetic construct
  REGION 1..208: note = synthetic sequence
  VARIANT 111: note = X is any amino acid other than Pro. In some cases, X is Ala
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTXSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 222 - protein, 224 aa, synthetic construct
  REGION 1..224: note = synthetic sequence
  VARIANT 61: note = X is any amino acid other than Asn. In some cases, X is Ala
APLKIQAYFNETADLPCQFANSQNQSLSELVVFWQDQENLVLNEVYLGKEKFDSVHSKYMXRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 223 - protein, 224 aa, synthetic construct
  REGION 1..224: note = synthetic sequence
  VARIANT 66: note = X is any amino acid other than Asp. In some cases, X is Ala
APLKIQAYFNETADLPCQFANSQNQSLSELVVFWQDQENLVLNEVYLGKEKFDSVHSKYMNRTSFXSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 224 - protein, 224 aa, synthetic construct
  REGION 1..224: note = synthetic sequence
  VARIANT 70: note = X is any amino acid other than Trp. In some cases, X is Ala
APLKIQAYFNETADLPCQFANSQNQSLSELVVFWQDQENLVLNEVYLGKEKFDSVHSKYMNRTSFDSDSXTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 225 - protein, 224 aa, synthetic construct
  REGION 1..224: note = synthetic sequence
  VARIANT 91: note = X is any amino acid other than His. In some cases, X is Ala
APLKIQAYFNETADLPCQFANSQNQSLSELVVFWQDQENLVLNEVYLGKEKFDSVHSKYMNRTSFDSDSWTLRLHNLQIKDKGLYQCIIHXKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 226 - protein, 110 aa, synthetic construct
  REGION 1..110: note = synthetic sequence
  VARIANT 61: note = where X is any amino acid other than Asn. In some cases, X is Ala
APLKIQAYFNETADLPCQFANSQNQSLSELVVFWQDQENLVLNEVYLGKEKFDSVHSKYMXRTSFDSDSWTLRLHNLQIKDKGLYQCIIHHKKPTGMIRIHQMNSELSVL

SEQ ID NO: 227 - protein, 110 aa, synthetic construct
  REGION 1..110: note = synthetic sequence
  VARIANT 66: note = X is any amino acid other than Asp. In some cases, X is Ala
APLKIQAYFNETADLPCQFANSQNQSLSELVVFWQDQENLVLNEVYLGKEKFDSVHSKYMNRTSFXSDSWTLRLHNLQIKDKGLYQCIIHHKKPTGMIRIHQMNSELSVL

SEQ ID NO: 228 - protein, 110 aa, synthetic construct
  REGION 1..110: note = synthetic sequence
  VARIANT 70: note = X is any amino acid other than Trp. In some cases, X is Ala
APLKIQAYFNETADLPCQFANSQNQSLSELVVFWQDQENLVLNEVYLGKEKFDSVHSKYMNRTSFDSDSXTLRLHNLQIKDKGLYQCIIHHKKPTGMIRIHQMNSELSVL

SEQ ID NO: 229 - protein, 110 aa, synthetic construct
  REGION 1..110: note = synthetic sequence
  VARIANT 91: note = X is any amino acid other than His. In some cases, X is Ala
APLKIQAYFNETADLPCQFANSQNQSLSELVVFWQDQENLVLNEVYLGKEKFDSVHSKYMNRTSFDSDSWTLRLHNLQIKDKGLYQCIIHXKKPTGMIRIHQMNSELSVL

SEQ ID NO: 230 - protein, 224 aa, synthetic construct
  REGION 1..224: note = synthetic sequence
  VARIANT 41: note = X is any amino acid other than Val. In some cases, X is Ala
APLKIQAYFNETADLPCQFANSQNQSLSELVVFWQDQENLXLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 231 - protein, 110 aa, synthetic construct
  REGION 1..110: note = synthetic sequence
  VARIANT 41: note = X is any amino acid other than Val. In some cases, X is Ala
APLKIQAYFNETADLPCQFANSQNQSLSELVVFWQDQENLXLNEVYLGKEKFDSVHSKYMNRTSFDSDSWTLRLHNLQIKDKGLYQCIIHHKKPTGMIRIHQMNSELSVL

SEQ ID NO: 232 - protein, 224 aa, synthetic construct
  REGION 1..224: note = synthetic sequence
  VARIANT 35: note = X is any amino acid other than Gln. In some cases, X is Ala
APLKIQAYFNETADLPCQFANSQNQSLSELVVFWXDQENLVLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 233 - protein, 110 aa, synthetic construct
  REGION 1..110: note = synthetic sequence
  VARIANT 35: note = X is any amino acid other than Gln. In some cases, X is Ala
APLKIQAYFNETADLPCQFANSQNQSLSELVVFWXDQENLVLNEVYLGKEKFDSVHSKYMNRTSFDSDSWTLRLHNLQIKDKGLYQCIIHHKKPTGMIRIHQMNSELSVL

SEQ ID NO: 234 - protein, 224 aa, synthetic construct
  REGION 1..224: note = synthetic sequence
  VARIANT 33: note = X is any amino acid other than Phe. In some cases, X is Ala
APLKIQAYFNETADLPCQFANSQNQSLSELVVXWQDQENLVLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 235 - protein, 110 aa, synthetic construct
  REGION 1..110: note = synthetic sequence
  VARIANT 33: note = X is any amino acid other than Phe. In some cases, X is Ala
APLKIQAYFNETADLPCQFANSQNQSLSELVVXWQDQENLVLNEVYLGKEKFDSVHSKYMNRTSFDSDSWTLRLHNLQIKDKGLYQCIIHHKKPTGMIRIHQMNSELSVL

SEQ ID NO: 236 - protein, 224 aa, synthetic construct
  REGION 1..224: note = synthetic sequence
  VARIANT 72: note = X is any amino acid other than Leu. In some cases, X is Ala
APLKIQAYFNETADLPCQFANSQNQSLSELVVFWQDQENLVLNEVYLGKEKFDSVHSKYMNRTSFDSDSWTXRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 237 - protein, 110 aa, synthetic construct
  REGION 1..110: note = synthetic sequence
  VARIANT 72: note = X is any amino acid other than Leu. In some cases, X is Ala
APLKIQAYFNETADLPCQFANSQNQSLSELVVFWQDQENLVLNEVYLGKEKFDSVHSKYMNRTSFDSDSWTXRLHNLQIKDKGLYQCIIHHKKPTGMIRIHQMNSELSVL

SEQ ID NO: 238 - protein, 224 aa, synthetic construct
  REGION 1..224: note = synthetic sequence
  VARIANT 59: note = X is any amino acid other than Tyr. In some cases, X is Ala
APLKIQAYFNETADLPCQFANSQNQSLSELVVFWQDQENLVLNEVYLGKEKFDSVHSKXMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 239 - protein, 110 aa, synthetic construct
  REGION 1..110: note = synthetic sequence
  VARIANT 59: note = X is any amino acid other than Tyr. In some cases, X is Ala
APLKIQAYFNETADLPCQFANSQNQSLSELVVFWQDQENLVLNEVYLGKEKFDSVHSKXMNRTSFDSDSWTLRLHNLQIKDKGLYQCIIHHKKPTGMIRIHQMNSELSVL

SEQ ID NO: 240 - protein, 224 aa, synthetic construct
  REGION 1..224: note = synthetic sequence
  VARIANT 61: note = X is any amino acid other than Asn, In some cases, X is Ala
  VARIANT 91: note = X is any amino acid other than His, In some cases, X is Ala
APLKIQAYFNETADLPCQFANSQNQSLSELVVFWQDQENLVLNEVYLGKEKFDSVHSKYMXRTSFDSDSWTLRLHNLQIKDKGLYQCIIHXKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 241 - protein, 110 aa, synthetic construct
  REGION 1..110: note = synthetic sequence
  VARIANT 61: note = X is any amino acid other than Asn. In some cases, X is Ala.
  VARIANT 91: note = X is any amino acid other than His. In some cases, X is Ala.
APLKIQAYFNETADLPCQFANSQNQSLSELVVFWQDQENLVLNEVYLGKEKFDSVHSKYMXRTSFDSDSWTLRLHNLQIKDKGLYQCIIHXKKPTGMIRIHQMNSELSVL

SEQ ID NO: 242 - protein, 224 aa, synthetic construct
  REGION 1..224: note = synthetic sequence
  VARIANT 66: note = X is any amino acid other than Asp. In some cases, X is Ala.
  VARIANT 91: note = X is any amino acid other than His. In some cases, X is Ala.
APLKIQAYFNETADLPCQFANSQNQSLSELVVFWQDQENLVLNEVYLGKEKFDSVHSKYMNRTSFXSDSWTLRLHNLQIKDKGLYQCIIHXKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 243 - protein, 110 aa, synthetic construct
  REGION 1..110: note = synthetic sequence
  VARIANT 66: note = X is any amino acid other than Asn. In some cases, X is Ala
  VARIANT 91: note = X is any amino acid other than His. In some cases, X is Ala
APLKIQAYFNETADLPCQFANSQNQSLSELVVFWQDQENLVLNEVYLGKEKFDSVHSKYMNRTSFXSDSWTLRLHNLQIKDKGLYQCIIHXKKPTGMIRIHQMNSELSVL

SEQ ID NO: 244 - protein, 224 aa, synthetic construct
  REGION 1..224: note = synthetic sequence
  VARIANT 61: note = X is any amino acid other than Asn. In some cases, X is Ala.
  VARIANT 66: note = X is any amino acid other than Asp. In some cases, X is Ala.
  VARIANT 91: note = X is any amino acid other than His. In some cases, X is Ala.
APLKIQAYFNETADLPCQFANSQNQSLSELVVFWQDQENLVLNEVYLGKEKFDSVHSKYMXRTSFXSDSWTLRLHNLQIKDKGLYQCIIHXKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 245 - protein, 110 aa, synthetic construct
  REGION 1..110: note = synthetic sequence
  VARIANT 61: note = X is any amino acid other than Asn. In some cases, X is Ala
  VARIANT 66: note = X is any amino acid other than Asp. In some cases, X is Ala
  VARIANT 91: note = X is any amino acid other than His. In some cases, X is Ala
APLKIQAYFNETADLPCQFANSQNQSLSELVVFWQDQENLVLNEVYLGKEKFDSVHSKYMXRTSFXSDSWTLRLHNLQIKDKGLYQCIIHXKKPTGMIRIHQMNSELSVL

SEQ ID NO: 246 - protein, 174 aa, Homo sapiens
  VARIANT 47: note = X is any amino acid other than Lys. In some cases, X is Ala
PAGLLDLRQGMFAQLVAQNVLLIDGPLSWYSDPGLAGVSLTGGLSYXEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 247 - protein, 174 aa, synthetic construct
  REGION 1..174: note = synthetic sequence
  VARIANT 147: note = X is any amino acid other than Gln. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWXLTQGATVLGLFRVTPEIPAGLPSPRSE

SEQ ID NO: 248 - protein, 174 aa, synthetic construct
  REGION 1..174: note = synthetic sequence
  VARIANT 11: note = X is any amino acid other than Met. In some cases, X is Ala
PAGLLDLRQGXFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 249 - protein, 174 aa, synthetic construct
  REGION 1..174: note = synthetic sequence
  VARIANT 12: note = X is any amino acid other than Phe. In some cases, X is Ala
PAGLLDLRQGMXAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 250 - protein, 174 aa, synthetic construct
  REGION 1..174: note = synthetic sequence
  VARIANT 14: note = X is any amino acid other than Gln. In some cases, X is Ala
PAGLLDLRQGMFAXLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 251 - protein, 174 aa, synthetic construct
  REGION 1..174: note = synthetic sequence
  VARIANT 15: note = X is any amino acid other than Leu. In some cases, X is Ala
PAGLLDLRQGMFAQXVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 252 - protein, 174 aa, synthetic construct
  REGION 1..174: note = synthetic sequence
  VARIANT 16: note = X is any amino acid other than Val. In some cases, X is Ala
PAGLLDLRQGMFAQLXAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 253 - protein, 174 aa, synthetic construct
  REGION 1..174: note = synthetic sequence
  VARIANT 18: note = X is any amino acid other than Gln. In some cases, X is Ala
PAGLLDLRQGMFAQLVAXNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 254 - protein, 174 aa, synthetic construct
  REGION 1..174: note = synthetic sequence
  VARIANT 19: note = X is any amino acid other than Asn. In some cases, X is Ala
PAGLLDLRQGMFAQLVAQXVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 255 - protein, 174 aa, synthetic construct
  REGION 1..174: note = synthetic sequence
  VARIANT 20: note = X is any amino acid other than Val. In some cases, X is Ala
PAGLLDLRQGMFAQLVAQNXLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 256 - protein, 174 aa, synthetic construct
  REGION 1..174: note = synthetic sequence
  VARIANT 21: note = X is any amino acid other than Leu. In some cases, X is Ala
PAGLLDLRQGMFAQLVAQNVXLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 257 - protein, 174 aa, synthetic construct
  REGION 1..174: note = synthetic sequence
  VARIANT 22: note = X is any amino acid other than Leu. In some cases, X is Ala
PAGLLDLRQGMFAQLVAQNVLXIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 258 - protein, 174 aa, synthetic construct
  REGION 1..174: note = synthetic sequence
  VARIANT 23: note = X is any amino acid other than Ile. In some cases, X is Ala
PAGLLDLRQGMFAQLVAQNVLLXDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 259 - protein, 174 aa, synthetic construct
  REGION 1..174: note = synthetic sequence
  VARIANT 24: note = X is any amino acid other than Asp. In some cases, X is Ala
PAGLLDLRQGMFAQLVAQNVLLIXGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 260 - protein, 174 aa, synthetic construct
  REGION 1..174: note = synthetic sequence
  VARIANT 25: note = X is any amino acid other than Gly. In some cases, X is Ala
PAGLLDLRQGMFAQLVAQNVLLIDXPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 261 - protein, 174 aa, synthetic construct
  REGION 1..174: note = synthetic sequence
  VARIANT 26: note = X is any amino acid other than Pro. In some cases, X is Ala
PAGLLDLRQGMFAQLVAQNVLLIDGXLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 262 - protein, 174 aa, synthetic construct
  REGION 1..174: note = synthetic sequence
  VARIANT 27: note = X is any amino acid other than Leu. In some cases, X is Ala
PAGLLDLRQGMFAQLVAQNVLLIDGPXSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 263 - protein, 174 aa, synthetic construct
  REGION 1..174: note = synthetic sequence
  VARIANT 28: note = X is any amino acid other than Ser. In some cases, X is Ala
PAGLLDLRQGMFAQLVAQNVLLIDGPLXWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 264 - protein, 174 aa, synthetic construct
  REGION 1..174: note = synthetic sequence
  VARIANT 29: note = X is any amino acid other than Trp. In some cases, X is Ala
PAGLLDLRQGMFAQLVAQNVLLIDGPLSXYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 265 - protein, 174 aa, synthetic construct
  REGION 1..174: note = synthetic sequence
  VARIANT 30: note = X is any amino acid other than Tyr. In some cases, X is Ala
PAGLLDLRQGMFAQLVAQNVLLIDGPLSWXSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 266 - protein, 174 aa, synthetic construct
  REGION 1..174: note = synthetic sequence
  VARIANT 31: note = X is any amino acid other than Ser. In some cases, X is Ala
PAGLLDLRQGMFAQLVAQNVLLIDGPLSWYXDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 267 - protein, 174 aa, synthetic construct
  REGION 1..174: note = synthetic sequence
  VARIANT 32: note = X is any amino acid other than Asp. In some cases, X is Ala
PAGLLDLRQGMFAQLVAQNVLLIDGPLSWYSXPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 268 - protein, 174 aa, synthetic construct
  REGION 1..174: note = synthetic sequence
  VARIANT 33: note = X is any amino acid other than Pro. In some cases, X is Ala
PAGLLDLRQGMFAQLVAQNVLLIDGPLSWYSDXGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 269 - protein, 174 aa, synthetic construct
  REGION 1..174: note = synthetic sequence
  VARIANT 34: note = X is any amino acid other than Gly. In some cases, X is Ala
PAGLLDLRQGMFAQLVAQNVLLIDGPLSWYSDPXLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 270 - protein, 174 aa, synthetic construct
  REGION 1..174: note = synthetic sequence
  VARIANT 35: note = X is any amino acid other than Leu. In some cases, X is Ala
PAGLLDLRQGMFAQLVAQNVLLIDGPLSWYSDPGXAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 271 - protein, 174 aa, synthetic construct
  REGION 1..174: note = synthetic sequence
  VARIANT 37: note = X is any amino acid other than Gly. In some cases, X is Ala
PAGLLDLRQGMFAQLVAQNVLLIDGPLSWYSDPGLAXVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 272 - protein, 174 aa, synthetic construct
  REGION 1..174: note = synthetic sequence
  VARIANT 38: note = X is any amino acid other than Val. In some cases, X is Ala
PAGLLDLRQGMFAQLVAQNVLLIDGPLSWYSDPGLAGXSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 273 - protein, 174 aa, synthetic construct
  REGION 1..174: note = synthetic sequence
  VARIANT 39: note = X is any amino acid other than Ser. In some cases, X is Ala
PAGLLDLRQGMFAQLVAQNVLLIDGPLSWYSDPGLAGVXLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 274 - protein, 174 aa, synthetic construct
  REGION 1..174: note = synthetic sequence
  VARIANT 40: note = X is any amino acid other than Leu. In some cases, X is Ala
PAGLLDLRQGMFAQLVAQNVLLIDGPLSWYSDPGLAGVSXTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 275 - protein, 174 aa, synthetic construct
  REGION 1..174: note = synthetic sequence
  VARIANT 41: note = X is any amino acid other than Thr. In some cases, X is Ala
PAGLLDLRQGMFAQLVAQNVLLIDGPLSWYSDPGLAGVSLXGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 276 - protein, 174 aa, synthetic construct
  REGION 1..174: note = synthetic sequence
  VARIANT 42: note = X is any amino acid other than Gly. In some cases, X is Ala
PAGLLDLRQGMFAQLVAQNVLLIDGPLSWYSDPGLAGVSLTXGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 277 - protein, 174 aa, synthetic construct
  REGION 1..174: note = synthetic sequence
  VARIANT 43: note = X is any amino acid other than Gly. In some cases, X is Ala
PAGLLDLRQGMFAQLVAQNVLLIDGPLSWYSDPGLAGVSLTGXLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 278 - protein, 174 aa, synthetic construct
  REGION 1..174: note = synthetic sequence
  VARIANT 44: note = X is any amino acid other than Leu. In some cases, X is Ala
PAGLLDLRQGMFAQLVAQNVLLIDGPLSWYSDPGLAGVSLTGGXSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 279 - protein, 174 aa, synthetic construct
  REGION 1..174: note = synthetic sequence
  VARIANT 45: note = X is any amino acid other than Ser. In some cases, X is Ala
PAGLLDLRQGMFAQLVAQNVLLIDGPLSWYSDPGLAGVSLTGGLXYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 280 - protein, 174 aa, synthetic construct
  REGION 1..174: note = synthetic sequence
  VARIANT 46: note = X is any amino acid other than Tyr. In some cases, X is Ala
PAGLLDLRQGMFAQLVAQNVLLIDGPLSWYSDPGLAGVSLTGGLSXKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 281 - protein, 174 aa, synthetic construct
  REGION 1..174: note = synthetic sequence
  VARIANT 48: note = X is any amino acid other than Glu. In some cases, X is Ala
PAGLLDLRQGMFAQLVAQNVLLIDGPLSWYSDPGLAGVSLTGGLSYKXDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 282 - protein, 174 aa, synthetic construct
  REGION 1..174: note = synthetic sequence
  VARIANT 49: note = X is any amino acid other than Asp. In some cases, X is Ala
PAGLLDLRQGMFAQLVAQNVLLIDGPLSWYSDPGLAGVSLTGGLSYKEXTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 283 - protein, 174 aa, synthetic construct
  REGION 1..174: note = synthetic sequence
  VARIANT 50: note = X is any amino acid other than Thr. In some cases, X is Ala
PAGLLDLRQGMFAQLVAQNVLLIDGPLSWYSDPGLAGVSLTGGLSYKEDXKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 284 - protein, 174 aa, synthetic construct
  REGION 1..174: note = synthetic sequence
  VARIANT 51: note = X is any amino acid other than Lys. In some cases, X is Ala
PAGLLDLRQGMFAQLVAQNVLLIDGPLSWYSDPGLAGVSLTGGLSYKEDTXELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 285 - protein, 174 aa, synthetic construct
  REGION 1..174: note = synthetic sequence
  VARIANT 52: note = X is any amino acid other than Glu. In some cases, X is Ala
PAGLLDLRQGMFAQLVAQNVLLIDGPLSWYSDPGLAGVSLTGGLSYKEDTKXLVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 286 - protein, 174 aa, synthetic construct
  REGION 1..174: note = synthetic sequence
  VARIANT 64: note = X is any amino acid other than Phe. In some cases, X is Ala
PAGLLDLRQGMFAQLVAQNVLLIDGPLSWYSDPGLAGVSLTGGLSYKEDTKELVVAKAGVYYVXFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 287 - protein, 174 aa, synthetic construct
  REGION 1..174: note = synthetic sequence
  VARIANT 65: note = X is any amino acid other than Phe. In some cases, X is Ala
PAGLLDLRQGMFAQLVAQNVLLIDGPLSWYSDPGLAGVSLTGGLSYKEDTKELVVAKAGVYYVFXQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 288 - protein, 174 aa, synthetic construct
  REGION 1..174: note = synthetic sequence
  VARIANT 66: note = X is any amino acid other than Gln. In some cases, X is Ala
PAGLLDLRQGMFAQLVAQNVLLIDGPLSWYSDPGLAGVSLTGGLSYKEDTKELVVAKAGVYYVFFXLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 289 - protein, 174 aa, synthetic construct
  REGION 1..174: note = synthetic sequence
  VARIANT 67: note = X is any amino acid other than Leu. In some cases, X is Ala
PAGLLDLRQGMFAQLVAQNVLLIDGPLSWYSDPGLAGVSLTGGLSYKEDTKELVVAKAGVYYVFFQXELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 290 - protein, 174 aa, synthetic construct
  REGION 1..174: note = synthetic sequence
  VARIANT 68: note = X is any amino acid other than Glu. In some cases, X is Ala
PAGLLDLRQGMFAQLVAQNVLLIDGPLSWYSDPGLAGVSLTGGLSYKEDTKELVVAKAGVYYVFFQLXLRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 291 - protein, 174 aa, synthetic construct
  REGION 1..174: note = synthetic sequence
  VARIANT 69: note = X is any amino acid other than Leu. In some cases, X is Ala
PAGLLDLRQGMFAQLVAQNVLLIDGPLSWYSDPGLAGVSLTGGLSYKEDTKELVVAKAGVYYVFFQLEXRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 292 - protein, 174 aa, synthetic construct
  REGION 1..174: note = synthetic sequence
  VARIANT 70: note = X is any amino acid other than Arg. In some cases, X is Ala
PAGLLDLRQGMFAQLVAQNVLLIDGPLSWYSDPGLAGVSLTGGLSYKEDTKELVVAKAGVYYVFFQLELXRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 293 - protein, 174 aa, synthetic construct
  REGION 1..174: note = synthetic sequence
  VARIANT 71: note = X is any amino acid other than Arg. In some cases, X is Ala
PAGLLDLRQGMFAQLVAQNVLLIDGPLSWYSDPGLAGVSLTGGLSYKEDTKELVVAKAGVYYVFFQLELRXVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 294 - protein, 174 aa, synthetic construct
  REGION 1..174: note = synthetic sequence
  VARIANT 72: note = X is any amino acid other than Val. In some cases, X is Ala
PAGLLDLRQGMFAQLVAQNVLLIDGPLSWYSDPGLAGVSLTGGLSYKEDTKELVVAKAGVYYVFFQLELRRXVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 295 - protein, 174 aa, synthetic construct
  REGION 1..174: note = synthetic sequence
  VARIANT 73: note = X is any amino acid other than Val. In some cases, X is Ala
PAGLLDLRQGMFAQLVAQNVLLIDGPLSWYSDPGLAGVSLTGGLSYKEDTKELVVAKAGVYYVFFQLELRRVXAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 296 - protein, 174 aa, synthetic construct
  REGION 1..174: note = synthetic sequence
  VARIANT 75: note = X is any amino acid other than Gly. In some cases, X is Ala
PAGLLDLRQGMFAQLVAQNVLLIDGPLSWYSDPGLAGVSLTGGLSYKEDTKELVVAKAGVYYVFFQLELRRVVAXEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 297 - protein, 174 aa, synthetic construct
  REGION 1..174: note = synthetic sequence
  VARIANT 76: note = X is any amino acid other than Glu. In some cases, X is Ala
PAGLLDLRQGMFAQLVAQNVLLIDGPLSWYSDPGLAGVSLTGGLSYKEDTKELVVAKAGVYYVFFQLELRRVVAGXGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 298 - protein, 174 aa, synthetic construct
  REGION 1..174: note = synthetic sequence
  VARIANT 77: note = X is any amino acid other than Gly. In some cases, X is Ala
PAGLLDLRQGMFAQLVAQNVLLIDGPLSWYSDPGLAGVSLTGGLSYKEDTKELVVAKAGVYYVFFQLELRRVVAGEXSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 299 - protein, 174 aa, synthetic construct
  REGION 1..174: note = synthetic sequence
  VARIANT 78: note = X is any amino acid other than Ser. In some cases, X is Ala
PAGLLDLRQGMFAQLVAQNVLLIDGPLSWYSDPGLAGVSLTGGLSYKEDTKELVVAKAGVYYVFFQLELRRVVAGEGXGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 300 - protein, 174 aa, synthetic construct
  REGION 1..174: note = synthetic sequence
  VARIANT 104: note = X is any amino acid other than Asp. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVXLPPASSEARNSAFGFQGRLLHLSAGQRLGVHLHTEARARHAWQLTQGATVLGLFRVTPEIPAGLPSPRSE

SEQ ID NO: 301 - protein, 174 aa, synthetic construct
  REGION 1..174: note = synthetic sequence
  VARIANT 105: note = X is any amino acid other than Leu. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDXPPASSEARNSAFGFQGRLLHLSAGQRLGVHLHTEARARHAWQLTQGATVLGLFRVTPEIPAGLPSPRSE

SEQ ID NO: 302 - protein, 174 aa, synthetic construct
  REGION 1..174: note = synthetic sequence
  VARIANT 106: note = X is any amino acid other than Pro. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLXPASSEARNSAFGFQGRLLHLSAGQRLGVHLHTEARARHAWQLTQGATVLGLFRVTPEIPAGLPSPRSE

SEQ ID NO: 303 - protein, 174 aa, synthetic construct
  REGION 1..174: note = synthetic sequence
  VARIANT 109: note = X is any amino acid other than Ser. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPAXSEARNSAFGFQGRLLHLSAGQRLGVHLHTEARARHAWQLTQGATVLGLFRVTPEIPAGLPSPRSE

SEQ ID NO: 304 - protein, 174 aa, synthetic construct
  REGION 1..174: note = synthetic sequence
  VARIANT 110: note = X is any amino acid other than Ser. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASXEARNSAFGFQGRLLHLSAGQRLGVHLHTEARARHAWQLTQGATVLGLFRVTPEIPAGLPSPRSE

SEQ ID NO: 305 - protein, 174 aa, synthetic construct
  REGION 1..174: note = synthetic sequence
  VARIANT 111: note = X is any amino acid other than Glu. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSXARNSAFGFQGRLLHLSAGQRLGVHLHTEARARHAWQLTQGATVLGLFRVTPEIPAGLPSPRSE

SEQ ID NO: 306 - protein, 174 aa, synthetic construct
  REGION 1..174: note = synthetic sequence
  VARIANT 113: note = X is any amino acid other than Arg. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEAXNSAFGFQGRLLHLSAGQRLGVHLHTEARARHAWQLTQGATVLGLFRVTPEIPAGLPSPRSE

SEQ ID NO: 307 - protein, 174 aa, synthetic construct
  REGION 1..174: note = synthetic sequence
  VARIANT 114: note = X is any amino acid other than Asn. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARXSAFGFQGRLLHLSAGQRLGVHLHTEARARHAWQLTQGATVLGLFRVTPEIPAGLPSPRSE

SEQ ID NO: 308 - protein, 174 aa, synthetic construct
  REGION 1..174: note = synthetic sequence
  VARIANT 115: note = X is any amino acid other than Ser. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNXAFGFQGRLLHLSAGQRLGVHLHTEARARHAWQLTQGATVLGLFRVTPEIPAGLPSPRSE

SEQ ID NO: 309 - protein, 174 aa, synthetic construct
  REGION 1..174: note = synthetic sequence
  VARIANT 117: note = X is any amino acid other than Phe. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAXGFQGRLLHLSAGQRLGVHLHTEARARHAWQLTQGATVLGLFRVTPEIPAGLPSPRSE

SEQ ID NO: 310 - protein, 174 aa, synthetic construct
  REGION 1..174: note = synthetic sequence
  VARIANT 130: note = X is any amino acid other than Gln. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGXRLGVHLHTEARARHAWQLTQGATVLGLFRVTPEIPAGLPSPRSE

SEQ ID NO: 311 - protein, 174 aa, synthetic construct
  REGION 1..174: note = synthetic sequence
  VARIANT 131: note = X is any amino acid other than Arg. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQXLGVHLHTEARARHAWQLTQGATVLGLFRVTPEIPAGLPSPRSE

SEQ ID NO: 312 - protein, 174 aa, synthetic construct
  REGION 1..174: note = synthetic sequence
  VARIANT 132: note = X is any amino acid other than Leu. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRXGVHLHTEARARHAWQLTQGATVLGLFRVTPEIPAGLPSPRSE

SEQ ID NO: 313 - protein, 174 aa, synthetic construct
  REGION 1..174: note = synthetic sequence
  VARIANT 133: note = X is any amino acid other than Gly. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLXVHLHTEARARHAWQLTQGATVLGLFRVTPEIPAGLPSPRSE

SEQ ID NO: 314 - protein, 174 aa, synthetic construct
  REGION 1..174: note = synthetic sequence
  VARIANT 134: note = X is any amino acid other than Val. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGXHLHTEARARHAWQLTQGATVLGLFRVTPEIPAGLPSPRSE

SEQ ID NO: 315 - protein, 174 aa, synthetic construct
  REGION 1..174: note = synthetic sequence
  VARIANT 135: note = X is any amino acid other than His. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVXLHTEARARHAWQLTQGATVLGLFRVTPEIPAGLPSPRSE

SEQ ID NO: 316 - protein, 174 aa, synthetic construct
  REGION 1..174: note = synthetic sequence
  VARIANT 136: note = X is any amino acid other than Leu. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHXHTEARARHAWQLTQGATVLGLFRVTPEIPAGLPSPRSE

SEQ ID NO: 317 - protein, 174 aa, synthetic construct
  REGION 1..174: note = synthetic sequence
  VARIANT 137: note = X is any amino acid other than His. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLXTEARARHAWQLTQGATVLGLFRVTPEIPAGLPSPRSE

SEQ ID NO: 318 - protein, 174 aa, synthetic construct
  REGION 1..174: note = synthetic sequence
  VARIANT 138: note = X is any amino acid other than Thr. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHXEARARHAWQLTQGATVLGLFRVTPEIPAGLPSPRSE

SEQ ID NO: 319 - protein, 174 aa, synthetic construct
  REGION 1..174: note = synthetic sequence
  VARIANT 139: note = X is any amino acid other than Glu. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTXARARHAWQLTQGATVLGLFRVTPEIPAGLPSPRSE

SEQ ID NO: 320 - protein, 174 aa, synthetic construct
  REGION 1..174: note = synthetic sequence
  VARIANT 141: note = X is any amino acid other than Arg. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEAXARHAWQLTQGATVLGLFRVTPEIPAGLPSPRSE

SEQ ID NO: 321 - protein, 174 aa, synthetic construct
  REGION 1..174: note = synthetic sequence
  VARIANT 143: note = X is any amino acid other than Arg. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARAXHAWQLTQGATVLGLFRVTPEIPAGLPSPRSE

SEQ ID NO: 322 - protein, 174 aa, synthetic construct
  REGION 1..174: note = synthetic sequence
  VARIANT 144: note = X is any amino acid other than His. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARXAWQLTQGATVLGLFRVTPEIPAGLPSPRSE

SEQ ID NO: 323 - protein, 174 aa, synthetic construct
  REGION 1..174: note = synthetic sequence
  VARIANT 146: note = X is any amino acid other than Trp. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAXQLTQGATVLGLFRVTPEIPAGLPSPRSE

SEQ ID NO: 324 - protein, 174 aa, synthetic construct
  REGION 1..174: note = synthetic sequence
  VARIANT 148: note = X is any amino acid other than Leu. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQXTQGATVLGLFRVTPEIPAGLPSPRSE

SEQ ID NO: 325 - protein, 174 aa, synthetic construct
  REGION 1..174: note = synthetic sequence
  VARIANT 149: note = X is any amino acid other than Thr. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLXQGATVLGLFRVTPEIPAGLPSPRSE

SEQ ID NO: 326 - protein, 174 aa, synthetic construct
  REGION 1..174: note = synthetic sequence
  VARIANT 150: note = X is any amino acid other than Gln. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTXGATVLGLFRVTPEIPAGLPSPRSE

SEQ ID NO: 327 - protein, 174 aa, synthetic construct
  REGION 1..174: note = synthetic sequence
  VARIANT 151: note = X is any amino acid other than Gly. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQXATVLGLFRVTPEIPAGLPSPRSE

SEQ ID NO: 328 - protein, 174 aa, synthetic construct
  REGION 1..174: note = synthetic sequence
  VARIANT 153: note = X is any amino acid other than Thr. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGAXVLGLFRVTPEIPAGLPSPRSE

SEQ ID NO: 329 - protein, 174 aa, synthetic construct
  REGION 1..174: note = synthetic sequence
  VARIANT 154: note = X is any amino acid other than Val. In some cases, X is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATXLGLFRVTPEIPAGLPSPRSE

SEQ ID NO: 330 - protein, 133 aa, synthetic construct
  REGION 1..133: note = synthetic sequence
  VARIANT 42: note = X is any amino acid other than Phe. In some cases, X is Ala. In some cases, X is Met. In some cases, X is Pro. In some cases, X is Ser. In some cases, X is Thr. In some cases, X is Trp. In some cases, X is Tyr. In some cases, X is Val. In some cases, X is His
APTSSSTKKTQLQLEHLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 331 - protein, 133 aa, synthetic construct
  REGION 1..133: note = synthetic sequence
  VARIANT 20: note = X is any amino acid other than Asp. In some cases, X is Ala
APTSSSTKKTQLQLEHLLLXLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 332 - protein, 133 aa, synthetic construct
  REGION 1..133: note = synthetic sequence
  VARIANT 15: note = X is any amino acid other than Glu. In some cases, X is Ala
APTSSSTKKTQLQLXHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 333 - protein, 133 aa, synthetic construct
  REGION 1..133: note = synthetic sequence
  VARIANT 16: note = X is any amino acid other than His. In some cases, X is Ala. In some cases, X is Thr. In some cases, X is Asn. In some cases, X is Cys. In some cases, X is Gln. In some cases, X is Met. In some cases, X is Val. In some cases, X is Trp
APTSSSTKKTQLQLEXLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 334 - protein, 133 aa, synthetic construct; identical to SEQ ID NO: 333
  REGION 1..133: note = synthetic sequence
  VARIANT 16: note = X is any amino acid other than His. In some cases, X is Ala. In some cases, X is Arg. In some cases, X is Asn. In some cases, X is Asp. In some cases, X is Cys. In some cases, X is Glu, Gln, Gly, Ile, Lys, Met, Phe, Pro, Ser, Thr, Tyr, Trp or Val

SEQ ID NO: 335 - protein, 133 aa, synthetic construct
  REGION 1..133: note = synthetic sequence
  VARIANT 45: note = X is any amino acid other than Tyr. In some cases, X is Ala
APTSSSTKKTQLQLEHLLLDLQMILNGINNYKNPKLTRMLTFKFXMPKKATELKHLQCLEEELKPLEEVLNLAQSKNFHLRPRDLISNINVIVLELKGSETTFMCEYADETATIVEFLNRWITFCQSIISTLT

SEQ ID NO: 336 - protein, 133 aa, synthetic construct
  REGION 1..133: note = synthetic sequence
  VARIANT 126: note = X is any amino acid other than Gln. In some cases, X is Ala
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCXSIISTLT

SEQ ID NO: 337 - protein, 133 aa, synthetic construct
  REGION 1..133: note = synthetic sequence
  VARIANT 16: note = X is any amino acid other than His. In some cases, X is Ala. In some cases, X is Thr.
  VARIANT 42: note = X is any amino acid other than Phe. In some cases, X is Ala
APTSSSTKKTQLQLEXLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 338 - protein, 133 aa, synthetic construct
  REGION 1..133: note = synthetic sequence
  VARIANT 20: note = X is any amino acid other than Asp. In some cases, X is Ala.
  VARIANT 42: note = X is any amino acid other than Phe. In some cases, X is Ala.
APTSSSTKKTQLQLEHLLLXLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 339 - protein, 133 aa, synthetic construct
  REGION 1..133: note = synthetic sequence
  VARIANT 15: note = X is any amino acid other than Glu. In some cases, X is Ala
  VARIANT 20: note = X is any amino acid other than Asp. In some cases, X is Ala
  VARIANT 42: note = X is any amino acid other than Phe. In some cases, X is Ala
APTSSSTKKTQLQLXHLLLXLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 340 - protein, 133 aa, synthetic construct
  REGION 1..133: note = synthetic sequence
  VARIANT 16: note = X is any amino acid other than His. In some cases, X is Ala
  VARIANT 20: note = X is any amino acid other than Asp. In some cases, X is Ala
  VARIANT 42: note = X is any amino acid other than Phe. In some cases, X is Ala
APTSSSTKKTQLQLEXLLLXLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 341 - protein, 133 aa, synthetic construct
  REGION 1..133: note = synthetic sequence
  VARIANT 20: note = X is any amino acid other than Asp. In some cases, X is Ala
  VARIANT 42: note = X is any amino acid other than Phe. In some cases, X is Ala
  VARIANT 126: note = X is any amino acid other than Gln. In some cases, X is Ala
APTSSSTKKTQLQLEHLLLXLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCXSIISTLT

SEQ ID NO: 342 - protein, 133 aa, synthetic construct
  REGION 1..133: note = synthetic sequence
  VARIANT 20: note = X is any amino acid other than Asp. In some cases, X is Ala
  VARIANT 42: note = X is any amino acid other than Phe. In some cases, X is Ala
  VARIANT 45: note = X is any amino acid other than Tyr. In some cases, X is Ala
APTSSSTKKTQLQLEHLLLXLQMILNGINNYKNPKLTRMLTXKFXMPKKATELKHLQCLEEELKPLEEVLNLAQSKNFHLRPRDLISNINVIVLELKGSETTFMCEYADETATIVEFLNRWITFCQSIISTLT

SEQ ID NO: 343 - protein, 133 aa, synthetic construct
  REGION 1..133: note = synthetic sequence
  VARIANT 16: note = X is any amino acid other than His. In some cases, X is Ala
  VARIANT 20: note = X is any amino acid other than Asp. In some cases, X is Ala
  VARIANT 42: note = X is any amino acid other than Phe. In some cases, X is Ala
  VARIANT 45: note = X is any amino acid other than Tyr. In some cases, X is Ala
APTSSSTKKTQLQLEXLLLXLQMILNGINNYKNPKLTRMLTXKFXMPKKATELKHLQCLEEELKPLEEVLNLAQSKNFHLRPRDLISNINVIVLELKGSETTFMCEYADETATIVEFLNRWITFCQSIISTLT

SEQ ID NO: 344 - protein, 133 aa, synthetic construct
  REGION 1..133: note = synthetic sequence
  VARIANT 20: note = X is any amino acid other than Asp. In some cases, X is Ala
  VARIANT 42: note = X is any amino acid other than Phe. In some cases, X is Ala
  VARIANT 45: note = X is any amino acid other than Tyr. In some cases, X is Ala
  VARIANT 126: note = X is any amino acid other than Gln. In some cases, X is Ala
APTSSSTKKTQLQLEHLLLXLQMILNGINNYKNPKLTRMLTXKFXMPKKATELKHLQCLEEELKPLEEVLNLAQSKNFHLRPRDLISNINVIVLELKGSETTFMCEYADETATIVEFLNRWITFCXSIISTLT

SEQ ID NO: 345 - protein, 133 aa, synthetic construct
  REGION 1..133: note = synthetic sequence
  VARIANT 16: note = X is any amino acid other than His. In some cases, X is Ala
  VARIANT 20: note = X is any amino acid other than Asp. In some cases, X is Ala
  VARIANT 42: note = X is any amino acid other than Phe. In some cases, X is Ala
  VARIANT 45: note = X is any amino acid other than Tyr. In some cases, X is Ala
  VARIANT 126: note = X is any amino acid other than Gln. In some cases, X is Ala
APTSSSTKKTQLQLEXLLLXLQMILNGINNYKNPKLTRMLTXKFXMPKKATELKHLQCLEEELKPLEEVLNLAQSKNFHLRPRDLISNINVIVLELKGSETTFMCEYADETATIVEFLNRWITFCXSIISTLT

SEQ ID NO: 346 - protein, 133 aa, synthetic construct
  REGION 1..133: note = synthetic sequence
  VARIANT 16: note = X is any amino acid other than His. In some cases, X is Ala
  VARIANT 42: note = X is any amino acid other than Phe. In some cases, X is Ala
  VARIANT 126: note = X is any amino acid other than Gln. In some cases, X is Ala
APTSSSTKKTQLQLEXLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCXSIISTLT

SEQ ID NO: 347 - protein, 223 aa, Homo sapiens
PPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 348 - protein, 5 aa, synthetic construct
  REGION 1..5: note = synthetic sequence
  REPEAT 1..5: note = The sequence repeats n times, where n is an integer of at least one
GSGGS

SEQ ID NO: 349 - protein, 4 aa, synthetic construct
  REGION 1..4: note = synthetic sequence
  REPEAT 1..4: note = The sequence repeats n times, where n is an integer of at least one
GGGS

SEQ ID NO: 350 - protein, 4 aa, synthetic construct
  REGION 1..4: note = synthetic sequence
GGSG

SEQ ID NO: 351 - protein, 5 aa, synthetic construct
  REGION 1..5: note = synthetic sequence
GGSGG

SEQ ID NO: 352 - protein, 5 aa, synthetic construct
  REGION 1..5: note = synthetic sequence
GSGSG

SEQ ID NO: 353 - protein, 5 aa, synthetic construct
  REGION 1..5: note = synthetic sequence
GSGGG

SEQ ID NO: 354 - protein, 5 aa, synthetic construct
  REGION 1..5: note = synthetic sequence
GGGSG

SEQ ID NO: 355 - protein, 5 aa, synthetic construct
  REGION 1..5: note = synthetic sequence
GSSSG

SEQ ID NO: 356 - protein, 5 aa, synthetic construct
  REGION 1..5: note = synthetic sequence
  REPEAT 1..5: note = The sequence repeats n times, where n is 1, 2, 3, 4, 5, 6, 7, 8, 9, or 10
GSSSS

SEQ ID NO: 357 - protein, 5 aa, synthetic construct
  REGION 1..5: note = synthetic sequence
  REPEAT 1..5: note = The sequence repeats n times, where n is 1, 2, 3, 4, 5, 6, 7, 8, 9, or 10
GGGGS

SEQ ID NO: 358 - protein, 5 aa, synthetic construct
  REGION 1..5: note = synthetic sequence
AAAGG

SEQ ID NO: 359 - protein, 9 aa, synthetic construct
  REGION 1..9: note = synthetic sequence
YPYDVPDYA

SEQ ID NO: 360 - protein, 8 aa, synthetic construct
  REGION 1..8: note = synthetic sequence
DYKDDDDK

SEQ ID NO: 361 - protein, 10 aa, synthetic construct
  REGION 1..10: note = synthetic sequence
EQKLISEEDL

SEQ ID NO: 362 - protein, 5 aa, synthetic construct
  REGION 1..5: note = synthetic sequence
HHHHH

SEQ ID NO: 363 - protein, 6 aa, synthetic construct
  REGION 1..6: note = synthetic sequence
HHHHHH

SEQ ID NO: 364 - protein, 8 aa, synthetic construct
  REGION 1..8: note = synthetic sequence
WSHPQFEK

SEQ ID NO: 365 - protein, 5 aa, synthetic construct
  REGION 1..5: note = synthetic sequence
RYIRS

SEQ ID NO: 366 - protein, 4 aa, synthetic construct
  REGION 1..4: note = synthetic sequence
FHHT

SEQ ID NO: 367 - protein, 17 aa, synthetic construct
  REGION 1..17: note = synthetic sequence
WEAAAREACCRECCARA

SEQ ID NO: 368 - protein, 8 aa, synthetic construct
  REGION 1..8: note = synthetic sequence
LEVLFQGP

SEQ ID NO: 369 - protein, 7 aa, synthetic construct
  REGION 1..7: note = synthetic sequence
ENLYTQS

SEQ ID NO: 370 - protein, 5 aa, synthetic construct
  REGION 1..5: note = synthetic sequence
DDDDK

SEQ ID NO: 371 - protein, 4 aa, synthetic construct
  REGION 1..4: note = synthetic sequence
LVPR

SEQ ID NO: 372 - protein, 22 aa, synthetic construct
  REGION 1..22: note = synthetic sequence
GSGATNFSLLKQAGDVEENPGP

SEQ ID NO: 373 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 374 - protein, 325 aa, Homo sapiens
STKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 375 - protein, 246 aa, Homo sapiens
HKPSNTKVDKRVELKTPLGDTTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 376 - protein, 383 aa, Homo sapiens
PTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 377 - protein, 276 aa, Homo sapiens
VTSTLTIKZSDWLGESMFTCRVDHRGLTFQQNASSMCVPDQDTAIRVFAIPPSFASIFLTKSTKLTCLVTDLTTYBSVTISWTREENGAVKTHTNISESHPNATFSAVGEASICEDBDWSGERFTCTVTHTDLPSPLKQTISRPKGVALHRPBVYLLPPARZZLNLRESATITCLVTGFSPADVFVEWMQRGEPLSPQKYVTSAPMPEPQAPGRYFAHSILTVSEEEWNTGGTYTCVVAHEALPNRVTERTVDKSTGKPTLYNVSLVMSDTAGTCY

SEQ ID NO: 378 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 379 - protein, 222 aa, Homo sapiens
ADPCDSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 380 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 381 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 382 - protein, 227 aa, Homo sapiens
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 383 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 384 - protein, 227 aa, Homo sapiens
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 385 - protein, 119 aa, unidentified
  REGION 1..119: note = Homo sapiens or Pan troglodytes
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 386 - protein, 119 aa, Macaca mulatta
MSRSVALAVLALLSLSGLEAIQRTPKIQVYSRHPPENGKPNFLNCYVSGFHPSDIEVDLLKNGEKMGKVEHSDLSFSKDWSFYLLYYTEFTPNEKDEYACRVNHVTLSGPRTVKWDRDM

SEQ ID NO: 387 - protein, 118 aa, Bos taurus
MARFVALVLLGLLSLSGLDAIQRPPKIQVYSRHPPEDGKPNYLNCYVYGFHPPQIEIDLLKNGEKIKSEQSDLSFSKDWSFYLLSHAEFTPNSKDQYSCRVKHVTLEQPRIVKWDRDL

SEQ ID NO: 388 - protein, 119 aa, Mus musculus
MARSVTLVFLVLVSLTGLYAIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDM

SEQ ID NO: 389 - protein, 275 aa, Homo sapiens
GSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 390 - protein, 275 aa, Homo sapiens
GSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWE

SEQ ID NO: 391 - protein, 275 aa, Homo sapiens
CSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWE

SEQ ID NO: 392 - protein, 274 aa, Mus musculus
GPHSLRYFVTAVSRPGLGEPRFIAVGYVDDTQFVRFDSDADNPRFEPRAPWMEQEGPEYWEEQTQRAKSDEQWFRVSLRTAQRYYNQSKGGSHTFQRMFGCDVGSDWRLLRGYQQFAYDGRDYIALNEDLKTWTAADTAALITRRKWEQAGDAEYYRAYLEGECVEWLRRYLELGNETLLRTDSPKAHVTYHPRSQVDVTLRCWALGFYPADITLTWQLNGEDLTQDMELVETRPAGDGTFQKWAAVVVPLGKEQNYTCHVHHKGLPEPLTLRW

SEQ ID NO: 393 - protein, 275 aa, Homo sapiens
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWE

SEQ ID NO: 394 - protein, 276 aa, Homo sapiens
GSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 395 - protein, 276 aa, Homo sapiens
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 396 - protein, 276 aa, Homo sapiens
GSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 397 - protein, 276 aa, Homo sapiens
GSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 398 - protein, 276 aa, Homo sapiens
GSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 399 - protein, 276 aa, Homo sapiens
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITQRKWEAARVAEQLRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 400 - protein, 276 aa, Homo sapiens
GSHSMRYFSTSVSRPGRGEPRFIAVGYVDDTQFVRFDSDAASQRMEPRAPWIEQEGPEYWDEETGKVKAQSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 401 - protein, 276 aa, Homo sapiens
GSHSMRYFYTSVSRPGRGEPRFIAVGYVDDTQFVRFDSDAASQRMEPRAPWIEQEGPEYWDRNTRKVKAQSQTDRVDLGTLRGYYNQSEDGSHTIQRMYGCDVGPDGRFLRGYQQDAYDGKDYIALNEDLRSWTAADMAAQITQRKWETAHEAEQWRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 402 - protein, 276 aa, Homo sapiens
GSHSMRYFDTAMSRPGRGEPRFISVGYVDDTQFVRFDSDAASPREEPRAPWIEQEGPEYWDRNTQIFKTNTQTDRESLRNLRGYYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 403 - protein, 276 aa, Homo sapiens
GSHSMRYFYTAMSRPGRGEPRFIAVGYVDDTQFVRFDSDAASPRMAPRAPWIEQEGPEYWDRNTQISKTNTQTYRESLRNLRGYYNQSEAGSHIIQRMYGCDVGPDGRLLRGYDQSAYDGKDYIALNEDLSSWTAADTAAQITQRKWEAAREAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 404 - protein, 276 aa, Homo sapiens
GSHSMRYFYTSVSRPGRGEPRFISVGYVDDTQFVRFDSDAASPREEPRAPWIEQEGPEYWDRNTQICKTNTQTYRENLRTALRYYNQSEAGSHTLQRMYGCDVGPDGRLLRGHNQFAYDGKDYIALNEDLSSWTAADTAAQITQRKWEAARVAEQLRTYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 405 - protein, 276 aa, Homo sapiens
GSHSMRYFHTAMSRPGRGEPRFITVGYVDDTLFVRFDSDATSPRKEPRAPWIEQEGPEYWDRETQISKTNTQTYRESLRNLRGYYNQSEAGSHTLQRMYGCDVGPDGRLLRGHNQYAYDGKDYIALNEDLRSWTAADTAAQISQRKLEAARVAEQLRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 406 - protein, 276 aa, Homo sapiens
GSHSMRYFYTAMSRPGRGEPRFIAVGYVDDTQFVRFDSDAASPRMAPRAPWIEQEGPEYWDRETQKYKRQAQTDRVSLRNLRGYYNQSEAGSHTLQRMYGCDVGPDGRLLRGHDQSAYDGKDYIALNEDLSSWTAADTAAQITQRKWEAAREAEQWRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 407 - protein, 276 aa, Homo sapiens
GSHSMRYFYTAMSRPGRGEPRFIAVGYVDDTQFVRFDSDAASPRTEPRAPWIEQEGPEYWDRNTQIFKTNTQTYRENLRIALRYYNQSEAGSHIIQRMYGCDLGPDGRLLRGHDQSAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 408 - protein, 276 aa, Homo sapiens
CSHSMKYFFTSVSRPGRGEPRFISVGYVDDTQFVRFDSDAASPRGEPRAPWVEQEGPEYWDRETQKYKRQAQTDRVSLRNLRGYYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWEP

SEQ ID NO: 409 - protein, 276 aa, Homo sapiens
GSHSMRYFYTAVSRPGRGEPHFIAVGYVDDTQFVRFDSDAASPRGEPRAPWVEQEGPEYWDRETQKYKRQAQTDRVSLRNLRGYYNQSEARSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEP

SEQ ID NO: 410 - protein, 276 aa, Homo sapiens
GSHSMRYFYTAVSRPGRGEPHFIAVGYVDDTQFVRFDSDAASPRGEPRAPWVEQEGPEYWDRETQKYKRQAQTDRVSLRNLRGYYNQSEAGSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEP

SEQ ID NO: 411 - protein, 276 aa, Homo sapiens
GSHSMRYFSTSVSWPGRGEPRFIAVGYVDDTQFVRFDSDAASPRGEPREPWVEQEGPEYWDRETQKYKRQAQADRVNLRKLRGYYNQSEDGSHTLQRMFGCDLGPDGRLLRGYNQFAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWKP

SEQ ID NO: 412 - protein, 276 aa, Homo sapiens
CSHSMRYFDTAVSRPGRGEPRFISVGYVDDTQFVRFDSDAASPRGEPRAPWVEQEGPEYWDRETQKYKRQAQADRVNLRKLRGYYNQSEDGSHTLQWMYGCDLGPDGRLLRGYDQSAYDGKDYIALNEDLRSWTAADTAAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEP

SEQ ID NO: 413 - protein, 276 aa, Homo sapiens
CSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEP

SEQ ID NO: 414 - protein, 276 aa, Homo sapiens
CSHSMRYFYTAVSRPGRGEPRFIAVGYVDDTQFVQFDSDAASPRGEPRAPWVEQEGPEYWDRETQKYKRQAQTDRVSLRNLRGYYNQSEAGSHTLQRMYGCDLGPDGRLLRGYNQFAYDGKDYIALNEDLRSWTAADTAAQITQRKWEAARTAEQLRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWGP

SEQ ID NO: 415 - protein, 276 aa, Homo sapiens
CSHSMRYFYTAVSRPGRGEPHFIAVGYVDDTQFVRFDSDAASPRGEPRAPWVEQEGPEYWDRETQNYKRQAQTDRVNLRKLRGYYNQSEAGSHIIQRMYGCDLGPDGRLLRGHDQLAYDGKDYIALNEDLRSWTAADTAAQITQRKWEAAREAEQLRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEP

SEQ ID NO: 416 - protein, 306 aa, SARS-CoV-2 [sequence omitted]

SEQ ID NO: 417 - protein, 75 aa, SARS-CoV-2
MYSFVSEETGTLIVNSVLLFLAFVVFLLVTLAILTALRLCAYCCNIVNVSLVKPSFYVYSRVKNLNSSRVPDLLV

SEQ ID NO: 418 - protein, 601 aa, SARS-CoV-2
AVGACVLCNSQTSLRCGACIRRPFLCCKCCYDHVISTSHKLVLSVNPYVCNAPGCDVTDVTQLYLGGMSYYCKSHKPPISFPLCANGQVFGLYKNTCVGSDNVTDFNAIATCDWTNAGDYILANTCTERLKLFAAETLKATEETFKLSYGIATVREVLSDRELHLSWEVGKPRPPLNRNYVFTGYRVTKNSKVQIGEYTFEKGDYGDAVVYRGTTTYKLNVGDYFVLTSHTVMPLSAPTLVPQEHYVRITGLYPTLNISDEFSSNVANYQKVGMQKYSTLQGPPGTGKSHFAIGLALYYPSARIVYTACSHAAVDALCEKALKYLPIDKCSRIIPARARVECFDKFKVNSTLEQYVFCTVNALPETTADIVVFDEISMATNYDLSVVNARLRAKHYVYIGDPAQLPAPRTLLTKGTLEPEYFNSVCRLMKTIGPDMFLGTCRRCPAEIVDTVSALVYDNKLKAHKDKSAQCFKMFYKGVITHDVSSAINRPQIGVVREFLTRNPAWRKAVFISPYNSQNAVASKILGLPTQTVDSSQGSEYDYVIFTQTTETAHSCNVNRFNVAITRAKVGILCIMSDRDLYDKLQFTSLEIPRRNVATLQ

SEQ ID NO: 419 - protein, 222 aa, SARS-CoV-2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 420 - protein, 180 aa, SARS-CoV-2
MESLVPGFNEKTHVQLSLPVLQVRDVLVRGFGDSVEEVLSEARQHLKDGTCGLVEVEKGVLPQLEQPYVFIKRSDARTAPHGHVMVELVAELEGIQYGRSGETLGVLVPHVGEIPVAYRKVLLRKNGNKGAGGHSYGADLKSFDLGDELGTDPYEDFQENWNTKHSSGVTRELMRELNGG

SEQ ID NO: 421 - protein, 638 aa, SARS-CoV-2
AYTRYVDNNFCGPDGYPLECIKDLLARAGKASCTLSEQLDFIDTKRGVYCCREHEHEIAWYTERSEKSYELQTPFEIKLAKKFDTFNGECPNFVFPLNSIIKTIQPRVEKKKLDGFMGRIRSVYPVASPNECNQMCLSTLMKCDHCGETSWQTGDFVKATCEFCGTENLTKEGATTCGYLPQNAVVKIYCPACHNSEVGPEHSLAEYHNESGLKTILRKGGRTIAFGGCVFSYVGCHNKCAYWVPRASANIGCNHTGVVGEGSEGLNDNLLEILQKEKVNINIVGDFKLNEEIAIILASFSASTSAFVETVKGLDYKAFKQIVESCGNFKVTKGKAKKGAWNIGEQKSILSPLYAFASEAARVVRSIFSRTLETAQNSVRVLQKAAITILDGISQYSLRLIDAMMFTSDLATNNLVVMAYITGGVVQLTSQWLTNIFGTVYEKLKPVLDWLEEKFKEGVEFLRDGWEIVKFISTCACEIVGGQIVTCAKEIKESVQTFFKLVNKFLALCADSIIIGGAKLKALNLGETFVTHSKGLYRKCVKSREETGLLMPLKAPKEIIFLEGETLPTEVLTEEVVLKTGDLQPLEQPTSEAVEAPLVGTPVCINGLMLLEIKDTEKYCALAPNMMVTNNTFTLKGG

SEQ ID NO: 422 - protein, 500 aa, SARS-CoV-2
KIVNNWLKQLIKVTLVFLFVAAIFYLITPVHVMSKHTDFSSEIIGYKAIDGGVTRDIASTDTCFANKHADFDTWFSQRGGSYTNDKACPLIAAVITREVGFVVPGLPGTILRTTNGDFLHFLPRVFSAVGNICYTPSKLIEYTDFATSACVLAAECTIFKDASGKPVPYCYDTNVLEGSVAYESLRPDTRYVLMDGSIIQFPNTYLEGSVRVVTTFDSEYCRHGTCERSEAGVCVSTSGRWVLNNDYYRSLPGVFCGVDAVNLLTNMFTPLIQPIGALDISASIVAGGIVAIVVTCLAYYFMRFRRAFGEYSHVVAFNTLLFLMSFTVLCLTPVYSFLPGVYSVIYLYLTFYLTNDVSFLAHIQWMVMFTPLVPFWITIAYIICISTKHFYWFFSNYLKRRVVFNGVSFSTFEEAALCTFLLNKEMYLKLRSDVLLPLTQYNRYLALYNKYKYFSGAMDTTSYREAACCHLAKALNDFSNSGSDVLYQPPQTSITSAVLQ

SEQ ID NO: 423 - protein, 290 aa, SARS-CoV-2
SAVKRTIKGTHHWLLLTILTSLLVLVQSTQWSLFFFLYENAFLPFAMGIIAMSAFAMMFVKHKHAFLCLFLLPSLATVAYFNMVYMPASWVMRIMTWLDMVDTSLSGFKLKDCVMYASAVVLLILMTARTVYDDGARRVWTLMNVLTLVYKVYYGNALDQAISMWALIISVTSNYSGVVTTVMFLARGIVFMCVEYCPIFFITGNTLQCIMLVYCFLGYFCTCYFGLFCLLNRYFRLTLGVYDYLVSTQEFRYMNSQGLLPPKNSIDAFKLNIKLLGVGGKPCIKVATVQ

SEQ ID NO: 424 - protein, 83 aa, SARS-CoV-2
SKMSDVKCTSVVLLSVLQQLRVESSSKLWAQCVQLHNDILLAKDTTEAFEKMVSLLSVLLSMQGAVDINKLCEEMLDNRATLQ

SEQ ID NO: 425 - protein, 198 aa, SARS-CoV-2
AIASEFSSLPSYAAFATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVIPDYNTYKNTCDGTTFTYASALWEIQQVVDADSKIVQLSEISMDNSPNLAWPLIVTALRANSAVKLQ

SEQ ID NO: 426 - protein, 113 aa, SARS-CoV-2
NNELSPVALRQMSCAAGTTQTACTDDNALAYYNTTKGGRFVLALLSDLQDLKWARFPKSDGTGTIYTELEPPCRFVTDTPKGPKVKYLYFIKGLNNLNRGMVLGSLAATVRLQ

SEQ ID NO: 427 - protein, 139 aa, SARS-CoV-2
AGNATEVPANSTVLSFCAFAVDAAKAYKDYLASGGQPITNCVKMLCTHTGTGQAITVTPEANMDQESFGGASCCLYCRCHIDHPNPKGFCDLKGKYVQIPTTCANDPVGFTLKNTVCTVCGMWKGYGCSCDQLREPMLQ

SEQ ID NO: 428 - protein, 527 aa, SARS-CoV-2
AENVTGLFKDCSKVITGLHPTQAPTHLSVDTKFKTEGLCVDIPGIPKDMTYRRLISMMGFKMNYQVNGYPNMFITREEAIRHVRAWIGFDVEGCHATREAVGTNLPLQLGFSTGVNLVAVPTGYVDTPNNTDFSRVSAKPPPGDQFKHLIPLMYKGLPWNVVRIKIVQMLSDTLKNLSDRVVFVLWAHGFELTSMKYFVKIGPERTCCLCDRRATCFSTASDTYACWHHSIGFDYVYNPFMIDVQQWGFTGNLQSNHDLYCQVHGNAHVASCDAIMTRCLAVHECFVKRVDWTIEYPIIGDELKINAACRKVQHMVVKAALLADKFPVLHDIGNPKAIKCVPQADVEWKFYDAQPCSDKAYKIEELFYSYATHSDKFTDGVCLFWNCNVDRYPANSIVCRFDTRVLSNLNLPGCDGGSLYVNKHAFHTPAFDKSAFVNLKQLPFFYYSDSPCESHGKQVVSDIDYVPLKSATCITRCNLGGAVCRHHANEYRLYLDAYNMMISAGFSLWVYKQFDTYNLWNTFTRLQ

SEQ ID NO: 429 - protein, 346 aa, SARS-CoV-2
SLENVAFNVVNKGHFDGQQGEVPVSIINNTVYTKVDGVDVELFENKTTLPVNVAFELWAKRNIKPVPEVKILNNLGVDIAANTVIWDYKRDAPAHISTIGVCSMTDIAKKPTETICAPLTVFFDGRVDGQVDLFRNARNGVLITEGSVKGLQPSVGPKQASLNGVTLIGEAVKTQFNYYKKVDGVVQQLPETYFTQSRNLQEFKPRSQMEIDFLELAMDEFIERYKLEGYAFEHIVYGDFSHSQLGGLHLLIGLAKRFKESPFELEDFIPMDSTVKNYFITDAQTGSSKCVCSVIDLLLDDFVEIIKSQDLSVVSKVVKVTIDYTEISFMLWCKDGHVETFYPKLQ

SEQ ID NO: 430 - protein, 298 aa, SARS-CoV-2
SSQAWQPGVAMPNLYKMQRMLLEKCDLQNYGDSATLPKGIMMNVAKYTQLCQYLNTLTLAVPYNMRVIHFGAGSDKGVAPGTAVLRQWLPTGTLLVDSDLNDFVSDADSTLIGDCATVHTANKWDLIISDMYDPKTKNVTKENDSKEGFFTYICGFIQQKLALGGSVAIKITEHSWNADLYKLMGHFAWWTAFVTNVNASSSEAFLIGCNYLGKPREQIDGYVMHANYIFWRNTNPIQLSSYSLFDMSKFPLKLRGTAVMSLKEGQINDMILSLLSKGRLIIRENNRVVISSDVLVNN

SEQ ID NO: 431 - protein, 419 aa, SARS-CoV-2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 432 - protein, 275 aa, SARS-CoV-2
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 433 - protein, 61 aa, SARS-CoV-2
MFHLVDFQVTIAEILLIIMRTFKVSIWNLDYIINLIIKNLSKSLTENKYSQLDEEQPMEID

SEQ ID NO: 434 - protein, 121 aa, SARS-CoV-2
MKIILFLALITLATCELYHYQECVRGTTVLLKEPCSSGTYEGNSPFHPLADNKFALTCFSTQFAFACPDGVKHVYQLRARSVSPKLFIRQEEVQELYSPIFLIVAAIVFITLCFTLKRKTE

SEQ ID NO: 435 - protein, 121 aa, SARS-CoV-2
MKFLVFLGIITTVAAFHQECSLQSCTQHQPYVVDDPCPIHFYSKWYIRVGARKSAPLIELCVDEAGSKSPIQYIDIGNYTVSCLPFTINCQEPKLGSLVVRCSFYEDFLEYHDVRVVLDFI

SEQ ID NO: 436 - protein, 38 aa, SARS-CoV-2
MGYINVFAFPFTIYSLLLCRMNSRNYIAQVDVVNFNLT

SEQ ID NO: 437 - protein, 1945 aa, SARS-CoV-2
APTKVTFGDDTVIEVQGYKSVNITFELDERIDKVLNEKCSAYTVELGTEVNEFACVVADAVIKTLQPVSELLTPLGIDLDEWSMATYYLFDESGEFKLASHMYCSFYPPDEDEEEGDCEEEEFEPSTQYEYGTEDDYQGKPLEFGATSAALQPEEEQEEDWLDDDSQQTVGQQDGSEDNQTTTIQTIVEVQPQLEMELTPVVQTIEVNSFSGYLKLTDNVYIKNADIVEEAKKVKPTVVVNAANVYLKHGGGVAGALNKATNNAMQVESDDYIATNGPLKVGGSCVLSGHNLAKHCLHVVGPNVNKGEDIQLLKSAYENFNQHEVLLAPLLSAGIFGADPIHSLRVCVDTVRTNVYLAVFDKNLYDKLVSSFLEMKSEKQVEQKIAEIPKEEVKPFITESKPSVEQRKQDDKKIKACVEEVTTTLEETKFLTENLLLYIDINGNLHPDSATLVSDIDITFLKKDAPYIVGDVVQEGVLTAVVIPTKKAGGTTEMLAKALRKVPTDNYITTYPGQGLNGYTVEEAKTVLKKCKSAFYILPSIISNEKQEILGTVSWNLREMLAHAEETRKLMPVCVETKAIVSTIQRKYKGIKIQEGVVDYGARFYFYTSKTTVASLINTLNDLNETLVTMPLGYVTHGLNLEEAARYMRSLKVPATVSVSSPDAVTAYNGYLTSSSKTPEEHFIETISLAGSYKDWSYSGQSTQLGIEFLKRGDKSVYYTSNPTTFHLDGEVITFDNLKTLLSLREVRTIKVFTTVDNINLHTQVVDMSMTYGQQFGPTYLDGADVTKIKPHNSHEGKTFYVLPNDDTLRVEAFEYYHTTDPSFLGRYMSALNHTKKWKYPQVNGLTSIKWADNNCYLATALLTLQQIELKFNPPALQDAYYRARAGEAANFCALILAYCNKTVGELGDVRETMSYLFQHANLDSCKRVLNVVCKTCGQQQTTLKGVEAVMYMGTLSYEQFKKGVQIPCTCGKQATKYLVQQESPFVMMSAPPAQYELKHGTFTCASEYTGNYQCGHYKHITSKETLYCIDGALLTKSSEYKGPITDVFYKENSYTTTIKPVTYKLDGVVCTEIDPKLDNYYKKDNSYFTEQPIDLVPNQPYPNASFDNFKFVCDNIKFADDLNQLTGYKKPASRELKVTFFPDLNGDVVAIDYKHYTPSFKKGAKLLHKPIVWHVNNATNKATYKPNTWCIRCLWSTKPVETSNSFDVLKSEDAQGMDNLACEDLKPVSEEVVENPTIQKDVLECNVKTTEVVGDIILKPANNSLKITEEVGHTDLMAAYVDNSSLTIKKPNELSRVLGLKTLATHGLAAVNSVPWDTIANYAKPFLNKVVSTTTNIVTRCLNRVCTNYMPYFFTLLLQLCTFTRSTNSRIKASMPTTIAKNTVKSVGKFCLEASFNYLKSPNFSKLINIIIWFLLLSVCLGSLIYSTAALGVLMSNLGMPSYCTGYREGYLNSTNVTIATYCTGSIPCSVCLSGLDSLDTYPSLETIQITISSFKWDLTAFGLVAEWFLAYILFTRFFYVLGLAAIMQLFFSYFAVHFISNSWLMWLIINLVQMAPISAMVRMYIFFASFYYVWKSYVHVVDGCNSSTCMMCYKRNRATRVECTTIVNGVRRSFYVYANGGKGFCKLHNWNCVNCDTFCAGSTFISDEVARDLSLQFKRPINPTDQSSYIVDSVTVKNGSIHLYFDKAGQKTYERHSLSHFVNLDNLRANNTKGSLPINVIVFDGKSKCEESSAKSASVYYSQLMCQPILLLDQALVSDVGDSAEVAVKMFDAYVNTFSSTFNVPMEKLKTLVATAEAELAKNVSLDNVLSTFISAARQGFVDSDVETKDVVECLKLSHQSDIEVTGDSCNNYMLTYNKVENMTPRDLGACIDCSARHINAQVAKSHNIALIWNVKDFMSLSEQLRKQIRSAAKKNNLPFKLTCATTRQVVNVVTTKIALKGG

SEQ ID NO: 438 - protein, 932 aa, SARS-CoV-2
SADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQ

SEQ ID NO: 439 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 440 - protein, 10 aa, SARS-CoV-2
TSEDMLNPNY

SEQ ID NO: 441 - protein, 14 aa, SARS-CoV-2
TGVHAGTDLEGNFY

SEQ ID NO: 442 - protein, 15 aa, SARS-CoV-2
DVTDVTQLYLGGMSY

SEQ ID NO: 443 - protein, 10 aa, SARS-CoV-2
ATEETFKLSY

SEQ ID NO: 444 - protein, 12 aa, SARS-CoV-2
ISDEFSSNVANY

SEQ ID NO: 445 - protein, 10 aa, SARS-CoV-2
TVDSSQGSEY

SEQ ID NO: 446 - protein, 10 aa, SARS-CoV-2
VAGDSGFAAY

SEQ ID NO: 447 - protein, 10 aa, SARS-CoV-2
LAMDEFIERY

SEQ ID NO: 448 - protein, 11 aa, SARS-CoV-2
ITEHSWNADLY

SEQ ID NO: 449 - protein, 12 aa, SARS-CoV-2
YVDNNFCGPDGY

SEQ ID NO: 450 - protein, 9 aa, SARS-CoV-2
FIDTKRGVY

SEQ ID NO: 451 - protein, 9 aa, SARS-CoV-2
YTERSEKSY

SEQ ID NO: 452 - protein, 14 aa, SARS-CoV-2
TSDLATNNLVVMAY

SEQ ID NO: 453 - protein, 11 aa, SARS-CoV-2
HTDFSSEIIGY

SEQ ID NO: 454 - protein, 9 aa, SARS-CoV-2
FSAVGNICY

SEQ ID NO: 455 - protein, 10 aa, SARS-CoV-2
FSNSGSDVLY

SEQ ID NO: 456 - protein, 10 aa, SARS-CoV-2
MADQAMTQMY

SEQ ID NO: 457 - protein, 14 aa, SARS-CoV-2
NTYKNTCDGTTFTY

SEQ ID NO: 458 - protein, 14 aa, SARS-CoV-2
TTQTACTDDNALAY

SEQ ID NO: 459 - protein, 13 aa, SARS-CoV-2
QTACTDDNALAYY

SEQ ID NO: 460 - protein, 10 aa, SARS-CoV-2
CTDDNALAYY

SEQ ID NO: 461 - protein, 14 aa, SARS-CoV-2
WARFPKSDGTGTIY

SEQ ID NO: 462 - protein, 14 aa, SARS-CoV-2
VTDTPKGPKVKYLY

SEQ ID NO: 463 - protein, 10 aa, SARS-CoV-2
YFTSDYYQLY

SEQ ID NO: 464 - protein, 14 aa, SARS-CoV-2
STDTGVEHVTFFIY

SEQ ID NO: 465 - protein, 11 aa, SARS-CoV-2
VVDDPCPIHFY

SEQ ID NO: 466 - protein, 8 aa, SARS-CoV-2
LTENLLLY

SEQ ID NO: 467 - protein, 14 aa, SARS-CoV-2
ALRKVPTDNYITTY

SEQ ID NO: 468 - protein, 11 aa, SARS-CoV-2
KVPTDNYITTY

SEQ ID NO: 469 - protein, 9 aa, SARS-CoV-2
VVDYGARFY

SEQ ID NO: 470 - protein, 11 aa, SARS-CoV-2
HTTDPSFLGRY

SEQ ID NO: 471 - protein, 11 aa, SARS-CoV-2
ITDVFYKENSY

SEQ ID NO: 472 - protein, 14 aa, SARS-CoV-2
FTEQPIDLVPNQPY

SEQ ID NO: 473 - protein, 11 aa, SARS-CoV-2
FADDLNQLTGY

SEQ ID NO: 474 - protein, 14 aa, SARS-CoV-2
ITEEVGHTDLMAAY

SEQ ID NO: 475 - protein, 12 aa, SARS-CoV-2
STDVVYRAFDIY

SEQ ID NO: 476 - protein, 11 aa, SARS-CoV-2
FVENPDILRVY

SEQ ID NO: 477 - protein, 13 aa, SARS-CoV-2
FVDGVPFVVSTGY

SEQ ID NO: 478 - protein, 10 aa, SARS-CoV-2
AISDYDYYRY

SEQ ID NO: 479 - protein, 9 aa, SARS-CoV-2
VVDKYFDCY

SEQ ID NO: 480 - protein, 14 aa, SARS-CoV-2
YSDVENPHLMGWDY

SEQ ID NO: 481 - protein, 12 aa, SARS-CoV-2
LSEMVMCGGSLY

SEQ ID NO: 482 - protein, 11 aa, SARS-CoV-2
STDGNKIADKY

SEQ ID NO: 483 - protein, 13 aa, SARS-CoV-2
NRDVDTDFVNEFY

SEQ ID NO: 484 - protein, 11 aa, SARS-CoV-2
DTDFVNEFYAY

SEQ ID NO: 485 - protein, 13 aa, SARS-CoV-2
LSDDAVVCFNSTY

SEQ ID NO: 486 - protein, 8 aa, SARS-CoV-2
YADVFHLY

SEQ ID NO: 487 - protein, 13 aa, SARS-CoV-2
YSVMLTNDNTSRY

SEQ ID NO: 488 - protein, 10 aa, SARS-CoV-2
FLVQAGNVQL

SEQ ID NO: 489 - protein, 9 aa, SARS-CoV-2
FLNRFTTTL

SEQ ID NO: 490 - protein, 12 aa, SARS-CoV-2
ALLEDEFTPFDV

SEQ ID NO: 491 - protein, 9 aa, SARS-CoV-2
SLVKPSFYV

SEQ ID NO: 492 - protein, 9 aa, SARS-CoV-2
KLSYGIATV

SEQ ID NO: 493 - protein, 9 aa, SARS-CoV-2
TLVPQEHYV

SEQ ID NO: 494 - protein, 9 aa, SARS-CoV-2
GLVEVEKGV

SEQ ID NO: 495 - protein, 9 aa, SARS-CoV-2
VMVELVAEL

SEQ ID NO: 496 - protein, 9 aa, SARS-CoV-2
TLGVLVPHV

SEQ ID NO: 497 - protein, 9 aa, SARS-CoV-2
YLASGGQPI

SEQ ID NO: 498 - protein, 9 aa, SARS-CoV-2
MLSDTLKNL

SEQ ID NO: 499 - protein, 9 aa, SARS-CoV-2
NLSDRVVFV

SEQ ID NO: 500 - protein, 9 aa, SARS-CoV-2
VLWAHGFEL

SEQ ID NO: 501 - protein, 9 aa, SARS-CoV-2
LLADKFPVL

SEQ ID NO: 502 - protein, 9 aa, SARS-CoV-2
YLDAYNMMI

SEQ ID NO: 503 - protein, 9 aa, SARS-CoV-2
MMISAGFSL

SEQ ID NO: 504 - protein, 9 aa, SARS-CoV-2
SLENVAFNV

SEQ ID NO: 505 - protein, 9 aa, SARS-CoV-2
SQLGGLHLL

SEQ ID NO: 506 - protein, 10 aa, SARS-CoV-2
LLLDDFVEII

SEQ ID NO: 507 - protein, 9 aa, SARS-CoV-2
YLNTLTLAV

SEQ ID NO: 508 - protein, 9 aa, SARS-CoV-2
TLIGDCATV

SEQ ID NO: 509 - protein, 12 aa, SARS-CoV-2
SLFDMSKFPLKL

SEQ ID NO: 510 - protein, 10 aa, SARS-CoV-2
GLNDNLLEIL

SEQ ID NO: 511 - protein, 9 aa, SARS-CoV-2
KLNEEIAII

SEQ ID NO: 512 - protein, 9 aa, SARS-CoV-2
RLIDAMMFT

SEQ ID NO: 513 - protein, 9 aa, SARS-CoV-2
YITGGVVQL

SEQ ID NO: 514 - protein, 9 aa, SARS-CoV-2
KLVNKFLAL

SEQ ID NO: 515 - protein, 9 aa, SARS-CoV-2
ALNLGETFV

SEQ ID NO: 516 - protein, 9 aa, SARS-CoV-2
YLITPVHVM

SEQ ID NO: 517 - protein, 9 aa, SARS-CoV-2
FLPRVFSAV

SEQ ID NO: 518 - protein, 9 aa, SARS-CoV-2
KLIEYTDFA

SEQ ID NO: 519 - protein, 9 aa, SARS-CoV-2
IVAGGIVAI

SEQ ID NO: 520 - protein, 9 aa, SARS-CoV-2
FLAHIQWMV

SEQ ID NO: 521 - protein, 9 aa, SARS-CoV-2
FLLNKEMYL

SEQ ID NO: 522 - protein, 9 aa, SARS-CoV-2
ILTSLLVLV

SEQ ID NO: 523 - protein, 9 aa, SARS-CoV-2
WLDMVDTSL

SEQ ID NO: 524 - protein, 9 aa, SARS-CoV-2
TLMNVLTLV

SEQ ID NO: 525 - protein, 9 aa, SARS-CoV-2
SMWALIISV

SEQ ID NO: 526 - protein, 9 aa, SARS-CoV-2
VLLSVLQQL

SEQ ID NO: 527 - protein, 9 aa, SARS-CoV-2
KLWAQCVQL

SEQ ID NO: 528 - protein, 9 aa, SARS-CoV-2
ALWEIQQVV

SEQ ID NO: 529 - protein, 9 aa, SARS-CoV-2
ALLSDLQDL

SEQ ID NO: 530 - protein, 9 aa, SARS-CoV-2
NVFAFPFTI

SEQ ID NO: 531 - protein, 9 aa, SARS-CoV-2
ALSKGVHFV

SEQ ID NO: 532 - protein, 9 aa, SARS-CoV-2
TVYSHLLLV

SEQ ID NO: 533 - protein, 9 aa, SARS-CoV-2
YLYALVYFL

SEQ ID NO: 534 - protein, 9 aa, SARS-CoV-2
LLYDANYFL

SEQ ID NO: 535 - protein, 9 aa, SARS-CoV-2
HLVDFQVTI

SEQ ID NO: 536 - protein, 9 aa, SARS-CoV-2
KLFIRQEEV

SEQ ID NO: 537 - protein, 11 aa, SARS-CoV-2
YLFDESGEFKL

SEQ ID NO: 538 - protein, 11 aa, SARS-CoV-2
YLKLTDNVYIK

SEQ ID NO: 539 - protein, 9 aa, SARS-CoV-2
FLKKDAPYI

SEQ ID NO: 540 - protein, 9 aa, SARS-CoV-2
TLNDLNETL

SEQ ID NO: 541 - protein, 10 aa, SARS-CoV-2
YLDGADVTKI

SEQ ID NO: 542 - protein, 9 aa, SARS-CoV-2
YLATALLTL

SEQ ID NO: 543 - protein, 9 aa, SARS-CoV-2
YLNSTNVTI

SEQ ID NO: 544 - protein, 11 aa, SARS-CoV-2
QMAPISAMVRM

SEQ ID NO: 545 - protein, 9 aa, SARS-CoV-2
YVWKSYVHV

SEQ ID NO: 546 - protein, 9 aa, SARS-CoV-2
ILLLDQALV

SEQ ID NO: 547 - protein, 9 aa, SARS-CoV-2
NLIDSYFVV

SEQ ID NO: 548 - protein, 10 aa, SARS-CoV-2
YTMADLVYAL

SEQ ID NO: 549 - protein, 9 aa, SARS-CoV-2
SLLMPILTL

SEQ ID NO: 550 - protein, 10 aa, SARS-CoV-2
KIFVDGVPFV

SEQ ID NO: 551 - protein, 9 aa, SARS-CoV-2
RLANECAQV

SEQ ID NO: 552 - protein, 11 aa, SARS-CoV-2
YLPYPDPSRIL

SEQ ID NO: 553 - protein, 9 aa, SARS-CoV-2
LMIERFVSL

SEQ ID NO: 554 - protein, 9 aa, SARS-CoV-2
MLDMYSVML

SEQ ID NO: 555 - protein, 9 aa, SARS-CoV-2
AMRPNFTIK

SEQ ID NO: 556 - protein, 9 aa, SARS-CoV-2
KLFAAETLK

SEQ ID NO: 557 - protein, 10 aa, SARS-CoV-2
YVFTGYRVTK

SEQ ID NO: 558 - protein, 10 aa, SARS-CoV-2
VVYRGTTTYK

SEQ ID NO: 559 - protein, 9 aa, SARS-CoV-2
ALKYLPIDK

SEQ ID NO: 560 - protein, 9 aa, SARS-CoV-2
VVNARLRAK

SEQ ID NO: 561 - protein, 9 aa, SARS-CoV-2
SLVPGFNEK

SEQ ID NO: 562 - protein, 9 aa, SARS-CoV-2
RLISMMGFK

SEQ ID NO: 563 - protein, 9 aa, SARS-CoV-2
SMMGFKMNY

SEQ ID NO: 564 - protein, 9 aa, SARS-CoV-2
VLHDIGNPK

SEQ ID NO: 565 - protein, 10 aa, SARS-CoV-2
SIINNTVYTK

SEQ ID NO: 566 - protein, 9 aa, SARS-CoV-2
GLQPSVGPK

SEQ ID NO: 567 - protein, 10 aa, SARS-CoV-2
GVAMPNLYKM

SEQ ID NO: 568 - protein, 9 aa, SARS-CoV-2
KMQRMLLEK

SEQ ID NO: 569 - protein, 9 aa, SARS-CoV-2
ALGGSVAIK

SEQ ID NO: 570 - protein, 10 aa, SARS-CoV-2
KTIQPRVEKK

SEQ ID NO: 571 - protein, 9 aa, SARS-CoV-2
KVTKGKAKK

SEQ ID NO: 572 - protein, 9 aa, SARS-CoV-2
NIFGTVYEK

SEQ ID NO: 573 - protein, 9 aa, SARS-CoV-2
IIIGGAKLK

SEQ ID NO: 574 - protein, 9 aa, SARS-CoV-2
VTNNTFTLK

SEQ ID NO: 575 - protein, 9 aa, SARS-CoV-2
TLKGGAPTK

SEQ ID NO: 576 - protein, 9 aa, SARS-CoV-2
TIFKDASGK

SEQ ID NO: 577 - protein, 9 aa, SARS-CoV-2
ALCTFLLNK

SEQ ID NO: 578 - protein, 10 aa, SARS-CoV-2
SMQGAVDINK

SEQ ID NO: 579 - protein, 10 aa, SARS-CoV-2
KLKKSLNVAK

SEQ ID NO: 580 - protein, 9 aa, SARS-CoV-2
TMLFTMLRK

SEQ ID NO: 581 - protein, 10 aa, SARS-CoV-2
VVIPDYNTYK

SEQ ID NO: 582 - protein, 9 aa, SARS-CoV-2
ALRANSAVK

SEQ ID NO: 583 - protein, 9 aa, SARS-CoV-2
ALAYYNTTK

SEQ ID NO: 584 - protein, 11 aa, SARS-CoV-2
RIFTIGTVTLK

SEQ ID NO: 585 - protein, 9 aa, SARS-CoV-2
SASKIITLK

SEQ ID NO: 586 - protein, 9 aa, SARS-CoV-2
ASKIITLKK

SEQ ID NO: 587 - protein, 9 aa, SARS-CoV-2
TVIEVQGYK

SEQ ID NO: 588 - protein, 9 aa, SARS-CoV-2
RIDKVLNEK

SEQ ID NO: 589 - protein, 9 aa, SARS-CoV-2
VLSGHNLAK

SEQ ID NO: 590 - protein, 9 aa, SARS-CoV-2
HVVGPNVNK

SEQ ID NO: 591 - protein, 10 aa, SARS-CoV-2
AVFDKNLYDK

SEQ ID NO: 592 - protein, 10 aa, SARS-CoV-2
KLMPVCVETK

SEQ ID NO: 593 - protein, 9 aa, SARS-CoV-2
AIVSTIQRK

SEQ ID NO: 594 - protein, 9 aa, SARS-CoV-2
TISLAGSYK

SEQ ID NO: 595 - protein, 10 aa, SARS-CoV-2
AVMYMGTLSY

SEQ ID NO: 596 - protein, 9 aa, SARS-CoV-2
VVENPTIQK

SEQ ID NO: 597 - protein, 10 aa, SARS-CoV-2
KASMPTTIAK

SEQ ID NO: 598 - protein, 9 aa, SARS-CoV-2
ATAEAELAK

SEQ ID NO: 599 - protein, 10 aa, SARS-CoV-2
RQVVNVVTTK

SEQ ID NO: 600 - protein, 9 aa, SARS-CoV-2
VVTTKIALK

SEQ ID NO: 601 - protein, 10 aa, SARS-CoV-2
KVAGFAKFLK

SEQ ID NO: 602 - protein, 10 aa, SARS-CoV-2
LLKDCPAVAK

SEQ ID NO: 603 - protein, 9 aa, SARS-CoV-2
AVAKHDFFK

SEQ ID NO: 604 - protein, 9 aa, SARS-CoV-2
RVYANLGER

SEQ ID NO: 605 - protein, 9 aa, SARS-CoV-2
KLFDRYFKY

SEQ ID NO: 606 - protein, 14 aa, SARS-CoV-2
TVFPPTSFGPLVRK

SEQ ID NO: 607 - protein, 9 aa, SARS-CoV-2
TSFGPLVRK

SEQ ID NO: 608 - protein, 9 aa, SARS-CoV-2
TVKPGNFNK

SEQ ID NO: 609 - protein, 9 aa, SARS-CoV-2
KSAGFPFNK

SEQ ID NO: 610 - protein, 9 aa, SARS-CoV-2
RLYYDSMSY

SEQ ID NO: 611 - protein, 12 aa, SARS-CoV-2
MTNRQFHQKLLK

SEQ ID NO: 612 - protein, 9 aa, SARS-CoV-2
ATVVIGTSK

SEQ ID NO: 613 - protein, 9 aa, SARS-CoV-2
LVASIKNFK

SEQ ID NO: 614 - protein, 9 aa, SARS-CoV-2
AIDAYPLTK

SEQ ID NO: 615 - protein, 10 aa, SARS-CoV-2
AVLDMCASLK

SEQ ID NO: 616 - protein, 9 aa, SARS-CoV-2
VTFQSAVKR

SEQ ID NO: 617 - protein, 9 aa, SARS-CoV-2
SSNVANYQK

SEQ ID NO: 618 - protein, 11 aa, SARS-CoV-2
STLQGPPGTGK

SEQ ID NO: 619 - protein, 9 aa, SARS-CoV-2
ASCDAIMTR

SEQ ID NO: 620 - protein, 9 aa, SARS-CoV-2
KTQFNYYKK

SEQ ID NO: 621 - protein, 9 aa, SARS-CoV-2
HSWNADLYK

SEQ ID NO: 622 - protein, 10 aa, SARS-CoV-2
SSYSLFDMSK

SEQ ID NO: 623 - protein, 10 aa, SARS-CoV-2
STSAFVETVK

SEQ ID NO: 624 - protein, 9 aa, SARS-CoV-2
RVVRSIFSR

SEQ ID NO: 625 - protein, 9 aa, SARS-CoV-2
GAMDTTSYR

SEQ ID NO: 626 - protein, 10 aa, SARS-CoV-2
SAVLQSGFRK

SEQ ID NO: 627 - protein, 9 aa, SARS-CoV-2
SAFAMMFVK

SEQ ID NO: 628 - protein, 12 aa, SARS-CoV-2
AVANGDSEVVLK

SEQ ID NO: 629 - protein, 9 aa, SARS-CoV-2
QTMLFTMLR

SEQ ID NO: 630 - protein, 10 aa, SARS-CoV-2
VVNAANVYLK

SEQ ID NO: 631 - protein, 11 aa, SARS-CoV-2
LVSDIDITFLK

SEQ ID NO: 632 - protein, 9 aa, SARS-CoV-2
LTAVVIPTK

SEQ ID NO: 633 - protein, 10 aa, SARS-CoV-2
STQLGIEFLK

SEQ ID NO: 634 - protein, 9 aa, SARS-CoV-2
GTLSYEQFK

SEQ ID NO: 635 - protein, 12 aa, SARS-CoV-2
SYTTTIKPVTYK

SEQ ID NO: 636 - protein, 9 aa, SARS-CoV-2
TSNSFDVLK

SEQ ID NO: 637 - protein, 9 aa, SARS-CoV-2
TTIAKNTVK

SEQ ID NO: 638 - protein, 13 aa, SARS-CoV-2
ASFNYLKSPNFSK

SEQ ID NO: 639 - protein, 12 aa, SARS-CoV-2
SSTFNVPMEKLK

SEQ ID NO: 640 - protein, 9 aa, SARS-CoV-2
GTSTDVVYR

SEQ ID NO: 641 - protein, 9 aa, SARS-CoV-2
RAFDIYNDK

SEQ ID NO: 642 - protein, 13 aa, SARS-CoV-2
KYKFVRIQPGQTF

SEQ ID NO: 643 - protein, 13 aa, SARS-CoV-2
SYYCKSHKPPISF

SEQ ID NO: 644 - protein, 11 aa, SARS-CoV-2
TYKLNVGDYFV

SEQ ID NO: 645 - protein, 9 aa, SARS-CoV-2
VYIGDPAQL

SEQ ID NO: 646 - protein, 9 aa, SARS-CoV-2
SYGADLKSF

SEQ ID NO: 647 - protein, 9 aa, SARS-CoV-2
VFVLWAHGF

SEQ ID NO: 648 - protein, 9 aa, SARS-CoV-2
SYATHSDKF

SEQ ID NO: 649 - protein, 9 aa, SARS-CoV-2
KFTDGVCLF

SEQ ID NO: 650 - protein, 9 aa, SARS-CoV-2
LYLDAYNMM

SEQ ID NO: 651 - protein, 11 aa, SARS-CoV-2
VYKQFDTYNLW

SEQ ID NO: 652 - protein, 14 aa, SARS-CoV-2
VFFDGRVDGQVDLF

SEQ ID NO: 653 - protein, 9 aa, SARS-CoV-2
RYKLEGYAF

SEQ ID NO: 654 - protein, 9 aa, SARS-CoV-2
TFFKLVNKF

SEQ ID NO: 655 - protein, 9 aa, SARS-CoV-2
VFLFVAAIF

SEQ ID NO: 656 - protein, 13 aa, SARS-CoV-2
TYLEGSVRVVTTF

SEQ ID NO: 657 - protein, 9 aa, SARS-CoV-2
YYRSLPGVF

SEQ ID NO: 658 - protein, 10 aa, SARS-CoV-2
GVYSVIYLYL

SEQ ID NO: 659 - protein, 11 aa, SARS-CoV-2
TFYLTNDVSFL

SEQ ID NO: 660 - protein, 9 aa, SARS-CoV-2
MFTPLVPFW

SEQ ID NO: 661 - protein, 10 aa, SARS-CoV-2
VFNGVSFSTF

SEQ ID NO: 662 - protein, 9 aa, SARS-CoV-2
LYQPPQTSI

SEQ ID NO: 663 - protein, 14 aa, SARS-CoV-2
VYMPASWVMRIMTW

SEQ ID NO: 664 - protein, 9 aa, SARS-CoV-2
MYASAVVLL

SEQ ID NO: 665 - protein, 9 aa, SARS-CoV-2
MFLARGIVF

SEQ ID NO: 666 - protein, 9 aa, SARS-CoV-2
IFFITGNTL

SEQ ID NO: 667 - protein, 10 aa, SARS-CoV-2
YDYLVSTQEF

SEQ ID NO: 668 - protein, 9 aa, SARS-CoV-2
TYASALWEI

SEQ ID NO: 669 - protein, 9 aa, SARS-CoV-2
YFIKGLNNL

SEQ ID NO: 670 - protein, 10 aa, SARS-CoV-2
GYINVFAFPF

SEQ ID NO: 671 - protein, 9 aa, SARS-CoV-2
LYLYALVYF

SEQ ID NO: 672 - protein, 9 aa, SARS-CoV-2
VYFLQSINF

SEQ ID NO: 673 - protein, 9 aa, SARS-CoV-2
YYQLYSTQL

SEQ ID NO: 674 - protein, 10 aa, SARS-CoV-2
QYIDIGNYTV

SEQ ID NO: 675 - protein, 10 aa, SARS-CoV-2
QGYKSVNITF

SEQ ID NO: 676 - protein, 9 aa, SARS-CoV-2
DYQGKPLEF

SEQ ID NO: 677 - protein, 9 aa, SARS-CoV-2
LYDKLVSSF

SEQ ID NO: 678 - protein, 9 aa, SARS-CoV-2
LYIDINGNL

SEQ ID NO: 679 - protein, 10 aa, SARS-CoV-2
VYYTSNPTTF

SEQ ID NO: 680 - protein, 10 aa, SARS-CoV-2
EYYHTTDPSF

SEQ ID NO: 681 - protein, 9 aa, SARS-CoV-2
SYLFQHANL

SEQ ID NO: 682 - protein, 10 aa, SARS-CoV-2
NYYKKDNSYF

SEQ ID NO: 683 - protein, 9 aa, SARS-CoV-2
DYKHYTPSF

SEQ ID NO: 684 - protein, 9 aa, SARS-CoV-2
NYMPYFFTL

SEQ ID NO: 685 - protein, 11 aa, SARS-CoV-2
AYILFTRFFYV

SEQ ID NO: 686 - protein, 9 aa, SARS-CoV-2
FFASFYYVW

SEQ ID NO: 687 - protein, 9 aa, SARS-CoV-2
VYANGGKGF

SEQ ID NO: 688 - protein, 10 aa, SARS-CoV-2
KMFDAYVNTF

SEQ ID NO: 689 - protein, 10 aa, SARS-CoV-2
AYVNTFSSTF

SEQ ID NO: 690 - protein, 12 aa, SARS-CoV-2
IYNDKVAGFAKF

SEQ ID NO: 691 - protein, 10 aa, SARS-CoV-2
SYFVVKRHTF

SEQ ID NO: 692 - protein, 9 aa, SARS-CoV-2
NFNKDFYDF

SEQ ID NO: 693 - protein, 9 aa, SARS-CoV-2
AYANSVFNI

SEQ ID NO: 694 - protein, 10 aa, SARS-CoV-2
HLYLQYIRKL

SEQ ID NO: 695 - protein, 9 aa, SARS-CoV-2
DYVYNPFMI

SEQ ID NO: 696 - protein, 8 aa, SARS-CoV-2
TYNLWNTF

SEQ ID NO: 697 - protein, 9 aa, SARS-CoV-2
NYLKRRVVF

SEQ ID NO: 698 - protein, 11 aa, SARS-CoV-2
NYSGVVTTVMF

SEQ ID NO: 699 - protein, 9 aa, SARS-CoV-2
AFPFTIYSL

SEQ ID NO: 700 - protein, 9 aa, SARS-CoV-2
PYNSVTSSI

SEQ ID NO: 701 - protein, 12 aa, SARS-CoV-2
YYRARAGEAANF

SEQ ID NO: 702 - protein, 11 aa, SARS-CoV-2
MYMGTLSYEQF

SEQ ID NO: 703 - protein, 12 aa, SARS-CoV-2
PYPNASFDNFKF

SEQ ID NO: 704 - protein, 9 aa, SARS-CoV-2
TYKPNTWCI

SEQ ID NO: 705 - protein, 9 aa, SARS-CoV-2
FYGGWHNML

SEQ ID NO: 706 - protein, 9 aa, SARS-CoV-2
QPGQTFSVL

SEQ ID NO: 707 - protein, 10 aa, SARS-CoV-2
SPSGVYQCAM

SEQ ID NO: 708 - protein, 10 aa, SARS-CoV-2
RPNFTIKGSF

SEQ ID NO: 709 - protein, 9 aa, SARS-CoV-2
TPHTVLQAV

SEQ ID NO: 710 - protein, 12 aa, SARS-CoV-2
KPRPPLNRNYVF

SEQ ID NO: 711 - protein, 8 aa, SARS-CoV-2
MPLSAPTL

SEQ ID NO: 712 - protein, 10 aa, SARS-CoV-2
IPARARVECF

SEQ ID NO: 713 - protein, 11 aa, SARS-CoV-2
APRTLLTKGTL

SEQ ID NO: 714 - protein, 10 aa, SARS-CoV-2
RPQIGVVREF

SEQ ID NO: 715 - protein, 10 aa, SARS-CoV-2
RNPAWRKAVF

SEQ ID NO: 716 - protein, 14 aa, SARS-CoV-2
SPYNSQNAVASKIL

SEQ ID NO: 717 - protein, 9 aa, SARS-CoV-2
IPRRNVATL

SEQ ID NO: 718 - protein, 9 aa, SARS-CoV-2
RTAPHGHVM

SEQ ID NO: 719 - protein, 10 aa, SARS-CoV-2
APHGHVMVEL

SEQ ID NO: 720 - protein, 10 aa, SARS-CoV-2
VPANSTVLSF

SEQ ID NO: 721 - protein, 10 aa, SARS-CoV-2
HPNPKGFCDL

SEQ ID NO: 722 - protein, 9 aa, SARS-CoV-2
HPTQAPTHL

SEQ ID NO: 723 - protein, 9 aa, SARS-CoV-2
TPAFDKSAF

SEQ ID NO: 724 - protein, 9 aa, SARS-CoV-2
LPVNVAFEL

SEQ ID NO: 725 - protein, 9 aa, SARS-CoV-2
KPVPEVKIL

SEQ ID NO: 726 - protein, 8 aa, SARS-CoV-2
APAHISTI

SEQ ID NO: 727 - protein, 12 aa, SARS-CoV-2
GPKQASLNGVTL

SEQ ID NO: 728 - protein, 8 aa, SARS-CoV-2
KPRSQMEI

SEQ ID NO: 729 - protein, 9 aa, SARS-CoV-2
LPKGIMMNV

SEQ ID NO: 730 - protein, 10 aa, SARS-CoV-2
VPYNMRVIHF

SEQ ID NO: 731 - protein, 11 aa, SARS-CoV-2
KPREQIDGYVM

SEQ ID NO: 732 - protein, 9 aa, SARS-CoV-2
QPRVEKKKL

SEQ ID NO: 733 - protein, 9 aa, SARS-CoV-2
MPLKAPKEI

SEQ ID NO: 734 - protein, 8 aa, SARS-CoV-2
APKEIIFL

SEQ ID NO: 735 - protein, 10 aa, SARS-CoV-2
VVPGLPGTIL

SEQ ID NO: 736 - protein, 10 aa, SARS-CoV-2
VPYCYDTNVL

SEQ ID NO: 737 - protein, 9 aa, SARS-CoV-2
RPDTRYVLM

SEQ ID NO: 738 - protein, 10 aa, SARS-CoV-2
TPLIQPIGAL

SEQ ID NO: 739 - protein, 10 aa, SARS-CoV-2
LPFAMGIIAM

SEQ ID NO: 740 - protein, 9 aa, SARS-CoV-2
IPLTTAAKL

SEQ ID NO: 741 - protein, 9 aa, SARS-CoV-2
SPNLAWPLI

SEQ ID NO: 742 - protein, 8 aa, SARS-CoV-2
SPVALRQM

SEQ ID NO: 743 - protein, 10 aa, SARS-CoV-2
TPKGPKVKYL

SEQ ID NO: 744 - protein, 9 aa, SARS-CoV-2
IPIQASLPF

SEQ ID NO: 745 - protein, 9 aa, SARS-CoV-2
APFLYLYAL

SEQ ID NO: 746 - protein, 13 aa, SARS-CoV-2
SPFHPLADNKFAL

SEQ ID NO: 747 - protein, 9 aa, SARS-CoV-2
RARSVSPKL

SEQ ID NO: 748 - protein, 12 aa, SARS-CoV-2
KPLEFGATSAAL

SEQ ID NO: 749 - protein, 9 aa, SARS-CoV-2
APLLSAGIF

SEQ ID NO: 750 - protein, 9 aa, SARS-CoV-2
APYIVGDVV

SEQ ID NO: 751 - protein, 10 aa, SARS-CoV-2
YPQVNGLTSI

SEQ ID NO: 752 - protein, 11 aa, SARS-CoV-2
KPASRELKVTF

SEQ ID NO: 753 - protein, 10 aa, SARS-CoV-2
TPSFKKGAKL

SEQ ID NO: 754 - protein, 9 aa, SARS-CoV-2
KPANNSLKI

SEQ ID NO: 755 - protein, 10 aa, SARS-CoV-2
KKPNELSRVL

SEQ ID NO: 756 - protein, 9 aa, SARS-CoV-2
MPYFFTLLL

SEQ ID NO: 757 - protein, 9 aa, SARS-CoV-2
TPRDLGACI

SEQ ID NO: 758 - protein, 9 aa, SARS-CoV-2
VAKSHNIAL

SEQ ID NO: 759 - protein, 10 aa, SARS-CoV-2
MVPHISRQRL

SEQ ID NO: 760 - protein, 10 aa, SARS-CoV-2
MPILTLTRAL

SEQ ID NO: 761 - protein, 9 aa, SARS-CoV-2
KPYIKWDLL

SEQ ID NO: 762 - protein, 9 aa, SARS-CoV-2
IPTITQMNL

SEQ ID NO: 763 - protein, 9 aa, SARS-CoV-2
QLRVIGHSM

SEQ ID NO: 764 - protein, 9 aa, SARS-CoV-2
TPKYKFVRI

SEQ ID NO: 765 - protein, 9 aa, SARS-CoV-2
YVYSRVKNL

SEQ ID NO: 766 - protein, 8 aa, SARS-CoV-2
VGKPRPPL

SEQ ID NO: 767 - protein, 9 aa, SARS-CoV-2
VGMQKYSTL

SEQ ID NO: 768 - protein, 9 aa, SARS-CoV-2
YDKLQFTSL

SEQ ID NO: 769 - protein, 9 aa, SARS-CoV-2
DLKGKYVQI

SEQ ID NO: 770 - protein, 9 aa, SARS-CoV-2
MTYRRLISM

SEQ ID NO: 771 - protein, 9 aa, SARS-CoV-2
VVRIKIVQM

SEQ ID NO: 772 - protein, 8 aa, SARS-CoV-2
HMVVKAAL

SEQ ID NO: 773 - protein, 9 aa, SARS-CoV-2
SLYVNKHAF

SEQ ID NO: 774 - protein, 9 aa, SARS-CoV-2
HANEYRLYL

SEQ ID NO: 775 - protein, 9 aa, SARS-CoV-2
NTFTRLQSL

SEQ ID NO: 776 - protein, 9 aa, SARS-CoV-2
SVGPKQASL

SEQ ID NO: 777 - protein, 8 aa, SARS-CoV-2
NVAKYTQL

SEQ ID NO: 778 - protein, 8 aa, SARS-CoV-2
FIQQKLAL

SEQ ID NO: 779 - protein, 9 aa, SARS-CoV-2
GAKLKALNL

SEQ ID NO: 780 - protein, 9 aa, SARS-CoV-2
HVMSKHTDF

SEQ ID NO: 781 - protein, 8 aa, SARS-CoV-2
FMRFRRAF

SEQ ID NO: 782 - protein, 9 aa, SARS-CoV-2
YLKLRSDVL

SEQ ID NO: 783 - protein, 9 aa, SARS-CoV-2
TQYNRYLAL

SEQ ID NO: 784 - protein, 9 aa, SARS-CoV-2
FVKHKHAFL

SEQ ID NO: 785 - protein, 9 aa, SARS-CoV-2
DGARRVWTL

SEQ ID NO: 786 - protein, 9 aa, SARS-CoV-2
EMLDNRATL

SEQ ID NO: 787 - protein, 9 aa, SARS-CoV-2
VLKKLKKSL

SEQ ID NO: 788 - protein, 9 aa, SARS-CoV-2
GPKVKYLYF

SEQ ID NO: 789 - protein, 9 aa, SARS-CoV-2
NNLNRGMVL

SEQ ID NO: 790 - protein, 9 aa, SARS-CoV-2
ITLKKRWQL

SEQ ID NO: 791 - protein, 9 aa, SARS-CoV-2
IIKNLSKSL

SEQ ID NO: 792 - protein, 9 aa, SARS-CoV-2
DGVKHVYQL

SEQ ID NO: 793 - protein, 8 aa, SARS-CoV-2
NLKTLLSL

SEQ ID NO: 794 - protein, 9 aa, SARS-CoV-2
TIKPVTYKL

SEQ ID NO: 795 - protein, 9 aa, SARS-CoV-2
ILKPANNSL

SEQ ID NO: 796 - protein, 8 aa, SARS-CoV-2
TIKKPNEL

SEQ ID NO: 797 - protein, 8 aa, SARS-CoV-2
NSRIKASM

SEQ ID NO: 798 - protein, 8 aa, SARS-CoV-2
KTYERHSL

SEQ ID NO: 799 - protein, 10 aa, SARS-CoV-2
NVPMEKLKTL

SEQ ID NO: 800 - protein, 8 aa, SARS-CoV-2
NVKDFMSL

SEQ ID NO: 801 - protein, 8 aa, SARS-CoV-2
SAAKKNNL

SEQ ID NO: 802 - protein, 9 aa, SARS-CoV-2
FAYTKRNVI

SEQ ID NO: 803 - protein, 9 aa, SARS-CoV-2
SAKNRARTV

SEQ ID NO: 804 - protein, 9 aa, SARS-CoV-2
IAATRGATV

SEQ ID NO: 805 - protein, 9 aa, SARS-CoV-2
SLSHRFYRL

SEQ ID NO: 806 - protein, 8 aa, SARS-CoV-2
YAYLRKHF

SEQ ID NO: 807 - protein, 9 aa, SARS-CoV-2
YLRKHFSMM

SEQ ID NO: 808 - protein, 9 aa, SARS-CoV-2
SIKNFKSVL

SEQ ID NO: 809 - protein, 9 aa, SARS-CoV-2
TANPKTPKY

SEQ ID NO: 810 - protein, 10 aa, SARS-CoV-2
FPLCANGQVF

SEQ ID NO: 811 - protein, 10 aa, SARS-CoV-2
YPTLNISDEF

SEQ ID NO: 812 - protein, 9 aa, SARS-CoV-2
FAIGLALYY

SEQ ID NO: 813 - protein, 8 aa, SARS-CoV-2
YPSARIVY

SEQ ID NO: 814 - protein, 11 aa, SARS-CoV-2
VPHVGEIPVAY

SEQ ID NO: 815 - protein, 9 aa, SARS-CoV-2
HVGEIPVAY

SEQ ID NO: 816 - protein, 10 aa, SARS-CoV-2
FAVDAAKAYK

SEQ ID NO: 817 - protein, 9 aa, SARS-CoV-2
EANMDQESF

SEQ ID NO: 818 - protein, 10 aa, SARS-CoV-2
IPGIPKDMTY

SEQ ID NO: 819 - protein, 9 aa, SARS-CoV-2
HSIGFDYVY

SEQ ID NO: 820 - protein, 10 aa, SARS-CoV-2
YPANSIVCRF

SEQ ID NO: 821 - protein, 9 aa, SARS-CoV-2
EAVKTQFNY

SEQ ID NO: 822 - protein, 8 aa, SARS-CoV-2
YAFEHIVY

SEQ ID NO: 823 - protein, 10 aa, SARS-CoV-2
IPMDSTVKNY

SEQ ID NO: 824 - protein, 9 aa, SARS-CoV-2
QAWQPGVAM

SEQ ID NO: 825 - protein, 9 aa, SARS-CoV-2
TILDGISQY

SEQ ID NO: 826 - protein, 9 aa, SARS-CoV-2
DASGKPVPY

SEQ ID NO: 827 - protein, 9 aa, SARS-CoV-2
NVLEGSVAY

SEQ ID NO: 828 - protein, 9 aa, SARS-CoV-2
SIIQFPNTY

SEQ ID NO: 829 - protein, 10 aa, SARS-CoV-2
FLPGVYSVIY

SEQ ID NO: 830 - protein, 9 aa, SARS-CoV-2
VPFWITIAY

SEQ ID NO: 831 - protein, 9 aa, SARS-CoV-2
LPLTQYNRY

SEQ ID NO: 832 - protein, 11 aa, SARS-CoV-2
FLLPSLATVAY

SEQ ID NO: 833 - protein, 9 aa, SARS-CoV-2
NALDQAISM

SEQ ID NO: 834 - protein, 9 aa, SARS-CoV-2
LAKDTTEAF

SEQ ID NO: 835 - protein, 9 aa, SARS-CoV-2
NPLLYDANY

SEQ ID NO: 836 - protein, 13 aa, SARS-CoV-2
FAFACPDGVKHVY

SEQ ID NO: 837 - protein, 9 aa, SARS-CoV-2
YPGQGLNGY

SEQ ID NO: 838 - protein, 11 aa, SARS-CoV-2
SPDAVTAYNGY

SEQ ID NO: 839 - protein, 9 aa, SARS-CoV-2
QVVDMSMTY

SEQ ID NO: 840 - protein, 14 aa, SARS-CoV-2
LPNDDTLRVEAFEY

SEQ ID NO: 841 - protein, 9 aa, SARS-CoV-2
NPPALQDAY

SEQ ID NO: 842 - protein, 10 aa, SARS-CoV-2
TKPVETSNSF

SEQ ID NO: 843 - protein, 9 aa, SARS-CoV-2
VPWDTIANY

SEQ ID NO: 844 - protein, 9 aa, SARS-CoV-2
YPSLETIQI

SEQ ID NO: 845 - protein, 9 aa, SARS-CoV-2
VARDLSLQF

SEQ ID NO: 846 - protein, 9 aa, SARS-CoV-2
SAKSASVYY

SEQ ID NO: 847 - protein, 9 aa, SARS-CoV-2
VPFVVSTGY

SEQ ID NO: 848 - protein, 9 aa, SARS-CoV-2
NAAISDYDY

SEQ ID NO: 849 - protein, 9 aa, SARS-CoV-2
FVVEVVDKY

SEQ ID NO: 850 - protein, 9 aa, SARS-CoV-2
NPHLMGWDY

SEQ ID NO: 851 - protein, 9 aa, SARS-CoV-2
FVSLAIDAY

SEQ ID NO: 852 - protein, 11 aa, SARS-CoV-2
YPLTKHPNQEY

SEQ ID NO: 853 - protein, 13 aa, SARS-CoV-2
HPNQEYADVFHLY

SEQ ID NO: 854 - protein, 11 aa, SARS-CoV-2
SDRELHLSWEV

SEQ ID NO: 855 - protein, 9 aa, SARS-CoV-2
VEKGVLPQL

SEQ ID NO: 856 - protein, 12 aa, SARS-CoV-2
GEIPVAYRKVLL

SEQ ID NO: 857 - protein, 10 aa, SARS-CoV-2
TEVPANSTVL

SEQ ID NO: 858 - protein, 10 aa, SARS-CoV-2
REAVGTNLPL

SEQ ID NO: 859 - protein, 10 aa, SARS-CoV-2
IEYPIIGDEL

SEQ ID NO: 860 - protein, 10 aa, SARS-CoV-2
VELFENKTTL

SEQ ID NO: 861 - protein, 9 aa, SARS-CoV-2
QEFKPRSQM

SEQ ID NO: 862 - protein, 10 aa, SARS-CoV-2
MEIDFLELAM

SEQ ID NO: 863 - protein, 9 aa, SARS-CoV-2
FELEDFIPM

SEQ ID NO: 864 - protein, 9 aa, SARS-CoV-2
VEIIKSQDL

SEQ ID NO: 865 - protein, 11 aa, SARS-CoV-2
YELQTPFEIKL

SEQ ID NO: 866 - protein, 13 aa, SARS-CoV-2
ACHNSEVGPEHSL

SEQ ID NO: 867 - protein, 9 aa, SARS-CoV-2
AEYHNESGL

SEQ ID NO: 868 - protein, 9 aa, SARS-CoV-2
NESGLKTIL

SEQ ID NO: 869 - protein, 9 aa, SARS-CoV-2
SEGLNDNLL

SEQ ID NO: 870 - protein, 10 aa, SARS-CoV-2
GEQKSILSPL

SEQ ID NO: 871 - protein, 10 aa, SARS-CoV-2
KEIKESVQTF

SEQ ID NO: 872 - protein, 11 aa, SARS-CoV-2
KEIIFLEGETL

SEQ ID NO: 873 - protein, 9 aa, SARS-CoV-2
GETLPTEVL

SEQ ID NO: 874 - protein, 11 aa, SARS-CoV-2
FSSEIIGYKAI

SEQ ID NO: 875 - protein, 10 aa, SARS-CoV-2
REVGFVVPGL

SEQ ID NO: 876 - protein, 13 aa, SARS-CoV-2
GEYSHVVAFNTLL

SEQ ID NO: 877 - protein, 12 aa, SARS-CoV-2
FLYENAFLPFAM

SEQ ID NO: 878 - protein, 10 aa, SARS-CoV-2
TEAFEKMVSL

SEQ ID NO: 879 - protein, 10 aa, SARS-CoV-2
KSEFDRDAAM

SEQ ID NO: 880 - protein, 11 aa, SARS-CoV-2
SEISMDNSPNL

SEQ ID NO: 881 - protein, 10 aa, SARS-CoV-2
QECVRGTTVL

SEQ ID NO: 882 - protein, 10 aa, SARS-CoV-2
YEGNSPFHPL

SEQ ID NO: 883 - protein, 9 aa, SARS-CoV-2
QELYSPIFL

SEQ ID NO: 884 - protein, 14 aa, SARS-CoV-2
YEDFLEYHDVRVVL

SEQ ID NO: 885 - protein, 9 aa, SARS-CoV-2
IEVQGYKSV

SEQ ID NO: 886 - protein, 11 aa, SARS-CoV-2
FELDERIDKVL

SEQ ID NO: 887 - protein, 9 aa, SARS-CoV-2
SELLTPLGI

SEQ ID NO: 888 - protein, 9 aa, SARS-CoV-2
SEDNQTTTI

SEQ ID NO: 889 - protein, 9 aa, SARS-CoV-2
VEVQPQLEM

SEQ ID NO: 890 - protein, 12 aa, SARS-CoV-2
LEMELTPVVQTI

SEQ ID NO: 891 - protein, 10 aa, SARS-CoV-2
YENFNQHEVL

SEQ ID NO: 892 - protein, 9 aa, SARS-CoV-2
HEVLLAPLL

SEQ ID NO: 893 - protein, 9 aa, SARS-CoV-2
NETLVTMPL

SEQ ID NO: 894 - protein, 9 aa, SARS-CoV-2
GEVITFDNL

SEQ ID NO: 895 - protein, 11 aa, SARS-CoV-2
SLREVRTIKVF

SEQ ID NO: 896 - protein, 9 aa, SARS-CoV-2
HEGKTFYVL

SEQ ID NO: 897 - protein, 10 aa, SARS-CoV-2
IELKFNPPAL

SEQ ID NO: 898 - protein, 9 aa, SARS-CoV-2
GEAANFCAL

SEQ ID NO: 899 - protein, 10 aa, SARS-CoV-2
YEQFKKGVQI

SEQ ID NO: 900 - protein, 12 aa, SARS-CoV-2
SEYKGPITDVFY

SEQ ID NO: 901 - protein, 9 aa, SARS-CoV-2
KENSYTTTI

SEQ ID NO: 902 - protein, 9 aa, SARS-CoV-2
TEVVGDIIL

SEQ ID NO: 903 - protein, 9 aa, SARS-CoV-2
NELSRVLGL

SEQ ID NO: 904 - protein, 10 aa, SARS-CoV-2
AEWFLAYILF

SEQ ID NO: 905 - protein, 11 aa, SARS-CoV-2
AEAELAKNVSL

SEQ ID NO: 906 - protein, 13 aa, SARS-CoV-2
AELAKNVSLDNVL

SEQ ID NO: 907 - protein, 9 aa, SARS-CoV-2
VENMTPRDL

SEQ ID NO: 908 - protein, 9 aa, SARS-CoV-2
HEETIYNLL

SEQ ID NO: 909 - protein, 9 aa, SARS-CoV-2
AESHVDTDL

SEQ ID NO: 910 - protein, 10 aa, SARS-CoV-2
HEFCSQHTML

SEQ ID NO: 911 - protein, 11 aa, SARS-CoV-2
YEAMYTPHTVL

SEQ ID NO: 912 - protein, 13 aa, SARS-CoV-2
REVLSDRELHLSW

SEQ ID NO: 913 - protein, 10 aa, SARS-CoV-2
GEYTFEKGDY

SEQ ID NO: 914 - protein, 9 aa, SARS-CoV-2
DEISMATNY

SEQ ID NO: 915 - protein, 10 aa, SARS-CoV-2
REFLTRNPAW

SEQ ID NO: 916 - protein, 8 aa, SARS-CoV-2
AELEGIQY

SEQ ID NO: 917 - protein, 8 aa, SARS-CoV-2
AENVTGLF

SEQ ID NO: 918 - protein, 11 aa, SARS-CoV-2
REEAIRHVRAW

SEQ ID NO: 919 - protein, 9 aa, SARS-CoV-2
FELTSMKYF

SEQ ID NO: 920 - protein, 10 aa, SARS-CoV-2
GEAVKTQFNY

SEQ ID NO: 921 - protein, 8 aa, SARS-CoV-2
TEISFMLW

SEQ ID NO: 922 - protein, 12 aa, SARS-CoV-2
KENDSKEGFFTY

SEQ ID NO: 923 - protein, 10 aa, SARS-CoV-2
REHEHEIAWY

SEQ ID NO: 924 - protein, 9 aa, SARS-CoV-2
FEIKLAKKF

SEQ ID NO: 925 - protein, 12 aa, SARS-CoV-2
SEVGPEHSLAEY

SEQ ID NO: 926 - protein, 10 aa, SARS-CoV-2
EEIAIILASF

SEQ ID NO: 927 - protein, 10 aa, SARS-CoV-2
YEKLKPVLDW

SEQ ID NO: 928 - protein, 10 aa, SARS-CoV-2
EEKFKEGVEF

SEQ ID NO: 929 - protein, 9 aa, SARS-CoV-2
LEIKDTEKY

SEQ ID NO: 930 - protein, 13 aa, SARS-CoV-2
SEAGVCVSTSGRW

SEQ ID NO: 931 - protein, 10 aa, SARS-CoV-2
ASEFSSLPSY

SEQ ID NO: 932 - protein, 9 aa, SARS-CoV-2
MDNSPNLAW

SEQ ID NO: 933 - protein, 9 aa, SARS-CoV-2
TELEPPCRF

SEQ ID NO: 934 - protein, 10 aa, SARS-CoV-2
GLEAPFLYLY

SEQ ID NO: 935 - protein, 14 aa, SARS-CoV-2
SEHDYQIGGYTEKW

SEQ ID NO: 936 - protein, 9 aa, SARS-CoV-2
EEHVQIHTI

SEQ ID NO: 937 - protein, 9 aa, SARS-CoV-2
DEWSMATYY

SEQ ID NO: 938 - protein, 10 aa, SARS-CoV-2
GEFKLASHMY

SEQ ID NO: 939 - protein, 14 aa, SARS-CoV-2
GDCEEEEFEPSTQY

SEQ ID NO: 940 - protein, 12 aa, SARS-CoV-2
EEEFEPSTQYEY

SEQ ID NO: 941 - protein, 9 aa, SARS-CoV-2
IEVNSFSGY

SEQ ID NO: 942 - protein, 9 aa, SARS-CoV-2
SEKQVEQKI

SEQ ID NO: 943 - protein, 11 aa, SARS-CoV-2
AEIPKEEVKPF

SEQ ID NO: 944 - protein, 13 aa, SARS-CoV-2
SNEKQEILGTVSW

SEQ ID NO: 945 - protein, 11 aa, SARS-CoV-2
QEGVVDYGARF

SEQ ID NO: 946 - protein, 12 aa, SARS-CoV-2
GELGDVRETMSY

SEQ ID NO: 947 - protein, 12 aa, SARS-CoV-2
SEYTGNYQCGHY

SEQ ID NO: 948 - protein, 11 aa, SARS-CoV-2
TEIDPKLDNYY

SEQ ID NO: 949 - protein, 11 aa, SARS-CoV-2
AEVAVKMFDAY

SEQ ID NO: 950 - protein, 14 aa, SARS-CoV-2
TVYSDVENPHLMGW

SEQ ID NO: 951 - protein, 11 aa, SARS-CoV-2
SDVENPHLMGW

SEQ ID NO: 952 - protein, 12 aa, SARS-CoV-2
AEIVDTVSALVY

SEQ ID NO: 953 - protein, 11 aa, SARS-CoV-2
RELNGGAYTRY

SEQ ID NO: 954 - protein, 11 aa, SARS-CoV-2
SYEDQDALFAY

SEQ ID NO: 955 - protein, 9 aa, SARS-CoV-2
TTDLEGNFY

SEQ ID NO: 956 - protein, 9 aa, SARS-CoV-2
TVDSQGSEY

SEQ ID NO: 957 - protein, 9 aa, SARS-CoV-2
ASDSGFAAY

SEQ ID NO: 958 - protein, 9 aa, SARS-CoV-2
VADSGFAAY

SEQ ID NO: 959 - protein, 9 aa, SARS-CoV-2
LADEFIERY

SEQ ID NO: 960 - protein, 9 aa, SARS-CoV-2
YVDNGPDGY

SEQ ID NO: 961 - protein, 9 aa, SARS-CoV-2
TSDLATNAY

SEQ ID NO: 962 - protein, 9 aa, SARS-CoV-2
HTDFSSEIY

SEQ ID NO: 963 - protein, 9 aa, SARS-CoV-2
FSNSGSDLY

SEQ ID NO: 964 - protein, 9 aa, SARS-CoV-2
NTDGTTFTY

SEQ ID NO: 965 - protein, 9 aa, SARS-CoV-2
TTDDNALAY

SEQ ID NO: 966 - protein, 9 aa, SARS-CoV-2
QTDNALAYY

SEQ ID NO: 967 - protein, 9 aa, SARS-CoV-2
CTDDNALYY

SEQ ID NO: 968 - protein, 9 aa, SARS-CoV-2
VTDTPKYLY

SEQ ID NO: 969 - protein, 9 aa, SARS-CoV-2
STDTGVEHY

SEQ ID NO: 970 - protein, 9 aa, SARS-CoV-2
VVDDPIHFY

SEQ ID NO: 971 - protein, 9 aa, SARS-CoV-2
ATDNYITTY

SEQ ID NO: 972 - protein, 9 aa, SARS-CoV-2
TTDPSFLRY

SEQ ID NO: 973 - protein, 9 aa, SARS-CoV-2
ITDYKENSY

SEQ ID NO: 974 - protein, 9 aa, SARS-CoV-2
FTEQPIDLY

SEQ ID NO: 975 - protein, 9 aa, SARS-CoV-2
FADDLNQLY

SEQ ID NO: 976 - protein, 9 aa, SARS-CoV-2
ITEEVGHAY

SEQ ID NO: 977 - protein, 9 aa, SARS-CoV-2
STDRAFDIY

SEQ ID NO: 978 - protein, 9 aa, SARS-CoV-2
ASDYDYYRY

SEQ ID NO: 979 - protein, 9 aa, SARS-CoV-2
YSDVENPHY

SEQ ID NO: 980 - protein, 9 aa, SARS-CoV-2
STDGIADKY

SEQ ID NO: 981 - protein, 9 aa, SARS-CoV-2
NTDFVNEFY

SEQ ID NO: 982 - protein, 9 aa, SARS-CoV-2
LSDDANSTY

SEQ ID NO: 983 - protein, 9 aa, SARS-CoV-2
YTNDNTSRY

SEQ ID NO: 984 - protein, 9 aa, SARS-CoV-2
LADAGFIQY

SEQ ID NO: 985 - protein, 9 aa, SARS-CoV-2
VTDEMIAQY

SEQ ID NO: 986 - protein, 9 aa, SARS-CoV-2
FLVAGNVQL

SEQ ID NO: 987 - protein, 9 aa, SARS-CoV-2
ALLEDEFTV

SEQ ID NO: 988 - protein, 9 aa, SARS-CoV-2
LLLDDFVEI

SEQ ID NO: 989 - protein, 9 aa, SARS-CoV-2
SLFDFPLKL

SEQ ID NO: 990 - protein, 9 aa, SARS-CoV-2
GLNDNLLEL

SEQ ID NO: 991 - protein, 9 aa, SARS-CoV-2
YLDGADVTI

SEQ ID NO: 992 - protein, 9 aa, SARS-CoV-2
YMADLVYAL

SEQ ID NO: 993 - protein, 9 aa, SARS-CoV-2
KIFDGVPFV

SEQ ID NO: 994 - protein, 9 aa, SARS-CoV-2
SMQGAVINK

SEQ ID NO: 995 - protein, 9 aa, SARS-CoV-2
KLKKSLVAK

SEQ ID NO: 996 - protein, 9 aa, SARS-CoV-2
KLMPVVETK

SEQ ID NO: 997 - protein, 9 aa, SARS-CoV-2
AVYMGTLSY

SEQ ID NO: 998 - protein, 9 aa, SARS-CoV-2
LLKDPAVAK

SEQ ID NO: 999 - protein, 9 aa, SARS-CoV-2
RIAPGQTGK

SEQ ID NO: 1000 - protein, 9 aa, SARS-CoV-2
KIYKTPPIK

SEQ ID NO: 1001 - protein, 9 aa, SARS-CoV-2
AVLDMASLK

SEQ ID NO: 1002 - protein, 9 aa, SARS-CoV-2
STSAFVTVK

SEQ ID NO: 1003 - protein, 9 aa, SARS-CoV-2
AVANGVVLK

SEQ ID NO: 1004 - protein, 9 aa, SARS-CoV-2
VVNAANVYK

SEQ ID NO: 1005 - protein, 9 aa, SARS-CoV-2
STLGIEFLK

SEQ ID NO: 1006 - protein, 9 aa, SARS-CoV-2
TILPVSMTK

SEQ ID NO: 1007 - protein, 9 aa, SARS-CoV-2
VFFDGRVLF

SEQ ID NO: 1008 - protein, 9 aa, SARS-CoV-2
GYINVFAFF

SEQ ID NO: 1009 - protein, 9 aa, SARS-CoV-2
NYSGVVTTF

SEQ ID NO: 1010 - protein, 9 aa, SARS-CoV-2
YYAGEAANF

SEQ ID NO: 1011 - protein, 9 aa, SARS-CoV-2
MYMGSYEQF

SEQ ID NO: 1012 - protein, 9 aa, SARS-CoV-2
RFPNITNLF

SEQ ID NO: 1013 - protein, 9 aa, SARS-CoV-2
AYIAIPTNF

SEQ ID NO: 1014 - protein, 9 aa, SARS-CoV-2
SPSGVYQAM

SEQ ID NO: 1015 - protein, 9 aa, SARS-CoV-2
RPNTIKGSF

SEQ ID NO: 1016 - protein, 9 aa, SARS-CoV-2
KPRPPLNVF

SEQ ID NO: 1017 - protein, 9 aa, SARS-CoV-2
IPARARVEF

SEQ ID NO: 1018 - protein, 9 aa, SARS-CoV-2
RPQIGVVEF

SEQ ID NO: 1019 - protein, 9 aa, SARS-CoV-2
SPYNSQNAL

SEQ ID NO: 1020 - protein, 9 aa, SARS-CoV-2
APHGHVMVL

SEQ ID NO: 1021 - protein, 9 aa, SARS-CoV-2
VPANSTVSF

SEQ ID NO: 1022 - protein, 9 aa, SARS-CoV-2
HPNPKGFDL

SEQ ID NO: 1023 - protein, 9 aa, SARS-CoV-2
GPKQAGVTL

SEQ ID NO: 1024 - protein, 9 aa, SARS-CoV-2
VPGLPGTIL

SEQ ID NO: 1025 - protein, 9 aa, SARS-CoV-2
VPYYDTNVL

SEQ ID NO: 1026 - protein, 9 aa, SARS-CoV-2
TPIQPIGAL

SEQ ID NO: 1027 - protein, 9 aa, SARS-CoV-2
KPRGQGVPI

SEQ ID NO: 1028 - protein, 9 aa, SARS-CoV-2
SPFHPLAAL

SEQ ID NO: 1029 - protein, 9 aa, SARS-CoV-2
YPQNGLTSI

SEQ ID NO: 1030 - protein, 9 aa, SARS-CoV-2
KPRELKVTF

SEQ ID NO: 1031 - protein, 9 aa, SARS-CoV-2
TPFKKGAKL

SEQ ID NO: 1032 - protein, 9 aa, SARS-CoV-2
KPNELSRVL

SEQ ID NO: 1033 - protein, 9 aa, SARS-CoV-2
MPILTTRAL

SEQ ID NO: 1034 - protein, 9 aa, SARS-CoV-2
FPLANGQVF

SEQ ID NO: 1035 - protein, 9 aa, SARS-CoV-2
YPLNISDEF

SEQ ID NO: 1036 - protein, 9 aa, SARS-CoV-2
IPIPKDMTY

SEQ ID NO: 1037 - protein, 9 aa, SARS-CoV-2
YPANSIVRF

SEQ ID NO: 1038 - protein, 9 aa, SARS-CoV-2
IPMDSTVKY

SEQ ID NO: 1039 - protein, 9 aa, SARS-CoV-2
FPDGVKHVY

SEQ ID NO: 1040 - protein, 8 aa, SARS-CoV-2
SPDAVTAY

SEQ ID NO: 1041 - protein, 9 aa, SARS-CoV-2
LPNDDTLEY

SEQ ID NO: 1042 - protein, 9 aa, SARS-CoV-2
YPLTPNQEY

SEQ ID NO: 1043 - protein, 9 aa, SARS-CoV-2
LPIGINITF

SEQ ID NO: 1044 - protein, 9 aa, SARS-CoV-2
IPIGAGISY

SEQ ID NO: 1045 - protein, 9 aa, SARS-CoV-2
FPAPHGVVF

SEQ ID NO: 1046 - protein, 9 aa, SARS-CoV-2
SEVIGAVIL

SEQ ID NO: 1047 - protein, 9 aa, SARS-CoV-2
KEITVATTL

SEQ ID NO: 1048 - protein, 9 aa, SARS-CoV-2
TEVPANTVL

SEQ ID NO: 1049 - protein, 9 aa, SARS-CoV-2
REAVGTNLL

SEQ ID NO: 1050 - protein, 9 aa, SARS-CoV-2
IEYPIGDEL

SEQ ID NO: 1051 - protein, 9 aa, SARS-CoV-2
MEIDFELAM

SEQ ID NO: 1052 - protein, 9 aa, SARS-CoV-2
YELQTPIKL

SEQ ID NO: 1053 - protein, 9 aa, SARS-CoV-2
AEVGPEHSL

SEQ ID NO: 1054 - protein, 9 aa, SARS-CoV-2
GEQKSILSL

SEQ ID NO: 1055 - protein, 9 aa, SARS-CoV-2
KEFLEGETL

SEQ ID NO: 1056 - protein, 9 aa, SARS-CoV-2
REVGFVVGL

SEQ ID NO: 1057 - protein, 9 aa, SARS-CoV-2
QEVRGTTVL

SEQ ID NO: 1058 - protein, 9 aa, SARS-CoV-2
YENSPFHPL

SEQ ID NO: 1059 - protein, 9 aa, SARS-CoV-2
LEMELTPVV

SEQ ID NO: 1060 - protein, 9 aa, SARS-CoV-2
YEFNQHEVL

SEQ ID NO: 1061 - protein, 9 aa, SARS-CoV-2
YEFKKGVQI

SEQ ID NO: 1062 - protein, 9 aa, SARS-CoV-2
HEFSQHTML

SEQ ID NO: 1063 - protein, 9 aa, SARS-CoV-2
YEYTPHTVL

SEQ ID NO: 1064 - protein, 9 aa, SARS-CoV-2
VEAEVQIRL

SEQ ID NO: 1065 - protein, 9 aa, SARS-CoV-2
SEAGVSGRW

SEQ ID NO: 1066 - protein, 9 aa, SARS-CoV-2
QELTDYKHW

SEQ ID NO: 1067 - protein, 9 aa, SARS-CoV-2
SEVTPSGTW

SEQ ID NO: 1068 - protein, 9 aa, SARS-CoV-2
TENPHLMGW

SEQ ID NO: 1069 - protein, 9 aa, SARS-CoV-2
RELNGGARY

SEQ ID NO: 1070 - protein, 10 aa, SARS-CoV-2
TTDPSFLGRY

SEQ ID NO: 1071 - protein, 365 aa, Homo sapiens
MAVMAPRTLLLLLSGALALTQTWAGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 1072 - protein, 365 aa, Homo sapiens
MAVMAPRTLLLLLSGALALTQTWAGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 1073 - protein, 365 aa, Homo sapiens [sequence omitted]

SEQ ID NO: 1074 - protein, 365 aa, Homo sapiens [sequence omitted]

SEQ ID NO: 1075 - protein, 362 aa, Homo sapiens
MLVMAPRTVLLLLSAALALTETWAGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 1076 - protein, 365 aa, Homo sapiens
MRVMAPRALLLLLSGGLALTETWACSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCK

SEQ ID NO: 1077 - protein, 944 aa, synthetic construct
  REGION 1..944: note = synthetic sequence
RLQSLQTYVGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 1078 - protein, 944 aa, synthetic construct
  REGION 1..944: note = synthetic sequence
YLQPRTFLLGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 1079 - protein, 284 aa, synthetic construct
  REGION 1..284: note = synthetic sequence
  VARIANT 89: note = Xaa can be any naturally occurring amino acid
  VARIANT 107: note = Xaa can be any naturally occurring amino acid
  VARIANT 157..158: note = Xaa can be any naturally occurring amino acid
  VARIANT 255: note = Xaa can be any naturally occurring amino acid
  VARIANT 267: note = Xaa can be any naturally occurring amino acid
GSHSLKYFHTSVSRPGRGEPRFISVGYVDDTQFVRFDNDAASPRMVPRAPWMEQEGSEYWDRETRSARDTAQIFRVNLRTLRGYYNQSXAGSHTLQWMHGCELGPDXRFLRGYEQFAYDGKDYLTLNEDLRSWTAVDTAAQISEQKSNDASEAEHQXXYLEDTCVEWLHKYLEKGKETLLHLEPPKTHVTHHPISDHEATLRCWALGFYPAEITLTWQQDGEGHTQDTELVETRPAGDGTFQKWAAVVVPSGEEXRYTCHVQHEGLXEPVTLRWKPASQPTIPI

SEQ ID NO: 1080 - protein, 284 aa, synthetic construct
  REGION 1..284: note = synthetic sequence
  VARIANT 7: note = Xaa can be any naturally occurring amino acid
  VARIANT 50: note = Xaa can be any naturally occurring amino acid
  VARIANT 251: note = Xaa can be any naturally occurring amino acid
  VARIANT 278: note = Xaa can be any naturally occurring amino acid
GSHSLRXFSTAVSRPGRGEPRYIAVEYVDDTQFLRFDSDAAIPRMEPREXWVEQEGPQYWEWTTGYAKANAQTDRVALRNLLRRYNQSEAGSHTLQGMNGCDMGPDGRLLRGYHQHAYDGKDYISLNEDLRSWTAADTVAQITQRFYEAEEYAEEFRTYLEGECLELLRRYLENGKETLQRADPPKAHVAHHPISDHEATLRCWALGFYPAEITLTWQRDGEEQTQDTELVETRPAGDGTFQKWAAVVVPXGEEQRYTCHVQHEGLPQPLILRWEQSXQPTIPI

SEQ ID NO: 1081 - protein, 284 aa, synthetic construct
  REGION 1..284: note = synthetic sequence
  VARIANT 13: note = Xaa can be any naturally occurring amino acid
  VARIANT 27: note = Xaa can be any naturally occurring amino acid
  VARIANT 31: note = Xaa can be any naturally occurring amino acid
  VARIANT 34: note = Xaa can be any naturally occurring amino acid
  VARIANT 54: note = Xaa can be any naturally occurring amino acid
  VARIANT 81: note = Xaa can be any naturally occurring amino acid
  VARIANT 100: note = Xaa can be any naturally occurring amino acid
  VARIANT 104..105: note = Xaa can be any naturally occurring amino acid
  VARIANT 110: note = Xaa can be any naturally occurring amino acid
  VARIANT 159: note = Xaa can be any naturally occurring amino acid
  VARIANT 169: note = Xaa can be any naturally occurring amino acid
  VARIANT 171: note = Xaa can be any naturally occurring amino acid
  VARIANT 178: note = Xaa can be any naturally occurring amino acid
  VARIANT 185: note = Xaa can be any naturally occurring amino acid
  VARIANT 219: note = Xaa can be any naturally occurring amino acid
  VARIANT 258: note = Xaa can be any naturally occurring amino acid
  VARIANT 275: note = Xaa can be any naturally occurring amino acid
GSHSMRYFSAAVXRPGRGEPRFIAMGXVDDXQFXRFDSDSACPRMEPRAPWVEXEGPEYWEEETRNTKAHAQTDRMNLQTXRGYYNQSEASSHTLQWMIXCDLXXDGRLXRGYEQYAYDGKDYLALNEDLRSWTAADTAAQISKRKCEAANVAEQRRAXLEGTCVEWLXRXLENGKEXLQRADPXKTHVTHHPVFDYEATLRCWALGFYPAEIILTWQXDGEDQTQDVELVETRPAGDGTFQKWAAVVVPSGEEQRYXCHVQHEGLPEPLMLRWXQSSLPTIPI

SEQ ID NO: 1082 - protein, 286 aa, synthetic construct
  REGION 1..286: note = synthetic sequence
  VARIANT 89: note = X is K or E
  VARIANT 108: note = X is R or G
  VARIANT 158: note = X is R or G
  VARIANT 159: note = X is A or V
  VARIANT 256: note = X is Q or P
  VARIANT 269: note = X is P or S
GSHSLKYFHTSVSRPGRGEPRFISVGYVDDTQFVRFDNDAASPRMVPRAPWMEQEGSEYWDRETRSARDTAQIFRVNLRTLRGYYNQSXLAGSHTLQWMHGCELGPDXRFLRGYEQFAYDGKDYLTLNEDLRSWTAVDTAAQISEQKSNDASEAEHQXXYLEDTCVEWLHKYLEKGKETLLHLEPPKTHVTHHPISDHEATLRCWALGFYPAEITLTWQQDGEGHTQDTELVETRPAGDGTFQKWAAVVVPSGEEXSRYTCHVQHEGLXEPVTLRWKPASQPTIPI

SEQ ID NO: 1083 - protein, 285 aa, synthetic construct
  REGION 1..285: note = synthetic sequence
  VARIANT 7: note = X is Y or F
  VARIANT 51: note = X is P or Q
  VARIANT 252: note = X is S or P
  VARIANT 279: note = X is P or L
GSHSLRXLFSTAVSRPGRGEPRYIAVEYVDDTQFLRFDSDAAIPRMEPREXWVEQEGPQYWEWTTGYAKANAQTDRVALRNLLRRYNQSEAGSHTLQGMNGCDMGPDGRLLRGYHQHAYDGKDYISLNEDLRSWTAADTVAQITQRFYEAEEYAEEFRTYLEGECLELLRRYLENGKETLQRADPPKAHVAHHPISDHEATLRCWALGFYPAEITLTWQRDGEEQTQDTELVETRPAGDGTFQKWAAVVVPXGEEQRYTCHVQHEGLPQPLILRWEQSXQPTIPI

SEQ ID NO: 1084 - protein, 286 aa, synthetic construct
  REGION 1..286: note = synthetic sequence
  VARIANT 13: note = X is S or F
  VARIANT 27: note = X is Y or H
  VARIANT 31: note = X is T, S or M
  VARIANT 34: note = X is L or V
  VARIANT 54: note = X is Q or R
  VARIANT 82: note = X is P or L
  VARIANT 101: note = X is G or D
  VARIANT 105: note = X is G or V
  VARIANT 106: note = X is S or C
  VARIANT 111: note = X is L or I
  VARIANT 160: note = X is Y or H
  VARIANT 170: note = X is H or R
  VARIANT 172: note = X is Y or H
  VARIANT 179: note = X is M or T
  VARIANT 186: note = X is P or A
  VARIANT 221: note = X is R, W or Q
  VARIANT 260: note = X is T or M
  VARIANT 277: note = X is K or E
GSHSMRYFSAAVXRPGRGEPRFIAMGXVDDXQFXRFDSDSACPRMEPRAPWVEXSEGPEYWEEETRNTKAHAQTDRMNLQTXRGYYNQSEASSHTLQWMIXCDLXXDGRLXRGYEQYAYDGKDYLALNEDLRSWTAADTAAQISKRKCEAANVAEQRRAXLEGTCVEWLXRXLENGKEXLQRADPXSKTHVTHHPVFDYEATLRCWALGFYPAEIILTWQXDGEDQTQDVELVETRPAGDGTFQKWAAVVVPSGEEQRYXCHVQHEGLPEPLMLRWXQSSLPTIPI

SEQ ID NO: 1085 - protein, 275 aa, Homo sapiens
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWK

SEQ ID NO: 1086 - protein, 275 aa, Homo sapiens
GSHSLKYFHTSVSRPGRGEPRFISVGYVDDTQFVRFDNDAASPRMVPRAPWMEQEGSEYWDRETRSARDTAQIFRVNLRTLRGCYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPCGDGTFQKWAAVVVPSGEEQRYTCHVQHEGLPEPVTLRWK

SEQ ID NO: 1087 - protein, 275 aa, Homo sapiens
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWK

SEQ ID NO: 1088 - protein, 275 aa, Homo sapiens
GSHSLKYFHTSVSRPGRGEPRFISVGYVDDTQFVRFDNDAASPRMVPRAPWMEQEGSEYWDRETRSARDTAQIFRVNLRTLRGCYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPCGDGTFQKWAAVVVPSGEEQRYTCHVQHEGLPEPVTLRWK

SEQ ID NO: 1089 - protein, 275 aa, Homo sapiens
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWK

SEQ ID NO: 1090 - protein, 275 aa, Homo sapiens
GSHSMRYFSAAVSRPGRGEPRFIAMGYVDDTQFVRFDSDSACPRMEPRAPWVEQEGPEYWEEETRNTKAHAQTDRMNLQTLRGCYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPCGDGTFQKWAAVVVPSGEEQRYTCHVQHEGLPEPLMLRWK

SEQ ID NO: 1091 - protein, 275 aa, Homo sapiens
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLIRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWK

SEQ ID NO: 1092 - protein, 275 aa, Homo sapiens
GSHSMRYFSAAVSRPGRGEPRFIAMGYVDDTQFVRFDSDSACPRMEPRAPWVEQEGPEYWEEETRNTKAHAQTDRMNLQTLRGCYNQSEASSHTLQWMIGCDLGSDGRLIRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPCGDGTFQKWAAVVVPSGEEQRYTCHVQHEGLPEPLMLRWK

SEQ ID NO: 1093 - protein, 9 aa, synthetic construct
  REGION 1..9: note = synthetic sequence
GILGFVFTL

SEQ ID NO: 1094 - protein, 9 aa, synthetic construct
  REGION 1..9: note = synthetic sequence
HPVGEADYF

(19) United States
(12) Patent Application Publication
(10) Pub. No.: US 2023/0338509 A1
(43) Pub. Date: Oct. 26, 2023

(54) T CELL MODULATORY POLYPEPTIDES AND METHODS OF USE THEREOF

(71) Applicant: Cue Biopharma, Inc., Boston, MA (US)

(72) Inventor: Anish Suri, Boston, MA (US)

(21) Appl. No.: 17/822,584

(22) Filed: Aug. 26, 2022

Related U.S. Application Data
(63) Continuation of application No. PCT/US2021/023722, filed on Mar. 23, 2021.
(60) Provisional application No. 62/993,900, filed on Mar. 24, 2020.

(51) Int. Cl. A61K 39/215

(52) U.S. Cl. CPC A61K 39/215; A61K 2039/605

(57) ABSTRACT

The present disclosure provides T cell modulatory polypeptides (TMPs) that comprise an immunomodulatory polypeptide, class I HLA polypeptides (a class I HLA heavy chain polypeptide and a β2 microglobulin polypeptide), and a Betacoronavirus (e.g., a SARS-CoV-2) peptide that presents an epitope to a T-cell receptor. A TMP is useful for modulating the activity of a T cell, and for modulating an immune response in an individual.

### CROSS-REFERENCE

This application is a continuation of PCT Application No. PCT/US2021/023722, filed Mar. 23, 2021, which_claims the benefit of U.S. Provisional Pat. Application No. 62/993,900, filed Mar. 24, 2020, which application is incorporated herein by reference in its entirety.

### INCORPORATION BY REFERENCE OF SEQUENCE LISTING PROVIDED AS A SEQUENCE LISTING XML FILE

A Sequence Listing is provided herewith as a Sequence Listing XML, “CUEB-131CON_SEQ_LIST” created on Jun. 26, 2023 and having a size of 1,231,190 bytes. The contents of the Sequence Listing XML are incorporated by reference herein in their entirety.

### INTRODUCTION

An adaptive immune response involves the engagement of the T cell receptor (TCR), present on the surface of a T cell, with a small peptide antigen non-covalently presented on the surface of an antigen presenting cell (APC) by a major histocompatibility complex (MHC; also referred to in humans as a human leukocyte antigen (HLA) complex). This engagement represents the immune system’s targeting mechanism and is a requisite molecular interaction for T cell modulation (activation or inhibition) and effector function. Following epitope-specific cell targeting, the targeted T cells are activated through engagement of costimulatory proteins found on the APC with counterpart costimulatory proteins the T cells. Both signals - epitope/TCR binding and engagement of APC costimulatory proteins with T cell costimulatory proteins - are required to drive T cell specificity and activation or inhibition. The TCR is specific for a given epitope; however, the costimulatory protein not epitope specific and instead is generally expressed on all T cells or on large T cell subsets.

### SUMMARY

The present disclosure provides T cell modulatory polypeptides (TMPs) that comprise an immunomodulatory polypeptide, class I HLA polypeptides (a class I HLA heavy chain polypeptide and a β2 microglobulin polypeptide), and a Betacoronavirus (e.g., a SARS-CoV-2) peptide that presents an epitope to a T-cell receptor. A TMP is useful for modulating the activity of a T cell, and for modulating an immune response in an individual.

### BRIEF DESCRIPTION OF THE DRAWINGS

FIGS. 1A-1F are schematic depictions of various TMPs of the present disclosure.

FIGS. 2A-2F are schematic depictions of various disulfide-linked TMPs of the present disclosure.

FIGS. 3A-3G provide amino acid sequences of immunoglobulin Fc polypeptides.

FIG. 4 provides a multiple amino acid sequence alignment of beta-2 microglobulin (β2M) precursors (i.e., including the leader sequence) from Homo sapiens (NP_004039.1; SEQ ID NO:385), Pan troglodytes (NP_001009066.1; SEQ ID NO:385), Macaca mulatta (NP_001040602.1; SEQ ID NO:386), Bos taurus (NP_776318.1; SEQ ID NO:387) and Mus musculus (NP_033865.2; SEQ ID NO:388). Amino acids 1-20 are a signal peptide.

FIGS. 5A-5C provide amino acid sequences of full-length human HLA heavy chains of alleles A*0101, A*1101, A*2402, and A*3303 (FIG. 7A); full-length human HLA heavy chain of allele B*0702 (FIG. 7B); and a full-length human HLA-C heavy chain (FIG. 7C).

FIG. 6 provides an alignment of eleven mature MHC class I heavy chain amino acid sequences without their leader sequences, transmembrane domains, and intracellular domains.

FIGS. 7A-7B provide an alignment of HLA-A heavy chain amino acid sequences (FIG. 7A) and a consensus sequence (FIG. 7B).

FIGS. 8A-8B provide an alignment of HLA-B heavy chain amino acid sequences (FIG. 8A) and a consensus sequence (FIG. 8B; SEQ ID NO:159).

FIGS. 9A-9B provide an alignment of HLA-C heavy chain amino acid sequences (FIG. 9A) and a consensus sequence (FIG. 9B).

FIG. 10 provides a consensus amino acid sequence for each of HLA-E, -F, and -G heavy chains. Variable amino acid (aa) positions are indicated as “X” residues sequentially numbered; the locations of amino acids 84, 139, and 236 are double underlined.

FIG. 11 provides an alignment of consensus amino acid sequences for HLA-A, -B, -C, -E, -F, and -G.

FIGS. 12A-12D provide schematic depictions of multiple disulfide-linked TMP of the present disclosure.

FIGS. 13A-13J provide amino acid sequences of SARS-CoV2-encoded polypeptides.

FIG. 14 provides CD8 T cell epitopes of SARS-CoV2-encoded polypeptides and primary HLA class I heavy chain allele restriction.

FIG. 15 provides CD8 T cell epitopes of SARS-CoV2-encoded polypeptides and HLA class I heavy chain allele restriction.

FIGS. 16A-16C provide schematic depictions of examples of configurations of disulfide-linked TMPs of the present disclosure.

FIG. 17 provides schematic depictions of examples of positions of immunomodulatory polypeptides in “split chain” TMPs of the present disclosure.

FIG. 18 provides schematic depictions of examples of positions of immunomodulatory polypeptides in “single-chain” TMPs of the present disclosure.

FIGS. 19A-19B provide amino acid sequences of exemplary TMPs.

FIGS. 20A-20D provide amino acid sequences of HLA-E heavy chains.

FIGS. 21A-21D provide amino acid sequences of HLA-G heavy chains.

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various convenient methods and computer programs (e.g., BLAST, T-COFFEE, MUSCLE, MAFFT, etc.), available over the world wide web at sites including ncbi.nlm.nili.gov/BLAST, ebi.ac.uk/Tools/msa/tcoffee/, ebi.ac.uk/Tools/msa/muscle/, mafft.cbrc.jp/alignment/software/. See, e.g., Altschul et al. (1990), J. Mol. Biol. 215:403-10.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

The term “immunological synapse” or “immune synapse” as used herein generally refers to the natural interface between two interacting immune cells of an adaptive immune response including, e.g., the interface between an antigen-presenting cell (APC) or target cell and an effector cell, e.g., a lymphocyte, an effector T cell, a natural killer cell, and the like. An immunological synapse between an APC and a T cell is generally initiated by the interaction of a T cell antigen receptor and major histocompatibility complex molecules, e.g., as described in Bromley et al., Annu Rev Immunol. 2001;19:375-96; the disclosure of which is incorporated herein by reference in its entirety.

“T cell” includes all types of immune cells expressing CD3, including T-helper cells (CD4⁺ cells), cytotoxic T-cells (CD8⁺ cells), T-regulatory cells (Treg), and NK-T cells.

The term “immunomodulatory polypeptide” (also referred to as a “co-stimulatory polypeptide”), as used herein, includes a polypeptide on an antigen presenting cell (APC) (e.g., a dendritic cell, a B cell, and the like) that specifically binds a cognate co-immunomodulatory polypeptide on a T cell, thereby providing a signal which, in addition to the primary signal provided by, for instance, binding of a TCR/CD3 complex with a major histocompatibility complex (MHC) polypeptide loaded with peptide, mediates a T cell response, including, but not limited to, proliferation, activation, differentiation, and the like. An immunomodulatory polypeptide can include, but is not limited to, a cytokine (e.g., IL-2), CD7, B7-1 (CD80), B7-2 (CD86), PD-L1, PD-L2, 4-1BBL, OX40L, Fas ligand (FasL), inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3.

As noted above, an “immunomodulatory polypeptide” (also referred to herein as a “MOD”) specifically binds a cognate co-immunomodulatory polypeptide on a T cell.

An “immunomodulatory domain” (“MOD”) of a TMP of the present disclosure binds a cognate co-immunomodulatory polypeptide, which may be present on a target T cell.

“Heterologous,” as used herein, means a nucleotide or polypeptide that is not found in the native nucleic acid or protein, respectively.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system.

The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

As used herein, the term “affinity” refers to the equilibrium constant for the reversible binding of two agents (e.g., an antibody and an antigen) and is expressed as a dissociation constant (K_{D}). As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” as used herein (e.g., with reference to binding of a TMP to a polypeptide (e.g., a T-cell receptor) on a T cell), refers to a non-covalent interaction between two molecules. Non-covalent binding refers to a direct association between two molecules, due to, for example, electrostatic, hydrophobic, ionic, and/or hydrogen-bond interactions, including interactions such as salt bridges and water bridges. “Affinity” refers to the strength of non-covalent binding, increased binding affinity being correlated with a lower K_{D}. “Non-specific binding” generally refers to binding of a ligand to a moiety other than its designated binding site or receptor. “Covalent binding” or “covalent bond,” as used herein, refers to the formation of one or more covalent chemical binds between two different molecules.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, i.e., arresting its development; and/or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired. Mammals include, e.g., humans, non-human primates, rodents (e.g., rats; mice), lagomorphs (e.g., rabbits), ungulates (e.g., cows, sheep, pigs, horses, goats, and the like), etc.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to a “TMP” includes a plurality of such polypeptides and reference to “the immunomodulatory polypeptide” includes reference to one or more immunomodulatory polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides multimeric T-cell modulatory polypeptides (TMPs) that comprise an immunomodulatory polypeptide and that comprise an epitope-presenting peptide. A TMP is useful for modulating the activity of a T cell, and for modulating an immune response in an individual.

### T-Cell Modulatory Polypeptides

The present disclosure provides TMPs comprising: a) a first polypeptide; and b) a second polypeptide, wherein the TMP comprises: i) a Betacoronavirus (e.g., SARS-CoV-2) peptide (defined below) that, when bound to major histocompatibility complex (MHC) polypeptides, presents an epitope to a T-cell receptor (TCR); ii) a first MHC polypeptide; iii) a second MHC polypeptide; and iv) one or more MODs; and optionally an immunoglobulin (Ig) Fc polypeptide or a non-Ig scaffold. TMP comprising a first polypeptide and a second polypeptide is also referred to herein as a “split-chain TMP” or a “heterodimeric TMP.” A TMP of the present disclosure is in some cases a single polypeptide chain; such a TMP is also referred to herein as a “single-chain TMP”.

For simplicity, the disclosure refers to “SARS-CoV-2”; however, it should be understood that disclosure relating to “SARS-CoV-2” can also apply to other members of the Betacoronavirus family.

In general, a T-cell modulatory polypeptide (TMP) comprises a polypeptide that preferentially binds to and activates target T cells bearing a T cell receptor (TCR) specific for an antigen of interest. For example, a TMP can comprise at least one heterodimer comprising 2 polypeptide chains: a) a first polypeptide comprising: i) a peptide epitope (e.g., a peptide that is at least 4 amino acids in length (e.g., from 4 amino acids to about 25 amino acids in length); and ii) first MHC polypeptide; b) a second polypeptide comprising a second MHC polypeptide, and c) at least one MOD, where the first and/or the second polypeptide comprises the MOD. A TMP also may be referred to as a “synTac” or an “Immuno-STAT™.”

The present disclosure provides a TMP, wherein the TMP is a heterodimer comprising: a) a first polypeptide comprising a first MHC polypeptide; and b) a second polypeptide comprising a second MHC polypeptide, wherein the first polypeptide or the second polypeptide comprises a SARS-CoV-2 peptide (e.g., a SARS-CoV-2 peptide having a length of a least 4 amino acids (e.g., from 4 amino acids to about 25 amino acids); where the SARS-CoV-2 peptide, when bound to an MHC complex, presents an epitope to a T-cell receptor); wherein the first polypeptide and/or the second polypeptide comprises one or more immunomodulatory polypeptides (“MODs”) that can be the same or different from one another; and optionally an Ig F c polypeptide or a non-Ig scaffold.

The present disclosure provides a TMP comprising a heterodimeric polypeptide comprising: a) a first polypeptide comprising: i) a SARS-CoV-2 peptide; and ii) a first MHC polypeptide; b) a second polypeptide comprising a second MHC polypeptide; and c) at least one MOD, where the first and/or the second polypeptide comprises the at least one (i.e., one or more) MOD. Optionally, the first or the second polypeptide comprises an Ig Fc polypeptide or a non-Ig scaffold. Optionally, at least one of the one or more MODS is a variant MOD that exhibits reduced affinity to a cognate co-immunomodulatory polypeptide (“co-MOD”) compared to the affinity of a corresponding wild-type MOD for the cognate co-MOD.

The present disclosure thus provides a TMP, wherein the TMP is:
A) a heterodimer comprising: a) a first polypeptide comprising a first MHC polypeptide; and b) a second polypeptide comprising a second MHC polypeptide, wherein the first polypeptide or the second polypeptide comprises a SARS-CoV-2 peptide; wherein the first polypeptide and/or the second polypeptide comprises one or more MODs that can be the same or different, and wherein at least one of the one or more MODs may be a wild-type MOD or a variant of a wild-type MOD; and wherein the first polypeptide or the second polypeptide optionally comprises an Ig Fc polypeptide or a non-Ig scaffold; or
B) a heterodimer comprising: a) a first polypeptide comprising a first MHC polypeptide; and b) a second polypeptide comprising a second MHC polypeptide, wherein the first polypeptide or the second polypeptide comprises a SARS-CoV-2 peptide; wherein the first polypeptide and/or the second polypeptide comprises one or more MODs that can be the same or different,
   wherein at least one of the one or more MODs is a variant MOD that exhibits reduced affinity to a cognate co-MOD compared to the affinity of a corresponding wild-type MOD for the cognate co-MOD; and
   wherein the first polypeptide or the second polypeptide optionally comprises an Ig Fc polypeptide or a non-Ig scaffold; or
C) a heterodimer comprising: a) a first polypeptide comprising, in order from N-terminus to C-terminus: i) a SARS-CoV-2 peptide; ii) a first MHC polypeptide; and b) a second polypeptide comprising, in order from N-terminus to C-terminus: i) a second MHC polypeptide; and ii) optionally an immunoglobulin (Ig) Fc polypeptide or a non-Ig scaffold, wherein the TMP comprises one or more MODs that can be the same or different, wherein at least one of the one or more MODs is: A) at the C-terminus of the first polypeptide; B) at the N-terminus of the second polypeptide; C) at the C-terminus of the second polypeptide; or D) at the C-terminus of the first polypeptide and at the N-terminus of the second polypeptide, and wherein at least one of the one or more MODs may be a wild-type MOD or a variant of a wild-type MOD; and
   optionally wherein at least one of the one or more MODs is a variant MOD that binds to its cognate co-MOD and exhibits reduced affinity to a cognate co-MOD compared to the affinity of a corresponding wild-type MOD for the cognate co-MOD.

The present disclosure provides a TMP comprising: a) a first polypeptide comprising, in order from N-terminus to C-terminus: i) a SARS-CoV-2 peptide; ii) a first MHC polypeptide; and b) a second polypeptide cowmprising, in order from N-terminus to C-terminus: i) a second MHC polypeptide; and ii) optionally an Ig Fc polypeptide or a non-Ig scaffold. A TMP of the present disclosure comprises one or more MODs, wherein at least one of the one or more MODs is: A) at the C-terminus of the first polypeptide; B) at the N-terminus of the second polypeptide; C) at the C-terminus of the second polypeptide; or D) at the C-terminus of the first polypeptide and at the N-terminus of the second polypeptide. At least one of the one or more MODs is a variant MOD that exhibits reduced affinity to a cognate co-MOD compared to the affinity of a corresponding wild-type MOD for the cognate co-MOD.

As noted above, in some cases, a TMP of the present disclosure is a single polypeptide chain. A single-chain TMP comprises: i) a SARS-CoV-2 peptide that, when bound to MHC polypeptides, presents an epitope to a TCR; ii) a first MHC polypeptide; iii) a second MHC polypeptide; and iv) one or more MODs; and optionally an Ig Fc polypeptide or a non-Ig scaffold.

In some cases, a single-chain TMP comprises, in order from N-terminus to C-terminus: i) a SARS-CoV-2 peptide; ii) a first MHC polypeptide; iii) a second MHC polypeptide; iv) one or more MODs; and v) an Ig Fc polypeptide. In some cases, a single-chain TMP comprises, in order from N-terminus to C-terminus: i) a SARS-CoV-2 peptide; ii) a first class I MHC polypeptide; iii) a second class I MHC polypeptide; iv) one or more MODs; and v) an Ig Fc polypeptide. In some cases, a single-chain TMP of the present disclosure comprises, in order from N-terminus to C-terminus: i) a SARS-CoV-2 peptide; ii) a β2M polypeptide; iii) a class I MHC heavy chain polypeptide; iv) one or more MODs; and v) an Ig Fc polypeptide.

In some cases, a single-chain TMP comprises, in order from N-terminus to C-terminus: i) a SARS-CoV-2 peptide; ii) a first MHC polypeptide; iii) a second MHC polypeptide; iv) an Ig Fc polypeptide; and v) one or more MODs. In some cases, a single-chain TMP comprises, in order from N-terminus to C-terminus: i) a SARS-CoV-2 peptide; ii) a first class I MHC polypeptide; iii) a second class I MHC polypeptide; iv) an Ig Fc polypeptide; and v) one or more MODs. In some cases, a single-chain TMP of the present disclosure comprises, in order from N-terminus to C-terminus: i) a SARS-CoV-2 peptide; ii) a β2M polypeptide; iii) a class I MHC heavy chain polypeptide; iv) an Ig Fc polypeptide; and v) one or more MODs.

In some cases, a single-chain TMP of the present disclosure comprises, in order from N-terminus to C-terminus: i) one or more MODs; ii) a SARS-CoV-2 peptide; iii) a first MHC polypeptide; iv) a second MHC polypeptide; and v) an Ig Fc polypeptide. In some cases, a single-chain TMP comprises, in order from N-terminus to C-terminus: i) one or more MODs; ii) a SARS-CoV-2 peptide; iii) a first class I MHC polypeptide; iv) a second class I MHC polypeptide; and v) an Ig Fc polypeptide. In some cases, a single-chain TMP of the present disclosure comprises, in order from N-terminus to C-terminus: i) one or more MODs; ii) a SARS-CoV-2 peptide; iii) a β2M polypeptide; iv) a class I MHC heavy chain polypeptide; and v) an Ig Fc polypeptide.

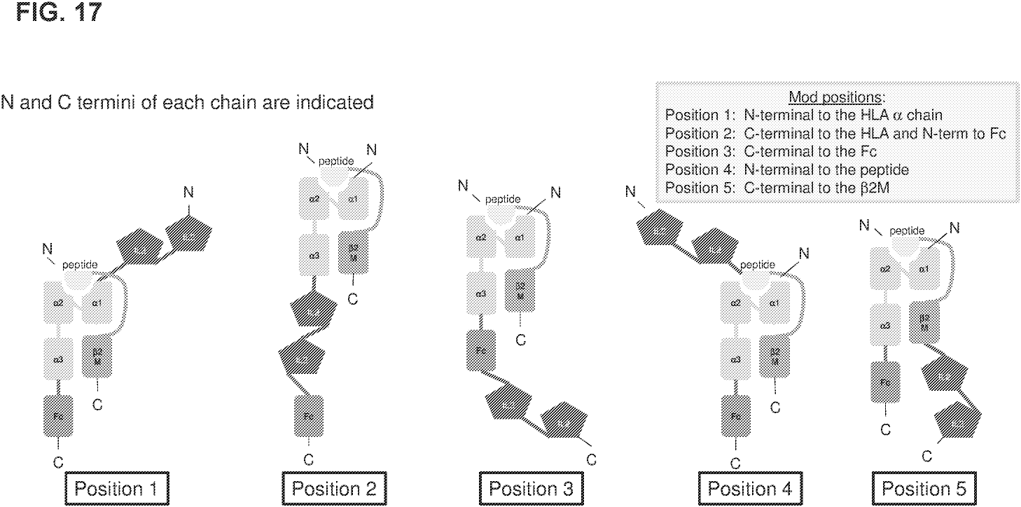

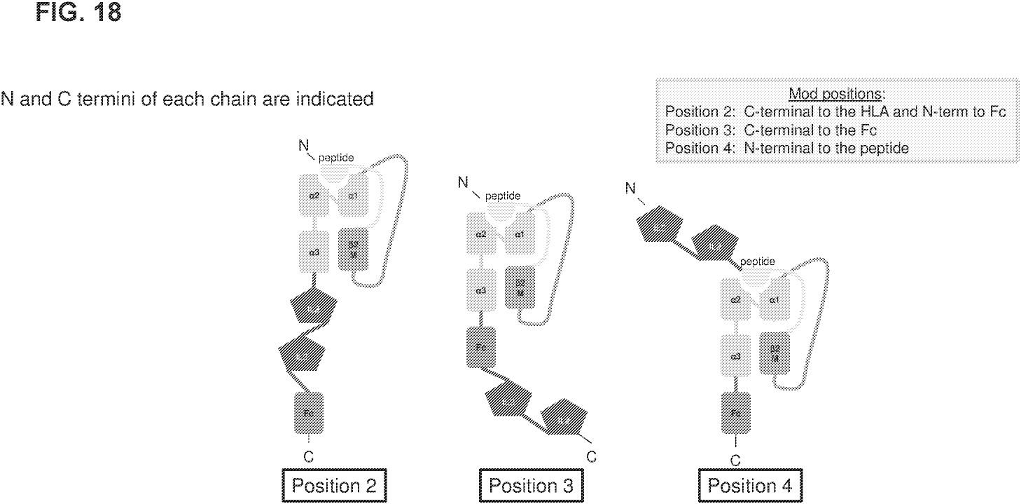

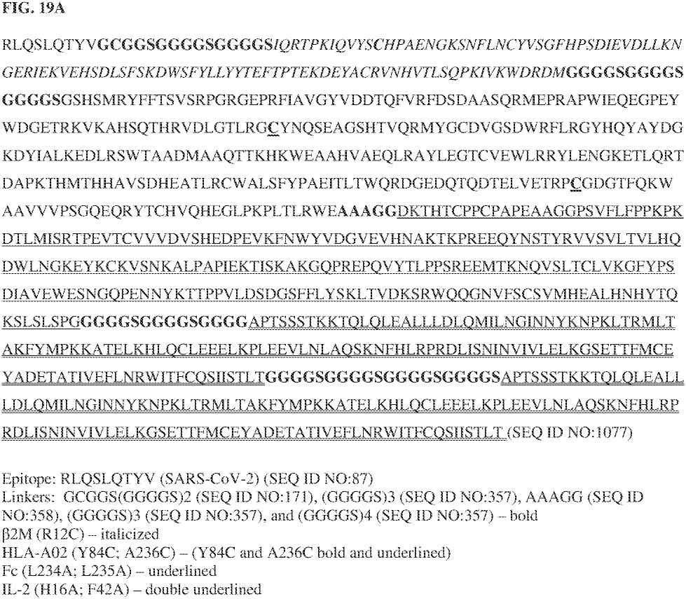

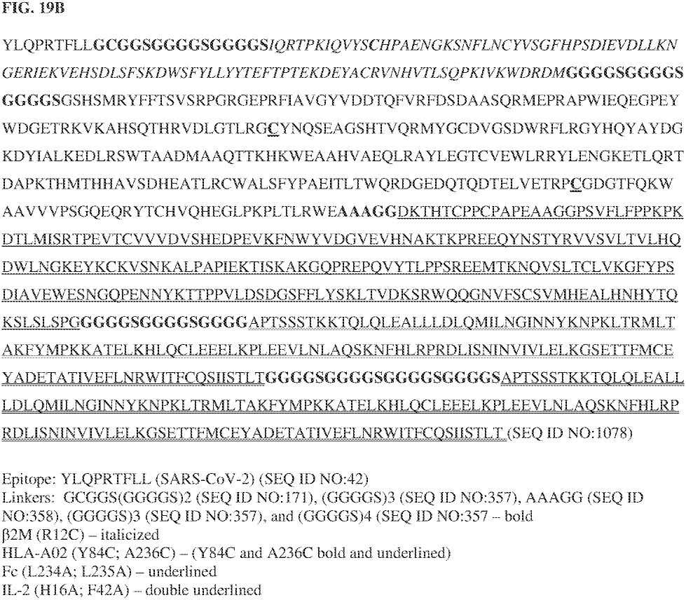

Amino acid sequences of non-limiting examples of single-chain TMPs are provided in FIG. 19A and FIG. 19B.

### Betacoronavirus Peptides

As discussed above, a TMP of the present disclosure comprises a Betacoronavirus (e.g., SARS-CoV-2) peptide that is typically at least about 4 amino acids in length, and presents a SARS-CoV-2 epitope to a T cell when in an MHC/peptide complex (e.g., an HLA/peptide complex).

A SARS-CoV-2 peptide present in a TMP can have a length of at least 4 amino acids, e.g., from 4 amino acids to about 25 amino acids in length (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length).

A SARS-CoV-2 epitope present in a TMP is a peptide specifically bound by a T-cell, i.e., the epitope is specifically bound by an epitope-specific T cell. An epitope-specific T cell binds an epitope having a reference amino acid sequence, but does not substantially bind an epitope that differs from the reference amino acid sequence. For example, an epitope-specific T cell binds an epitope having a reference amino acid sequence, and binds an epitope that differs from the reference amino acid sequence, if at all, with an affinity that is less than 10⁻⁶ M, less than 10⁻⁵ M, or less than 10⁻⁴ M. An epitope-specific T cell can bind an epitope for which it is specific with an affinity of at least 10⁻⁷ M, at least 10⁻⁸ M, at least 10⁻⁹ M, or at least 10⁻¹⁰ M.

The peptide epitope present in a TMP of the present disclosure is a peptide of a Betacoronavirus-encoded polypeptide. In some cases, the peptide epitope is a SARS-CoV-2 peptide (i.e., a peptide of a SARS-CoV-2-encoded polypeptide). In some cases, the peptide epitope is a SARS-CoV-2 peptide from a SARS-CoV-2-encoded surface glycoprotein. In some cases, the peptide epitope is a SARS-CoV-2 peptide from a SARS-CoV-2-encoded membrane glycoprotein. In some cases, the peptide epitope is a SARS-CoV-2 peptide from a SARS-CoV-2-encoded nucleocapsid phosphoprotein.

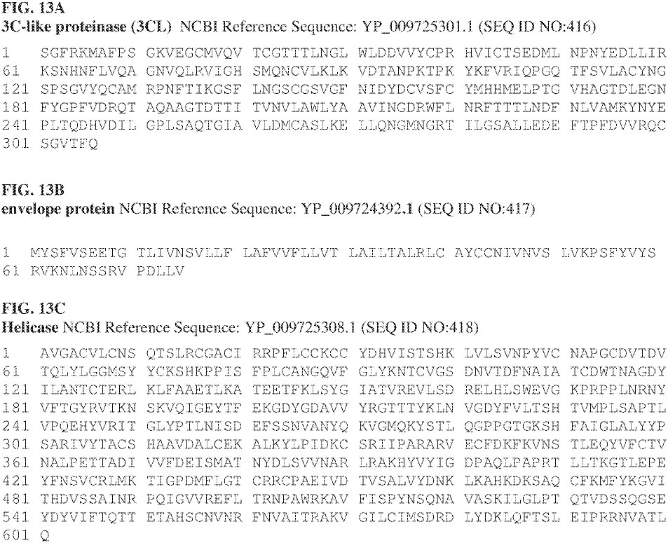

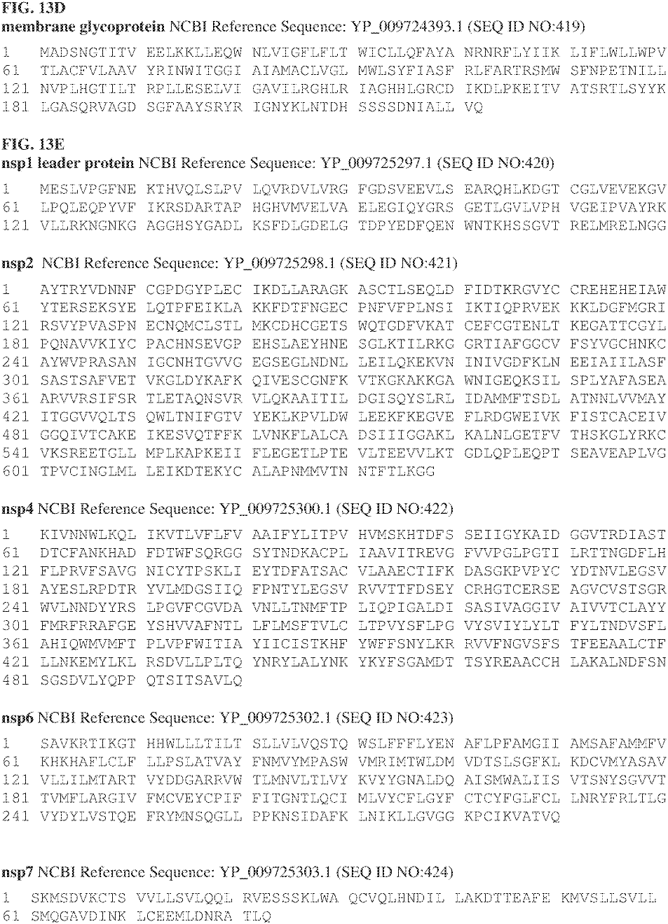

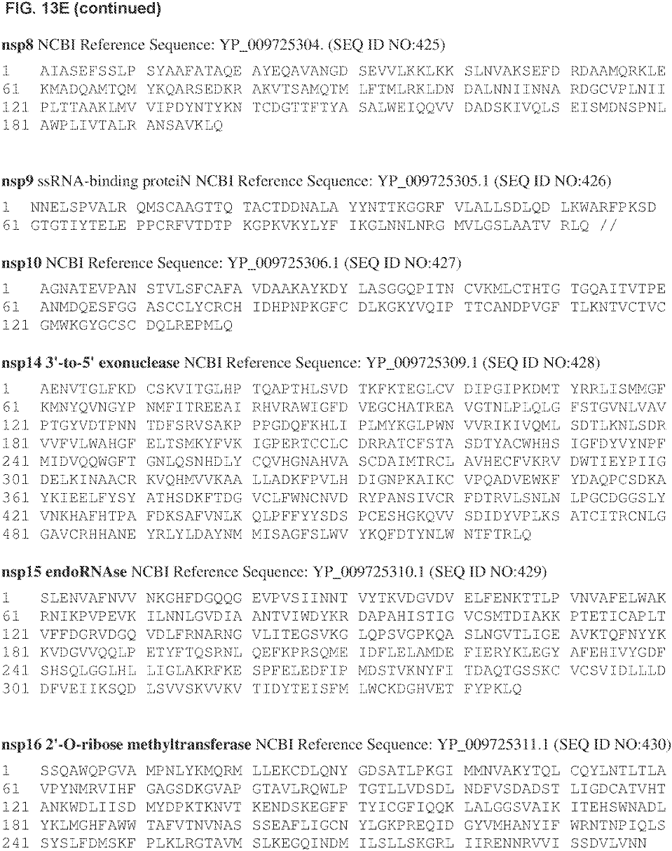

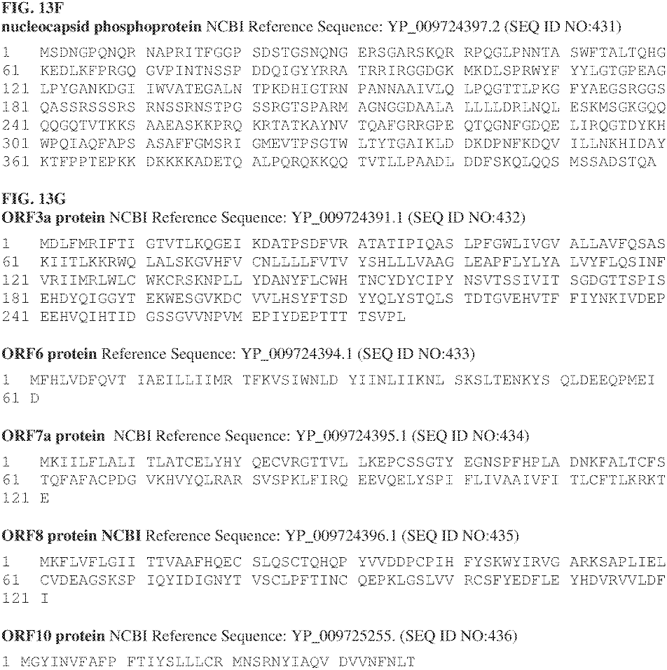

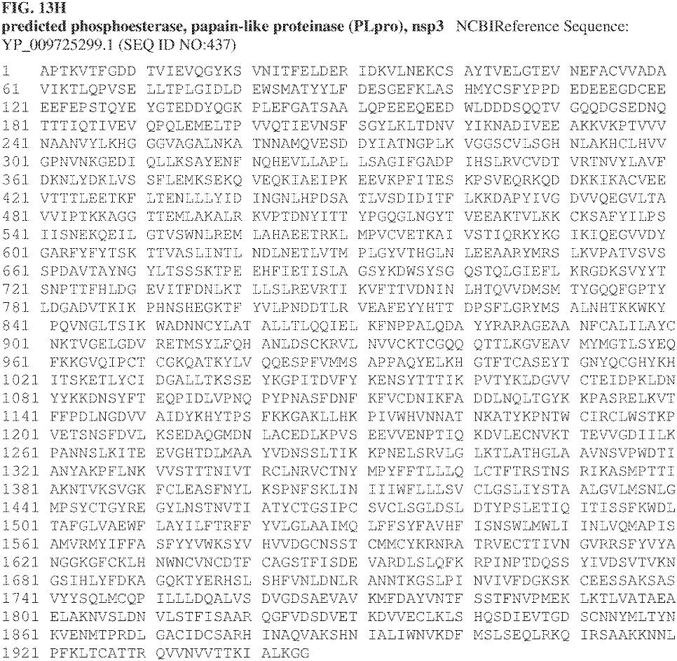

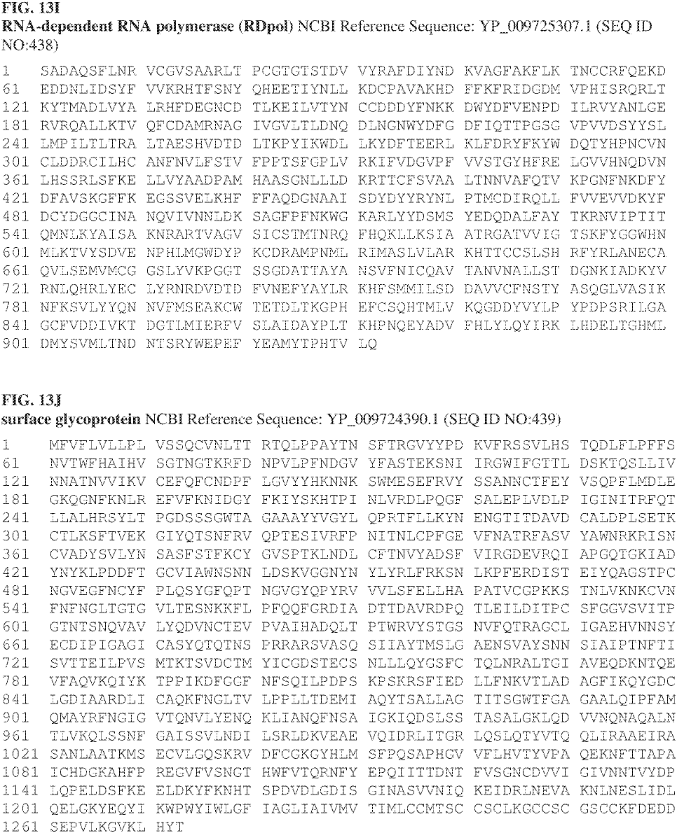

In some cases, a peptide present in a TMP of the present disclosure is a peptide of from 4 amino acids (aa) to 25 aa in length (e.g., 4 aa, 5 aa, 6 aa, 7, aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa in length) of a polypeptide comprising an amino acid sequence having at least 50%, at least 60^, at least 70%, at least 75%, at least 80%, at least 85%, at least 90%, at least 95%, at least 98%, at least 99%, or 100%, amino acid sequence identity to any one of the SARS-CoV-2 polypeptides depicted in FIGS. 13A-13J. In some cases, a peptide present in a TMP of the present disclosure is a peptide of from 4 amino acids (aa) to 25 aa in length (e.g., 4 aa, 5 aa, 6 aa, 7, aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa in length) of one of the polypeptides depicted in FIGS. 13A-13J.

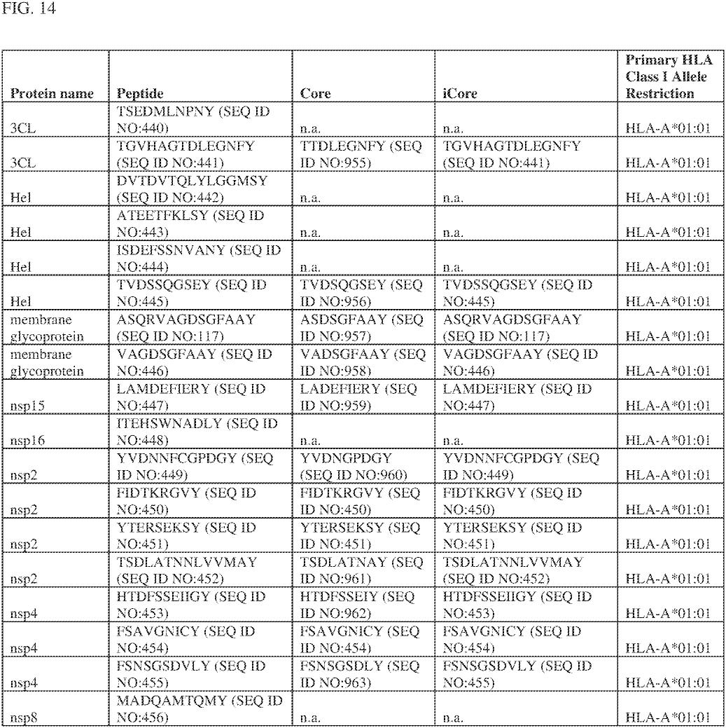

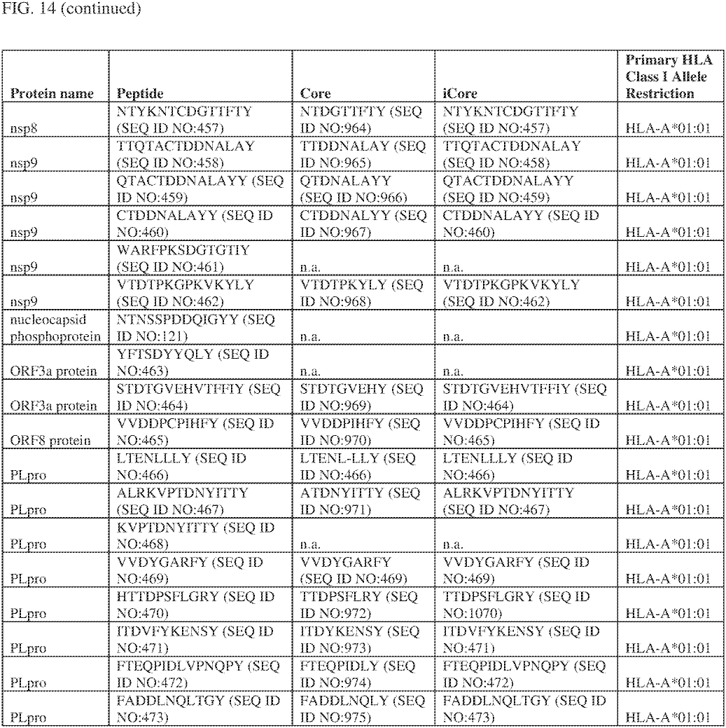

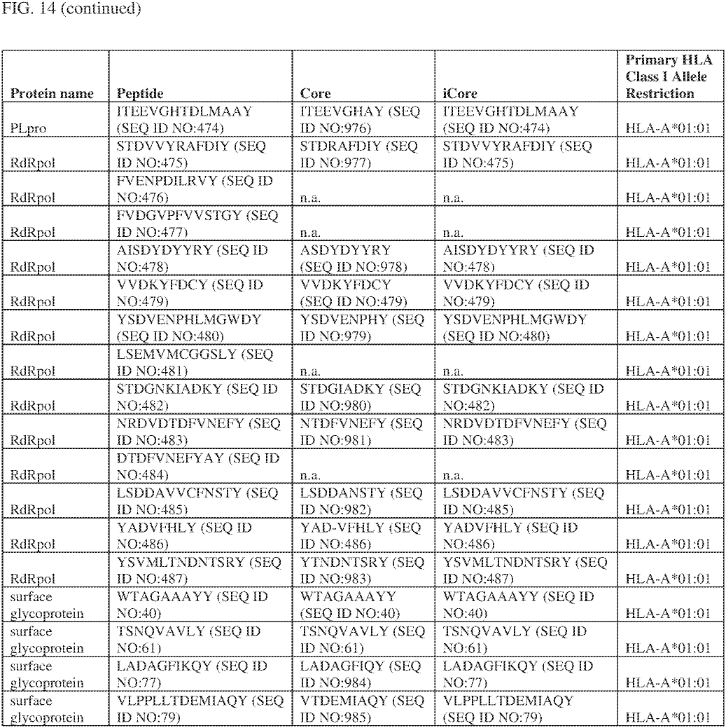

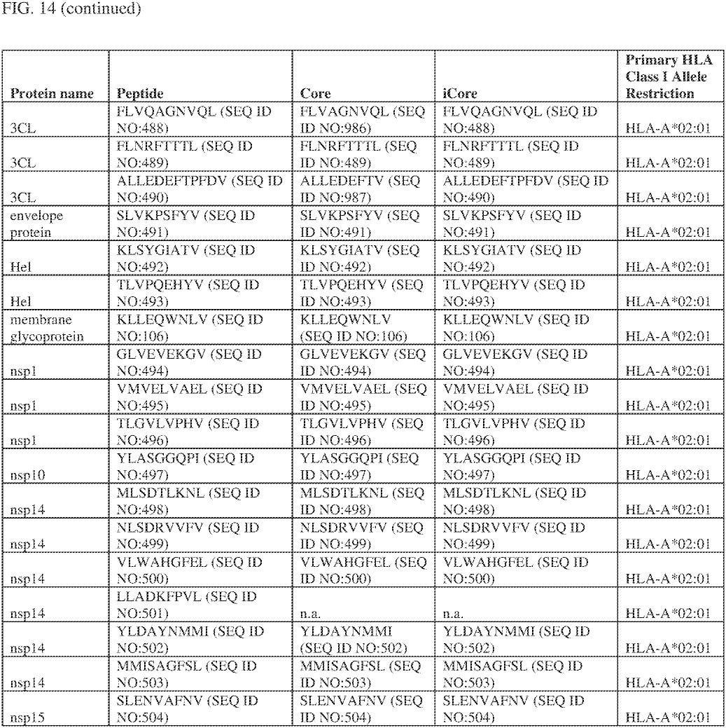

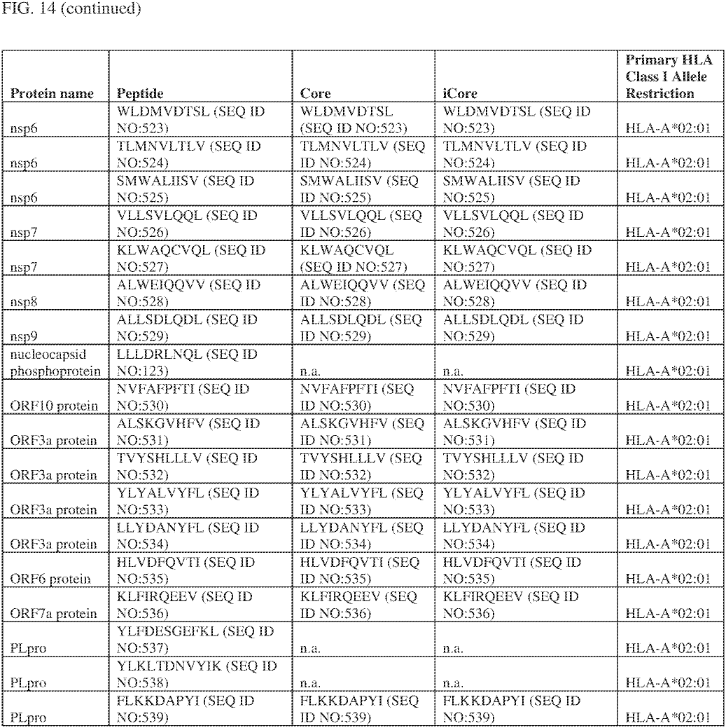

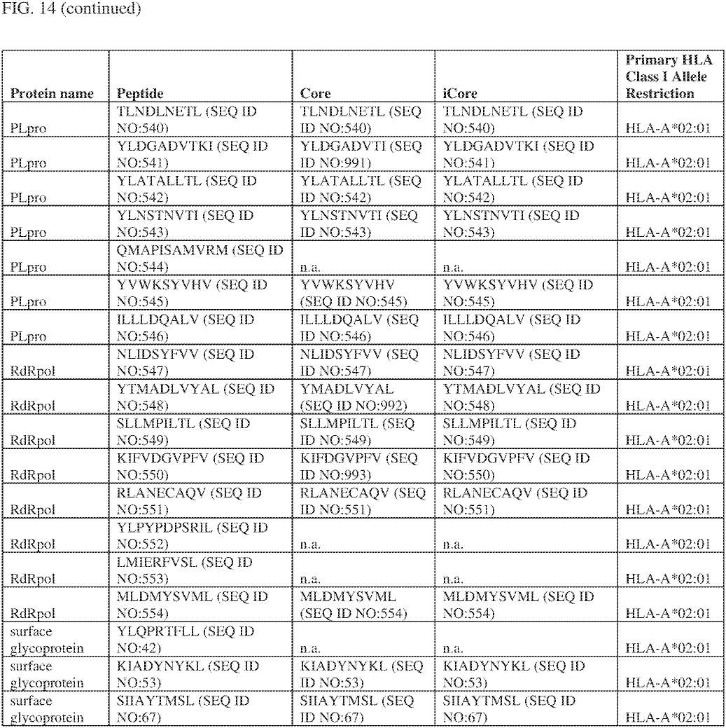

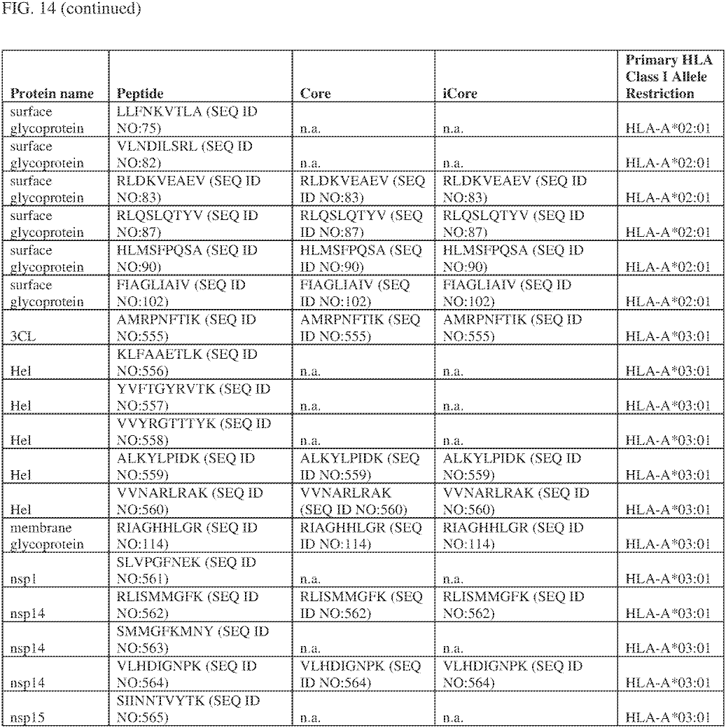

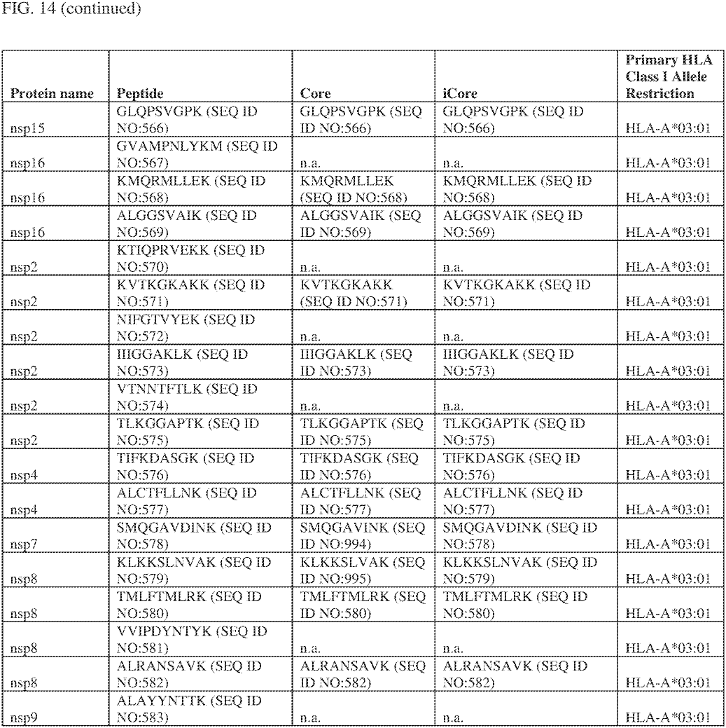

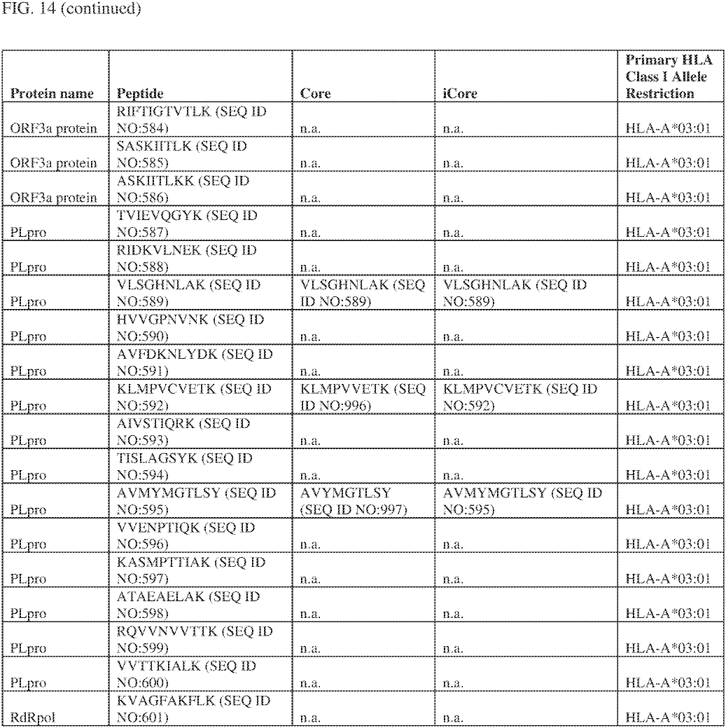

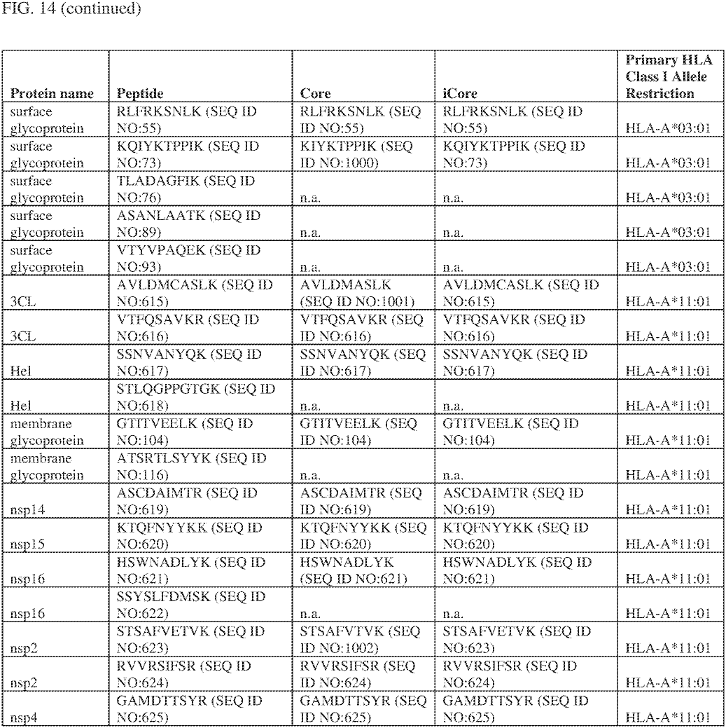

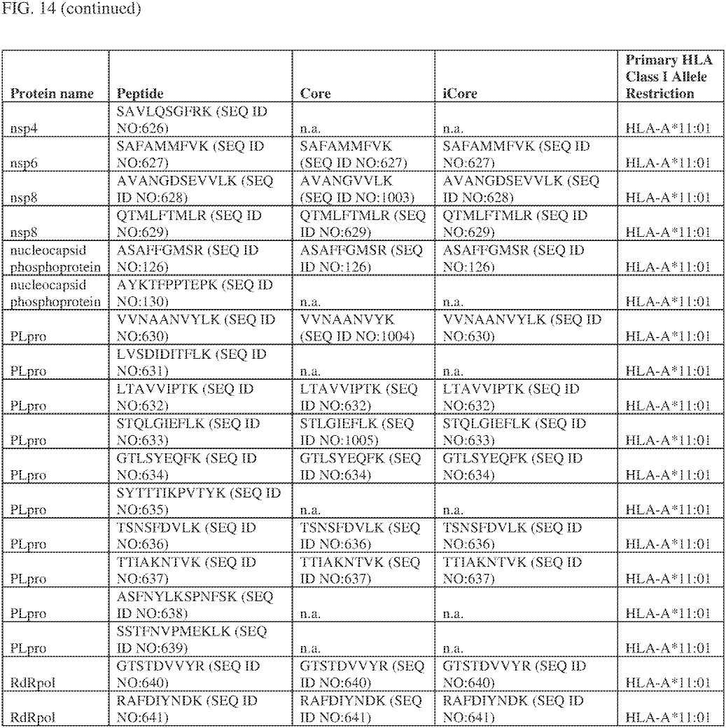

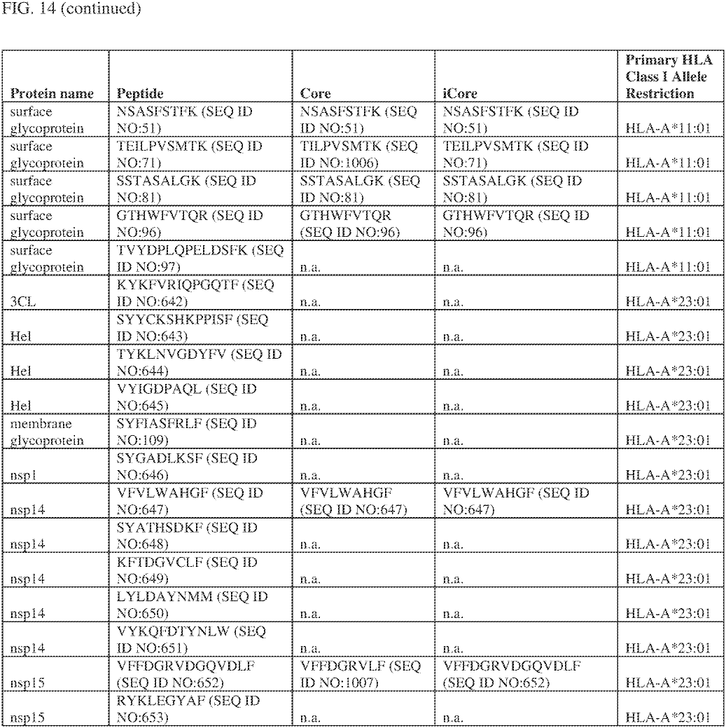

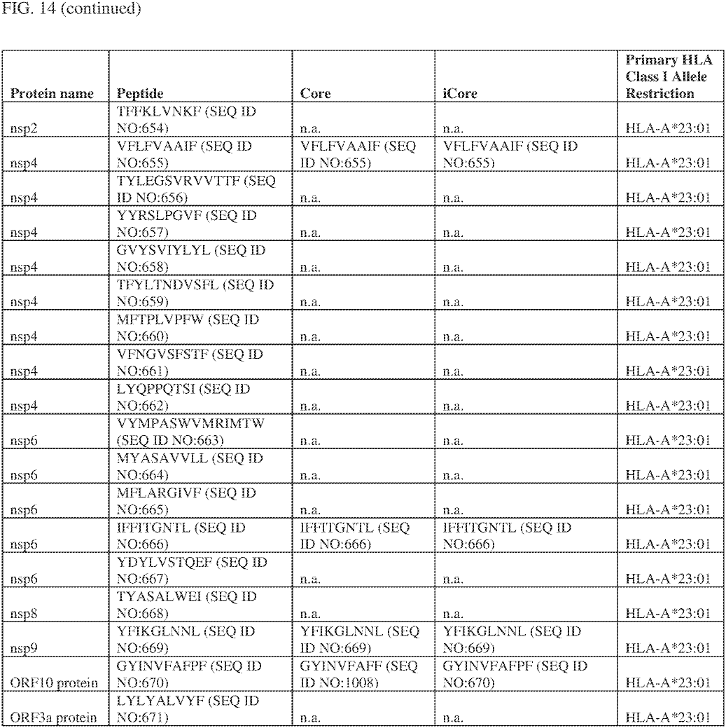

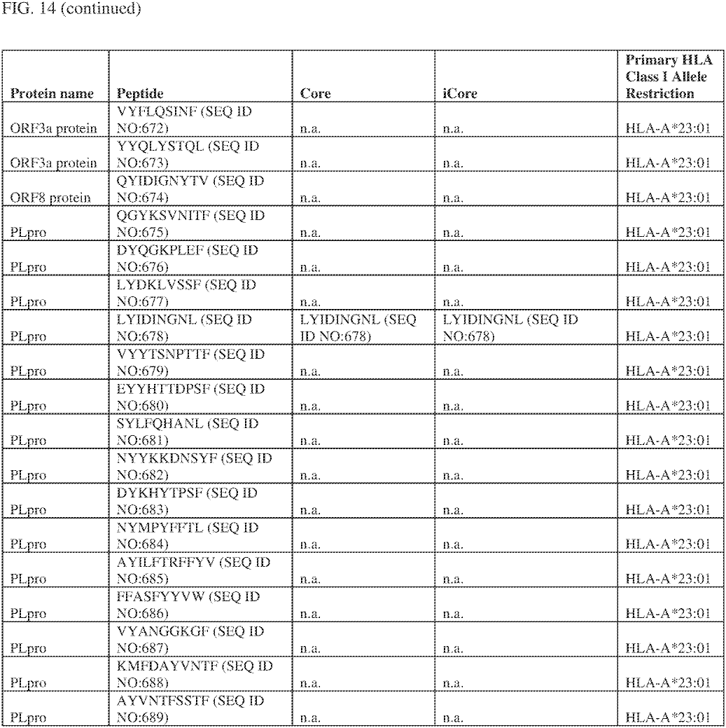

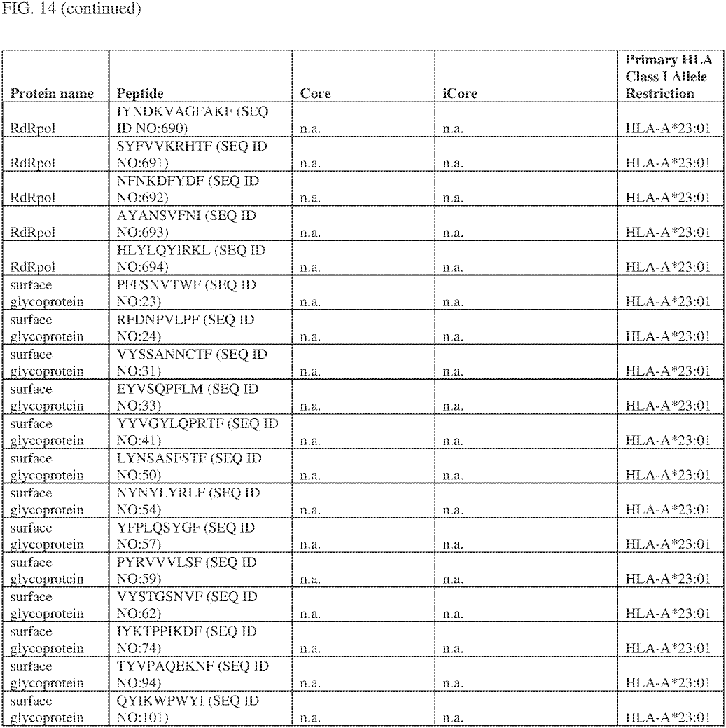

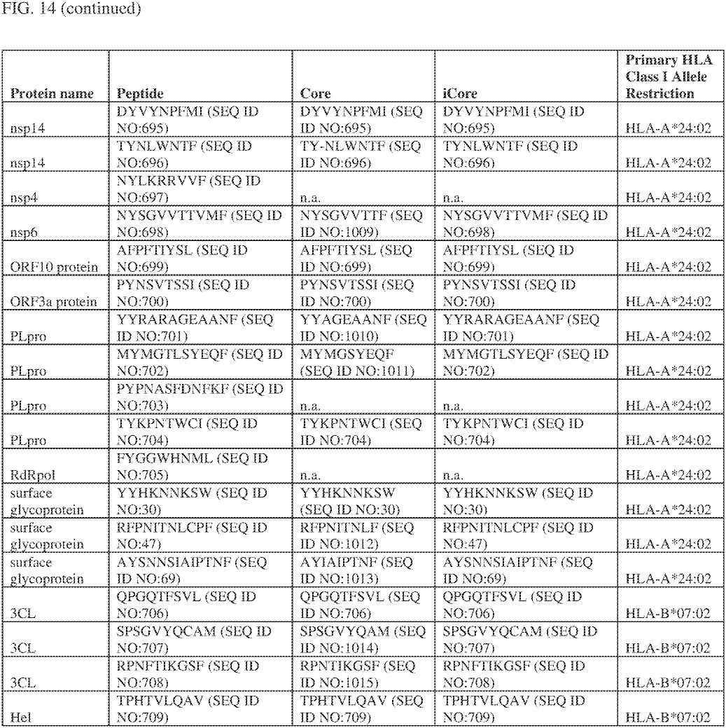

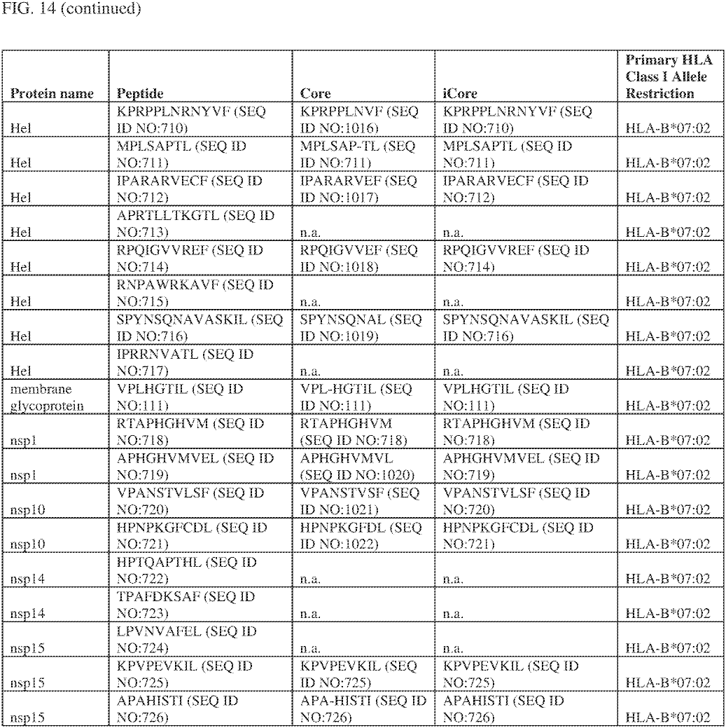

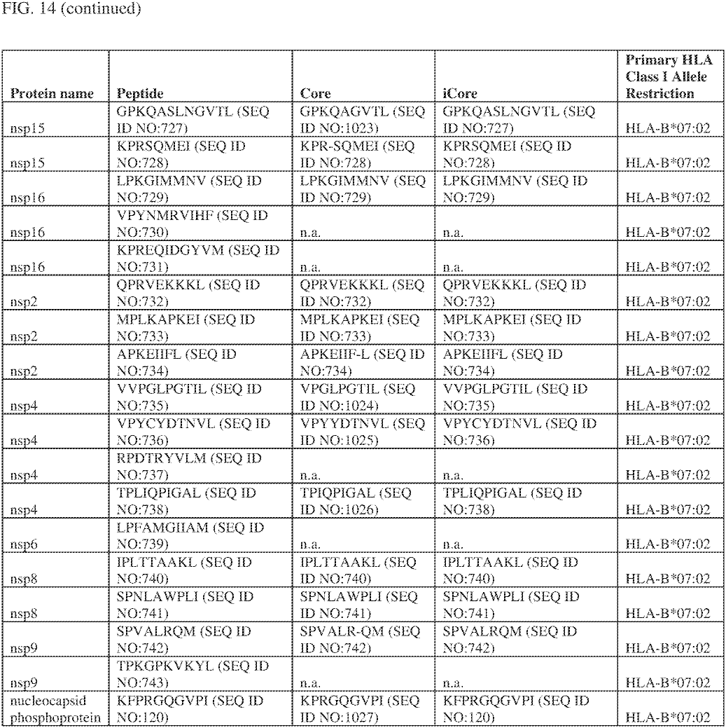

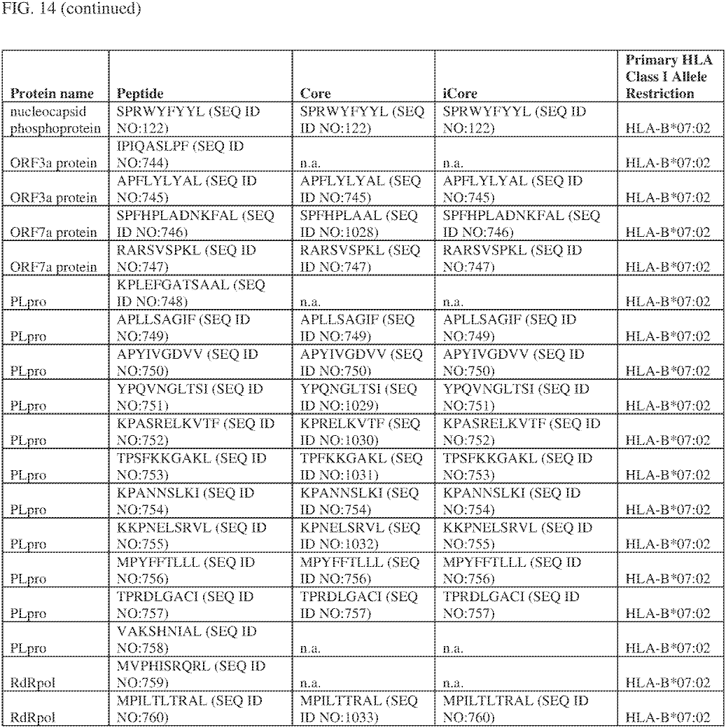

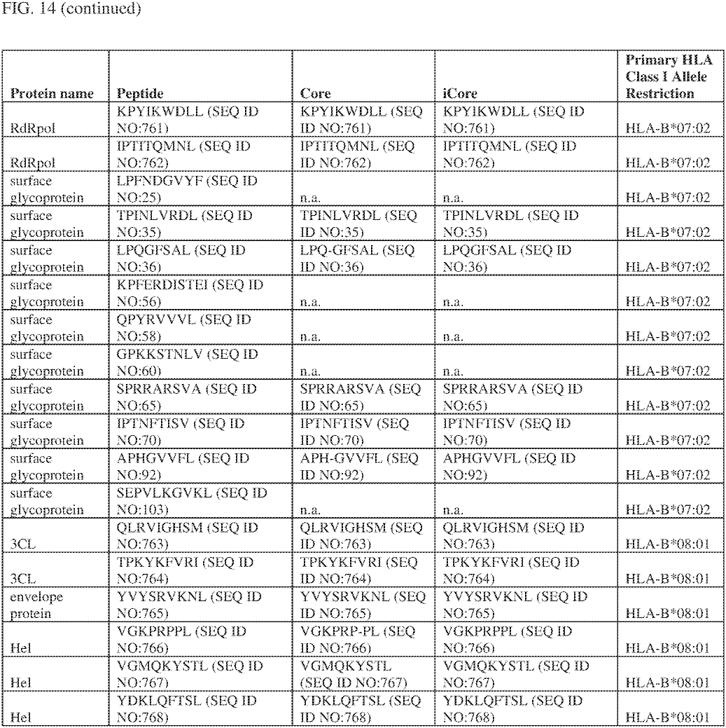

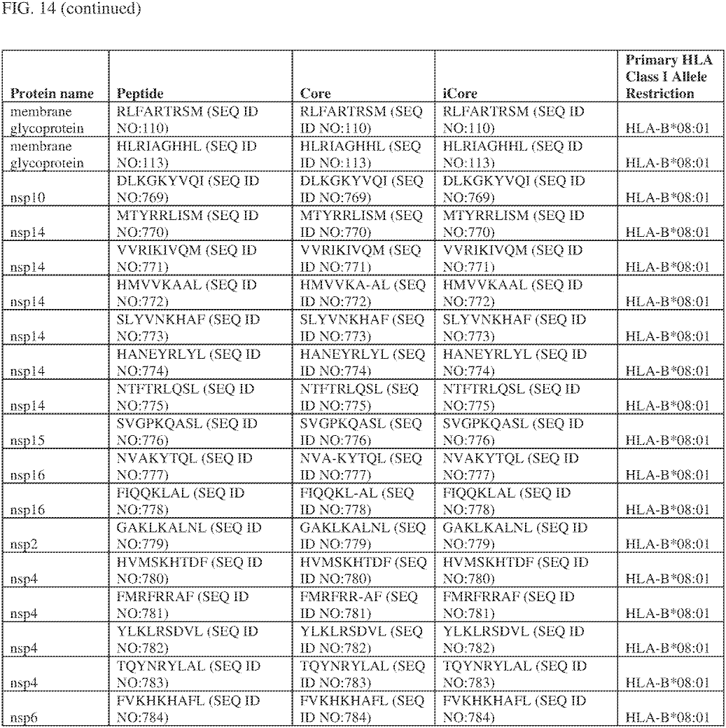

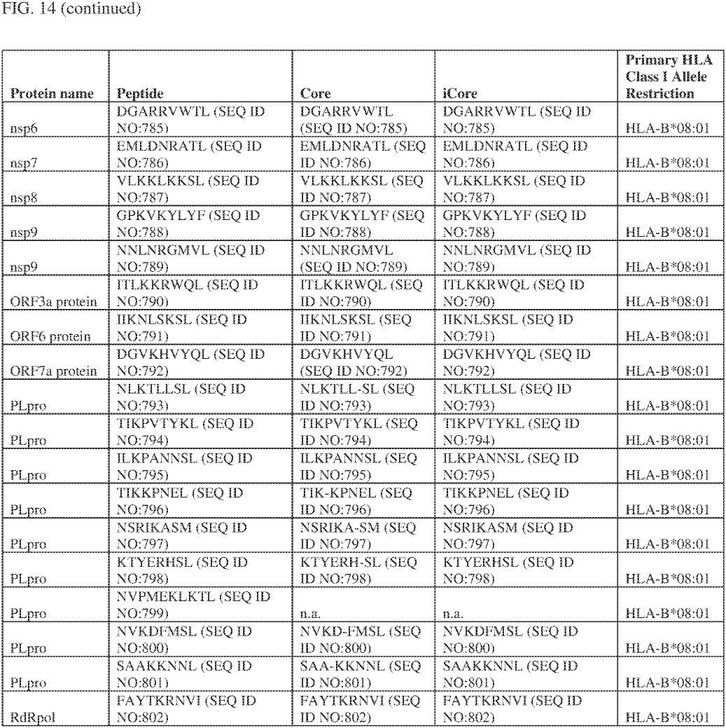

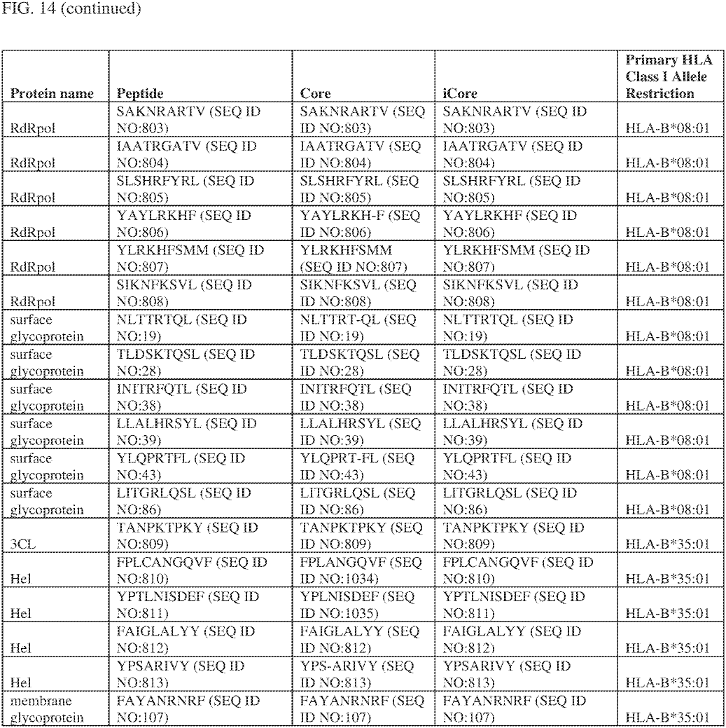

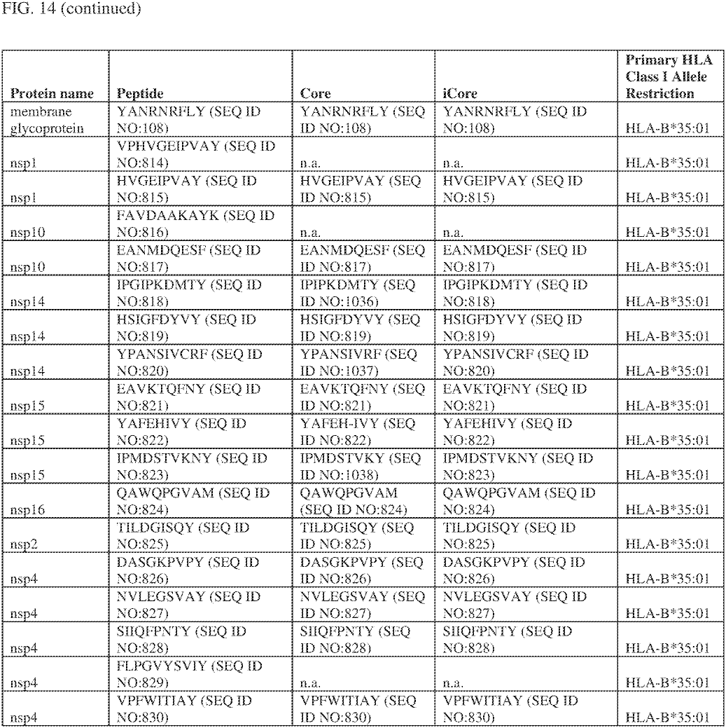

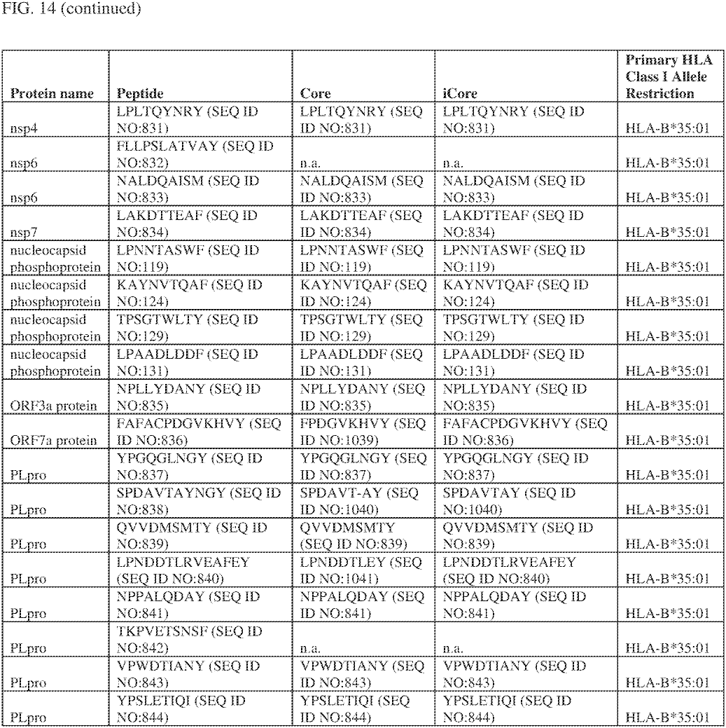

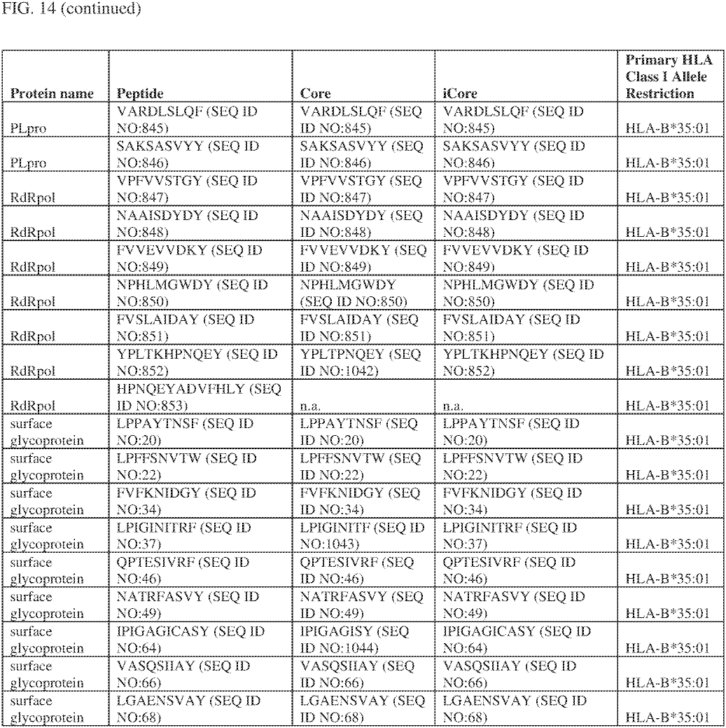

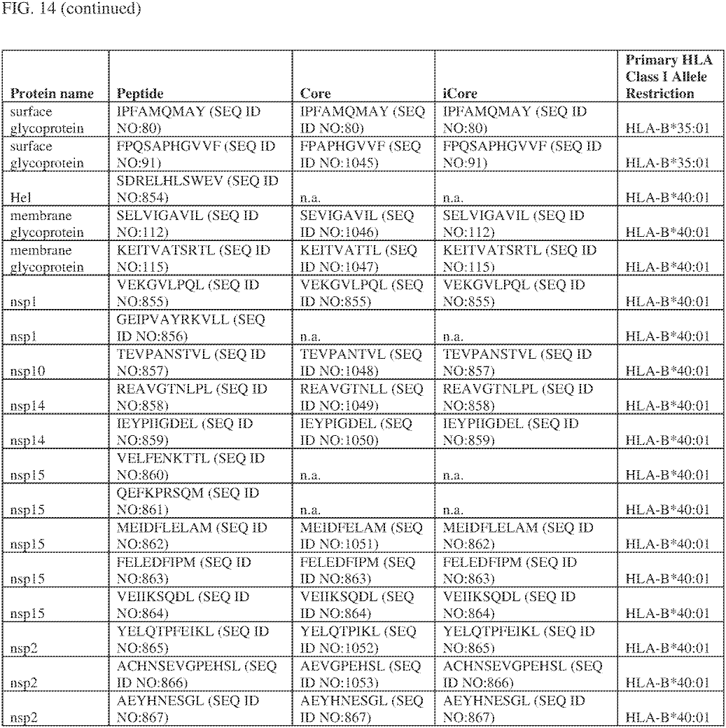

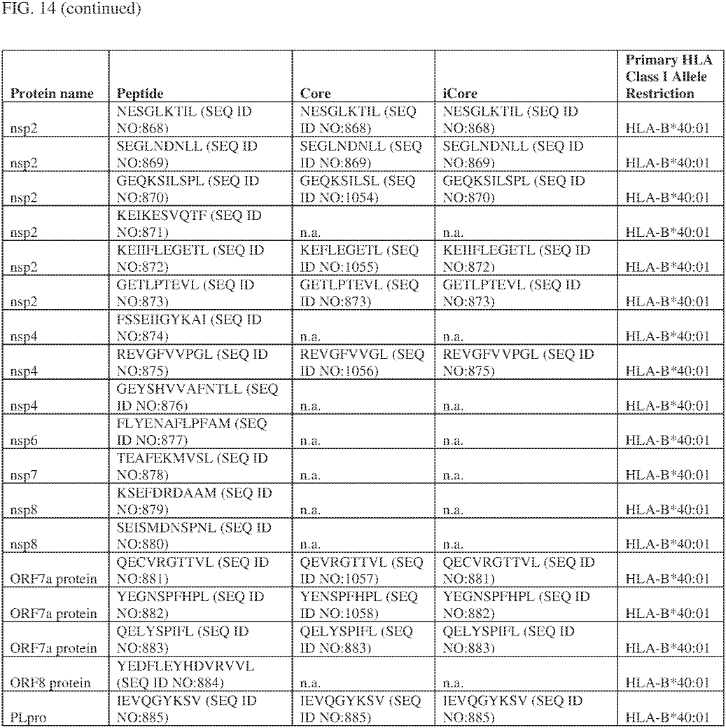

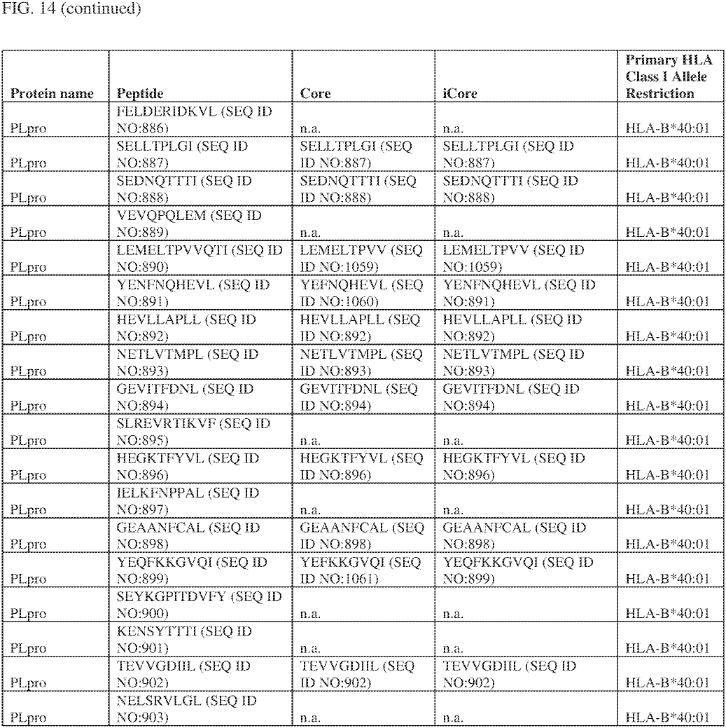

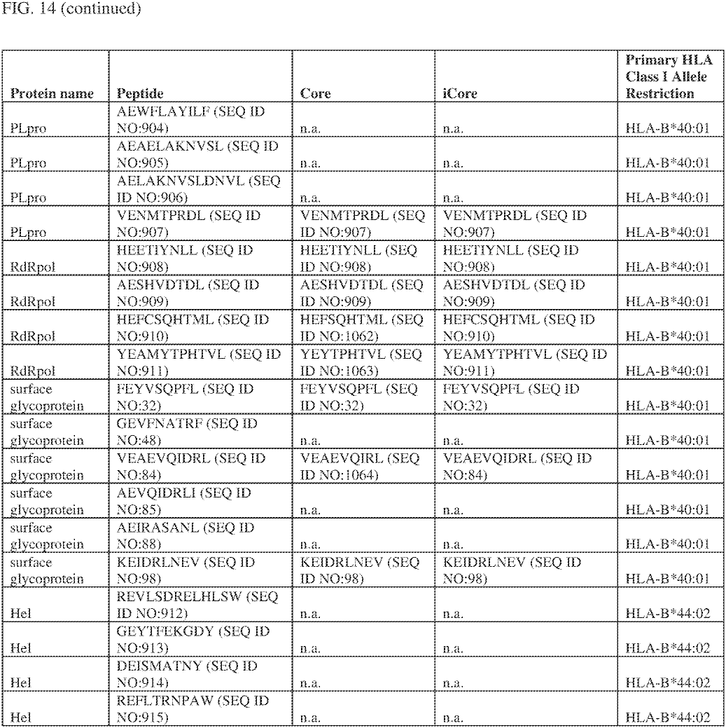

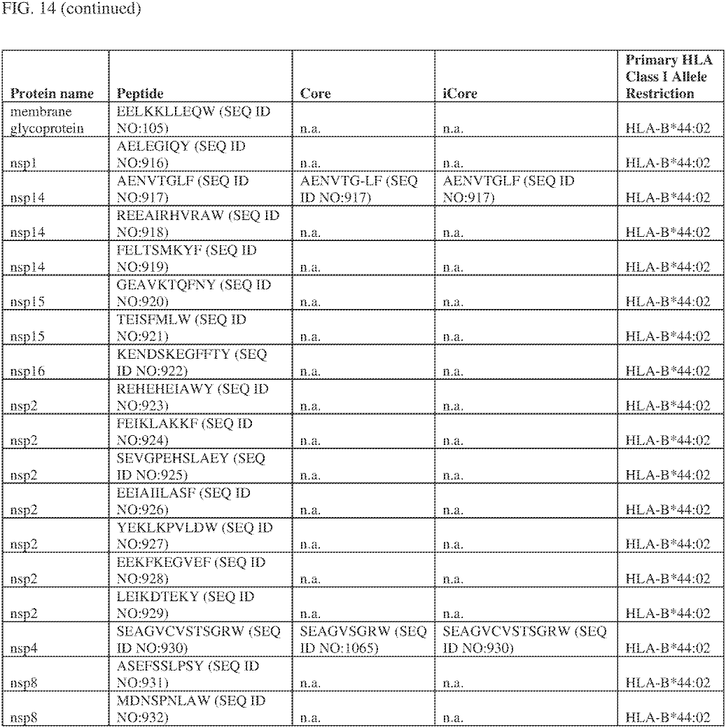

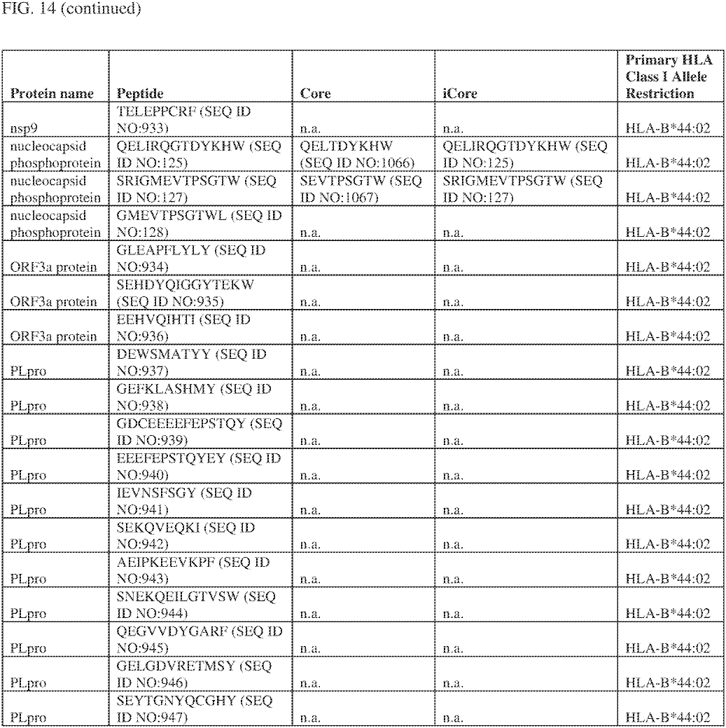

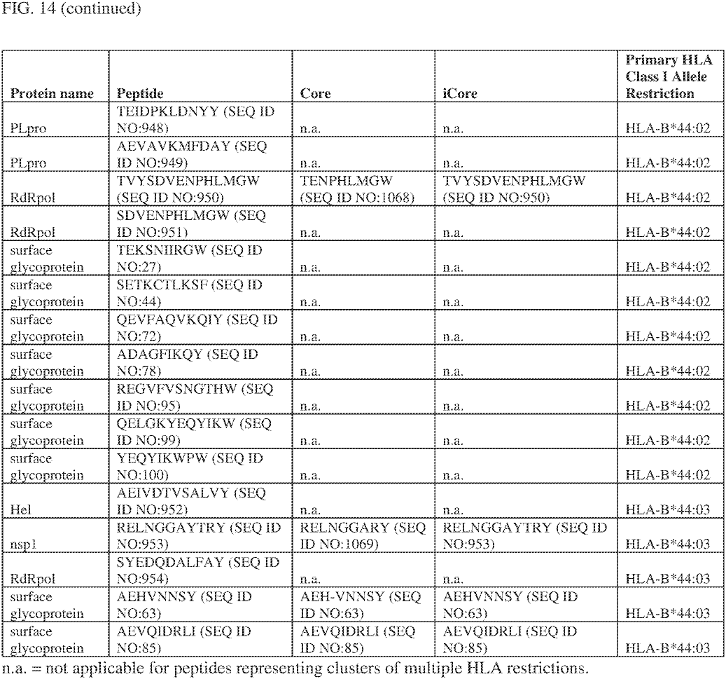

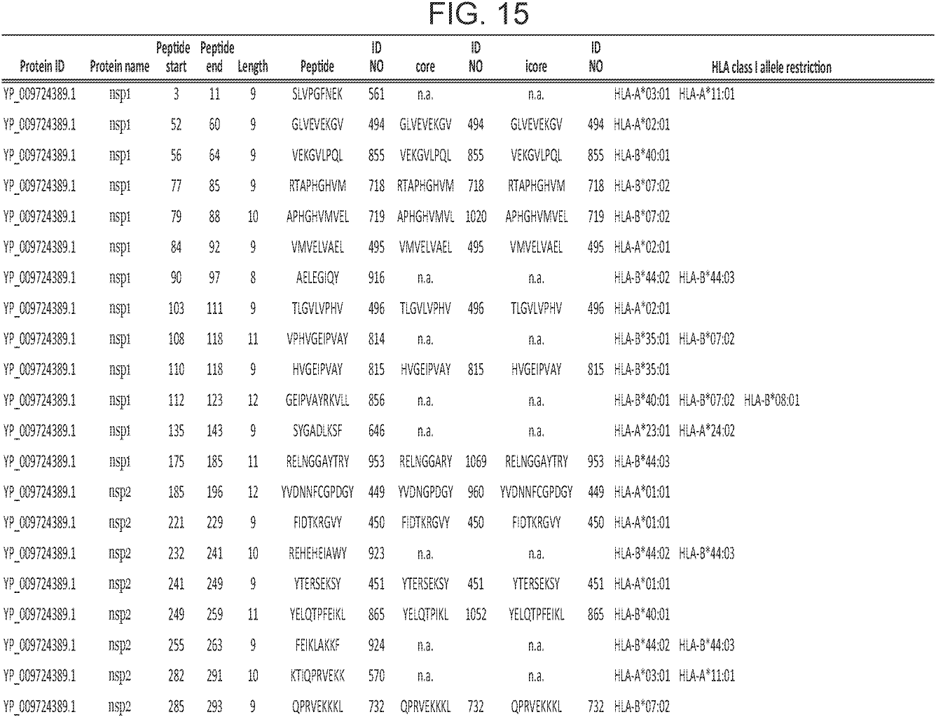

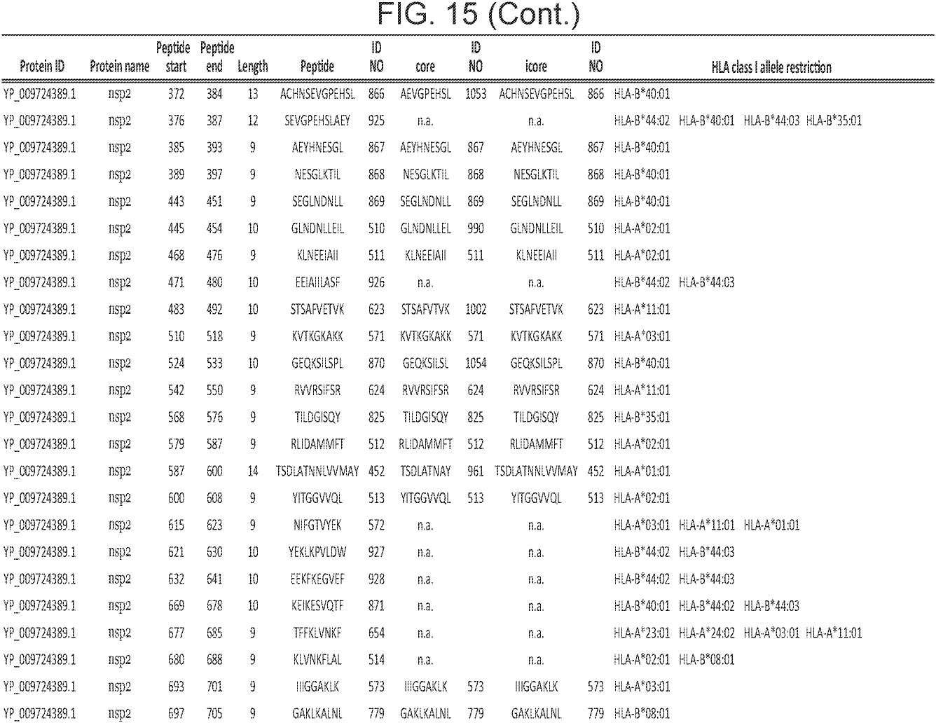

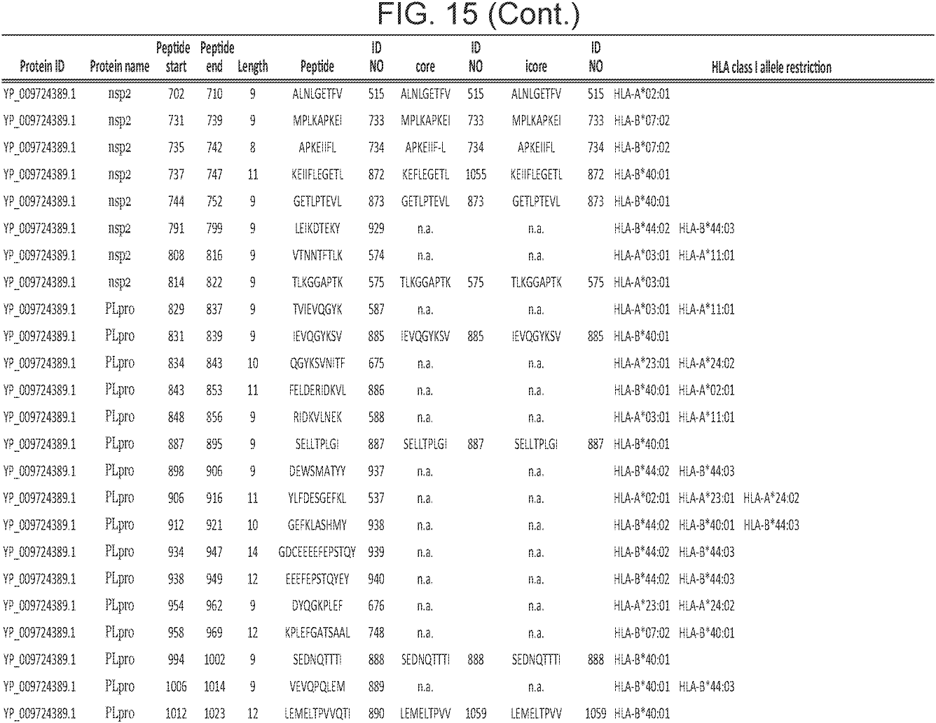

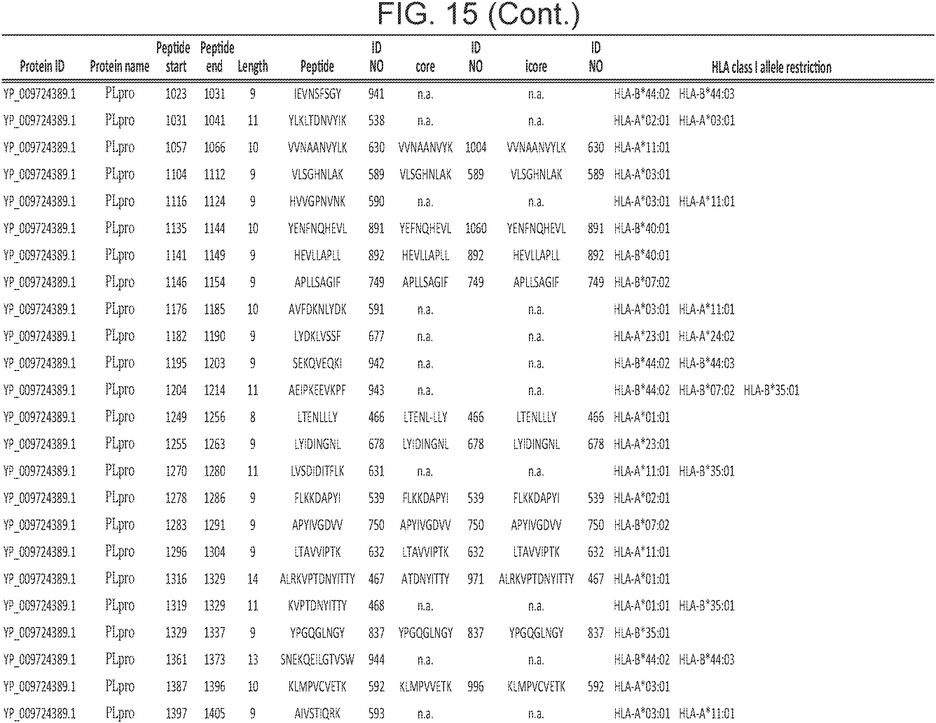

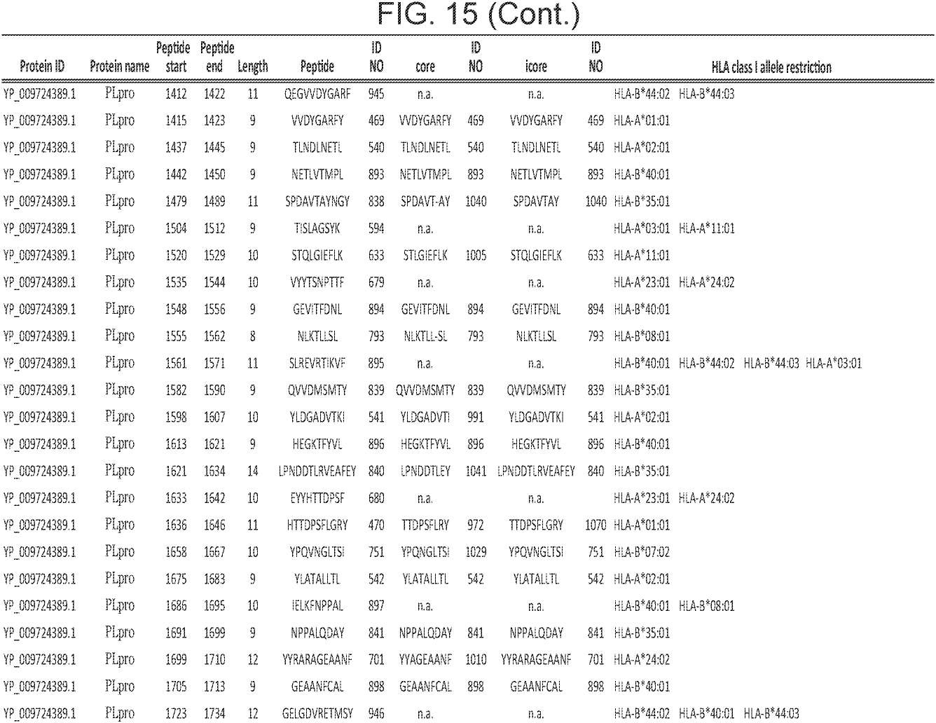

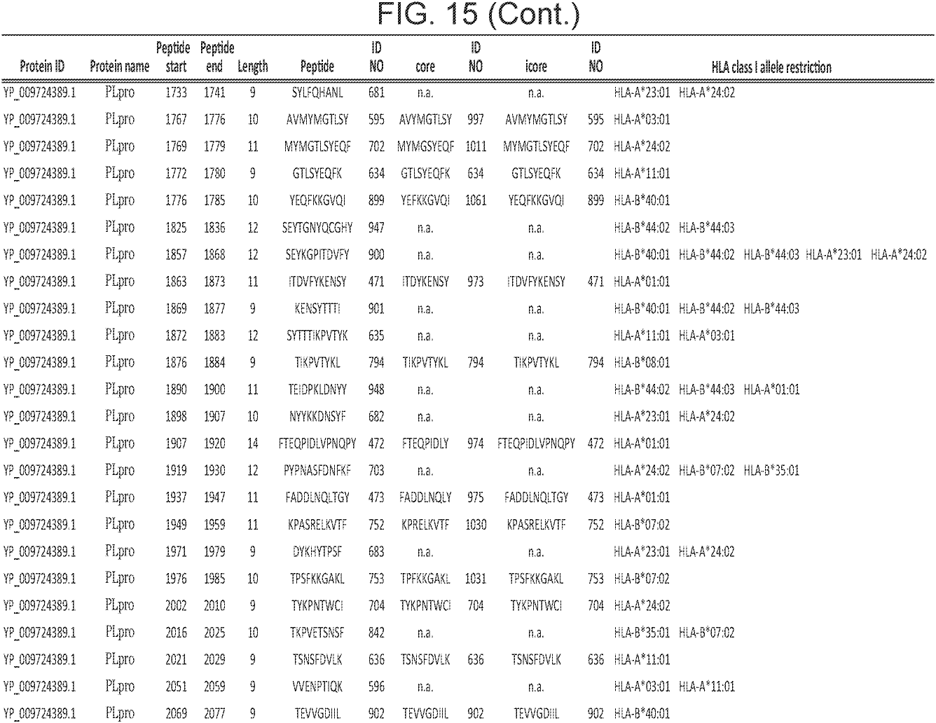

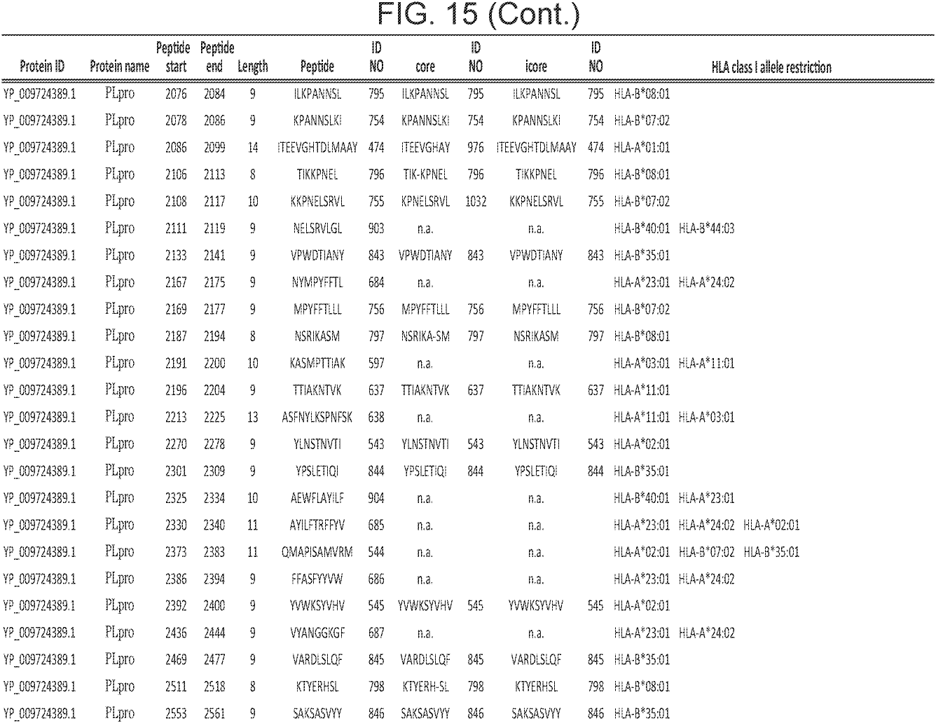

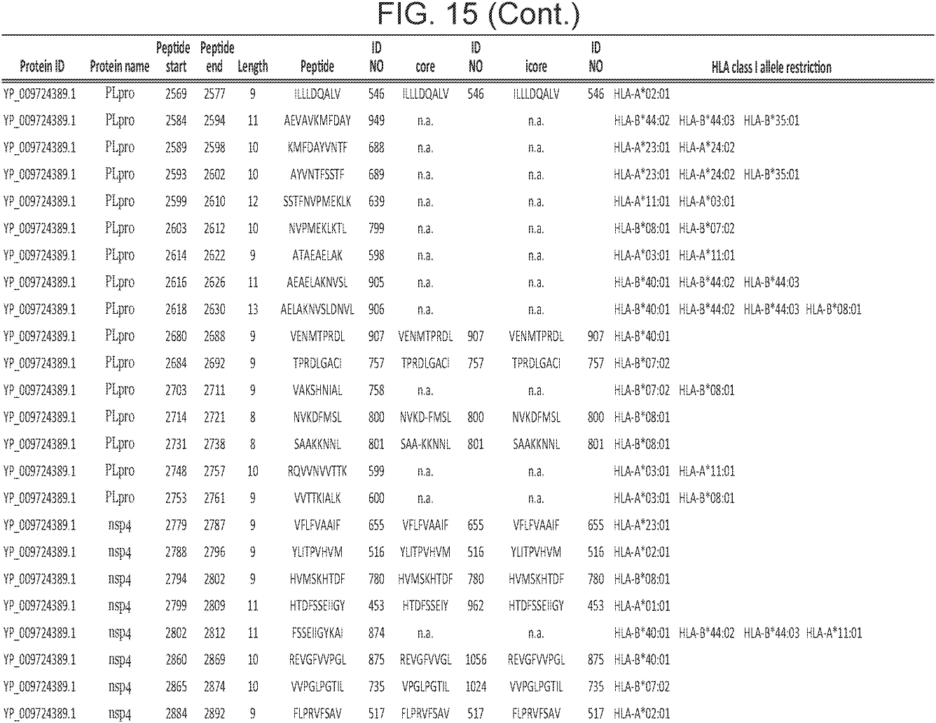

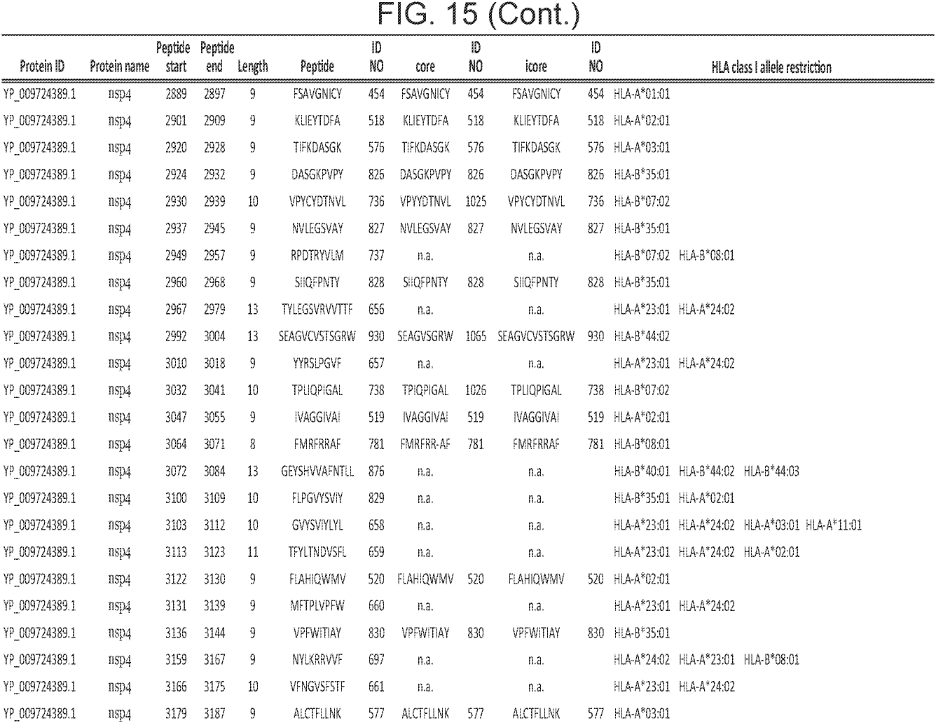

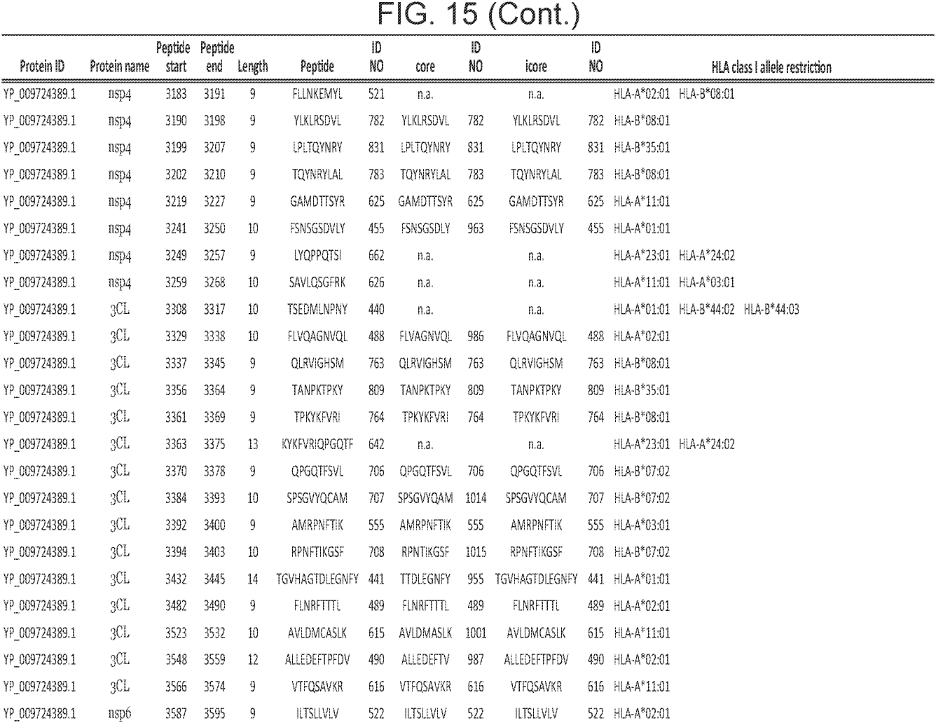

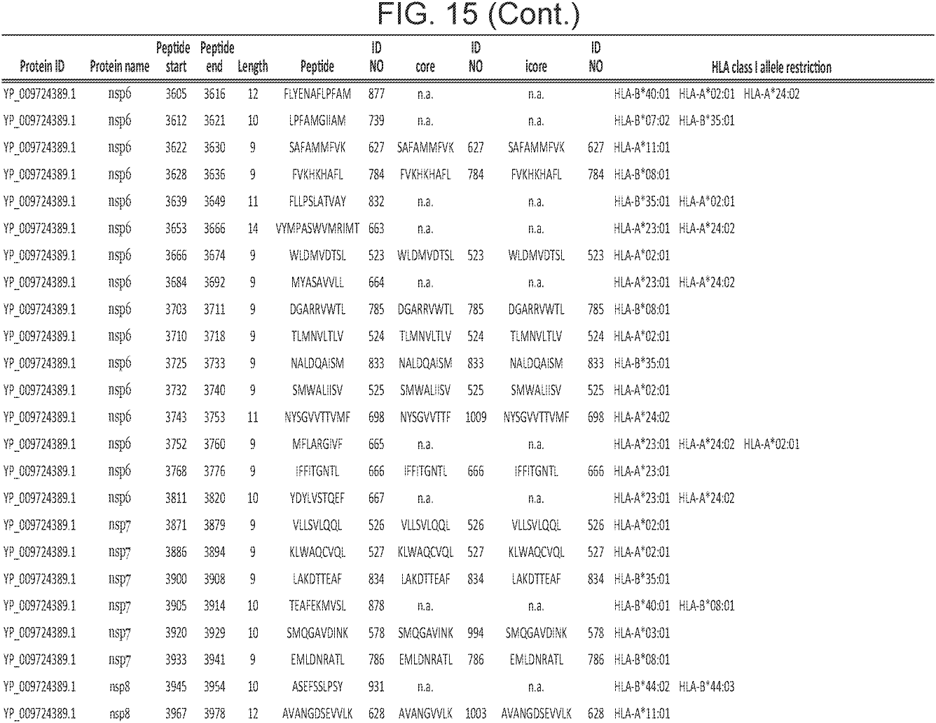

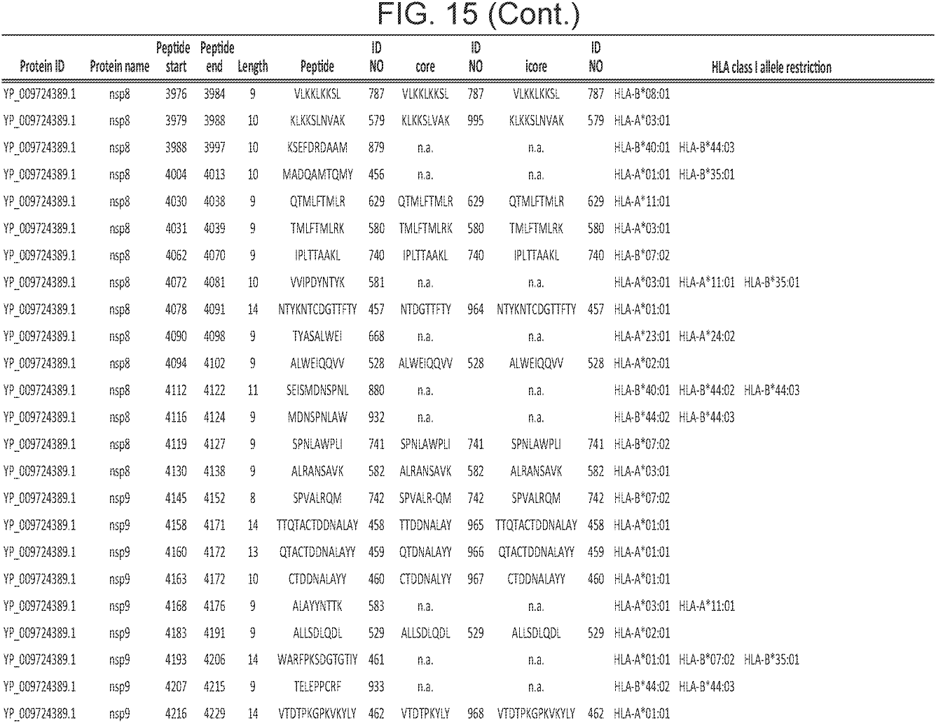

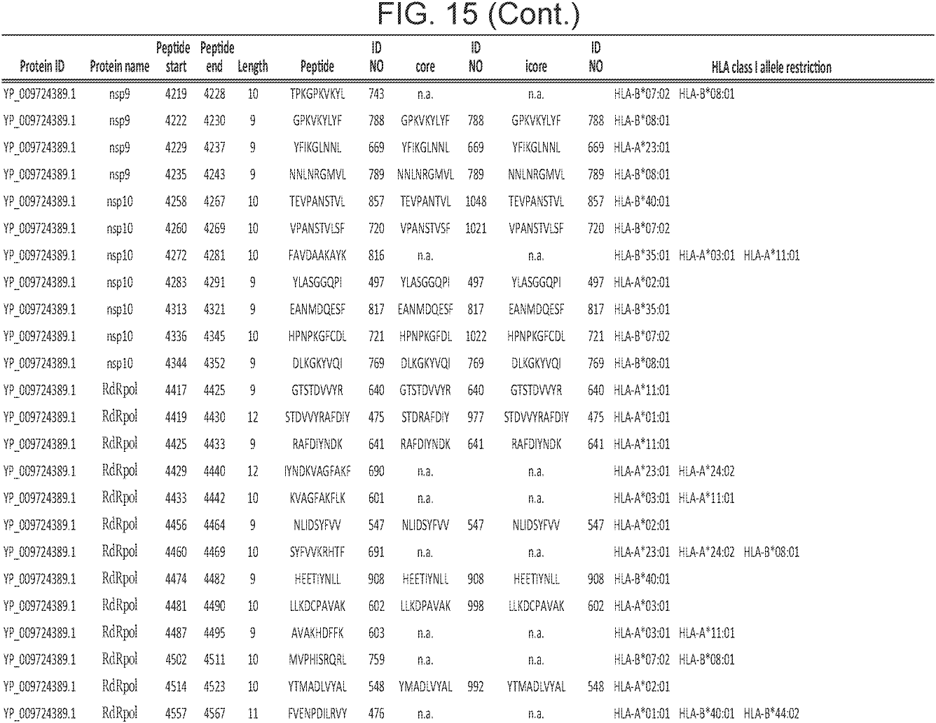

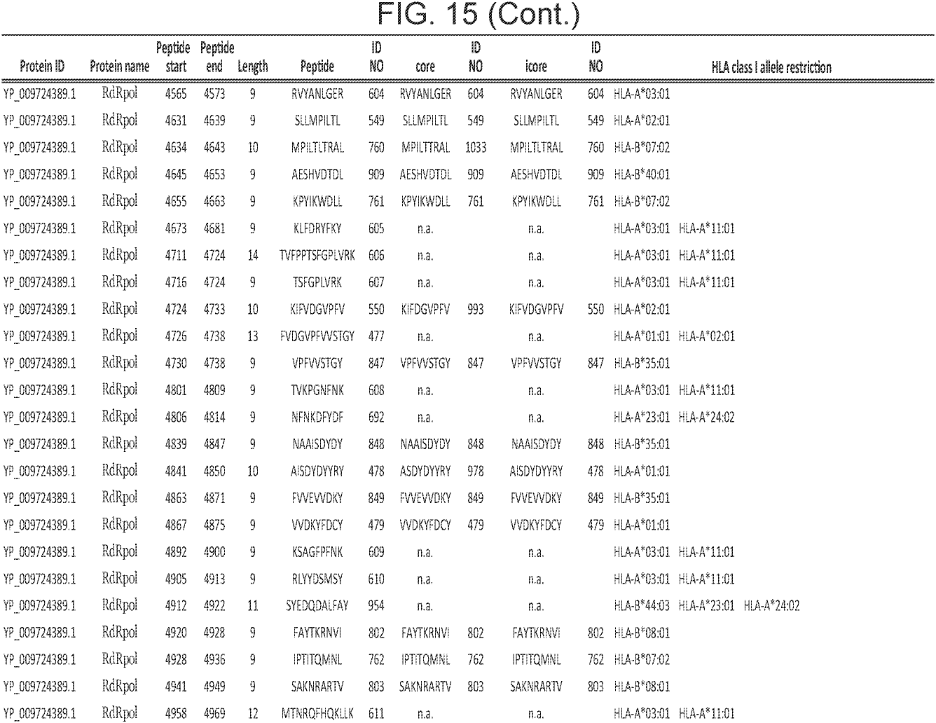

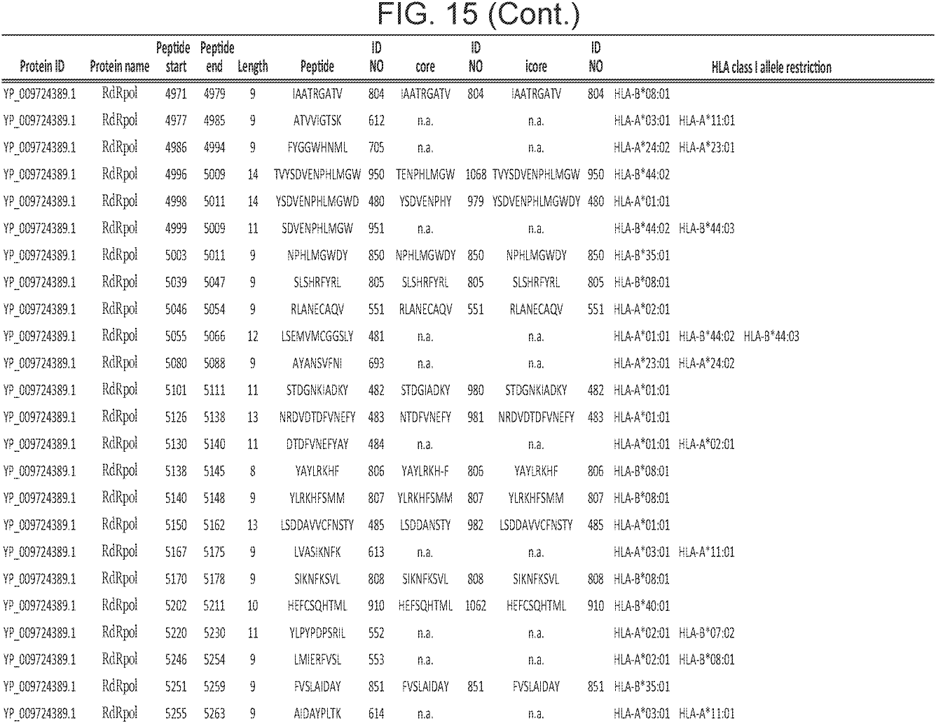

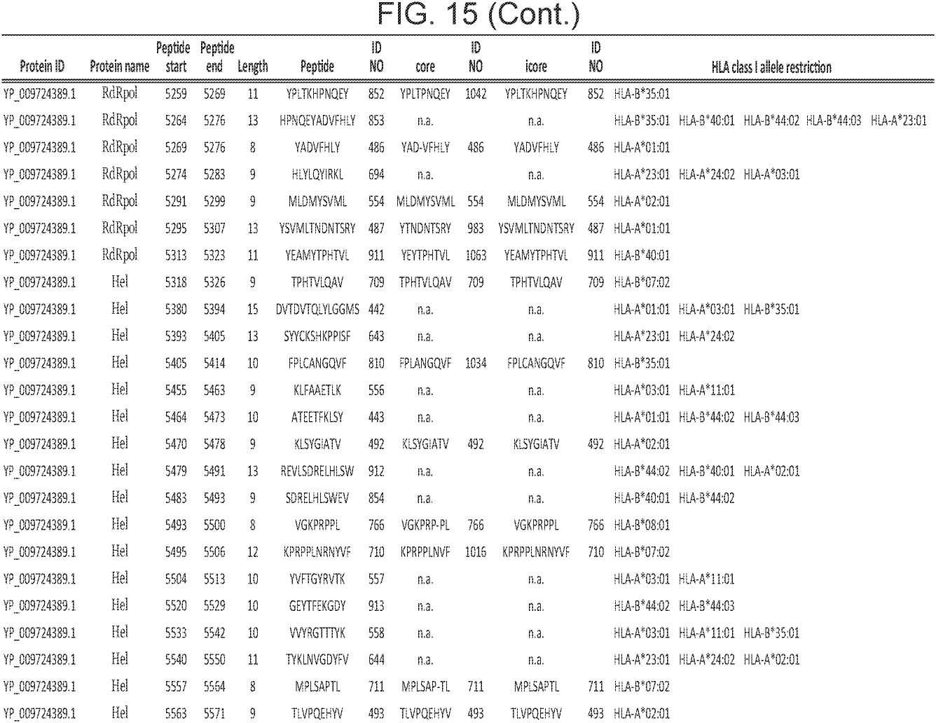

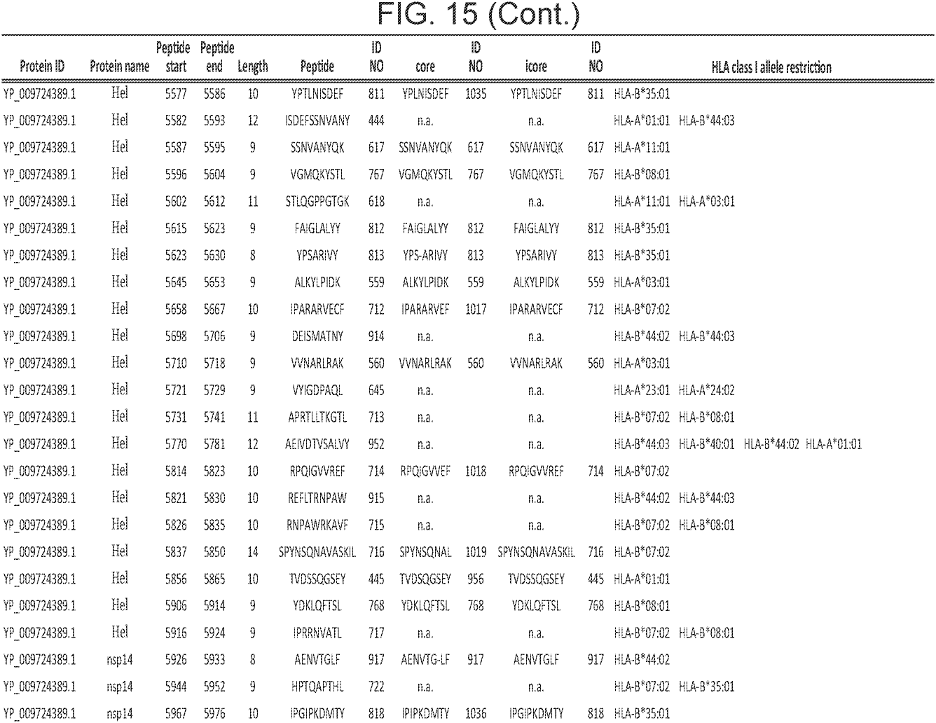

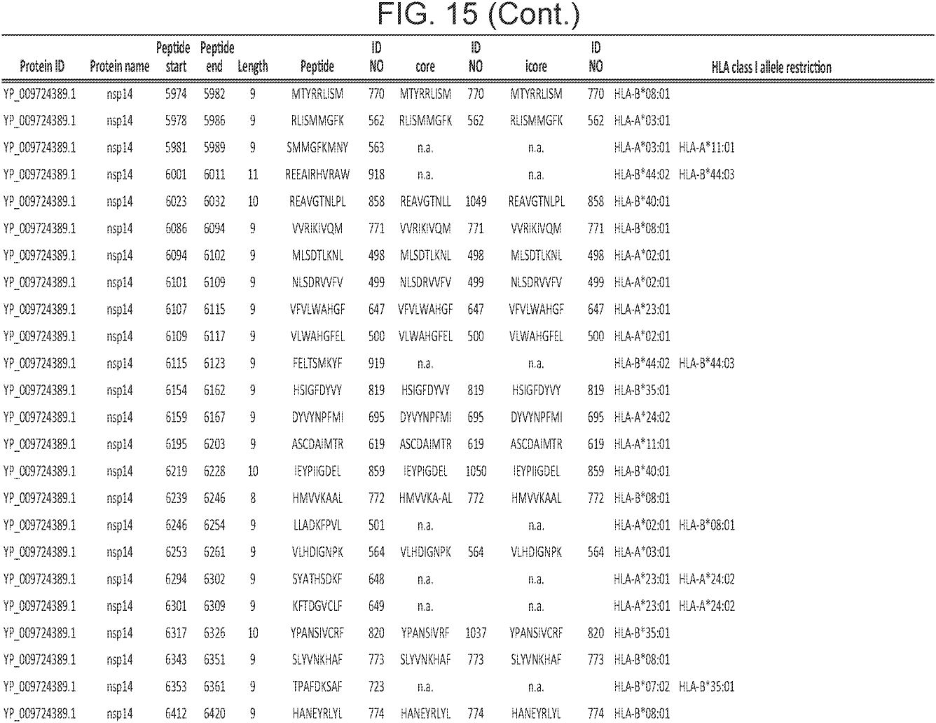

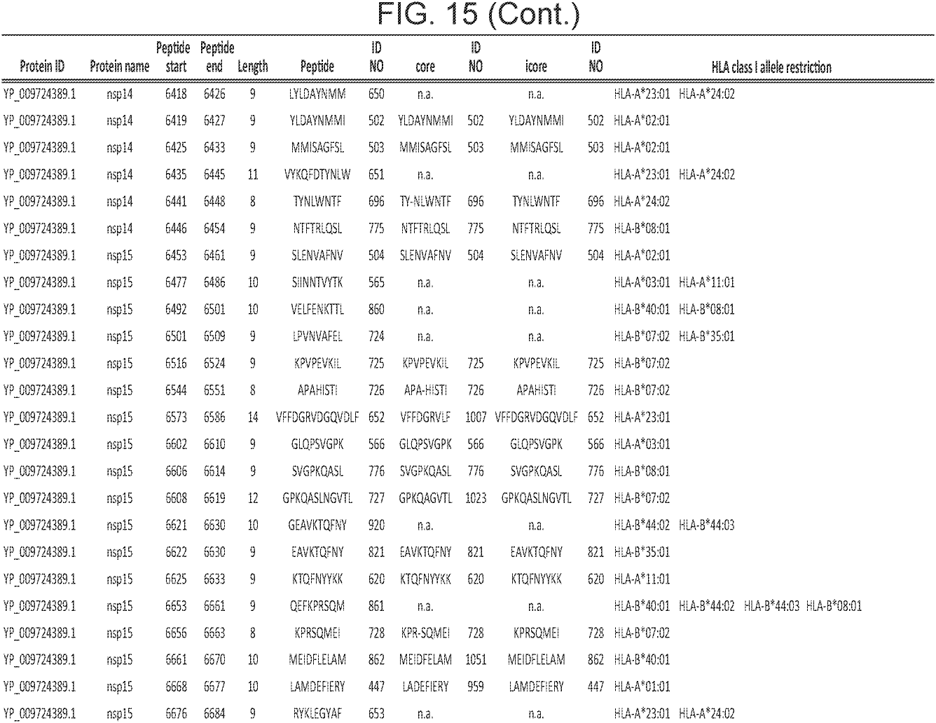

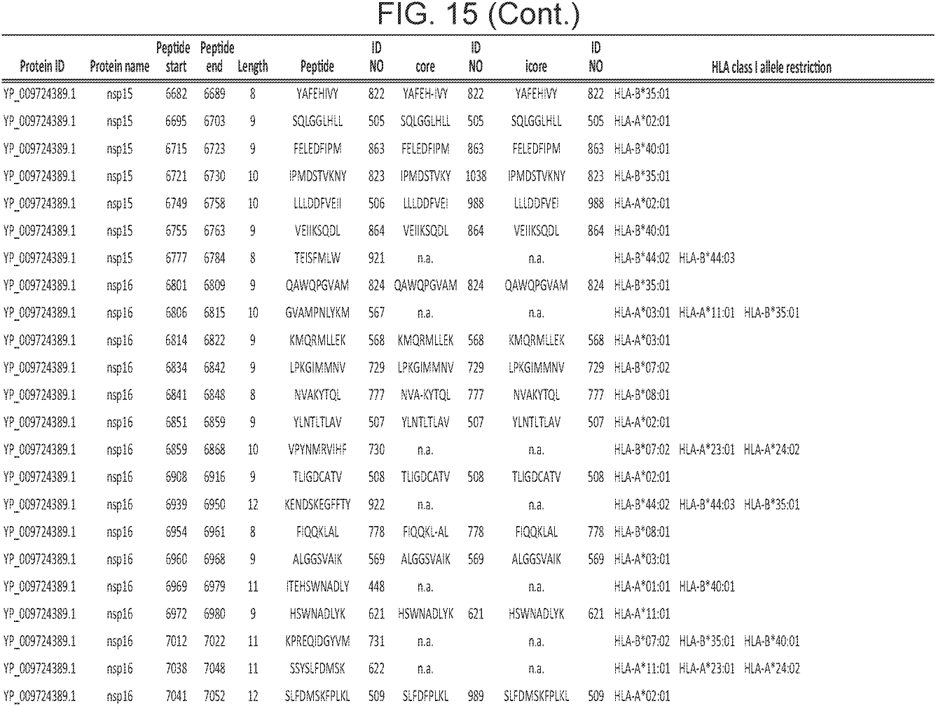

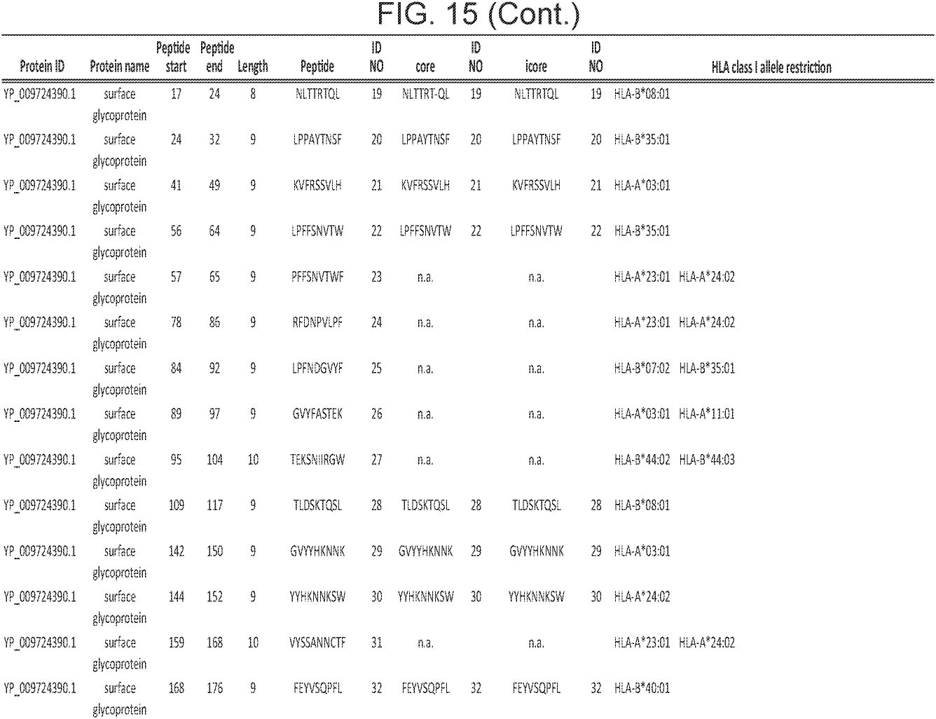

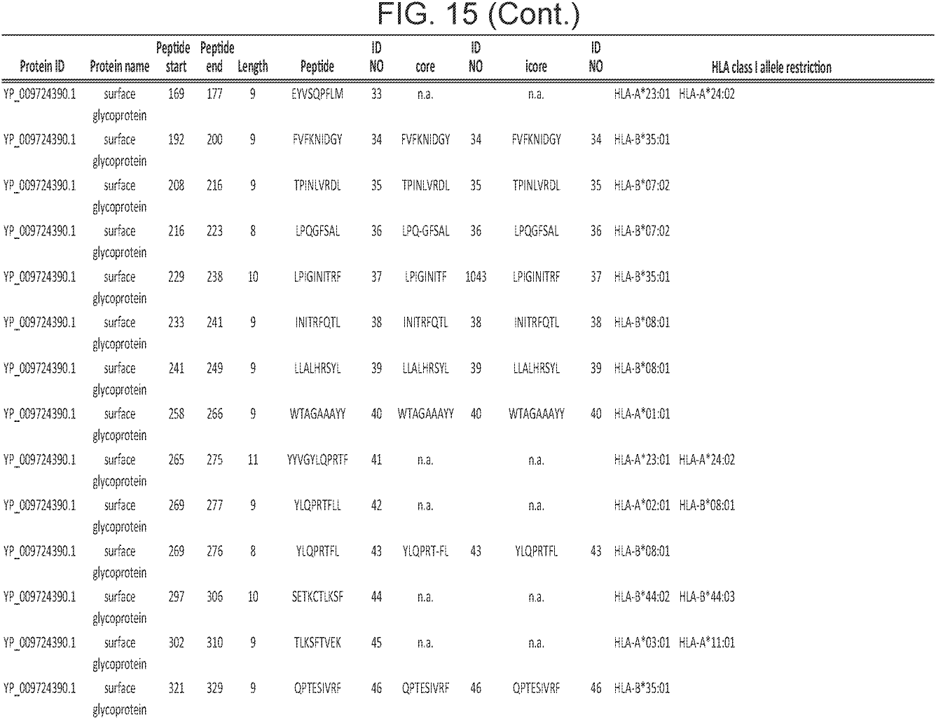

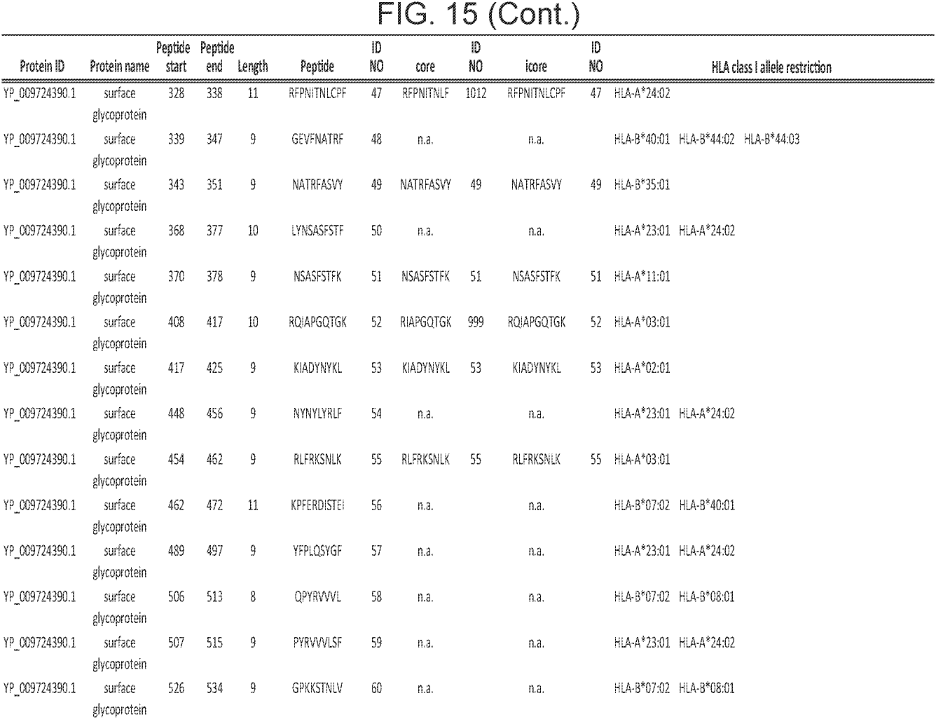

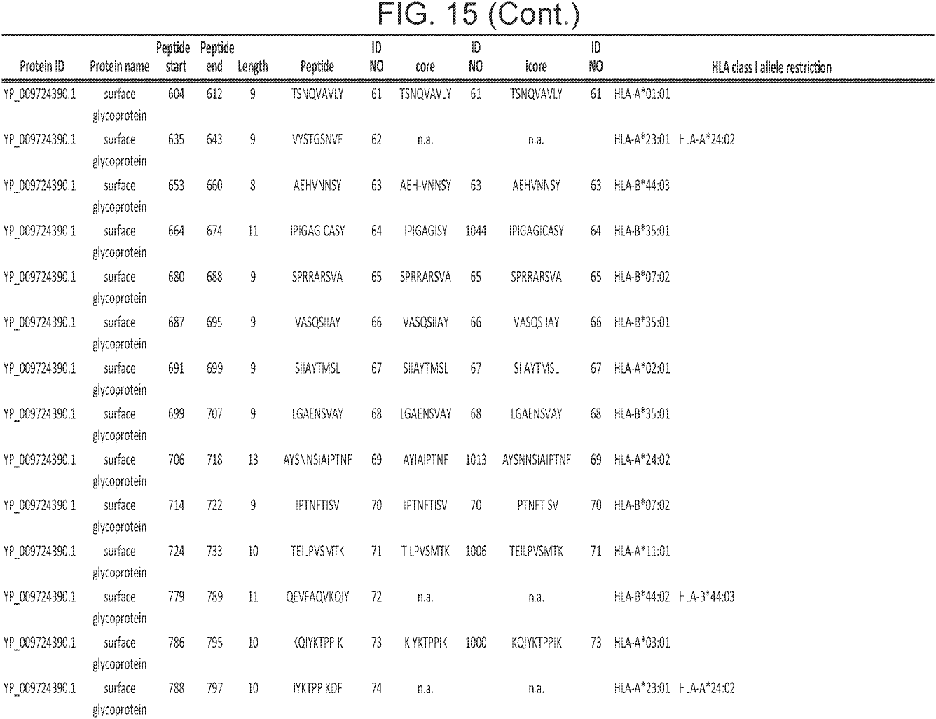

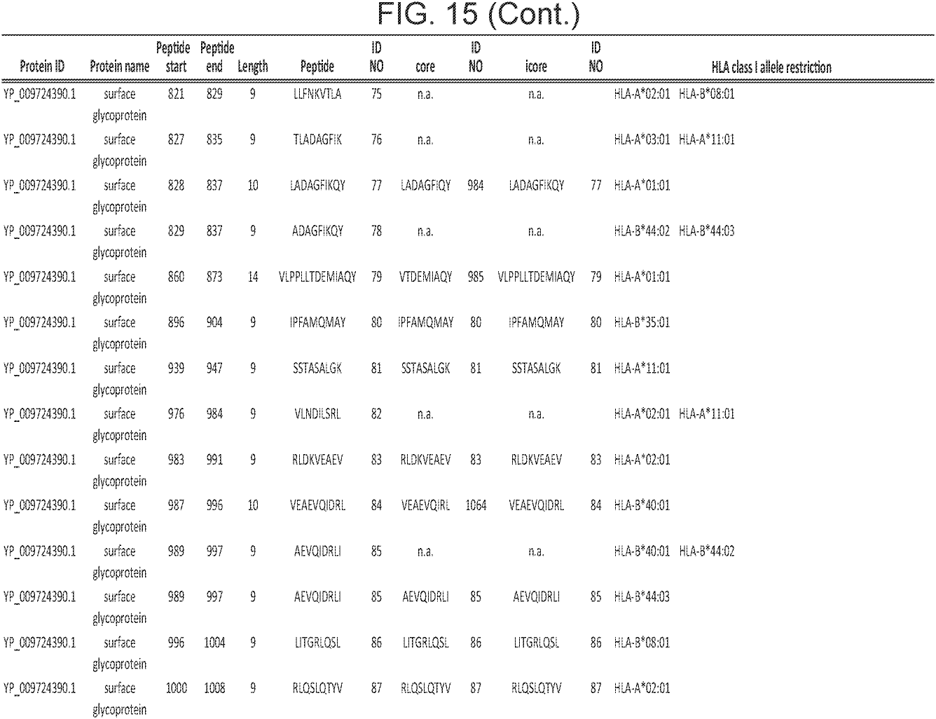

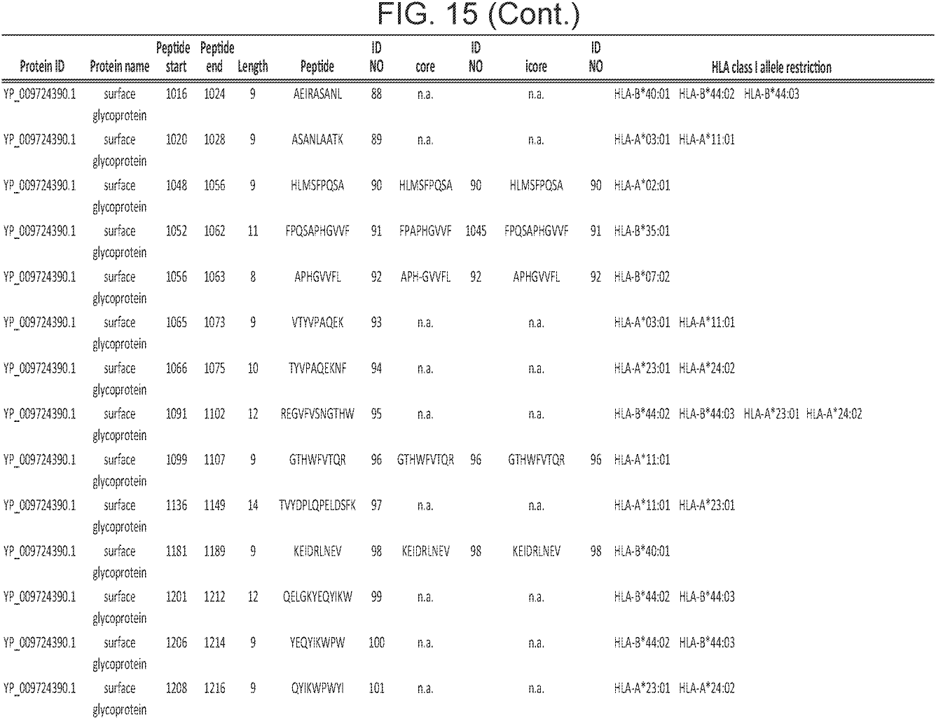

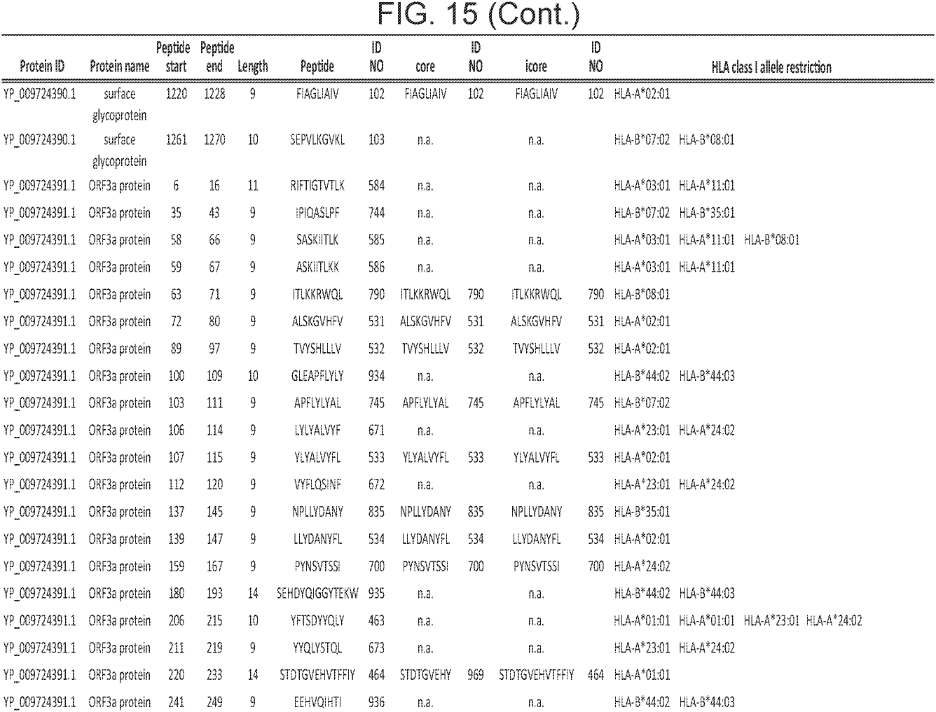

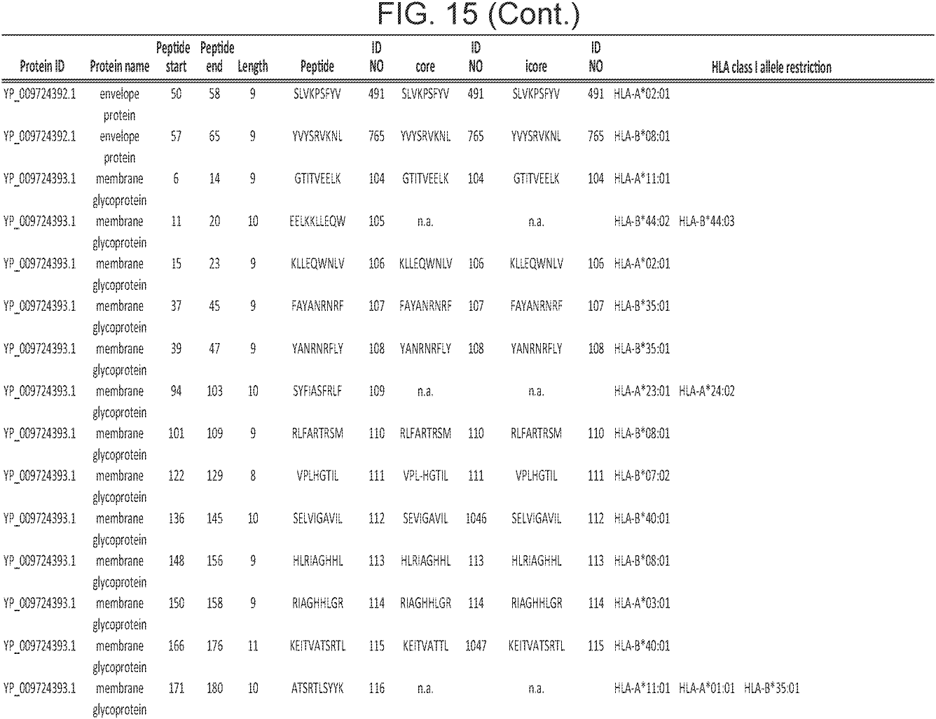

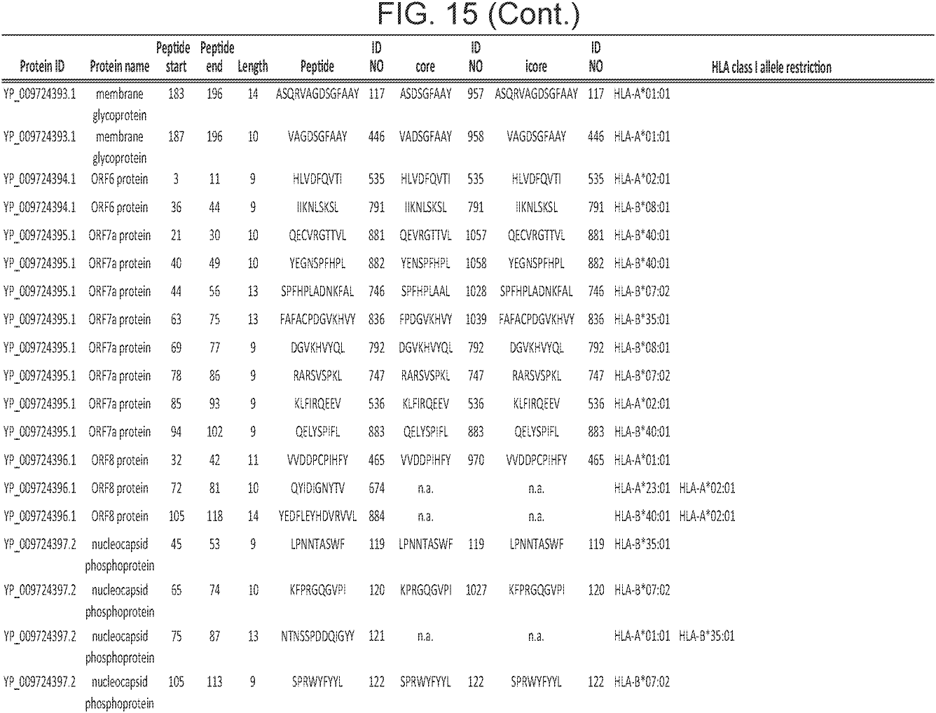

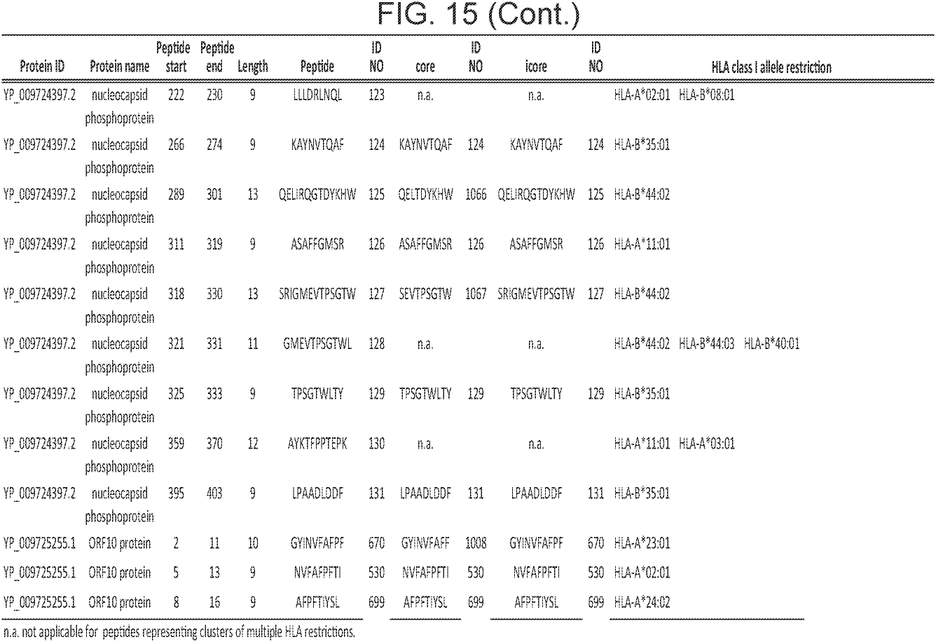

In some cases, a peptide present in a TMP is any one of the peptides depicted in FIG. 14. In some cases, a peptide present in a TMP is any one of the peptides depicted in FIG. 15.

In some cases, the peptide epitope is a SARS-CoV-2 peptide from a SARS-CoV-2-encoded surface glycoprotein. In some cases, a peptide present in a TMP is a peptide of from 4 amino acids (aa) to 25 aa in length (e.g., 4 aa, 5 aa, 6 aa, 7, aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa in length) of a polypeptide comprising an amino acid sequence having at least 50%, at least 60%, at least 70%, at least 75%, at least 80%, at least 85%, at least 90%, at least 95%, at least 98%, at least 99%, or 100%, amino acid sequence identity to the SARS-CoV-2 surface glycoprotein depicted in FIG. 13J. In some cases, a peptide present in a TMP is a peptide of from 4 amino acids (aa) to 25 aa in length (e.g., 4 aa, 5 aa, 6 aa, 7, aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa in length) of the SARS-CoV-2 surface glycoprotein depicted in FIG. 13J.

In some cases, a peptide present in a TMP is a SARS-CoV-2 peptide from a SARS-CoV-2-encoded surface glycoprotein and is selected from the group consisting of: NLTTRTQL (SEQ ID NO:19), LPPAYTNSF (SEQ ID NO:20), KVFRSSVLH (SEQ ID NO:21), LPFFSNVTW (SEQ ID NO:22), PFFSNVTWF (SEQ ID NO:23), RFDNPVLPF (SEQ ID NO:24), LPFNDGVYF (SEQ ID NO:25), GVYFASTEK (SEQ ID NO:26), TEKSNIIRGW (SEQ ID NO:27), TLDSKTQSL (SEQ ID NO:28), GVYYHKNNK (SEQ ID NO:29), YYHKNNKSW (SEQ ID NO:30), VYSSANNCTF (SEQ ID NO:31), FEYVSQPFL (SEQ ID NO:32), EYVSQPFLM (SEQ ID NO:33), FVFKNIDGY (SEQ ID NO:34), TPINLVRDL (SEQ ID NO:35), LPQGFSAL (SEQ ID NO:36), LPIGINITRF (SEQ ID NO:37), INITRFQTL (SEQ ID NO:38), LLALHRSYL (SEQ ID NO:39), WTAGAAAYY (SEQ ID NO:40), YYVGYLQPRTF (SEQ ID NO:41), YLQPRTFLL (SEQ ID NO:42), YLQPRTFL (SEQ ID NO:43), SETKCTLKSF (SEQ ID NO:44), TLKSFTVEK (SEQ ID NO:45), QPTESIVRF (SEQ ID NO:46), RFPNITNLCPF (SEQ ID NO:47), GEVFNATRF (SEQ ID NO:48), NATRFASVY (SEQ ID NO:49), LYNSASFSTF (SEQ ID NO:50), NSASFSTFK (SEQ ID NO:51), RQIAPGQTGK (SEQ ID NO:52), KIADYNYKL (SEQ ID NO:53), NYNYLYRLF (SEQ ID NO:54), RLFRKSNLK (SEQ ID NO:55), KPFERDISTEI (SEQ ID NO:56), YFPLQSYGF (SEQ ID NO:57), QPYRVVVL (SEQ ID NO:58), PYRVVVLSF (SEQ ID NO:59), GPKKSTNLV (SEQ ID NO:60), TSNQVAVLY (SEQ ID NO:61), VYSTGSNVF (SEQ ID NO:62), AEHVNNSY (SEQ ID NO:63), IPIGAGICASY (SEQ ID NO:64), SPRRARSVA (SEQ ID NO:65), VASQSIIAY (SEQ ID NO:66), SIIAYTMSL (SEQ ID NO:67), LGAENSVAY (SEQ ID NO:68), AYSNNSIAIPTNF (SEQ ID NO:69), IPTNFTISV (SEQ ID NO:70), TEILPVSMTK (SEQ ID NO:71), QEVFAQVKQIY (SEQ ID NO:72), KQIYKTPPIK (SEQ ID NO:73), IYKTPPIKDF (SEQ ID NO:74), LLFNKVTLA (SEQ ID NO:75), TLADAGFIK (SEQ ID NO:76), LADAGFIKQY (SEQ ID NO:77), ADAGFIKQY (SEQ ID NO:78), VLPPLLTDEMIAQY (SEQ ID NO:79), IPFAMQMAY (SEQ ID NO:80), SSTASALGK (SEQ ID NO:81), VLNDILSRL (SEQ ID NO:82), RLDKVEAEV (SEQ ID NO:83), VEAEVQIDRL (SEQ ID NO:84), AEVQIDRLI (SEQ ID NO:85), LITGRLQSL (SEQ ID NO:86), RLQSLQTYV (SEQ ID NO:87), AEIRASANL (SEQ ID NO:88), ASANLAATK (SEQ ID NO:89), HLMSFPQSA (SEQ ID NO:90), FPQSAPHGVVF (SEQ ID NO:91), APHGVVFL (SEQ ID NO:92), VTYVPAQEK (SEQ ID NO:93), TYVPAQEKNF (SEQ ID NO:94), REGVFVSNGTHW (SEQ ID NO:95), GTHWFVTQR (SEQ ID NO:96), TVYDPLQPELDSFK (SEQ ID NO:97), KEIDRLNEV (SEQ ID NO:98), QELGKYEQYIKW (SEQ ID NO:99), YEQYIKWPW (SEQ ID NO:100), QYIKWPWYI (SEQ ID NO:101), FIAGLIAIV (SEQ ID NO:102), and SEPVLKGVKL (SEQ ID NO:103).

In some cases, the peptide epitope is a SARS-CoV-2 peptide from a SARS-CoV-2-encoded membrane glycoprotein. In some cases, a peptide present in a TMP is a peptide of from 4 amino acids (aa) to 25 aa in length (e.g., 4 aa, 5 aa, 6 aa, 7, aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa in length) of a polypeptide comprising an amino acid sequence having at least 50%, at least 60%, at least 70%, at least 75%, at least 80%, at least 85%, at least 90%, at least 95%, at least 98%, at least 99%, or 100%, amino acid sequence identity to the SARS-CoV-2 membrane glycoprotein depicted in FIG. 13D. In some cases, a peptide present in a TMP is a peptide of from 4 amino acids (aa) to 25 aa in length (e.g., 4 aa, 5 aa, 6 aa, 7, aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa in length) of the SARS-CoV-2 membrane glycoprotein depicted in FIG. 13D.

In some cases, a peptide present in a TMP of the present disclosure is a SARS-CoV-2 peptide from a SARS-CoV-2-encoded membrane glycoprotein and is selected from the group consisting of: GTITVEELK (SEQ ID NO:104), EELKKLLEQW (SEQ ID NO:105), KLLEQWNLV (SEQ ID NO:106), FAYANRNRF (SEQ ID NO:107), YANRNRFLY (SEQ ID NO:108), SYFIASFRLF (SEQ ID NO:109), RLFARTRSM (SEQ ID NO:110), VPLHGTIL (SEQ ID NO:111), SELVIGAVIL (SEQ ID NO:112), HLRIAGHHL (SEQ ID NO:113), RIAGHHLGR (SEQ ID NO:114), KEITVATSRTL (SEQ ID NO:115), ATSRTLSYYK (SEQ ID NO:116), ASQRVAGDSGFAAY (SEQ ID NO:117), and VAGDSGFAAY (SEQ ID NO:118).

In some cases, the peptide epitope is a SARS-CoV-2 peptide from a SARS-CoV-2-encoded nucleocapsid phosphoprotein. In some cases, a peptide present in a TMP is a peptide of from 4 amino acids (aa) to 25 aa in length (e.g., 4 aa, 5 aa, 6 aa, 7, aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa in length) of a polypeptide comprising an amino acid sequence having at least 50%, at least 60%, at least 70%, at least 75%, at least 80%, at least 85%, at least 90%, at least 95%, at least 98%, at least 99%, or 100%, amino acid sequence identity to the SARS-CoV-2 nucleocapsid phosphoprotein depicted in FIG. 13F. In some cases, a peptide present in a TMP is a peptide of from 4 amino acids (aa) to 25 aa in length (e.g., 4 aa, 5 aa, 6 aa, 7, aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa in length) of the SARS-CoV-2 nucleocapsid phosphoprotein depicted in FIG. 13F.

In some cases, the peptide epitope is a SARS-CoV-2 peptide from a SARS-CoV-2-encoded nucleocapsid phosphoprotein and is selected from the group consisting of: LPNNTASWF (SEQ ID NO:119), KFPRGQGVPI (SEQ ID NO:120), NTNSSPDDQIGYY (SEQ ID NO:121), SPRWYFYYL (SEQ ID NO:122), LLLDRLNQL (SEQ ID NO:123), KAYNVTQAF (SEQ ID NO:124), QELIRQGTDYKHW (SEQ ID NO:125), ASAFFGMSR (SEQ ID NO:126), SRIGMEVTPSGTW (SEQ ID NO:127), GMEVTPSGTWL (SEQ ID NO:128), TPSGTWLTY (SEQ ID NO:129), AYKTFPPTEPK (SEQ ID NO:130), and LPAADLDDF (SEQ ID NO:131).

In some cases, the peptide epitope is RLQSLQTYV (SEQ ID NO:87). In some cases, the peptide epitope is YLQPRTFLL (SEQ ID NO:42).

### MHC Polypeptides

As noted above, a TMP of the present disclosure includes MHC polypeptides. For the purposes of the instant disclosure, the term “major histocompatibility complex (MHC) polypeptides” is meant to include MHC polypeptides of various species, including human MHC (also referred to as human leukocyte antigen (HLA)) polypeptides, rodent (e.g., mouse, rat, etc.) MHC polypeptides, and MHC polypeptides of other mammalian species (e.g., lagomorphs, non-human primates, canines, felines, ungulates (e.g., equines, bovines, ovines, caprines, etc.), and the like. The term “MHC polypeptide” is meant to include Class I MHC polypeptides (e.g., β-2 microglobulin and MHC class I heavy chain).

In some cases, the first MHC polypeptide is an MHC Class I β2M (β2M) polypeptide, and the second MHC polypeptide is an MHC Class I heavy chain (H chain) (“MHC-H”)). In other instances, the first MHC polypeptide is an MHC Class I heavy chain polypeptide; and the second MHC polypeptide is a β2M polypeptide. In some cases, both the β2M and MHC-H chain are of human origin; i.e., the MHC-H chain is an HLA heavy chain, or a variant thereof. Unless expressly stated otherwise, a TMP of the present disclosure does not include membrane anchoring domains (transmembrane regions) of an MHC Class I heavy chain, or a part of MHC Class I heavy chain sufficient to anchor the resulting TMP to a cell (e.g., eukaryotic cell such as a mammalian cell) in which it is expressed. In some cases, the MHC Class I heavy chain present in a TMP of the present disclosure does not include a signal peptide, a transmembrane domain, or an intracellular domain (cytoplasmic tail) associated with a native MHC Class I heavy chain. Thus, e.g., in some cases, the MHC Class I heavy chain present in a TMP of the present disclosure includes only the α1, α2, and α3 domains of an MHC Class I heavy chain. In some cases, the MHC Class I heavy chain present in a TMP of the present disclosure has a length of from about 270 amino acids (aa) to about 290 aa. In some cases, the MHC Class I heavy chain present in a TMP of the present disclosure has a length of 270 aa, 271 aa, 272 aa, 273 aa, 274 aa, 275 aa, 276 aa, 277 aa, 278 aa, 279 aa, 280 aa, 281 aa, 282 aa, 283 aa, 284 aa, 285 aa, 286 aa, 287 aa, 288 aa, 289 aa, or 290 aa.

In some cases, an MHC polypeptide of a TMP is a human MHC polypeptide, where human MHC polypeptides are also referred to as “human leukocyte antigen” (“HLA”) polypeptides. In some cases, an MHC polypeptide of a TMP is a Class I HLA polypeptide, e.g., a P2-microglobulin polypeptide, or a Class I HLA heavy chain polypeptide. Class I HLA heavy chain polypeptides include HLA-A heavy chain polypeptides, HLA-B heavy chain polypeptides, HLA-C heavy chain polypeptides, HLA-E heavy chain polypeptides, HLA-F heavy chain polypeptides, and HLA-G heavy chain polypeptides.

### MHC Class I Heavy Chains

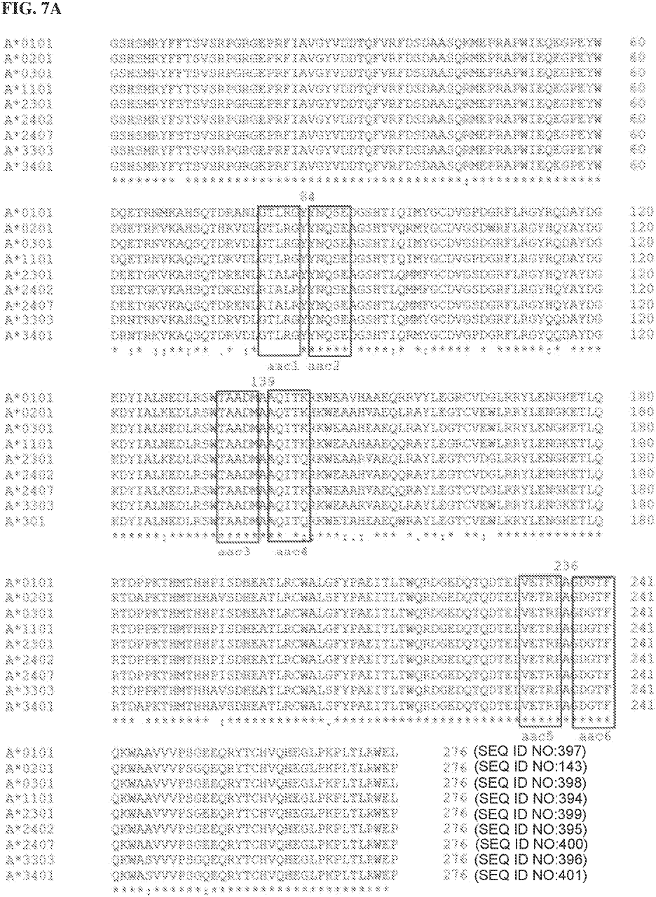

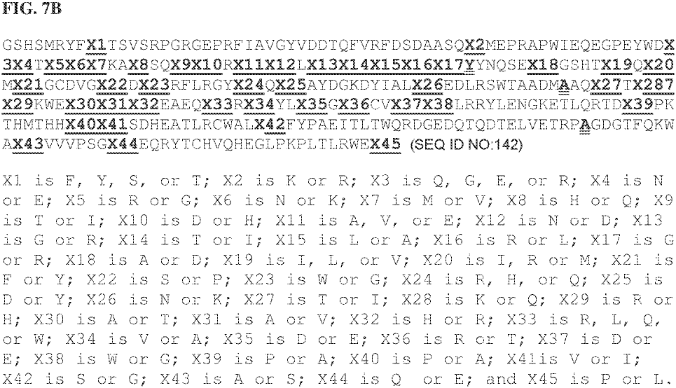

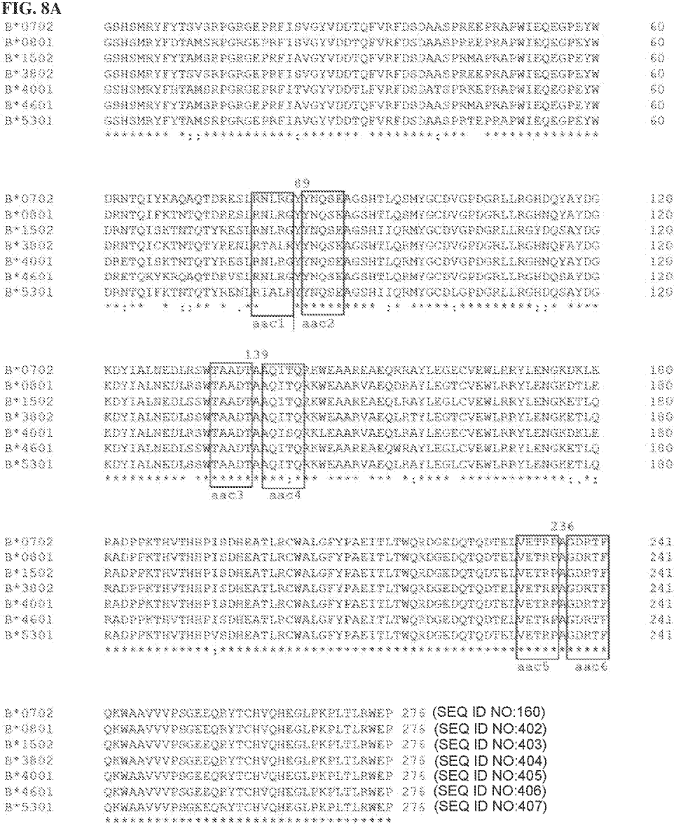

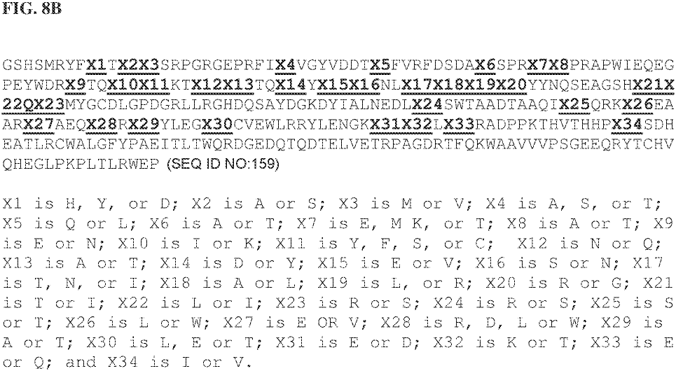

In some cases, an MHC Class I heavy chain polypeptide present in a TMP of the present disclosure comprises an amino acid sequence having at least 75%, at least 80%, at least 85%, at least 90%, at least 95%, at least 98%, at least 99%, or 100%, amino acid sequence identity to all or part (e.g., 50, 75, 100, 150, 200, or 250 contiguous amino acids) of the amino acid sequence of any of the human HLA heavy chain polypeptides depicted in FIGS. 7-13. In some cases, the MHC Class I heavy chain has a length of 270 aa, 271 aa, 272 aa, 273 aa, 274 aa, 275 aa, 276 aa, 277 aa, 278 aa, 279 aa, 280 aa, 281 aa, 282 aa, 283 aa, 284 aa, 285 aa, 286 aa, 287 aa, 288 aa, 289 aa, or 290 aa. In some cases, an MHC Class I heavy chain polypeptide present in a TMP of the present disclosure comprises 1-30, 1-5, 5-10, 10-15, 15-20, 20-25 or 25-30 amino acid insertions, deletions, and/or substitutions (in addition to those locations indicated as being variable in the heavy chain consensus sequences) of any one of the amino acid sequences depicted in FIGS. 7-13. In some cases, the MHC Class I heavy chain does not include transmembrane or cytoplasmic domains. As an example, a MHC Class I heavy chain polypeptide of a TMP of the present disclosure can comprise an amino acid sequence having at least 75%, at least 80%, at least 85%, at least 90%, at least 95%, at least 98%, at least 99%, or 100%, amino acid sequence identity to amino acids 25-300 (lacking all, or substantially all, of the leader, transmembrane and cytoplasmic sequence) or amino acids 25-365 (lacking the leader) of a human HLA-A heavy chain polypeptides depicted in any one of FIGS. 5A, 5B, and 5C.

FIGS. 5A, 5B and 5C provide amino acid sequences of human leukocyte antigen (HLA) Class I heavy chain polypeptides. Signal sequences, amino acids 1-24, are bolded and underlined. FIG. 5A entry: 3A.1 is the HLA-A heavy chain (HLA-A*01:01:01:01 or A*0101) (NCBI accession NP_001229687.1), SEQ ID NO:1071; entry 3A.2 is from HLA-A*1101 SEQ ID NO:1072; entry 3A.3 is from HLA-A*2402 SEQ ID NO:1073 and entry 3A.4 is from HLA-A*3303 SEQ ID NO:1074. FIG. 5B provides the sequence HLA-B*07:02:01 (HLA-B*0702) NCBI GenBank Accession NP_005505.2 (see also GenBank Accession AUV50118.1.). FIG. 5C provides the sequence HLA- C*0701 (GenBank Accession NP_001229971.1) (HLA-C*07:01:01:01 or HLA-Cw*070101, HLA-Cw*07 see GenBank Accession CAO78194.1).

FIG. 6 provides an alignment of eleven mature MHC class I heavy chain amino acid sequences without their leader sequences or transmembrane domains or intracellular domains. The aligned sequences are human HLA-A, HLA-B, and HLA-C, a mouse H2K protein sequence, three variants of HLA-A (var.1, var. 2C, and var. 2CP), and 3 human HLA-A variants (HLA-A*1101; HLA-A*2402; and HLA-A*3303). Indicated in the alignment are the locations (84 and 139 of the mature proteins) where cysteine residues may be introduced (e.g., by substitution) for the formation of a disulfide bond to stabilize the MHC H chain - β2M complex. Also shown in the alignment is position 236 (of the mature polypeptide), which may be substituted by a cysteine residue that can form an inter-chain disulfide bond with β2M (e.g., at aa 12). An arrow appears above each of those locations and the residues are bolded. The seventh HLA-A sequence shown in the alignment (var. 2c), shows the sequence of variant 2 substituted with C residues at positions 84, 139 and 236. The boxes flanking residues 84, 139 and 236 show the groups of five amino acids on either sides of those six sets of five residues, denoted aac1 (for “amino acid cluster 1”), aac2 (for “amino acid cluster 2”), aac3 (for “amino acid cluster 3”), aac4 (for “amino acid cluster 4”), aac5 (for “amino acid cluster 5”), and aac6 (for “amino acid cluster 6”), that may be replaced by 1 to 5 amino acids selected independently from (i) any naturally occurring amino acid or (ii) any naturally occurring amino acid except proline or glycine.

With regard to FIG. 6, in some cases: i) aac1 (amino acid cluster 1) may be the amino acid sequence GTLRG (SEQ ID NO:132) or that sequence with one or two amino acids deleted or substituted with other naturally occurring amino acids (e.g., L replaced by I, V, A or F); ii) aac2 (amino acid cluster 2) may be the amino acid sequence YNQSE (SEQ ID NO:133) or that sequence with one or two amino acids deleted or substituted with other naturally occurring amino acids (e.g., N replaced by Q, Q replaced by N, and/or E replaced by D); iii) aac3 (amino acid cluster 3) may be the amino acid sequence TAADM (SEQ ID NO:134) or that sequence with one or two amino acids deleted or substituted with other naturally occurring amino acids (e.g., T replaced by S, A replaced by G, D replaced by E, and/or M replaced by L, V, or I); iv) aac4 (amino acid cluster 4) may be the amino acid sequence AQTTK (SEQ ID NO:135) or that sequence with one or two amino acids deleted or substituted with other naturally occurring amino acids (e.g., A replaced by G, Q replaced by N, or T replaced by S, and/or K replaced by R or Q); v) aac5 (amino acid cluster 5) may be the amino acid sequence VETRP (SEQ ID NO:136) or that sequence with one or two amino acids deleted or substituted with other naturally occurring amino acids (e.g., V replaced by I or L, E replaced by D, T replaced by S, and/or R replaced by K); and/or vi) aac6 (amino acid cluster 6) may be the amino acid sequence GDGTF (SEQ ID NO:137) or that sequence with one or two amino acids deleted or substituted with other naturally occurring amino acids (e.g., D replaced by E, T replaced by S, or F replaced by L, W, or Y).

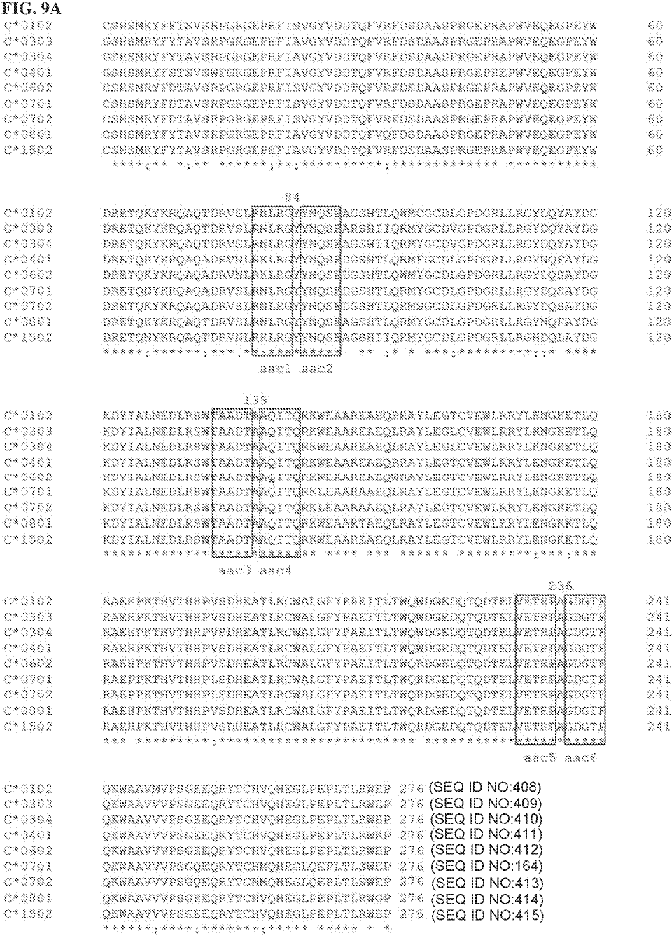

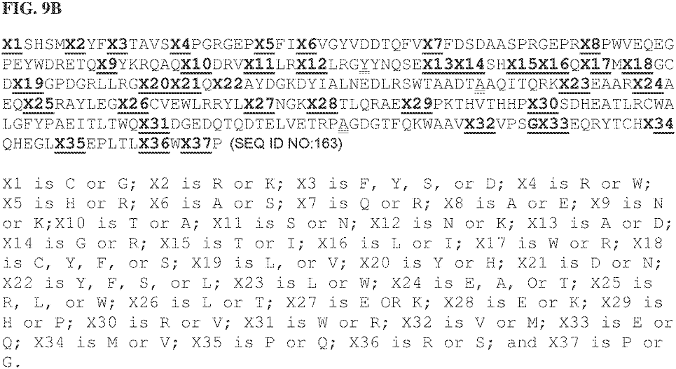

FIGS. 7-9 provide alignments of mature HLA class I heavy chain amino acid sequences (without leader sequences or transmembrane domains or intracellular domains). The aligned amino acid sequences in FIG. 7A are HLA-A class I heavy chains of the following alleles: A*0101, A*0201, A*0301, A*1101, A*2301, A*2402, A*2407, A*3303, and A*3401. The aligned amino acid sequences in FIG. 8A are HLA-B class I heavy chains of the following alleles: B*0702, B*0801, B*1502, B*3802, B*4001, B*4601, and B*5301. The aligned amino acid sequences in FIG. 9A are HLA-C class I heavy chains of the following alleles: C*0102, C*0303, C*0304, C*0401, C*0602, C*0701, C*0801, and C*1502. Indicated in the alignments are the locations (84 and 139 of the mature proteins) where cysteine residues may be introduced (e.g., by substitution) for the formation of a disulfide bond to stabilize the HLA H chain - β2M complex. Also shown in the alignment is position 236 (of the mature polypeptide), which may be substituted by a cysteine residue that can form an inter-chain disulfide bond with β2M (e.g., at aa 12). The boxes flanking residues 84, 139 and 236 show the groups of five amino acids on either sides of those six sets of five residues, denoted aac1 (for “amino acid cluster 1”), aac2 (for “amino acid cluster 2”), aac3 (for “amino acid cluster 3”), aac4 (for “amino acid cluster 4”), aac5 (for “amino acid cluster 5”), and aac6 (for “amino acid cluster 6”), that may be replaced by 1 to 5 amino acids selected independently from (i) any naturally occurring amino acid or (ii) any naturally occurring amino acid except proline or glycine.

FIGS. 7A, 8A, and 9A provide alignments of the amino acid sequences of mature HLA-A, -B, and -C class I heavy chains, respectively. The sequences are provided for the extracellular portion of the mature protein (without leader sequences or transmembrane domains or intracellular domains). As described in FIG. 6, the positions of aa residues 84, 139, and 236 and their flanking residues (aac1 to aac6) that may be replaced by 1 to 5 amino acids selected independently from (i) any naturally occurring amino acid or (ii) any naturally occurring amino acid except proline or glycine ae also shown. FIGS. 7B, 8B, and 9B provide consensus amino acid sequences for the HLA-A, -B, and -C sequences, respectively, provide in FIGS. 7A, 8A, and 9A. The consensus sequences show the variable amino acid positions as “X” residues sequentially numbered and the locations of amino acids 84, 139 and 236 double underlined.

With regard to FIG. 7A, in some cases: i) aac1 (amino acid cluster 1) may be the amino acid sequence GTLRG (SEQ ID NO:132) or that sequence with one or two amino acids deleted or substituted with other naturally occurring amino acids (e.g., L replaced by I, V, A or F); ii) aac2 (amino acid cluster 2) may be the amino acid sequence YNQSE (SEQ ID NO:133) or that sequence with one or two amino acids deleted or substituted with other naturally occurring amino acids (e.g., N replaced by Q, Q replaced by N, and/or E replaced by D); iii) aac3 (amino acid cluster 3) may be the amino acid sequence TAADM (SEQ ID NO:134) or that sequence with one or two amino acids deleted or substituted with other naturally occurring amino acids (e.g., T replaced by S, A replaced by G, D replaced by E, and/or M replaced by L, V, or I); iv) aac4 (amino acid cluster 4) may be the amino acid sequence AQTTK (SEQ ID NO:135) or that sequence with one or two amino acids deleted or substituted with other naturally occurring amino acids (e.g., A replaced by G, Q replaced by N, or T replaced by S, and or K replaced by R or Q); v) aac5 (amino acid cluster 5) may be the amino acid sequence VETRP (SEQ ID NO:136) or that sequence with one or two amino acids deleted or substituted with other naturally occurring amino acids (e.g., V replaced by I or L, E replaced by D, T replaced by S, and/or R replaced by K); and/or vi) aac6 (amino acid cluster 6) may be the amino acid sequence GDGTF (SEQ ID NO:137) or that sequence with one or two amino acids deleted or substituted with other naturally occurring amino acids (e.g., D replaced by E, T replaced by S, or F replaced by L, W, or Y).

With regard to FIG. 8A, in some cases: i) aac1 (amino acid cluster 1) may be the amino acid sequence RNLRG (SEQ ID NO:138) or that sequence with one or two amino acids deleted or substituted with other naturally occurring amino acids (e.g., N replaced by T or I; and/or L replaced by A; and/or the second R replaced by L; and/or the G replaced by R); ii) aac2 (amino acid cluster 2) may be the amino acid sequence YNQSE (SEQ ID NO:133) or that sequence with one or two amino acids deleted or substituted with other naturally occurring amino acids (e.g., N replaced by Q, Q replaced by N, and/or E replaced by D); iii) aac3 (amino acid cluster 3) may be the amino acid sequence TAADT (SEQ ID NO:139) or that sequence with one or two amino acids deleted or substituted with other naturally occurring amino acids (e.g., the first T replaced by S; and/or A replaced by G; and/or D replaced by E; and/or the second T replaced by S); iv) aac4 (amino acid cluster 4) may be the amino acid sequence AQITQ (SEQ ID NO:140) or that sequence with one or two amino acids deleted or substituted with other naturally occurring amino acids (e.g., A replaced by G; and/or the first Q replaced by N; and/or I replaced by L or V; and/or the T replaced by S; and/or the second Q replaced by N); v) aac5 (amino acid cluster 5) may be the amino acid sequence VETRP (SEQ ID NO:136) or that sequence with one or two amino acids deleted or substituted with other naturally occurring amino acids (e.g., V replaced by I or L, E replaced by D, T replaced by S, and/or R replaced by K); and/or vi) aac6 (amino acid cluster 6) may be the amino acid sequence GDRTF (SEQ ID NO:141) or that sequence with one or two amino acids deleted or substituted with other naturally occurring amino acids (e.g., D replaced by E; and/or T replaced by S; and/or R replaced by K or H; and/or F replaced by L, W, or Y).

With regard to FIG. 9A, in some cases: i) aac1 (amino acid cluster 1) may be the amino acid sequence RNLRG (SEQ ID NO:138) or that sequence with one or two amino acids deleted or substituted with other naturally occurring amino acids (e.g., N replaced by K; and/or L replaced by A or I; and/or the second R replaced by H; and/or the G replaced by T or S); ii) aac2 (amino acid cluster 2) may be the amino acid sequence YNQSE (SEQ ID NO:133) or that sequence with one or two amino acids deleted or substituted with other naturally occurring amino acids (e.g., N replaced by Q, Q replaced by N, and/or E replaced by D); iii) aac3 (amino acid cluster 3) may be the amino acid sequence TAADT (SEQ ID NO:139) or that sequence with one or two amino acids deleted or substituted with other naturally occurring amino acids (e.g., the first T replaced by S; and/or A replaced by G; and/or D replaced by E; and/or the second T replaced by S); iv) aac4 (amino acid cluster 4) may be the amino acid sequence AQITQ (SEQ ID NO:140) or that sequence with one or two amino acids deleted or substituted with other naturally occurring amino acids (e.g., A replaced by G; and/or the first Q replaced by N; and/or I replaced by L; and/or the second Q replaced by N or K); v) aac5 (amino acid cluster 5) may be the amino acid sequence VETRP (SEQ ID NO:136) or that sequence with one or two amino acids deleted or substituted with other naturally occurring amino acids (e.g., V replaced by I or L, E replaced by D, T replaced by S, and/or R replaced by K or H); and/or vi) aac6 (amino acid cluster 6) may be the amino acid sequence GDGTF (SEQ ID NO:137) or that sequence with one or two amino acids deleted or substituted with other naturally occurring amino acids (e.g., D replaced by E; and/or T replaced by S; and/or F replaced by L, W, or Y).

### Hla-a

In some cases, a TMP of the present disclosure comprises an HLA-A heavy chain polypeptide. The HLA-A heavy chain peptide sequences, or portions thereof, that may be that may be incorporated into a TMP of the present disclosure include, but are not limited to, the alleles: A*0101, A*0201, A*0301, A*1101, A*2301, A*2402, A*2407, A*3303, and A*3401, which are aligned without all, or substantially all, of the leader, transmembrane and cytoplasmic sequences in FIG. 7A. Any of those alleles may comprise a mutation at one or more of positions 84, 139 and/or 236 (as shown in FIG. 7A) selected from: a tyrosine to alanine at position 84 (Y84A); a tyrosine to cysteine at position 84 (Y84C); an alanine to cysteine at position 139 (A139C); and an alanine to cysteine substitution at position 236 (A236C). In addition, HLA-A sequence having at least 75% (e.g., at least 80%, at least 85%, at least 90%, at least 95%, at least 98%, at least 99%) or 100% amino acid sequence identity to all or part (e.g., 50, 75, 100, 150, 200, or 250 contiguous amino acids) of the sequence of those HLA-A alleles may also be employed (e.g., it may comprise 1-25, 1-5, 5-10, 10-15, 15-20, 20-25, or 25-30 amino acid insertions, deletions, and/or substitutions).

In some cases, a TMP of the present disclosure comprises an HLA-A heavy chain polypeptide comprising the following HLA-A consensus amino acid sequence:

GSHSMRYFX1 TSVSRPGRGEPRFIAVGYVDDTQFVRFDSDAASQX2 MEPR
APWIEQEGPEYWDX3X4 TX5X6X7 KAX8 SQX9X10 RX11X12 LX13X14X 15X16X17 YYNQSEX18 GSHTX19 0X20 MX21 GCDVGX22 DX23 RFLRGY X24 QX25 AYDGKDYIALX26 EDLRSWTAADMAAQX 27 TX287X29 KWEX3 0X31X32 EAEQX33 RX34 YLX35 GX36 CVX37X38 LRRYLENGKETLQRT
DX3 9 PKTHMTHHX40X41 SDHEATLRCWALX42 FYPAEITLTWQRDGEDQ
TQDTELVETRPAGDGTFQKWAX43 VVVPSGX44 EQRYTCHVQHEGLPKPL
TLRWEX45 (SEQ ID NO:142),

wherein X1 is F, Y, S, or T; X2 is K or R; X3 is Q, G, E, or R; X4 is N or E; X5 is R or G; X6 is N or K; X7 is M or V; X8 is H or Q; X9 is T or I; XIO is D or H; X11 is A, V, or E; X12 is N or D; X13 is G or R; X14 is ‘I’ or I; X15 is L or A; X16 is R or L; X17 is G or R; X18 is A or D; X19 is I, L, or V; X20 is I, R or M; X21 is F or Y; X22 is 5 or P; X23 is W or G; X24 is R, H, or Q; X25 is D or Y; X26 is N or K; X27 is T or I; X28 is K or Q; X29 is R or H; X30 is A or T; X31 is A or V; X32 is H or R; X33 is R, L, Q, or W; X34 is V or A; X35 is D or E; X36 is R or T; X37 is D or E; X38 is W or G; X39 is P or A; X40 is P or A; X41 is V or I; X42 is S or G; X43 is A or S; X44 is Q or E; and X45 is P or L.

As one example, an MHC Class I heavy chain polypeptide of a TMP can comprise an amino acid sequence having at least 75%, at least 80%, at least 85%, at least 90%, at least 95%, at least 98%, at least 99%, or 100%, amino acid sequence identity to the following human HLA-A heavy chain amino acid sequence:

GSHSMRYFFTSVSRPGRGEPRFIAVGYVDDTQFVRFDSDAASQRMEPRAP
WIEQEGPEYWDGETRKVKAHSQTHRVDLGTLRGYYNQSEAGSHTVQRMYG
CDVGSDWRFLRGYHQYAYDGKDYIALKEDLRSWTAADMAAQTTKHKWEAA
HVAEQLRAYLEGTCVEWLRRYLENGKETLQRTDAPKTHMTHHAVSDHEAT
LRCWALSFYPAEITLTWQRDGEDQTQDTELVETRPAGDGTFQKWAAVVVP
SGQEQRYTCHVQHEGLPKPLTLRWEP (SEQ ID NO:143).

In some cases, an HLA-A heavy chain polypeptide suitable for inclusion in a TMP of the present disclosure comprises the following amino acid sequence:

GSHSMRYFFTSVSRPGRGEPRFIAVGYVDDTQFVRFDSDAASQRMEPRAP
WIEQEGPEYWDGETRKVKAHSQTHRVDLGTLRGYYNQSEAGSHTVQRMYG
CDVGSDWRFLRGYHQYAYDGKDYIALKEDLRSWTAADMAAQTTKHKWEAA
HVAEQLRAYLEGTCVEWLRRYLENGKETLQRTDAPKTHMTHHAVSDHEAT
LRCWALSFYPAEITLTWQRDGEDQTQDTELVETRPAGDGTFQKWAAVVVP
SGQEQRYTCHVQHEGLPKPLTLRWEP (SEQ ID NO:143).

This HLA-A heavy chain polypeptide is also referred to as “HLA-A*0201” or simply “HLA-A02.” In some cases, the C-terminal Pro is not included in a TMP of the present disclosure. For example, in some cases, an HLA-A02 polypeptide suitable for inclusion in a TMP of the present disclosure comprises the following amino acid sequence:

GSHSMRYFFTSVSRPGRGEPRFIAVGYVDDTQFVRFDSDAASQRMEPRAP
WIEQEGPEYWDGETRKVKAHSQTHRVDLGTLRGYYNQSEAGSHTVQRMYG
CDVGSDWRFLRGYHQYAYDGKDYIALKEDLRSWTAADMAAQTTKHKWEAA
HVAEQLRAYLEGTCVEWLRRYLENGKETLQRTDAPKTHMTHHAVSDHEAT
LRCWALSFYPAEITLTWQRDGEDQTQDTELVETRPAGDGTFQKWAAVVVP
SGQEQRYTCHVQHEGLPKPLTLRWE (SEQ ID NO:144).

### Hla-a (y84a; A236c)

In some cases, the MHC Class I heavy chain polypeptide comprises Y84A and A236C substitutions. For example, in some cases, the MHC Class I heavy chain polypeptide comprises an amino acid sequence having at least 75%, at least 80%, at least 85%, at least 90%, at least 95%, at least 98%, at least 99%, or 100%, amino acid sequence identity to the following human HLA-A heavy chain (Y84A; A236C) amino acid sequence:

GSHSMRYFFTSVSRPGRGEPRFIAVGYVDDTQFVRFDSDAASQRMEPRAP
WIEQEGPEYWDGETRKVKAHSQTHRVDLGTLRGA YNQSEAGSHTVQRMYG
CDVGSDWRFLRGYHQYAYDGKDYIALKEDLRSWTAADMAAQTTKHKWEAA
HVAEQLRAYLEGTCVEWLRRYLENGKETLQRTDAPKTHMTHHAVSDHEAT
LRCWALSFYPAEITLTWQRDGEDQTQDTELVETRPC GDGTFQKWAAVVVP
SGQEQRYTCHVQHEGLPKPLTLRWEP (SEQ ID NO:145),

where amino acid 84 is Ala and amino acid 236 is Cys. In some cases, the Cys-236 forms an interchain disulfide bond with Cys-12 of a variant β2M polypeptide that comprises an R12C substitution.

In some cases, an HLA-A heavy chain polypeptide suitable for inclusion in a TMP of the present disclosure is an HLA-A02 (Y84A; A236C) polypeptide comprising the following amino acid sequence:

GSHSMRYFFTSVSRPGRGEPRFIAVGYVDDTQFVRFDSDAASQRMEPRAP
WIEQEGPEYWDGETRKVKAHSQTHRVDLGTLRGA YNQSEAGSHTVQRMYG
CDVGSDWRFLRGYHQYAYDGKDYIALKEDLRSWTAADMAAQTTKHKWEAA
HVAEQLRAYLEGTCVEWLRRYLENGKETLQRTDAPKTHMTHHAVSDHEAT
LRCWALSFYPAEITLTWQRDGEDQTQDTELVETRPC GDGTFQKWAAVVVP
SGQEQRYTCHVQHEGLPKPLTLRWEP (SEQ ID NO:145).

In some cases, an HLA-A heavy chain polypeptide suitable for inclusion in a TMP of the present disclosure is an HLA-A02 (Y84A; A236C) polypeptide comprising the following amino acid sequence:

GSHSMRYFFTSVSRPGRGEPRFIAVGYVDDTQFVRFDSDAASQRMEPRAP
WIEQEGPEYWDGETRKVKAHSQTHRVDLGTLRGA YNQSEAGSHTVQRMYG
CDVGSDWRFLRGYHQYAYDGKDYIALKEDLRSWTAADMAAQTTKHKWEAA
HVAEQLRAYLEGTCVEWLRRYLENGKETLQRTDAPKTHMTHHAVSDHEAT
LRCWALSFYPAEITLTWQRDGEDQTQDTELVETRPC GDGTFQKWAAVVVP
SGQEQRYTCHVQHEGLPKPLTLRWE (SEQ ID NO:146).

### Hla-a (y84c; A139c)

In some cases, the MHC Class I heavy chain polypeptide comprises Y84C and A139C substitutions. For example, in some cases, the MHC Class I heavy chain polypeptide comprises an amino acid sequence having at least 75%, at least 80%, at least 85%, at least 90%, at least 95%, at least 98%, at least 99%, or 100%, amino acid sequence identity to the following human HLA-A heavy chain (Y84C; A139C) amino acid sequence:

GSHSMRYFFTSVSRPGRGEPRFIAVGYVDDTQFVRFDSDAASQRMEPRAP
WIEQEGPEYWDGETRKVKAHSQTHRVDLGTLRGC YNQSEAGSHTVQRMYG
CDVGSDWRFLRGYHQYAYDGKDYIALKEDLRSWTAADMC AQTTKHKWEAA
HVAEQLRAYLEGTCVEWLRRYLENGKETLQRTDAPKTHMTHHAVSDHEAT
LRCWALSFYPAEITLTWQRDGEDQTQDTELVETRPAGDGTFQKWAAVVVP
SGQEQRYTCHVQHEGLPKPLTLRWEP (SEQ ID NO:147),

where amino acid 84 is Cys and amino acid 139 is Cys. In some cases, Cys-84 forms an intrachain disulfide bond with Cys-139.

### Hla-a (y84c; A139c; A236c)

In some cases, a MHC Class I heavy chain polypeptide suitable for inclusion in a TMP of the present disclosure comprises an amino acid sequence having at least 75%, at least 80%, at least 85%, at least 90%, at least 95%, at least 98%, at least 99%, or 100%, amino acid sequence identity to the following human HLA-A heavy chain (Y84C; A139C; A236C) amino acid sequence:

GSHSMRYFFTSVSRPGRGEPRFIAVGYVDDTQFVRFDSDAASQRMEPRAP
WIEQEGPEYWDGETRKVKAHSQTHRVDLGTLRGC YNQSEAGSHTVQRMYG
CDVGSDWRFLRGYHQYAYDGKDYIALKEDLRSWTAADMC AQTTKHKWEAA
HVAEQLRAYLEGTCVEWLRRYLENGKETLQRTDAPKTHMTHHAVSDHEAT
LRCWALSFYPAEITLTWQRDGEDQTQDTELVETRPC GDGTFQKWAAVVVP
SGQEQRYTCHVQHEGLPKPLTLRWEP (SEQ ID NO:148),

where amino acid 84 is Cys, amino acid 139 is Cys, and amino acid 236 is Cys. In some cases, Cys-84 forms an intrachain disulfide bondwith Cys-139. The Cys at amino acid 236 can form a disulfide bond with a Cys residue in a second polypeptide chain. For example, the Cys at amino acid 236 can form a disulfide bond with the Cys-12 residue in a β2M polypeptide comprising an R12C substitution.

In some cases, an HLA-A heavy chain polypeptide suitable for inclusion in a TMP of the present disclosure is an HLA-A02 (Y84C; A139C; A236C) polypeptide comprising the following amino acid sequence:

GSHSMRYFFTSVSRPGRGEPRFIAVGYVDDTQFVRFDSDAASQRMEPRAP
WIEQEGPEYWDGETRKVKAHSQTHRVDLGTLRGC YNQSEAGSHTVQRMYG
CDVGSDWRFLRGYHQYAYDGKDYIALKEDLRSWTAADMC AQTTKHKWEAA
HVAEQLRAYLEGTCVEWLRRYLENGKETLQRTDAPKTHMTHHAVSDHEAT
LRCWALSFYPAEITLTWQRDGEDQTQDTELVETRPC GDGTFQKWAAVVVP
SGQEQRYTCHVQHEGLPKPLTLRWEP (SEQ ID NO:148).

### HLA-All (HLA-A*1101)

As one non-limiting example, an MHC Class I heavy chain polypeptide of a TMP can comprise an amino acid sequence having at least 75%, at least 80%, at least 85%, at least 90%, at least 95%, at least 98%, at least 99%, or 100%, amino acid sequence identity to the following human HLA-A11 heavy chain amino acid sequence:

GSHSMRYFYTSVSRPGRGEPRFIAVGYVDDTQFVRFDSDAASQRMEPRAP
WIEQEGPEYWDQETRNVKAQSQTDRVDLGTLRGYYNQSEDGSHTIQIMYG
CDVGPDGRFLRGYRQDAYDGKDYIALNEDLRSWTAADMAAQITKRKWEAA
HAAEQQRAYLEGTCVEWLRRYLENGKETLQRTDPPKTHMTHHPISDHEAT
LRCWALGFYPAEITLTWQRDGEDQTQDTELVETRPAGDGTFQKWAAVVVP
SGEEQRYTCHVQHEGLPKPLTLRWE (SEQ ID NO:149).

Such an MHC Class I heavy chain may be prominent in Asian populations, including populations of individuals of Asian descent.

### Hla-a11 (y84a; A236c)

As one non-limiting example, in some cases, the MHC Class I heavy chain polypeptide is an HLA-A11 allele that comprises Y84A and A236C substitutions. For example, in some cases, the MHC Class I heavy chain polypeptide comprises an amino acid sequence having at least 75%, at least 80%, at least 85%, at least 90%, at least 95%, at least 98%, at least 99%, or 100%, amino acid sequence identity to the following human HLA-A A11 heavy chain (Y84A; A236C) amino acid sequence:

GSHSMRYFYTSVSRPGRGEPRFIAVGYVDDTQFVRFDSDAASQRMEPRAP
WIEQEGPEYWDQETRNVKAQSQTDRVDLGTLRGA YNQSEDGSHTIQIMYG
CDVGPDGRFLRGYRQDAYDGKDYIALNEDLRSWTAADMAAQITKRKWEAA
HAAEQQRAYLEGTCVEWLRRYLENGKETLQRTDPPKTHMTHHPISDHEAT
LRCWALGFYPAEITLTWQRDGEDQTQDTELVETRPC GDGTFQKWAAVVVP
SGEEQRYTCHVQHEGLPKPLTLRWE (SEQ ID NO:150),

where amino acid 84 is Ala and amino acid 236 is Cys. In some cases, the Cys-236 forms an interchain disulfide bond with Cys-12 of a variant β2M polypeptide that comprises an R12C substitution.

### Hla-a24 (hla-a *2402)

As one non-limiting example, an MHC Class I heavy chain polypeptide of a TMP of the present disclosure can comprise an amino acid sequence having at least 75%, at least 80%, at least 85%, at least 90%, at least 95%, at least 98%, at least 99%, or 100%, amino acid sequence identity to the following human HLA-A24 heavy chain amino acid sequence:

GSHSMRYFSTSVSRPGRGEPRFIAVGYVDDTQFVRFDSDAASQRMEPRAP
WIEQEGPEYWDEETGKVKAHSQTDRENLRIALRY YNQSEAGSHTLQMMFG
CDVGSDGRFLRGYHQYAYDGKDYIALKEDLRSWTAADMAAQITKRKWEAA
HVAEQQRAYLEGTCVDGLRRYLENGKETLQRTDPPKTHMTHHPISDHEAT
LRCWALGFYPAEITLTWQRDGEDQTQDTELVETRPA GDGTFQKWAAVVVP
SGEEQRYTCHVQHEGLPKPLTLRWEPSSQPTVPIVGIIAGLVLLGAVITG
AVVAAVMWRRNSSDRKGGSYSQAASSDSAQGSDVSLTACKV (SEQ ID
NO:151).

Such an MHC Class I heavy chain may be prominent in Asian populations, including populations of individuals of Asian descent. In some cases, amino acid 84 is an Ala. In some cases, amino acid 84 is a Cys. In some cases, amino acid 236 is a Cys. In some cases, amino acid 84 is an Ala and amino acid 236 is a Cys. In some cases, amino acid 84 is a Cys and amino acid 236 is a Cys.

As one non-limiting example, an MHC Class I heavy chain polypeptide of a TMP of the present disclosure can comprise an amino acid sequence having at least 75%, at least 80%, at least 85%, at least 90%, at least 95%, at least 98%, at least 99%, or 100%, amino acid sequence identity to the following human HLA-A24 (also referred to as HLA-A*2402) heavy chain amino acid sequence:

GSHSMRYFSTSVSRPGRGEPRFIAVGYVDDTQFVRFDSDAASQRMEPRAP
WIEQEGPEYWDEETGKVKAHSQTDRENLRIALRYYNQSEAGSHTLQMMFG
CDVGSDGRFLRGYHQYAYDGKDYIALKEDLRSWTAADMAAQITKRKWEAA
HVAEQQRAYLEGTCVDGLRRYLENGKETLQRTDPPKTHMTHHPISDHEAT
LRCWALGFYPAEITLTWQRDGEDQTQDTELVETRPAGDGTFQKWAAVVVP
SGEEQRYTCHVQHEGLPKPLTLRWEPSSQPTVPIVGIIAGLVLLGAVITG
AVVAAVMWRRNSSDRKGGSYSQAASSDSAQGSDVSLTACKV (SEQ ID
NO:151).

Such an MHC Class I heavy chain may be prominent in Asian populations, including populations of individuals of Asian descent. In some cases, amino acid 84 is an Ala. In some cases, amino acid 84 is a Cys. In some cases, amino acid 236 is a Cys. In some cases, amino acid 84 is an Ala and amino acid 236 is a Cys. In some cases, amino acid 84 is a Cys and amino acid 236 is a Cys.

In some cases, an MHC Class I heavy chain polypeptide of a TMP of the present disclosure can comprise an amino acid sequence having at least 75%, at least 80%, at least 85%, at least 90%, at least 95%, at least 98%, at least 99%, or 100%, amino acid sequence identity to the following human HLA-A24 (also referred to as HLA-A*2402) heavy chain amino acid sequence:

GSHSMRYFSTSVSRPGRGEPRFIAVGYVDDTQFVRFDSDAASQRMEPRAP
WIEQEGPEYWDEETGKVKAHSQTDRENLRIALRY YNQSEAGSHTLQMMFG
CDVGSDGRFLRGYHQYAYDGKDYIALKEDLRSWTAADMAAQITKRKWEAA
HVAEQQRAYLEGTCVDGLRRYLENGKETLQRTDPPKTHMTHHPISDHEAT
LRCWALGFYPAEITLTWQRDGEDQTQDTELVETRPA GDGTFQKWAAVVVP
SGEEQRYTCHVQHEGLPKPLTLRWE (SEQ ID NO:152),

where amino acid 84 is Tyr and amino acid 236 is Ala (amino acids 84 and 236 are bold and underlined); and where the MHC Class I heavy chain has a length of about 275 amino acids.

In some cases, an MHC Class I heavy chain polypeptide of a TMP of the present disclosure can comprise an amino acid sequence having at least 75%, at least 80%, at least 85%, at least 90%, at least 95%, at least 98%, at least 99%, or 100%, amino acid sequence identity to the following human HLA-A24 (also referred to as HLA-A*2402) heavy chain amino acid sequence:

GSHSMRYFSTSVSRPGRGEPRFIAVGYVDDTQFVRFDSDAASQRMEPRAP
WIEQEGPEYWDEETGKVKAHSQTDRENLRIALRA YNQSEAGSHTLQMMFG
CDVGSDGRFLRGYHQYAYDGKDYIALKEDLRSWTAADMAAQITKRKWEAA
HVAEQQRAYLEGTCVDGLRRYLENGKETLQRTDPPKTHMTHHPISDHEAT
LRCWALGFYPAEITLTWQRDGEDQTQDTELVETRPA GDGTFQKWAAVVVP
SGEEQRYTCHVQHEGLPKPLTLRWE (SEQ ID NO:153),

where amino acid 84 is Ala and amino acid 236 is Ala (amino acids 84 and 236 are bold and underlined); and where the MHC Class I heavy chain has a length of about 275 amino acids.

In some cases, an MHC Class I heavy chain polypeptide of a TMP of the present disclosure can comprise an amino acid sequence having at least 75%, at least 80%, at least 85%, at least 90%, at least 95%, at least 98%, at least 99%, or 100%, amino acid sequence identity to the following human HLA-A24 (also referred to as HLA-A*2402) heavy chain amino acid sequence:

GSHSMRYFSTSVSRPGRGEPRFIAVGYVDDTQFVRFDSDAASQRMEPRAP
WIEQEGPEYWDEETGKVKAHSQTDRENLRIALRY YNQSEAGSHTLQMMFG
CDVGSDGRFLRGYHQYAYDGKDYIALKEDLRSWTAADMAAQITKRKWEAA
HVAEQQRAYLEGTCVDGLRRYLENGKETLQRTDPPKTHMTHHPISDHEAT
LRCWALGFYPAEITLTWQRDGEDQTQDTELVETRPC GDGTFQKWAAVVVP
SGEEQRYTCHVQHEGLPKPLTLRWE (SEQ ID NO:154),

where amino acid 84 is Tyr and amino acid 236 is Cys (amino acids 84 and 236 are bold and underlined); and where the MHC Class I heavy chain has a length of about 275 amino acids.

In some cases, an MHC Class I heavy chain polypeptide of a TMP of the present disclosure can comprise an amino acid sequence having at least 75%, at least 80%, at least 85%, at least 90%, at least 95%, at least 98%, at least 99%, or 100%, amino acid sequence identity to the following human HLA-A24 (also referred to as HLA-A*2402) heavy chain amino acid sequence:

GSHSMRYFSTSVSRPGRGEPRFIAVGYVDDTQFVRFDSDAASQRMEPRAP
WIEQEGPEYWDEETGKVKAHSQTDRENLRIALRA YNQSEAGSHTLQMMFG
CDVGSDGRFLRGYHQYAYDGKDYIALKEDLRSWTAADMAAQITKRKWEAA
HVAEQQRAYLEGTCVDGLRRYLENGKETLQRTDPPKTHMTHHPISDHEAT
LRCWALGFYPAEITLTWQRDGEDQTQDTELVETRPC GDGTFQKWAAVVVP
SGEEQRYTCHVQHEGLPKPLTLRWE (SEQ ID NO:155),

where amino acid 84 is Ala and amino acid 236 is Cys (amino acids 84 and 236 are bold and underlined); and where the MHC Class I heavy chain has a length of about 275 amino acids.

In some cases, an MHC Class I heavy chain polypeptide of a TMP of the present disclosure can comprise an amino acid sequence having at least 75%, at least 80%, at least 85%, at least 90%, at least 95%, at least 98%, at least 99%, or 100%, amino acid sequence identity to the following human HLA-A24 (also referred to as HLA-A*2402) heavy chain amino acid sequence:

GSHSMRYFSTSVSRPGRGEPRFIAVGYVDDTQFVRFDSDAASQRMEPRAP
WIEQEGPEYWDEETGKVKAHSQTDRENLRIALRC YNQSEAGSHTLQMMFG
CDVGSDGRFLRGYHQYAYDGKDYIALKEDLRSWTAADMAAQITKRKWEAA
HVAEQQRAYLEGTCVDGLRRYLENGKETLQRTDPPKTHMTHHPISDHEAT
LRCWALGFYPAEITLTWQRDGEDQTQDTELVETRPA GDGTFQKWAAVVVP
SGEEQRYTCHVQHEGLPKPLTLRWE (SEQ ID NO:156),

where amino acid 84 is Cys and amino acid 236 is Ala (amino acids 84 and 236 are bold and underlined); and where the MHC Class I heavy chain has a length of about 275 amino acids.

In some cases, an MHC Class I heavy chain polypeptide of a TMP of the present disclosure can comprise an amino acid sequence having at least 75%, at least 80%, at least 85%, at least 90%, at least 95%, at least 98%, at least 99%, or 100%, amino acid sequence identity to the following human HLA-A24 (also referred to as HLA-A*2402) heavy chain amino acid sequence:

GSHSMRYFSTSVSRPGRGEPRFIAVGYVDDTQFVRFDSDAASQRMEPRAP
WIEQEGPEYWDEETGKVKAHSQTDRENLRIALRC YNQSEAGSHTLQMMFG
CDVGSDGRFLRGYHQYAYDGKDYIALKEDLRSWTAADMAAQITKRKWEAA
HVAEQQRAYLEGTCVDGLRRYLENGKETLQRTDPPKTHMTHHPISDHEAT
LRCWALGFYPAEITLTWQRDGEDQTQDTELVETRPC GDGTFQKWAAVVVP
SGEEQRYTCHVQHEGLPKPLTLRWE (SEQ ID NO:157),

where amino acid 84 is Cys and amino acid 236 is Cys (amino acids 84 and 236 are bold and underlined); and where the MHC Class I heavy chain has a length of about 275 amino acids.

### Hla-a33 (hla-a*3303)

As one non-limiting example, an MHC Class I heavy chain polypeptide of a TMP of the present disclosure can comprise an amino acid sequence having at least 75%, at least 80%, at least 85%, at least 90%, at least 95%, at least 98%, at least 99%, or 100%, amino acid sequence identity to the following human HLA-A33 heavy chain amino acid sequence:

GSHSMRYFTTSVSRPGRGEPRFIAVGYVDDTQFVRFDSDAASQRMEPRAP
WIEQEGPEYWDRNTRNVKAHSQIDRVDLGTLRGYYNQSEAGSHTIQMMYG
CDVGSDGRFLRGYQQDAYDGKDYIALNEDLRSWTAADMAAQITQRKWEAA
RVAEQLRAYLEGTCVEWLRRYLENGKETLQRTDPPKTHMTHHAVSDHEAT
LRCWALSFYPAEITLTWQRDGEDQTQDTELVETRPAGDGTFQKWASVVVP
SGQEQRYTCHVQHEGLPKPLTLRWEPSSQPTIPIVGIIAGLVLFGAVFAG
AVVAAVRWRRKSSDRKGGSYSQAASSDSAQGSDMSLTACKV (SEQ ID
NO:158).

Such an MHC Class I heavy chain may be prominent in Asian populations, including populations of individuals of Asian descent. In some cases, amino acid 84 is an Ala. In some cases, amino acid 84 is a Cys. In some cases, amino acid 236 is a Cys. In some cases, amino acid 84 is an Ala and amino acid 236 is a Cys. In some cases, amino acid 84 is an Cys and amino acid 236 is a Cys.

### Hla-b

In some cases, a TMP of the present disclosure comprises an HLA-B heavy chain polypeptide. The HLA-B heavy chain peptide sequences, or portions thereof, that may be that may be incorporated into a TMP of the present disclosure include, but are not limited to, the alleles: B*0702, B*0801, B*1502, B*3802, B*4001, B*4601, and B*5301, which are aligned without all, or substantially all, of the leader, transmembrane and cytoplasmic sequences in FIG. 8A. Any of those alleles may comprise a mutation at one or more of positions 84, 139 and/or 236 (as shown in FIG. 8A) selected from: a tyrosine to alanine at position 84 (Y84A); a tyrosine to cysteine at position 84 (Y84C); an alanine to cysteine at position 139 (A139C); and an alanine to cysteine substitution at position 236 (A236C). In addition, a HLA-B polypeptide comprising an amino acid sequence having at least 75% (e.g., at least 80%, at least 85%, at least 90%, at least 95%, at least 98%, at least 99%) or 100% amino acid sequence identity to all or part (e.g., 50, 75, 100, 150, 200, or 250 contiguous amino acids) of the sequence of those HLA-B alleles may also be employed (e.g., it may comprise 1-25, 1-5, 5-10, 10-15, 15-20, 20-25, or 25-30 amino acid insertions, deletions, and/or substitutions).

In some cases, a TMP of the present disclosure comprises an HLA-B heavy chain polypeptide comprising the following HLA-B consensus amino acid sequence:

GSHSMRYFX1 TX2X3 SRPGRGEPRFIX4 VGYVDDTX5 FVRFDSDAX6 SP
RX7X8 PRAPWIEQEGPEYWDRX9 TQX10X11 KTX12X13 TQX14YX15X1 6 NLX17X15X19X20 YYNQSEAGSHX21X22 QX23 MYGCDLGPDGRLLRG
HDQSAYDGKDYIALNEDLX24 SWTAADTAAQIX25 QRKX26 EAARX27 AE
QX28 RX29 YLEGX30 CVEWLRRYLENGKX31X32LX33 RADPPKTHVTHH
PX34 SDHEATLRCWALGFYPAEITLTWQRDGEDQTQDTELVETRPAGDRT
FQKWAAVVVPSGEEQRYTCHVQHEGLPKPLTLRWEP (SEQ ID NO:15
9),

wherein X1 is H, Y, or D; X2 is A or S; X3 is M or V; X4 is A, S, or T; X5 is Q or L; X6 is A or T; X7 is E, M K, or T; X8 is A or T; X9 is E or N; X10 is I or K; X11 is Y, F, S, or C; X12 is N or Q; X13 is A or T; X14 is D or Y; X15 is E or V; X16 is S or N; X17 is T, N, or I; X18 is A or L; X19 is L, or R; X20 is R or G; X21 is T or I; X22 is L or I; X23 is R or S; X24 is R or S; X25 is S or T; X26 is L or W; X27 is E OR V; X28 is R, D, L or W; X29 is A or T; X30 is L, E or T; X31 is E or D; X32 is K or T; X33 is E or Q; and X34 is I or V.

As an example, an MHC Class I heavy chain polypeptide of a TMP of the present disclosure can comprise an amino acid sequence having at least 75%, at least 80%, at least 85%, at least 90%, at least 95%, at least 98%, at least 99%, or 100%, amino acid sequence identity to the following human HLA-B heavy chain amino acid sequence:

GSHSMRYFYTSVSRPGRGEPRFISVGYVDDTQFVRFDSDAASPREEPRAP
WIEQEGPEYWDRNTQIYKAQAQTDRESLRNLRGYYNQSEAGSHTLQSMYG
CDVGPDGRLLRGHDQYAYDGKDYIALNEDLRSWTAADTAAQITQRKWEAA
REAEQRRAYLEGECVEWLRRYLENGKDKLERADPPKTHVTHHPISDHEAT
LRCWALGFYPAEITLTWQRDGEDQTQDTELVETRPAGDRTFQKWAAVVVP
SGEEQRYTCHVQHEGLPKPLTLRWEP (SEQ ID NO:160).

### Hla-b (y84a; A236c)

As one non-limiting example, in some cases, the MHC Class I heavy chain polypeptide is an HLA-B polypeptide that comprises Y84A and A236C substitutions. For example, in some cases, the MHC Class I heavy chain polypeptide comprises an amino acid sequence having at least 75%, at least 80%, at least 85%, at least 90%, at least 95%, at least 98%, at least 99%, or 100%, amino acid sequence identity to the following human HLA-B heavy chain (Y84A; A236C) amino acid sequence:

GSHSMRYFYTSVSRPGRGEPRFISVGYVDDTQFVRFDSDAASPREEPRAP
WIEQEGPEYWDRNTQIYKAQAQTDRESLRNLRGA YNQSEAGSHTLQSMYG
CDVGPDGRLLRGHDQYAYDGKDYIALNEDLRSWTAADTAAQITQRKWEAA
REAEQRRAYLEGECVEWLRRYLENGKDKLERADPPKTHVTHHPISDHEAT
LRCWALGFYPAEITLTWQRDGEDQTQDTELVETRPC GDRTFQKWAAVVVP
SGEEQRYTCHVQHEGLPKPLTLRWEP (SEQ ID NO:161),

where amino acid 84 is Ala and amino acid 236 is Cys. In some cases, the Cys-236 forms an interchain disulfide bond with Cys-12 of a variant β2M polypeptide that comprises an R12C substitution.

### Hla-b (y84c; A139c)

In some cases, the MHC Class I heavy chain polypeptide comprises Y84C and A139C substitutions. For example, in some cases, the MHC Class I heavy chain polypeptide comprises an amino acid sequence having at least 75%, at least 80%, at least 85%, at least 90%, at least 95%, at least 98%, at least 99%, or 100%, amino acid sequence identity to the following human HLA-B heavy chain (Y84C; A139C) amino acid sequence:

GSHSMRYFYTSVSRPGRGEPRFISVGYVDDTQFVRFDSDAASPREEPRAP
WIEQEGPEYWDRNTQIYKAQAQTDRESLRNLRGC YNQSEAGSHTLQSMYG
CDVGPDGRLLRGHDQYAYDGKDYIALNEDLRSWTAADTC AQITQRKWEAA
REAEQRRAYLEGECVEWLRRYLENGKDKLERADPPKTHVTHHPISDHEAT
LRCWALGFYPAEITLTWQRDGEDQTQDTELVETRPAGDRTFQKWAAVVVP
SGEEQRYTCHVQHEGLPKPLTLRWEP (SEQ ID NO:162),

where amino acid 84 is Cys and amino acid 139 is Cys. In some cases, Cys-84 forms an intrachain disulfide bond with Cys-139.

### Hla-b*0702

As an example, in some cases, a MHC Class I heavy chain polypeptide present in a TMP of the present disclosure comprises an amino acid sequence of HLA-B*0702 (SEQ ID NO:160) in FIG. 8A, or a sequence having at least 75% (e.g., at least 80%, at least 85%, at least 90%, at least 95%, at least 98%, at least 99%) or 100%, amino acid sequence identity to all or part (e.g., 50, 75, 100, 150, 200, or 250 contiguous amino acids) of that sequence (e.g., it may comprise 1-25, 1-5, 5-10, 10-15, 15-20, 20-25, or 25-30 amino acid insertions, deletions, and/or substitutions). In some cases, where the HLA-B heavy chain polypeptide of TMP of the present disclosure has less than 100% identity to the sequence labeled HLA-B in FIG. 6, or labeled “B*0702 in FIG. 8A, it may comprise a mutation at one or more of positions 84, 139 and/or 236 selected from: a tyrosine to alanine substitution at position 84 (Y84A); a tyrosine to cysteine substitution at position 84 (Y84C); an alanine to cysteine at position 139 (A139C); and an alanine to cysteine substitution at position 236 (A236C). In some cases, the HLA-B heavy chain polypeptide of TMP of the present disclosure comprises Y84A and A236C substitutions. In some cases, the HLA-B*0702 heavy chain polypeptide of TMP of the present disclosure comprises Y84C and A139C substitutions. In some cases, the HLA-B heavy chain polypeptide of TMP of the present disclosure comprises Y84C, A139C, and A236C substitutions.

### Hla-c

In some cases, a TMP of the present disclosure comprises an HLA-C heavy chain polypeptide. The HLA-C heavy chain polypeptide, or portions thereof, that may be that may be incorporated into a TMP of the present disclosure include, but are not limited to, the alleles: C*0102, C*0303, C*0304, C*0401, C*0602, C*0701, C*0801, and C*1502, which are aligned without all, or substantially all, of the leader, transmembrane and cytoplasmic sequences in FIG. 9A. Any of those alleles may comprise a mutation at one or more of positions 84, 139 and/or 236 (as shown in FIG. 9A) selected from: a tyrosine to alanine substitution at position 84 (Y84A); a tyrosine to cysteine substitution at position 84 (Y84C); an alanine to cysteine substitution at position 139 (A139C); and an alanine to cysteine substitution at position 236 (A236C). In addition, an HLA-C polypeptide comprising an amino acid sequence having at least 75% (e.g., at least 80%, at least 85%, at least 90%, at least 95%, at least 98%, at least 99%) or 100% amino acid sequence identity to all or part (e.g., 50, 75, 100, 150, 200, or 250 contiguous amino acids) of the sequence of those HLA-C alleles may also be employed (e.g., it may comprise 1-25, 1-5, 5-10, 10-15, 15-20, 20-25, or 25-30 amino acid insertions, deletions, and/or substitutions).

In some cases, a TMP of the present disclosure comprises an HLA-C heavy chain polypeptide comprising the following HLA-C consensus amino acid sequence:

X1 SHSMX2 YFX3 TAVSX4 PGRGEPX5 FIX6 VGYVDDTQFVX7 FDSDAAS
PRGEPRX8 PWVEQEGPEYWDRETQX9 YKRQAQX10 DRVX11 LRX12 LRGY
YNQSEX13X14 SHX15X16 QX 17 MX18 GCDX19 GPDGRLLRGX20X21 QX 22 AYDGKDYIALNEDLRSWTAADTAAQITQRKX23 EAARX24 AEQX25 RA
YLEGX26 CVEWLRRYLX27 NGKX28 TLQRAEX29 PKTHVTHHPX30 SDHE
ATLRCWALGFYPAEITLTWQX31 DGEDQTQDTELVETRPAGDGTFQKWAA
VX32 VPSGX33 EQRYTCHX34 QHEGLX35 EPLTLX36 WX37 P (SEQ ID
NO:163),

wherein X1 is C or G; X2 is R or K; X3 is F, Y, S, or D; X4 is R or W; X5 is H or R; X6 is A or S; X7 is Q or R; X8 is A or E; X9 is N or K; X10 is T or A; X11 is S or N; X12 is N or K; X13 is A or D; X14 is G or R; X15 is T or I; X16 is L or I; X17 is W or R; X18 is C, Y, F, or S; X19 is L, or V; X20 is Y or H; X21 is D or N; X22 is Y, F, S, or L; X23 is L or W; X24 is E, A, Or T; X25 is R, L, or W; X26 is L or T; X27 is E OR K; X28 is E or K; X29 is H or P; X30 is R or V; X31 is W or R; X32 is V or M; X33 is E or Q; X34 is M or V; X35 is P or Q; X36 is R or S; and X37 is P or G.

As an example, an MHC Class I heavy chain polypeptide of a TMP of the present disclosure can comprise an amino acid sequence having at least 75%, at least 80%, at least 85%, at least 90%, at least 95%, at least 98%, at least 99%, or 100%, amino acid sequence identity to the following human HLA-C heavy chain amino acid sequence:

CSHSMRYFDTAVSRPGRGEPRFISVGYVDDTQFVRFDSDAASPRGEPRAP
WVEQEGPEYWDRETQNYKRQAQADRVSLRNLRGYYNQSEDGSHTLQRMYG
CDLGPDGRLLRGYDQSAYDGKDYIALNEDLRSWTAADTAAQITQRKLEAA
RAAEQLRAYLEGTCVEWLRRYLENGKETLQRAEPPKTHVTHHPLSDHEAT
LRCWALGFYPAEITLTWQRDGEDQTQDTELVETRPAGDGTFQKWAAVVVP
SGQEQRYTCHMQHEGLQEPLTLSWEP (SEQ ID NO:164).

### Hla-c (y84a; A236c)

As one non-limiting example, in some cases, the MHC Class I heavy chain polypeptide is an HLA-C polypeptide that comprises Y84A and A236C substitutions. For example, in some cases, the MHC Class I heavy chain polypeptide comprises an amino acid sequence having at least 75%, at least 80%, at least 85%, at least 90%, at least 95%, at least 98%, at least 99%, or 100%, amino acid sequence identity to the following human HLA-C heavy chain (Y84A; A236C) amino acid sequence:

CSHSMRYFDTAVSRPGRGEPRFISVGYVDDTQFVRFDSDAASPRGEPRAP
WVEQEGPEYWDRETQNYKRQAQADRVSLRNLRGA YNQSEDGSHTLQRMYG
CDLGPDGRLLRGYDQSAYDGKDYIALNEDLRSWTAADTAAQITQRKLEAA
RAAEQLRAYLEGTCVEWLRRYLENGKETLQRAEPPKTHVTHHPLSDHEAT
LRCWALGFYPAEITLTWQRDGEDQTQDTELVETRPC GDGTFQKWAAVVVP
SGQEQRYTCHMQHEGLQEPLTLSWEP (SEQ ID NO:165),

where amino acid 84 is Ala and amino acid 236 is Cys. In some cases, the Cys-236 forms an interchain disulfide bond with Cys-12 of a variant β2M polypeptide that comprises an R12C substitution.

### Hla-c (y84c; A139c)

In some cases, the MHC Class I heavy chain polypeptide comprises Y84C and A139C substitutions. For example, in some cases, the MHC Class I heavy chain polypeptide comprises an amino acid sequence having at least 75%, at least 80%, at least 85%, at least 90%, at least 95%, at least 98%, at least 99%, or 100%, amino acid sequence identity to the following human HLA-C heavy chain (Y84C; A139C) amino acid sequence:

CSHSMRYFDTAVSRPGRGEPRFISVGYVDDTQFVRFDSDAASPRGEPRAP
WVEQEGPEYWDRETQNYKRQAQADRVSLRNLRGC YNQSEDGSHTLQRMYG
CDLGPDGRLLRGYDQSAYDGKDYIALNEDLRSWTAADTC AQITQRKLEAA
RAAEQLRAYLEGTCVEWLRRYLENGKETLQRAEPPKTHVTHHPLSDHEAT
LRCWALGFYPAEITLTWQRDGEDQTQDTELVETRPAGDGTFQKWAAVVVP
SGQEQRYTCHMQHEGLQEPLTLSWEP (SEQ ID NO:166),

where amino acid 84 is Cys and amino acid 139 is Cys. In some cases, Cys-84 forms an intrachain disulfide bond with Cys-139.

### Hla-c*0701

In some cases, a MHC Class I heavy chain polypeptide of a TMP of the present disclosure comprises an amino acid sequence of HLA-C*0701 of FIG. 9A (labeled HLA-C in FIG. 6), or an amino acid sequence having at least 75% (e.g., at least 80%, at least 85%, at least 90%, at least 95%, at least 98%, at least 99%) or 100% amino acid sequence identity to all or part (e.g., 50, 75, 100, 150, 200, or 250 contiguous amino acids) of that sequence (e.g., it may comprise 1-25, 1-5, 5-10, 10-15, 15-20, 20-25, or 25-30 amino acid insertions, deletions, and/or substitutions). In some cases, where the HLA-C heavy chain polypeptide of a TMP of the present disclosure has less than 100% identity to the sequence labeled HLA-C*0701 in FIG. 9A, it may comprise a mutation at one or more of positions 84, 139 and/or 236 selected from: a tyrosine to alanine substitution at position 84 (Y84A); a tyrosine to cysteine substitution at position 84 (Y84C); an alanine to cysteine at position 139 (A139C); and an alanine to cysteine substitution at position 236 (A236C). In some cases, the HLA-C heavy chain polypeptide of a TMP of the present disclosure comprises Y84A and A236C substitutions. In some cases, the HLA-C*0701 heavy chain polypeptide of a T-Cell-MMP or its epitope conjugate comprises Y84C and A139C substitutions. In some cases, the HLA-C heavy chain polypeptide of a TMP of the present disclosure comprises Y84C, A139C, and A236C substitutions.

### Non-Classical HLA-E, -F, and -G MHC Class I Heavy Chains

In some cases, a TMP of the present disclosure comprises a non-classical MHC Class I heavy chain polypeptide. Among the non-classical HLA heavy chain polypeptides, or portions thereof, that may be that may be incorporated into a TMP of the present disclosure include, but are not limited to, those of HLA-E, -F, and -G alleles. Amino acid sequences for HLA-E, -F, and -G heavy chain polypeptides, (and the HLA-A, B and C alleles) may be found on the world wide web hla.alleles.org/ nomenclature/index.html, the European Bioinformatics Institute (www(dot)ebi(dot)ac(dot)uk), which is part of the European Molecular Biology Laboratory (EMBL), and at the National Center for Biotechnology Information (www(dot)ncbi(dot)nlm(dot)nih(dot)gov).

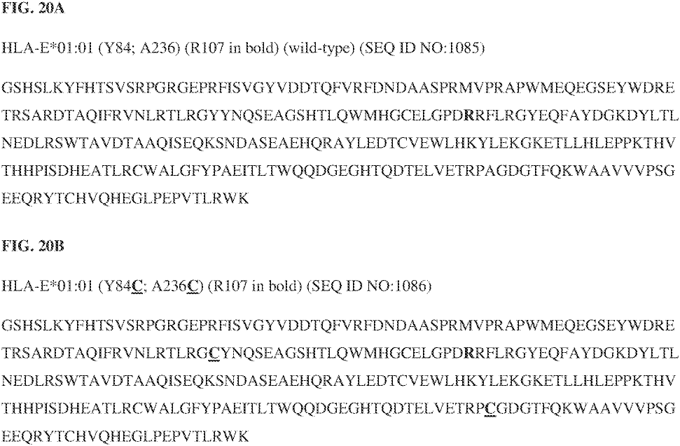

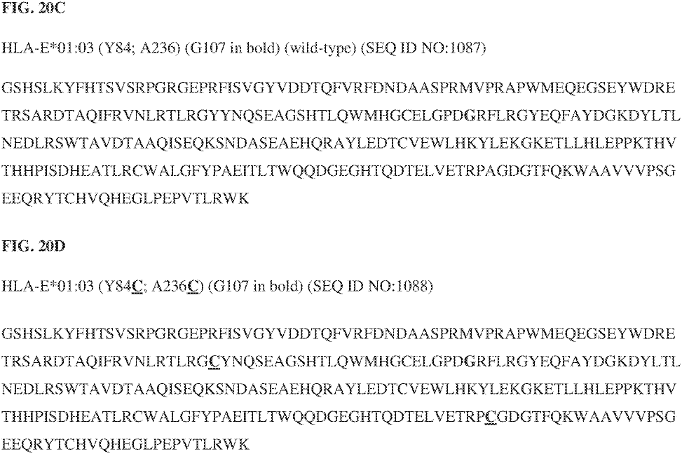

Non-limiting examples of suitable HLA-E alleles include, but are not limited to, HLA-E*0101 (HLA-E*01:01:01:01), HLA-E*01:03(HLA-E*01:03:01:01), HLA-E*01:04, HLA-E*01:05, HLA-E*01:06, HLA-E*01:07, HLA-E*01:09, and HLA-E*01:10. For example, amino acid sequences of suitable HLA-E heavy chain polypeptides are provided in FIGS. 20A-20D, where FIG. 20A provides the amino acid sequence of HLA-E*01:01 (wild-type); FIG. 20B provides the amino acid sequence of HLA-E*01:01 with Y84C and A2346C substitutions; FIG. 20C provides the amino acid sequence of HLA-E*01:03 (wild-type); and FIG. 20D provides the amino acid sequence of HLA-E*01:03 with Y84C and A2346C substitutions.

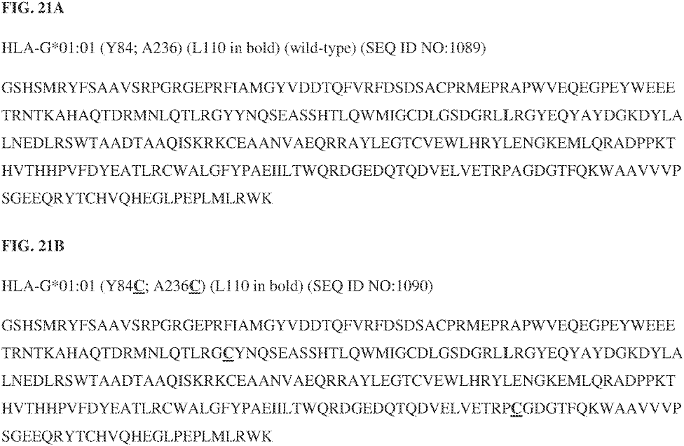

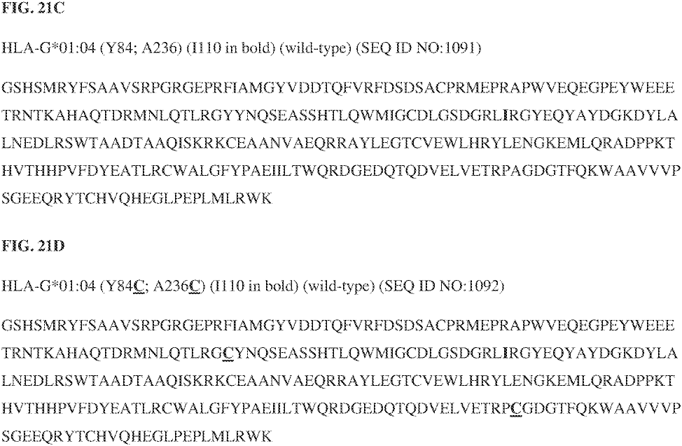

Non-limiting examples of suitable HLA-F alleles include, but are not limited to, HLA-F*0101 (HLA-F*01:01:01:01), HLA-F*01:02, HLA-F*01:03(HLA-F*01:03:01:01), HLA-F*01:04, HLA-F*01:05, and HLA-F*01:06. Non-limiting examples of suitable HLA-G alleles include, but are not limited to, HLA-G*0101 (HLA-G*01:01:01:01), HLA-G*01:02, HLA-G*01:03(HLA-G*01:03:01:01), HLA-G*01:04 (HLA-G*01:04:01:01), HLA-G*01:06, HLA-G*01:07, HLA-G*01:08, HLA-G*01:09: HLA-G*01:10, HLA-G*01:10, HLA-G*01:11, HLA-G*01:12, HLA-G*01:14, HLA-G*01:15, HLA-G*01:16, HLA-G*01:17, HLA-G*01:18: HLA-G*01:19, HLA-G*01:20, and HLA-G*01:22.. For example, amino acid sequences of suitable HLA-G heavy chain polypeptides are provided in FIGS. 21A-21D, where FIG. 21A provides the amino acid sequence of HLA-G*01:01 (wild-type); FIG. 21B provides the amino acid sequence of HLA-G*01:01 with Y84C and A2346C substitutions; FIG. 21C provides the amino acid sequence of HLA-G*01:04 (wild-type); and FIG. 21D provides the amino acid sequence of HLA-G*01 :04 with Y84C and A2346C substitutions.

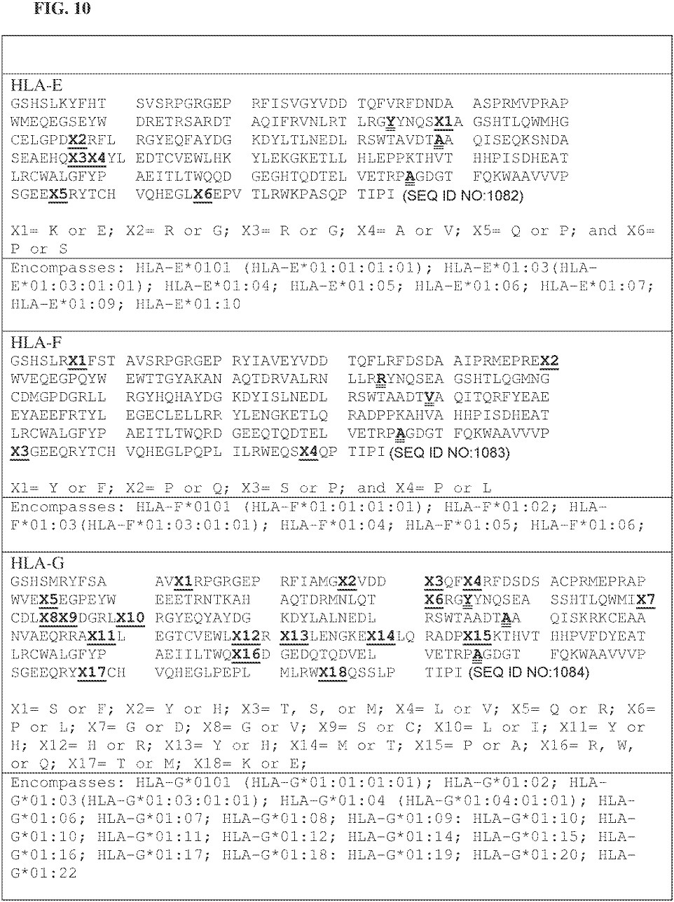

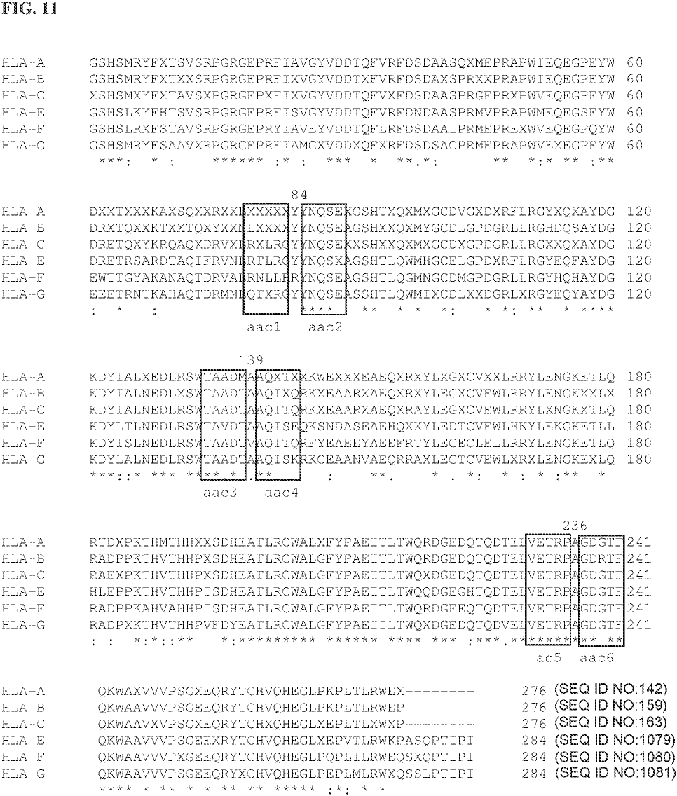

Consensus sequences for those HLA E, -F and -G alleles without all, or substantially all, of the leader, transmembrane and cytoplasmic sequences are provided in FIG. 10, and aligned with consensus sequences of the above-mentioned HLA-A, -B and -C alleles in FIG. 11.

FIG. 10 provides a consensus sequence for each of HLA-E, -F, and -G with the variable aa positions indicated as “X” residues sequentially numbered and the locations of aas 84, 139 and 236 double underlined.

FIG. 11 provides an alignment of the consensus amino acid sequences for HLA-A, -B, -C, -E, -F, and -G, which are given in FIGS. 7-11. Variable residues in each sequence are listed as “X” with the sequential numbering removed. As indicated in FIG. 6, the locations of aas 84, 139 and 236 are indicated with their flanking five-amino acid clusters that may be replaced by 1 to 5 amino acids selected independently from (i) any naturally occurring amino acid or (ii) any naturally occurring amino acid except proline or glycine are also shown.

Any of the above-mentioned HLA-E, -F, and/or -G alleles may comprise a substitution at one or more of positions 84, 139 and/or 236 as shown in FIG. 11 for the consensus sequences. In some cases, the substitutions may be selected from a: position 84 tyrosine to alanine (Y84A) or cysteine (Y84C), or, in the case of HLA-F, an R84A or R84C substitution; a position 139 alanine to cysteine (A139C), or, in the case of HLA-F, a V139C; and an alanine to cysteine substitution at position 236 (A236C). In addition, an HLA-E, -F and /or -G sequence having at least 75% (e.g., at least 80%, at least 85%, at least 90%, at least 95%, at least 98%, at least 99%) or 100% amino acid sequence identity to all or part (e.g., 50, 75, 100, 150, 200, or 250 contiguous amino acids) of any of the consensus sequences of set forth in FIG. 11 may also be employed (e.g., the sequences may comprise 1-25, 1-5, 5-10, 10-15, 15-20, 20-25, or 25-30 amino acid insertions, deletions, and/or substitutions in addition to changes at variable residues listed therein).

### Mouse H2K

In some cases, a MHC Class I heavy chain polypeptide present in a TMP of the present disclosure comprises an amino acid sequence of MOUSE H2K (SEQ ID NO:392) (MOUSE H2K in FIG. 6), or a sequence having at least 75%, at least 80%, at least 85%, at least 90%, at least 95%, at least 98%, at least 99%, or 100% amino acid sequence identity to all or part (e.g., 50, 75, 100, 150, 200, or 250 contiguous amino acids) of that sequence (e.g., it may comprise 1-25, 1-5, 5-10, 10-15, 15-20, 20-25, or 25-30 amino acid insertions, deletions, and/or substitutions). In some cases, where the MOUSE H2K heavy chain polypeptide of a TMP of the present disclosure has less than 100% identity to the sequence labeled MOUSE H2K in FIG. 6, it may comprise a mutation at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine substitution at position 236 (A236C). In some cases, the MOUSE H2K heavy chain polypeptide of a TMP of the present disclosure comprises Y84A and A236C substitutions. In some cases, the MOUSE H2K heavy chain polypeptide of a TMP of the present disclosure comprises Y84C and A139C substitutions. In some cases, the MOUSE H2K heavy chain polypeptide of a TMP of the present disclosure comprises Y84C, A139C and A236C substitutions.

### Exemplary Combinations

Table 1, below, presents various combinations of MHC Class I heavy chain sequence modifications that can be incorporated in a TMP of the present disclosure.

TABLE 1

| Entry | HLA Heavy Chain Sequence | Sequence Identity Range [symbol] | Specific Substitutions at aa positions 84, 139 and/or 236 |
|---|---|---|---|
| 1 | HLA-A Consensus (FIG. 7B) | 75%-99.8%, 80%-99.8%, 85%-99.8%, 90%-99.8%, 95%-99.8%, 98%-99.8%, or 99%-99.8%; or 1-25, 1-5, 5-10, 10-15, 15-20, or 20-25 aa insertions, deletions, and/or substitutions (not counting variable residues) | None; Y84C; Y84A; A139C; A236C; (Y84A & A236C); (Y84C & A139C); or (Y84C, A139C & A236C) |
| 2 | A*0101, A*0201, A*0301, A*1101, | 75%-99.8%, 80%-99.8%, 85%-99.8%, 90%-99.8%, 95%-99.8%, 98%-99.8%, | None; Y84C; Y84A; A139C; A236C; |
| | A*2402, A*2301, A*2402, A*2407, A*3303, or A*3401 (FIG. 7A) | or 99%-99.8%; or 1-25, 1-5, 5-10, 10-15, 15-20, or 20-25 aa insertions, deletions, and/or substitutions | (Y84A & A236C); (Y84C & A139C); or (Y84C, A139C & A236C) |
| 3 | HLA-B Consensus (FIG. 8B) | 75%-99.8%, 80%-99.8%, 85%-99.8%, 90%-99.8%, 95%-99.8%, 98%-99.8%, or 99%-99.8%; or 1-25, 1-5, 5-10, 10-15, 15-20, or 20-25 aa insertions, deletions, and/or substitutions (not counting variable residues) | None; Y84C; Y84A; A139C; A236C; (Y84A & A236C); (Y84C & A139C); or (Y84C, A139C & A236C) |
| 4 | B*0702, B*0801, B*1502, B*3802, B*4001, B*4601, or B*5301 (FIG. 8A) | 75%-99.8%, 80%-99.8%, 85%-99.8%, 90%-99.8%, 95%-99.8%, 98%-99.8%, or 99%-99.8%; or 1-25, 1-5, 5-10, 10-15, 15-20, or 20-25 aa insertions, deletions, and/or substitutions | None; Y84C; Y84A; A139C; A236C; (Y84A & A236C); (Y84C & A139C); or (Y84C, A139C & A236C) |
| 5 | HLA-C Consensus (FIG. 9B) | 75%-99.8%, 80%-99.8%, 85%-99.8%, 90%-99.8%, 95%-99.8%, 98%-99.8%, or 99%-99.8%; or 1-25, 1-5, 5-10, 10-15, 15-20, or 20-25 aa insertions, deletions, and/or substitutions (not counting variable residues) | None; Y84C; Y84A; A139C; A236C; (Y84A & A236C); (Y84C & A139C); or (Y84C, A139C & A236C) |
| 6 | C*0102, C*0303, C*0304, C*0401, C*0602, C*0701, C*0801, or C*1502 (FIG. 9A) | 75%-99.8%, 80%-99.8%, 85%-99.8%, 90%-99.8%, 95%-99.8%, 98%-99.8%, or 99%-99.8%; or 1-25, 1-5, 5-10, 10-15, 15-20, or 20-25 aa insertions, deletions, and/or substitutions | None; Y84C; Y84A; A139C; A236C; (Y84A & A236C); (Y84C & A139C); or (Y84C, A139C & A236C) |
| 7 | HLA-E, F, or G Consensus (FIG. 10 ) | 75%-99.8%, 80%-99.8%, 85%-99.8%, 90%-99.8%, 95%-99.8%, 98%-99.8%, or 99%-99.8%; or 1-25, 1-5, 5-10, 10-15, 15-20, or 20-25 aa insertions, deletions, and/or substitutions (not counting variable residues) | None; Y84C; Y84A; A139C; A236C; (Y84A & A236C); (Y84C & A139C); or (Y84C, A139C & A236C) |
| 8 | MOUSE H2K (FIG. 6 ) | 75%-99.8%, 80%-99.8%, 85%-99.8%, 90%-99.8%, 95%-99.8%, 98%-99.8%, or 99%-99.8%; or 1-25, 1-5, 5-10, 10-15, 15-20, or 20-25 aa insertions, deletions, and/or substitutions | None; Y84C; Y84A; A139C; A236C; (Y84A & A236C); (Y84C & A139C); or (Y84C, A139C & A236C) |

[symbol]

The Sequence Identity Range is the permissible range in sequence identity of an MHC-H polypeptide sequence incorporated into a TMP relative to the corresponding portion of the sequences listed in FIGS. 6-11 not counting the variable residues in the consensus sequences.

### Beta-2 Microglobulin

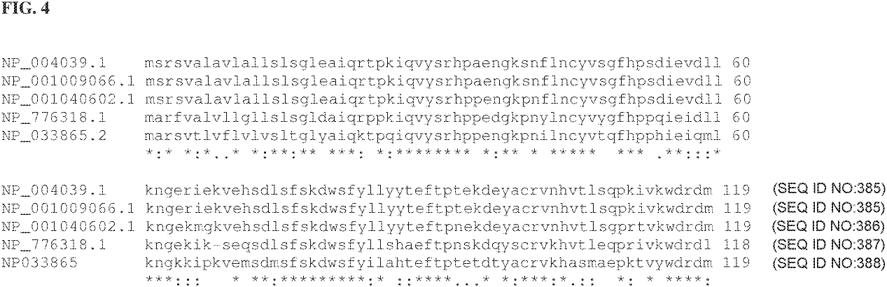

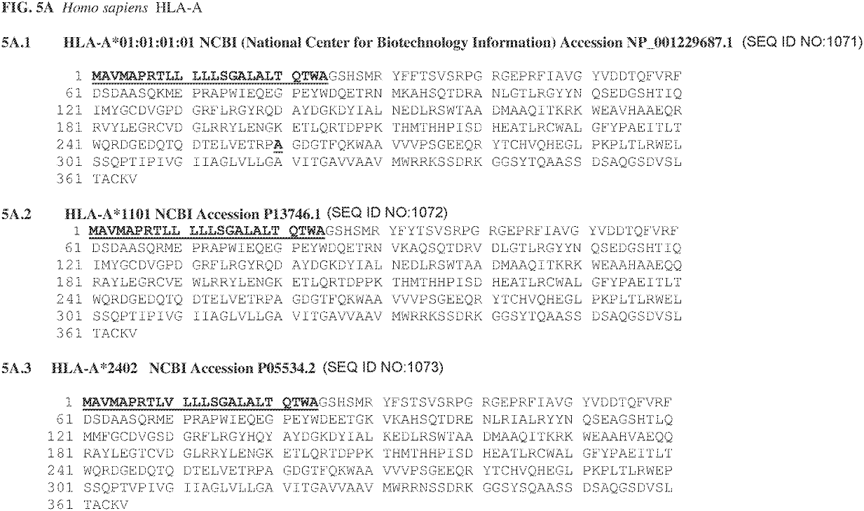

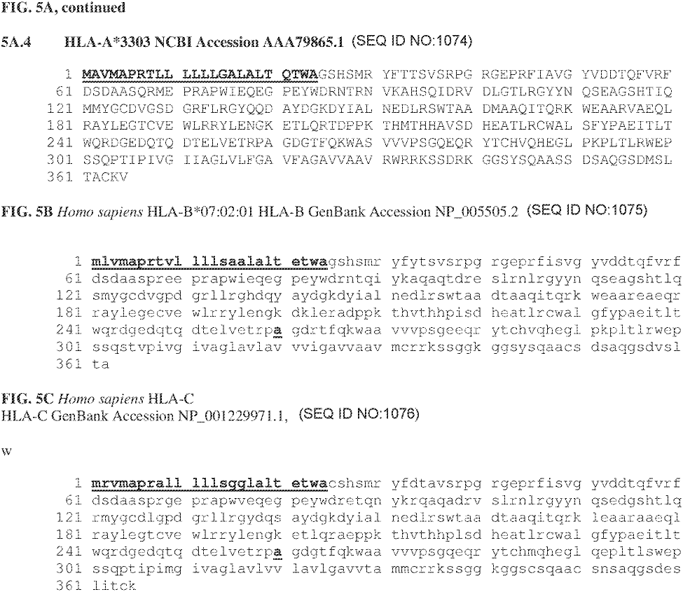

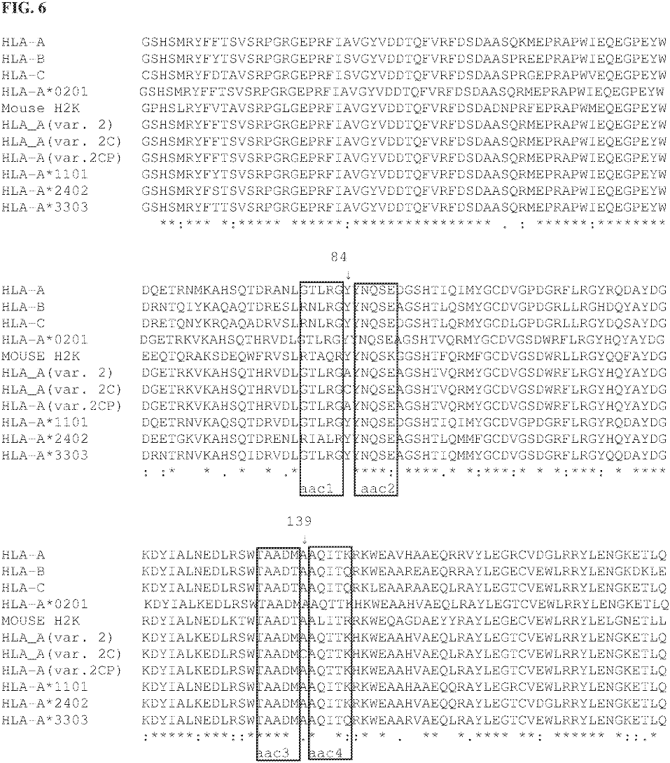

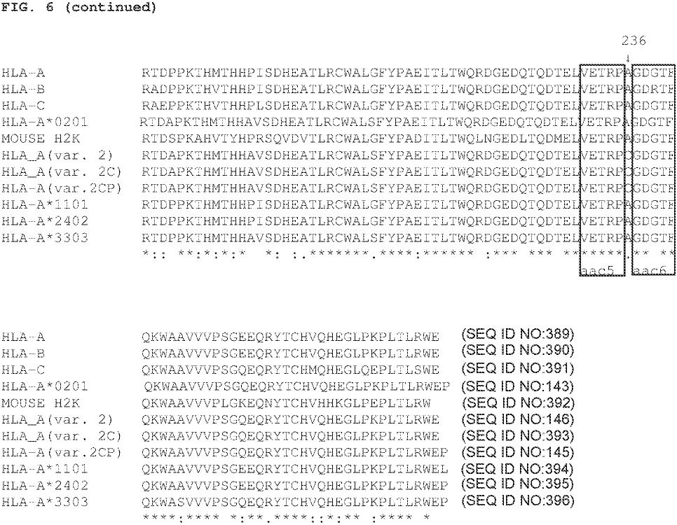

A β2-microglobulin (β2M) polypeptide of a TMP of the present disclosure can be a human β2M polypeptide, a non-human primate β2M polypeptide, a murine β2M polypeptide, and the like. In some instances, a β2M polypeptide comprises an amino acid sequence having at least 75%, at least 80%, at least 85%, at least 90%, at least 95%, at least 98%, at least 99%, or 100%, amino acid sequence identity to a β2M amino acid sequence depicted in FIG. 6. In some instances, a β2M polypeptide comprises an amino acid sequence having at least 75%, at least 80%, at least 85%, at least 90%, at least 95%, at least 98%, at least 99%, or 100%, amino acid sequence identity to amino acids 21 to 119 of a β2M amino acid sequence depicted in FIG. 4.

In some cases, a suitable β2M polypeptide comprises the following amino acid sequence:

IQRTPKIQVY SCHPAENGKS NFLNCYVSGF HPSDIEVDLLKNGERIEKVE HSDLSFSKDW SFYLLYYTEF TPTEKDEYAC RVNHVTLSQP KIVKWDRDM (SEQ ID NO:167); and the HLA Class I heavy chain polypeptide comprises the following amino acid sequence:

GSHSMRYFFTSVSRPGRGEPRFIAVGYVDDTQFVRFDSDAASQRMEPRAPWIEQ EGPEYWDGETRKVKAHSQTHRVDL(aal){C}(aa2)AGSHTVQRMYGCDVGSDWRFLRGYHQYA YDGKDYIALKEDLRSW(aa3){C}(aa4))HKWEAAHVAEQLRAYLEGTCVEWLRRYLENGKETLQ RTDAPKTHMTHHAVSDHEATLRCWALSFYPAEITLTWQRDGEDQTQDTEL(aa5)I(aa6)QKWAA VVVPSGQEQRYTCHVQHEGLPKPLTLRWEP (SEQ ID NO:168), where the cysteine residues indicated as {C} form an disulfide bond between the α1 and α2-1 helices and the residue forms a disulfide bond with the β2M polypeptide cysteine at position 12. In the sequence above, “aa1” is “amino acid cluster 1”; “aa2” is “amino acid cluster 2”; “aa3” is “amino acid cluster 3”; “aa4” is “amino acid cluster 4”; “aa5” is “amino acid cluster 5”; and “aa6” is “amino acid cluster 6”; see, e.g., FIG. 8. Each occurrence of aa1, aa2, aa3, aa4, aa5, and aa6 is and independently selected to be 1-5 amino acid residues, wherein the amino acid residues are i) selected independently from any naturally occurring (e.g., encoded) amino acid or ii) any naturally occurring amino acid except proline or glycine.

In some cases, an MHC polypeptide comprises a single amino acid substitution relative to a reference MHC polypeptide (where a reference MHC polypeptide can be a wild-type MHC polypeptide), where the single amino acid substitution substitutes an amino acid with a cysteine (Cys) residue. Such cysteine residues, when present in an MHC polypeptide of a first polypeptide of a TMP of the present disclosure, can form a disulfide bond with a cysteine residue present in a second polypeptide chain of a TMP of the present disclosure.

In some cases, a first MHC polypeptide in a first polypeptide of a TMP of the present disclosure, and/or the second MHC polypeptide in the second polypeptide of a TMP of the present disclosure, includes an amino acid substitution to substitute an amino acid with a cysteine, where the substituted cysteine in the first MHC polypeptide forms a disulfide bond with a cysteine in the second MHC polypeptide, where a cysteine in the first MHC polypeptide forms a disulfide bond with the substituted cysteine in the second MHC polypeptide, or where the substituted cysteine in the first MHC polypeptide forms a disulfide bond with the substituted cysteine in the second MHC polypeptide.

For example, in some cases, one of following pairs of residues in an HLA β2-microglobulin and an HLA Class I heavy chain is substituted with cysteines (where residue numbers are those of the mature polypeptide): 1) β2M residue 12, HLA Class I heavy chain residue 236; 2) β2M residue 12, HLA Class I heavy chain residue 237; 3) β2M residue 8, HLA Class I heavy chain residue 234; 4) β2M residue 10, HLA Class I heavy chain residue 235; 5) β2M residue 24, HLA Class I heavy chain residue 236; 6) β2M residue 28, HLA Class I heavy chain residue 232; 7) β2M residue 98, HLA Class I heavy chain residue 192; 8) β2M residue 99, HLA Class I heavy chain residue 234; 9) β2M residue 3, HLA Class I heavy chain residue 120; 10) β2M residue 31, HLA Class I heavy chain residue 96; 11) β2M residue 53, HLA Class I heavy chain residue 35; 12) β2M residue 60, HLA Class I heavy chain residue 96; 13) β2M residue 60, HLA Class I heavy chain residue 122; 14) β2M residue 63, HLA Class I heavy chain residue 27; 15) β2M residue Arg3, HLA Class I heavy chain residue Gly120; 16) β2M residue His31, HLA Class I heavy chain residue Gln96; 17) β2M residue Asp53, HLA Class I heavy chain residue Arg35; 18) β2M residue Trp60, HLA Class I heavy chain residue Gln96; 19) β2M residue Trp60, HLA Class I heavy chain residue Asp122; 20) β2M residue Tyr63, HLA Class I heavy chain residue Tyr27; 21) β2M residue Lys6, HLA Class I heavy chain residue Glu232; 22) β2M residue Gln8, HLA Class I heavy chain residue Arg234; 23) β2M residue Tyr10, HLA Class I heavy chain residue Pro235; 24) β2M residue Ser11, HLA Class I heavy chain residue Gln242; 25) β2M residue Asn24, HLA Class I heavy chain residue Ala236; 26) β2M residue Ser28, HLA Class I heavy chain residue Glu232; 27) β2M residue Asp98, HLA Class I heavy chain residue His192; and 28) β2M residue Met99, HLA Class I heavy chain residue Arg234. The amino acid numbering of the MHC/HLA Class I heavy chain is in reference to the mature MHC/HLA Class I heavy chain, without a signal peptide. For example, in some cases, residue 236 of the mature HLA-A amino acid sequence is substituted with a Cys. In some cases, residue 236 of the mature HLA-B amino acid sequence is substituted with a Cys. In some cases, residue 236 of the mature HLA-C amino acid sequence is substituted with a Cys. In some cases, residue 32 (corresponding to Arg-12 of mature β2M) of an amino acid sequence depicted in FIG. 4 is substituted with a Cys.

In some cases, a β2M polypeptide comprises the amino acid sequence: IQRTPKIQVY SRHPAENGKS NFLNCYVSGF HPSDIEVDLLKNGERIEKVE HSDLSFSKDW SFYLLYYTEF TPTEKDEYAC RVNHVTLSQP KIVKWDRDM (SEQ ID NO:169). In some cases, a β2M polypeptide comprises the amino acid sequence: IQRTPKIQVY SCHPAENGKS NFLNCYVSGF HPSDIEVDLLKNGERIEKVE HSDLSFSKDW SFYLLYYTEF TPTEKDEYAC RVNHVTLSQP KIVKWDRDM (SEQ ID NO:167).

In some cases, an HLA Class I heavy chain polypeptide comprises the amino acid sequence:

GSHSMRYFFTSVSRPGRGEPRFIAVGYVDDTQFVRFDSDAASQRMEPRAP
WIEQEGPEYWDGETRKVKAHSQTHRVDLGTLRGYYNQSEAGSHTVQRMYG
CDVGSDWRFLRGYHQYAYDGKDYIALKEDLRSWTAADMAAQTTKHKWEAA
HVAEQLRAYLEGTCVEWLRRYLENGKETLQRTDAPKTHMTHHAVSDHEAT
LRCWALSFYPAEITLTWQRDGEDQTQDTELVETRPA GDGTFQKWAAVYVP
SGQEQRYTCHVQHEGLPKPLTLRWEP (SEQ ID NO:143).

In some cases, an HLA Class I heavy chain polypeptide comprises the amino acid sequence:

GSHSMRYFFTSVSRPGRGEPRFIAVGYVDDTQFVRFDSDAASQRMEPRAP
WIEQEGPEYWDGETRKVKAHSQTHRVDLGTLRGYYNQSEAGSHTVQRMYG
CDVGSDWRFLRGYHQYAYDGKDYIALKEDLRSWTAADMAAQTTKHKWEAA
HVAEQLRAYLEGTCVEWLRRYLENGKETLQRTDAPKTHMTHHAVSDHEAT
LRCWALSFYPAEITLTWQRDGEDQTQDTELVETRPC GDGTFQKWAAVVVP
SGQEQRYTCHVQHEGLPKPLTLRWEP (SEQ ID NO:170).

In some cases, an HLA Class I heavy chain polypeptide comprises the amino acid sequence:

GSHSMRYFFTSVSRPGRGEPRFIAVGYVDDTQFVRFDSDAASQRMEPRAP
WIEQEGPEYWDGETRKVKAHSQTHRVDLGTLRGA YNQSEAGSHTVQRMYG
CDVGSDWRFLRGYHQYAYDGKDYIALKEDLRSWTAADMAAQTTKHKWEAA
HVAEQLRAYLEGTCVEWLRRYLENGKETLQRTDAPKTHMTHHAVSDHEAT
LRCWALSFYPAEITLTWQRDGEDQTQDTELVETRPC GDGTFQKWAAVVVP
SGQEQRYTCHVQHEGLPKPLTLRWE(SEQ ID NO:146).

In some cases, the β2M polypeptide comprises the following amino acid sequence:

IQRTPKIQVY SC HPAENGKS NFLNCYVSGF HPSDIEVDLLKNGERIE
KVEHSDLSFSKDW SFYLLYYTEF TPTEKDEYAC RVNHVTLSQP KIV
KWDRDM (SEQ ID NO:167);

and the HLA Class I heavy chain polypeptide of a TMP of the present disclosure comprises the following amino acid sequence:

GSHSMRYFFTSVSRPGRGEPRFIAVGYVDDTQFVRFDSDAASQRMEPRA
PWIEQEGPEYWDGETRKVKAHSQTHRVDLGTLRGYYNQSEAGSHTVQRMY
GCDVGSDWRFLRGYHQYAYDGKDYIALKEDLRSWTAADMAAQTTKHKWEA
AHVAEQLRAYLEGTCVEWLRRYLENGKETLQRTDAPKTHMTHHAVSDHEA
TLRCWALSFYPAEITLTWQRDGEDQTQDTELVETRPC GDGTFQKWAAVVV
PSGQEQRYTCHVQHEGLPKPLTLRWEP (SEQ ID NO:170),

where the Cys residues that are underlined and in bold form a disulfide bond with one another in the TMP.

In some cases, the β2M polypeptide comprises the amino acid sequence:

IQRTPKIQVYSC HPAENGKSNFLNCYVSGFHPSDIEVDLLKNGERIEKVE
HSDLSFSKDWSFYLLYYTEFTPTEKDEYACRVNHVTLSQPKIVKWDRDM
(SEQ ID NO:167).

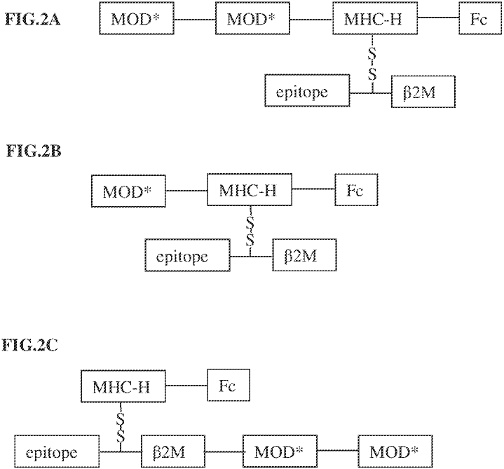

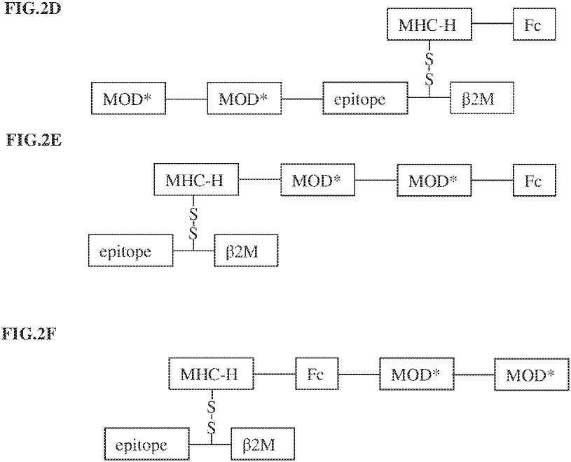

In some cases, the first polypeptide and the second polypeptide of a TMP of the present disclosure are disulfide linked to one another through: i) a Cys residue present in a linker connecting the peptide epitope and a β2M polypeptide in the first polypeptide chain; and ii) a Cys residue present in an MHC Class I heavy chain in the second polypeptide chain. In some cases, the Cys residue present in the MHC Class I heavy chain is a Cys introduce as a Y84C substitution. In some cases, the linker connecting the peptide epitope and the β2M polypeptide in the first polypeptide chain is GCGGS(GGGGS)n (SEQ ID NO:171), where n is 1, 2, 3, 4, 5, 6, 7, 8, or 9. For example, in some cases, the linker comprises the amino acid sequence GCGGSGGGGSGGGGSGGGGS (SEQ ID NO:172). As another example, the linker comprises the amino acid sequence GCGGSGGGGSGGGGS (SEQ ID NO:173). Examples of disulfide-linked first and second polypeptides of a TMP of the present disclosure are depicted schematically in FIGS. 2A-2F.

### Multiple Disulfide Bonded TMPs

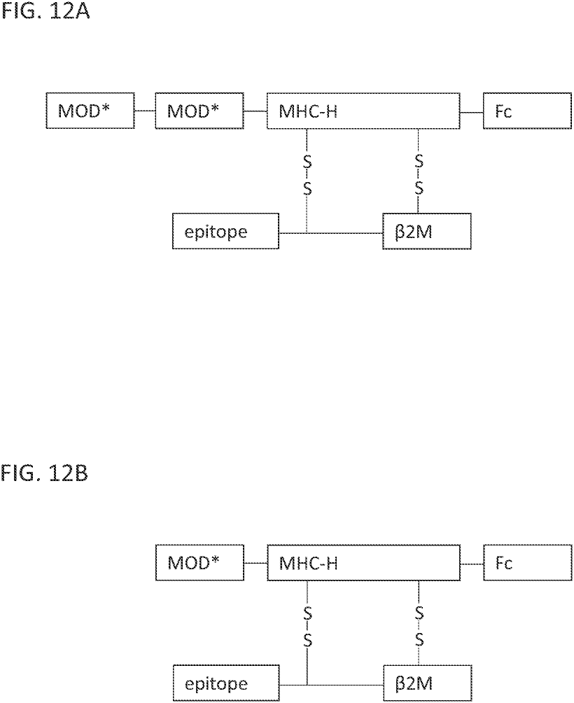

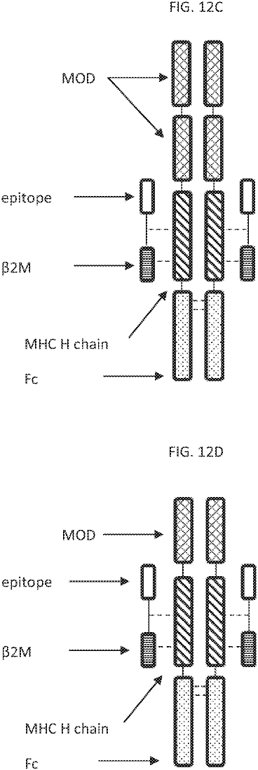

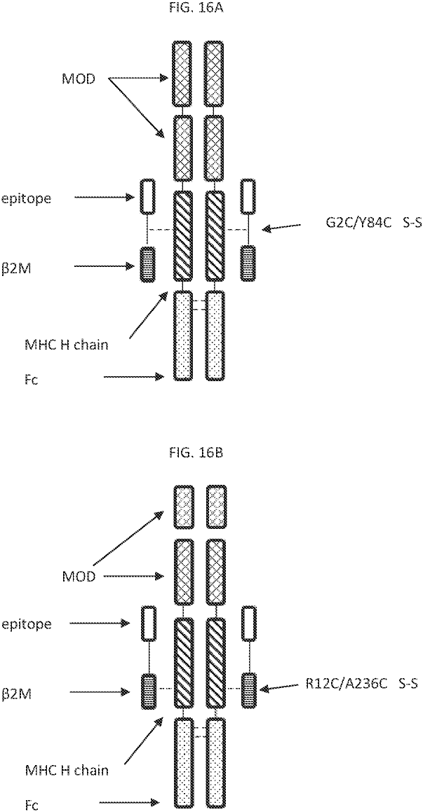

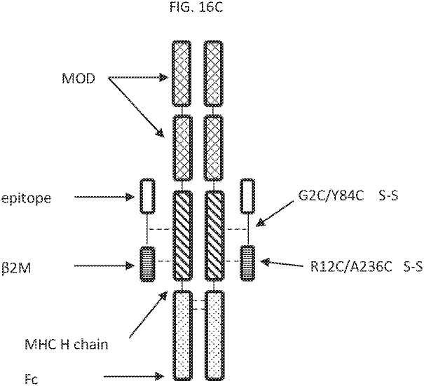

In some cases, the first polypeptide and the second polypeptide of a TMP are linked to one another by at least two disulfide bonds (i.e., two interchain disulfide bonds). Examples of such multiple disulfide-linked TMP are depicted schematically in FIGS. 12A and 12B; and in FIG. 16C. In addition, where a TMP comprises an IgFc polypeptide, a heterodimeric TMP can be dimerized, such that disulfide bonds link the IgFc polypeptides in the two heterodimeric TMPs. Such an arrangement is depicted schematically in FIGS. 12C and 12D, where disulfide bonds are represented by dashed lines. Unless otherwise stated, the at least two disulfide bonds described in the multiple disulfide-linked TMPPs in this section are not referring to disulfide bonds linking IgFc polypeptides in dimerized TMPs.

As noted above, in some cases, the first polypeptide and the second polypeptide of a TMP of the present disclosure are linked to one another by at least two disulfide bonds (i.e., two interchain disulfide bonds). For example, in some instances, the first polypeptide and the second polypeptide of a TMP of the present disclosure are linked to one another by 2 interchain disulfide bonds. As another example, in some instances, the first polypeptide and the second polypeptide of a TMP of the present disclosure are linked to one another by 3 interchain disulfide bonds. As another example, in some instances, the first polypeptide and the second polypeptide of a TMP of the present disclosure are linked to one another by 4 interchain disulfide bonds.

In some cases where a peptide epitope in a first polypeptide of a TMP of the present disclosure is linked to a β2M polypeptide by a linker comprising a Cys, at least one of the at least two disulfide bonds links a Cys in the linker to a Cys in an MHC Class I heavy chain in the second polypeptide. In some cases, where a peptide epitope in a first polypeptide of a TMP of the present disclosure is linked to an MHC Class I heavy chain polypeptide by a linker, at least one of the at least two disulfide bonds links a Cys in the linker to a Cys in a β2M polypeptide present in the second polypeptide.

In some cases, a multiple disulfide-linked TMP of the present disclosure (e.g., a double disulfide-linked TMP) exhibits increased stability, compared to a control TMP that includes only one of the at least two disulfide bonds. In some cases, a multiple disulfide-linked TMP (e.g., a double disulfide-linked TMP) of the present disclosure exhibits increased in vitro stability, compared to a control TMP that includes only one of the at least two disulfide bonds. For example, in some cases, a multiple disulfide-linked TMP of the present disclosure (e.g., a double disulfide-linked TMP) exhibits at least 5%, at least 10%, at least 15%, at least 20%, at least 25%, at least 50%, at least 2-fold, at least 5-fold, or at least 10-fold, greater in vitro stability, compared to a control TMP that includes only one of the at least two disulfide bonds.

Whether a multiple disulfide-linked TMP of the present disclosure exhibits increased in vitro stability, compared to a control TMP that includes only one of the at least two disulfide bonds, can be determined by measuring the amount disulfide-linked heterodimeric TMP present in a sample over time and/or under a specified condition and/or during purification of the TMP.

For example, in some cases, a multiple disulfide-linked TMP (e.g., a double disulfide-linked TMP) of the present disclosure exhibits at least 5%, at least 10%, at least 15%, at least 20%, at least 25%, at least 50%, at least 2-fold, at least 5-fold, or at least 10-fold, greater in vitro stability, compared to a control TMP that includes only one of the at least two disulfide bonds, when the TMP is stored at 37° C. for a period of time (e.g., for a period of time of from about 1 week to about 2 weeks, from about 2 weeks to about 4 weeks, or from about 4 weeks to about 2 months). For example, in some cases, the amount of disulfide-linked heterodimeric TMP remaining after storing a multiple disulfide-linked TMP (e.g., a double disulfide-linked TMP) of the present disclosure in vitro at 37° C. for 28 days is at least at least 5%, at least 10%, at least 15%, at least 20%, at least 25%, at least 50%, at least 2-fold, at least 5-fold, or at least 10-fold, greater than the amount of disulfide-linked heterodimeric TMP remaining after storing a control TMP (a TMP that includes only one of the at least two disulfide bonds present in the multiple disulfide-linked TMP) in vitro at 37° C. for 28 days.

In some cases, a multiple disulfide-linked TMP of the present disclosure exhibits increased in vivo stability, compared to a control TMP that includes only one of the at least two disulfide bonds. For example, in some cases, a multiple disulfide-linked TMP of the present disclosure exhibits at least 5%, at least 10%, at least 15%, at least 20%, at least 25%, at least 50%, at least 2-fold, at least 5-fold, or at least 10-fold, greater in vivo stability, compared to a control TMP that includes only one of the at least two disulfide bonds.

In some cases, the presence of two disulfide bonds in a multiple disulfide-linked TMP of the present disclosure (e.g., a double disulfide-linked TMP) provides for increased production of disulfide-linked heterodimeric TMP, compared to the amount of disulfide-linked heterodimeric TMP produced when the TMP is a control TMP that includes only one of the at least two disulfide bonds. For example, a multiple disulfide-linked TMP of the present disclosure (e.g., a double disulfide-linked TMP) can be produced in a mammalian cell in in vitro cell culture, where the mammalian cell is cultured in a liquid cell culture medium. The TMP can be secreted into the cell culture medium. The cells can be lysed, generating a cell lysate, and the TMP can be present in the cell lysate. The TMP can be purified from the cell culture medium and/or the cell lysate. For example, where the TMP comprises an IgG1 Fc polypeptide, the cell culture medium and/or the cell lysate can be contacted with immobilized protein A (e.g., the cell culture medium and/or the cell lysate can be applied to a protein A column, where protein A is immobilized onto beads). TMP present in the cell culture medium and/or the cell lysate becomes bound to the immobilized protein A. After washing the column to remove unbound materials, the bound TMP is eluted, generating a protein A eluate. The amount of disulfide-linked heterodimeric TMP present in the protein A eluate is a least 0.5%, at least 1%, at least 2%, at least 3%, at least 4%, at least 5%, at least 6%, at least 7%, at least 8%, at least 9%, or at least 10%, higher than the amount of disulfide-linked heterodimeric TMP present in the protein A eluate when the TMP is a control TMP that includes only one of the at least two disulfide bonds present in the multiple disulfide-linked TMP (e.g., a double disulfide-linked TMP). In some cases, the percent of the total TMP protein in the eluate that is non-aggregated disulfide-linked heterodimeric TMP is at least 70%, at least 75%, at least 80%, at least 85%, at least 90%, at least 95%, or at least 99%. The protein A eluate can be subjected to size exclusion chromatography (SEC) and/or one or more other additional purification steps.

In some cases, a TMP comprises at least one heterodimer comprising: a) a first polypeptide comprising: i) a SARS-CoV-2 peptide, where the SARS-CoV-2 peptide has a length of at least 4 amino acids, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, and peptides within a range of from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length); and ii) first MHC polypeptide; b) a second polypeptide comprising a second MHC polypeptide, and c) at least one MOD, where the first and/or the second polypeptide comprises the MOD, and where the heterodimer comprises at least two disulfide bonds (e.g., two disulfide bonds) between the first polypeptide and the second polypeptide (e.g., the heterodimer comprises: i) a first disulfide bond linking the first polypeptide and the second polypeptide; and ii) a second disulfide bond linking the first polypeptide and the second polypeptide). Expressed another way, the first polypeptide comprises a first Cys residue that forms a disulfide bond (a first disulfide bond) with a first Cys residue in the second polypeptide; and the first polypeptide comprises a second Cys residue that forms a disulfide bond (a second disulfide bond) with a second Cys residue in the second polypeptide.

In some cases, a TMP comprises: a) a first polypeptide comprising, in order from N-terminus to C-terminus: i) a SARS-CoV-2 peptide; ii) a peptide linker; and iii) a β2M polypeptide; and b) a second polypeptide comprising an MHC Class I heavy chain polypeptide, where one or both of the first and the second polypeptides comprises at least one MOD, where the TMP comprises: a) a first disulfide linkage between: i) a Cys present in the linker between the SARS-CoV-2 peptide and the β2M polypeptide; and ii) a first Cys introduced into the MHC Class I heavy chain polypeptide; and b) at least a second disulfide linkage between the first polypeptide and the second polypeptide, where the at least a second disulfide linkage is between: i) a Cys in the first polypeptide that is C-terminal to the Cys present in the linker; and ii) a Cys in the second polypeptide that is C-terminal to the first Cys introduced into the MHC Class I heavy chain polypeptide.

A multiple disulfide-linked TMP of the present disclosure (e.g., a double disulfide-linked TMP) can comprise, for example: a) a first polypeptide comprising: i) a SARS-CoV-2 peptide (e.g., a SARS-CoV-2 peptide of from 4 amino acids to about 25 amino acids in length, that is bound by a TCR when the peptide is complexed with MHC polypeptides); and ii) a first MHC polypeptide, where the first polypeptide comprises a peptide linker between the SARS-CoV-2 peptide and the first MHC polypeptide, where the peptide linker comprises a Cys residue, and where the first MHC polypeptide is a β2M polypeptide that comprises an amino acid substitution that introduces a Cys residue; b) and a second polypeptide comprising a second MHC polypeptide, where the second MHC polypeptide is a Class I heavy chain comprising a Y84C substitution and an A236C substitution, based on the amino acid numbering of HLA-A*0201 (depicted in FIG. 7A), or at corresponding positions in another Class I heavy chain allele, where the TMP comprises a disulfide bond between the Cys residue in the peptide linker and the Cys residue at amino acid position 84 of the Class I heavy chain or corresponding position of another Class I heavy chain allele, and where the TMP comprises a disulfide bond between the introduced Cys residue in the β2M polypeptide and the Cys at amino acid position 236 of the Class I heavy chain or corresponding position of another Class I heavy chain allele; and c) at least one MOD, where the first and/or the second polypeptide comprises the at least one MOD. Examples are depicted schematically in FIG. 12A and FIG. 12B.

In some cases, the peptide linker comprises the amino acid sequence GCGGS (SEQ ID NO:177). In some cases, the peptide linker comprises the amino acid sequence GCGGS(GGGGS)n (SEQ ID NO:171), where n is an integer from 1 to 10. In some cases, the peptide linker comprises the amino acid sequence GCGGS(GGGGS)n (SEQ ID NO:171), where n is 1. In some cases, the peptide linker comprises the amino acid sequence GCGGS(GGGGS)n (SEQ ID NO:171), where n is 2. In some cases, the peptide linker comprises the amino acid sequence GCGGS(GGGGS)n (SEQ ID NO:171), where n is 3. In some cases, the peptide linker comprises the amino acid sequence GCGGS(GGGGS)n (SEQ ID NO:171), where n is 4. In some cases, the peptide linker comprises the amino acid sequence GCGGS(GGGGS)n (SEQ ID NO:171), where n is 5. In some cases, the peptide linker comprises the amino acid sequence GCGGS(GGGGS)n (SEQ ID NO:171), where n is 6. In some cases, the peptide linker comprises the amino acid sequence GCGGS(GGGGS)n (SEQ ID NO:171), where n is 7. In some cases, the peptide linker comprises the amino acid sequence GCGGS(GGGGS)n (SEQ ID NO:171), where n is 8. In some cases, the peptide linker comprises the amino acid sequence GCGGS(GGGGS)n (SEQ ID NO:171), where n is 9. In some cases, the peptide linker comprises the amino acid sequence GCGGS(GGGGS)n (SEQ ID NO:171), where n is 10.

In some cases, the peptide linker comprises the amino acid sequence CGGGS (SEQ ID NO:174). In some cases, the peptide linker comprises the amino acid sequence CGGGS(GGGGS)n (SEQ ID NO:175), where n is an integer from 1 to 10. In some cases, the peptide linker comprises the amino acid sequence CGGGS(GGGGS)n (SEQ ID NO:175), where n is 1. In some cases, the peptide linker comprises the amino acid sequence CGGGS(GGGGS)n (SEQ ID NO:175), where n is 2. In some cases, the peptide linker comprises the amino acid sequence CGGGS(GGGGS)n (SEQ ID NO:175), where n is 3. In some cases, the peptide linker comprises the amino acid sequence CGGGS(GGGGS)n (SEQ ID NO:175), where n is 4. In some cases, the peptide linker comprises the amino acid sequence CGGGS(GGGGS)n (SEQ ID NO:175), where n is 5. In some cases, the peptide linker comprises the amino acid sequence CGGGS(GGGGS)n (SEQ ID NO:175), where n is 6. In some cases, the peptide linker comprises the amino acid sequence CGGGS(GGGGS)n (SEQ ID NO:175), where n is 7. In some cases, the peptide linker comprises the amino acid sequence CGGGS(GGGGS)n (SEQ ID NO:175), where n is 8. In some cases, the peptide linker comprises the amino acid sequence CGGGS(GGGGS)n (SEQ ID NO:175), where n is 9. In some cases, the peptide linker comprises the amino acid sequence CGGGS(GGGGS)n (SEQ ID NO:175), where n is 10.

The following are non-limiting examples of MHC Class I heavy chain comprising a Y84C substitution and an A236C substitution, based on the amino acid numbering of HLA-A*0201 (depicted in FIG. 7A), or at corresponding positions in another Class I heavy chain allele.

### Hla-a

In some cases, a multiple disulfide-linked TMP of the present disclosure (e.g., a double disulfide-linked TMP) comprises: a) a first polypeptide comprising: i) a SARS-CoV-2 peptide (e.g., a SARS-CoV-2 peptide of from 4 amino acids to 25 amino acids in length, that is bound by a TCR when the peptide is complexed with MHC polypeptides; and ii) a first MHC polypeptide, where the first polypeptide comprises a peptide linker between the peptide and the first MHC polypeptide, where the peptide linker comprises a Cys residue, and where the first MHC polypeptide is a β2M polypeptide that comprises an amino acid substitution that introduces a Cys residue; and b) a second polypeptide comprising an HLA-A MHC Class I heavy chain comprising an amino acid sequence having at least 60%, at least 70%, at least 80%, at least 90%, at least 95%, at least 98%, at least 99%, or 100%, amino acid sequence identity to the following amino acid sequence:
GSHSMRYFFTSVSRPGRGEPRFIAVGYVDDTQFVRFDSDAASQRMEPRAPWIEQEGPEYWDGET RKVKAHSQTHRVDLGTLRGCYNQSEAGSHTVQRMYGCDVGSDWRFLRGYHQYAYDGKDYIA LKEDLRSWTAADMAAQTTKHKWEAAHVAEQLRAYLEGTCVEWLRRYLENGKETLQRTDAPK THMTHHAVSDHEATLRCWALSFYPAEITLTWQRDGEDQTQDTELVETRPCGDGTFQKWAAVV VPSGQEQRYTCHVQHEGLPKPLTLRWEP (SEQ ID NO:176), where amino acid 84 is a Cys and amino acid 236 is a Cys; and c) at least one MOD, where the first and/or the second polypeptide comprises the at least one MOD. In some cases, the peptide linker comprises the amino acid sequence GCGGS (SEQ ID NO:177). In some cases, the peptide linker comprises the amino acid sequence GCGGS(GGGGS)n (SEQ ID NO:171), where n is an integer from 1 to 10. In some cases, the β2M polypeptide comprises an R12C substitution. For example, the β2M polypeptide can comprises an amino acid sequence having at least 90%, at least 95%, at least 98%, at least 99%, or 100%, amino acid sequence identity to the following amino acid sequence:
IQRTPKIQVYSCHPAENGKSNFLNCYVSGFHPSDIEVDLLKNGERIEKVEHSDLSFSKDWSFYLL YYTEFTPTEKDEYACRVNHVTLSQPKIVKWDRDM (SEQ ID NO:167), where amino acid 12 is a Cys. The at least one MOD can be a cytokine, a 4-1BBL polypeptide, a B7-1 polypeptide; a B7-2 polypeptide, an ICOS-L polypeptide, an OX-40L polypeptide, a CD80 polypeptide, a CD86 polypeptide, a PD-L1 polypeptide, a FasL polypeptide, or a PD-L2 polypeptide. In some cases, the at least one MOD is a reduced affinity variant, as described elsewhere herein. In some cases, the first or the second polypeptide comprises an Ig Fc polypeptide.

In some cases, a multiple disulfide-linked TMP of the present disclosure (e.g., a double disulfide-linked TMP) comprises an HLA-A Class I heavy chain polypeptide. In some cases, the HLA-A heavy chain polypeptide present in a multiple disulfide-linked TMP of the present disclosure (e.g., a double disulfide-linked TMP) comprises an amino acid sequence having at least 95%, at least 98%, or at least 99%, amino acid sequence identity to the HLA-A*0101, HLA-A*0201, HLA-A*0202, HLA-A*1101, HLA-A*2301, HLA-A*2402, HLA-A*2407, HLA-A*3303, or HLA-A*3401 amino acid sequence depicted in FIG. 7A, where the HLA-A heavy chain polypeptide comprises Y84C and A236C substitutions.

### Hla-a*0101 (y84c; A236c)

In some cases, the HLA-A heavy chain polypeptide present in a multiple disulfide-linked TMP of the present disclosure (e.g., a double disulfide-linked TMP) comprises an amino acid sequence having at least 95%, at least 98%, at least 99%, or 100%, amino acid sequence identity to the following HLA-A*0101 (Y84C; A236C) amino acid sequence:

GSHSMRYFFTSVSRPGRGEPRFIAVGYVDDTQFVRFDSDAASQKMEPRAP
WIEQEGPEYWDQETRNMKAHSQTDRANLGTLRGCYNQSEDGSHTIQIMYG
CDVGPDGRFLRGYRQDAYDGKDYIALNEDLRSWTAADMAAQITKRKWEAV
HAAEQRRVYLEGRCVDGLRRYLENGKETLQRTDPPKTHMTHHPISDHEAT
LRCWALGFYPAEITLTWQRDGEDQTQDTELVETRPCGDGTFQKWAAVVVP
SGEEQRYTCHVQHEGLPKPLTLRWE (SEQ ID NO:178),

where amino acid 84 is a Cys and amino acid 236 is a Cys.

### Hla-a*0201 (y84c; A236c)

In some cases, the HLA-A heavy chain polypeptide present in a multiple disulfide-linked TMP of the present disclosure (e.g., a double disulfide-linked TMP) comprises an amino acid sequence having at least 95%, at least 98%, at least 99%, or 100%, amino acid sequence identity to the following HLA-A*0201 (Y84C; A236C) amino acid sequence:

GSHSMRYFFTSVSRPGRGEPRFIAVGYVDDTQFVRFDSDAASQRMEPRAP
WIEQEGPEYWDGETRKVKAHSQTHRVDLGTLRGCYNQSEAGSHTVQRMYG
CDVGSDWRFLRGYHQYAYDGKDYIALKEDLRSWTAADMAAQTTKHKWEAA
HVAEQLRAYLEGTCVEWLRRYLENGKETLQRTDAPKTHMTHHAVSDHEAT
LRCWALSFYPAEITLTWQRDGEDQTQDTELVETRPCGDGTFQKWAAVVVP
SGQEQRYTCHVQHEGLPKPLTLRWEP (SEQ ID NO:176),

where amino acid 84 is a Cys and amino acid 236 is a Cys.

### Hla-a*0202 (y84c; A236c)

In some cases, the HLA-A heavy chain polypeptide present in a multiple disulfide-linked TMP of the present disclosure (e.g., a double disulfide-linked TMP) comprises an amino acid sequence having at least 95%, at least 98%, at least 99%, or 100%, amino acid sequence identity to the following HLA-A*0202 (Y84C; A236C) amino acid sequence:

GSHSMRYFFTSVSRPGRGEPRFIAVGYVDDTQFVRFDSDAASQRMEPRAP
WIEQEGPEYWDGETRKVKAHSQTHRVDLGTLRGCYNQSEAGSHTVQRMYG
CDVGSDWRFLRGYHQYAYDGKDYIALKEDLRSWTAADMAAQTTKHKWEAA
HVAEQLRAYLEGTCVEWLRRYLENGKETLQRTDAPKTHMTHHAVSDHEAT
LRCWALSFYPAEITLTWQRDGEDQTQDTELVETRPCGDGTFQKWAAVVVP
SGQEQRYTCHVQHEGLPKPLTLRWE (SEQ ID NO:179),

where amino acid 84 is a Cys and amino acid 236 is a Cys.

### Hla-a*1101 (y84c; A236c)

In some cases, the HLA-A heavy chain polypeptide present in a multiple disulfide-linked TMP of the present disclosure (e.g., a double disulfide-linked TMP) comprises an amino acid sequence having at least 95%, at least 98%, at least 99%, or 100%, amino acid sequence identity to the following HLA-A*1101 (Y84C; A236C) amino acid sequence:

GSHSMRYFYTSVSRPGRGEPRFIAVGYVDDTQFVRFDSDAASQRMEPRAP
WIEQEGPEYWDQETRNVKAQSQTDRVDLGTLRGCYNQSEDGSHTIQIMYG
CDVGPDGRFLRGYRQDAYDGKDYIALNEDLRSWTAADMAAQITKRKWEAA
HAAEQQRAYLEGRCVEWLRRYLENGKETLQRTDPPKTHMTHHPISDHEAT
LRCWALGFYPAEITLTWQRDGEDQTQDTELVETRPCGDGTFQKWAAVVVP
SGEEQRYTCHVQHEGLPKPLTLRWE (SEQ ID NO:180),

where amino acid 84 is a Cys and amino acid 236 is a Cys.

### Hla-a*2301 (y84c; A236c)

In some cases, the HLA-A heavy chain polypeptide present in a multiple disulfide-linked TMP of the present disclosure (e.g., a double disulfide-linked TMP) comprises an amino acid sequence having at least 95%, at least 98%, at least 99%, or 100%, amino acid sequence identity to the following HLA-A*2301 (Y84C; A236C) amino acid sequence:

GSHSMRYFSTSVSRPGRGEPRFIAVGYVDDTQFVRFDSDAASQRMEPRAP
WIEQEGPEYWDEETGKVKAHSQTDRENLRIALRCYNQSEAGSHTLQMMFG
CDVGSDGRFLRGYHQYAYDGKDYIALKEDLRSWTAADMAAQITQRKWEAA
RVAEQLRAYLEGTCVDGLRRYLENGKETLQRTDPPKTHMTHHPISDHEAT
LRCWALGFYPAEITLTWQRDGEDQTQDTELVETRPCGDGTFQKWAAVVVP
SGEEQRYTCHVQHEGLPKPLTLRWE (SEQ ID NO:181),

where amino acid 84 is a Cys and amino acid 236 is a Cys.

### Hla-a*2402 (y84c; A236c)

In some cases, the HLA-A heavy chain polypeptide present in a multiple disulfide-linked TMP of the present disclosure (e.g., a double disulfide-linked TMP) comprises an amino acid sequence having at least 95%, at least 98%, at least 99%, or 100%, amino acid sequence identity to the following HLA-A*2402 (Y84C; A236C) amino acid sequence:

GSHSMRYFSTSVSRPGRGEPRFIAVGYVDDTQFVRFDSDAASQRMEPRAP
WIEQEGPEYWDEETGKVKAHSQTDRENLRIALRCYNQSEAGSHTLQMMFG
CDVGSDGRFLRGYHQYAYDGKDYIALKEDLRSWTAADMAAQITKRKWEAA
HVAEQQRAYLEGTCVDGLRRYLENGKETLQRTDPPKTHMTHHPISDHEAT
LRCWALGFYPAEITLTWQRDGEDQTQDTELVETRPCGDGTFQKWAAVVVP
SGEEQRYTCHVQHEGLPKPLTLRWE (SEQ ID NO:157),

where amino acid 84 is a Cys and amino acid 236 is a Cys.

### Hla-a*2407 (y84c; A236c)

In some cases, the HLA-A heavy chain polypeptide present in a multiple disulfide-linked TMP of the present disclosure (e.g., a double disulfide-linked TMP) comprises an amino acid sequence having at least 95%, at least 98%, at least 99%, or 100%, amino acid sequence identity to the following HLA-A*2407 (Y84C; A236C) amino acid sequence:

GSHSMRYFSTSVSRPGRGEPRFIAVGYVDDTQFVRFDSDAASQRMEPRAP
WIEQEGPEYWDEETGKVKAQSQTDRENLRIALRCYNQSEAGSHTLQMMFG
CDVGSDGRFLRGYHQYAYDGKDYIALKEDLRSWTAADMAAQITKRKWEAA
HVAEQQRAYLEGTCVDGLRRYLENGKETLQRTDPPKTHMTHHPISDHEAT
LRCWALGFYPAEITLTWQRDGEDQTQDTELVETRPCGDGTFQKWAAVVVP
SGEEQRYTCHVQHEGLPKPLTLRWE (SEQ ID NO:182),

where amino acid 84 is a Cys and amino acid 236 is a Cys.

### Hla-a*3303 (y84c; A236c)

In some cases, the HLA-A heavy chain polypeptide present in a multiple disulfide-linked TMP of the present disclosure (e.g., a double disulfide-linked TMP) comprises an amino acid sequence having at least 95%, at least 98%, at least 99%, or 100%, amino acid sequence identity to the following HLA-A*3303 (Y84C; A236C) amino acid sequence:

GSHSMRYFTTSVSRPGRGEPRFIAVGYVDDTQFVRFDSDAASQRMEPRA
PWIEQEGPEYWDRNTRNVKAHSQIDRVDLGTLRGCYNQSEAGSHTIQMMY
GCDVGSDGRFLRGYQQDAYDGKDYIALNEDLRSWTAADMAAQITQRKWEA
ARVAEQLRAYLEGTCVEWLRRYLENGKETLQRTDPPKTHMTHHAVSDHEA
TLRCWALSFYPAEITLTWQRDGEDQTQDTELVETRPCGDGTFQKWASVVV
PSGQEQRYTCHVQHEGLPKPLTLRWE (SEQ ID NO:183),

where amino acid 84 is a Cys and amino acid 236 is a Cys.

### Hla-a*3401 (y84c; A236c)

In some cases, the HLA-A heavy chain polypeptide present in a multiple disulfide-linked TMP of the present disclosure (e.g., a double disulfide-linked TMP) comprises an amino acid sequence having at least 95%, at least 98%, at least 99%, or 100%, amino acid sequence identity to the following HLA-A*3401 (Y84C; A236C) amino acid sequence:

GSHSMRYFYTSVSRPGRGEPRFIAVGYVDDTQFVRFDSDAASQRMEPRAP
WIEQEGPEYWDRNTRKVKAQSQTDRVDLGTLRGCYNQSEDGSHTIQRMYG
CDVGPDGRFLRGYQQDAYDGKDYIALNEDLRSWTAADMAAQITQRKWETA
HEAEQWRAYLEGTCVEWLRRYLENGKETLQRTDAPKTHMTHHAVSDHEAT
LRCWALSFYPAEITLTWQRDGEDQTQDTELVETRPCGDGTFQKWASVVVP
SGQEQRYTCHVQHEGLPKPLTLRWE (SEQ ID NO:184),

where amino acid 84 is a Cys and amino acid 236 is a Cys.

### Hla-b

In some cases, a multiple disulfide-linked TMP of the present disclosure (e.g., a double disulfide-linked TMP) comprises: a) a first polypeptide comprising: i) a SARS-CoV-2 peptide of at least 4 amino acids in length (e.g., a SARS-CoV-2 peptide of from 4 amino acids to about 25 amino acids in length), that is bound by a TCR when the peptide is complexed with MHC polypeptides); and ii) a first MHC polypeptide, where the first polypeptide comprises a peptide linker between the peptide and the first MHC polypeptide, where the peptide linker comprises a Cys residue, and where the first MHC polypeptide is a β2M polypeptide that comprises an amino acid substitution that introduces a Cys residue; and b) a second polypeptide comprising an HLA-B MHC Class I heavy chain comprising an amino acid sequence having at least 60%, at least 70%, at least 80%, at least 90%, at least 95%, at least 98%, at least 99%, or 100%, amino acid sequence identity to the following amino acid sequence:
GSHSMRYFYTSVSRPGRGEPRFISVGYVDDTQFVRFDSDAASPREEPRAPWIEQEGPEYWDRNT QIYKAQAQTDRESLRNLRGCYNQSEAGSHTLQSMYGCDVGPDGRLLRGHDQYAYDGKDYIAL NEDLRSWTAADTAAQITQRKWEAAREAEQRRAYLEGECVEWLRRYLENGKDKLERADPPKTH VTHHPISDHEATLRCWALGFYPAEITLTWQRDGEDQTQDTELVETRPCGDRTFQKWAAVVVPS GEEQRYTCHVQHEGLPKPLTLRWEP (SEQ ID NO:185), where amino acid 84 is a Cys and amino acid 236 is a Cys; and c) at least one MOD, where the first and/or the second polypeptide comprises the at least one MOD. In some cases, the peptide linker comprises the amino acid sequence GCGGS (SEQ ID NO:177). In some cases, the peptide linker comprises the amino acid sequence GCGGS(GGGGS)n (SEQ ID NO:171), where n is an integer from 1 to 10. In some cases, the β2M polypeptide comprises an R12C substitution. For example, the β2M polypeptide can comprises an amino acid sequence having at least 90%, at least 95%, at least 98%, at least 99%, or 100%, amino acid sequence identity to the following amino acid sequence:
IQRTPKIQVYSCHPAENGKSNFLNCYVSGFHPSDIEVDLLKNGERIEKVEHSDLSFSKDWSFYLL YYTEFTPTEKDEYACRVNHVTLSQPKIVKWDRDM (SEQ ID NO:167), where amino acid 12 is a Cys. The at least one MOD can be a cytokine (e.g., IL-2), a 4-1BBL polypeptide, an ICOS-L polypeptide, an OX-40L polypeptide, a CD80 polypeptide, a CD86 polypeptide, a PD-L1 polypeptide, a FasL polypeptide, or a PD-L2 polypeptide. In some cases, the at least one MOD is a reduced affinity variant, as described elsewhere herein. In some cases, the first or the second polypeptide comprises an Ig Fc polypeptide.

In some cases, a multiple disulfide-linked TMP comprises an HLA-B Class I heavy chain polypeptide. In some cases, the HLA-B heavy chain polypeptide present in a multiple disulfide-linked TMP of the present disclosure (e.g., a double disulfide-linked TMP) comprises an amino acid sequence having at least 95%, at least 98%, or at least 99%, amino acid sequence identity to the HLA-B*0702, HLA-B*0801, HLA-B*1502, HLA-B*3802, HLA-B*4001, HLA-B*4601, or HLA-B*5301 amino acid sequence depicted in FIG. 8A, where the HLA-B heavy chain polypeptide comprises Y84C and A236C substitutions.

### Hla-b*0702 (y84c; A236c)

In some cases, the HLA-B heavy chain polypeptide present in a multiple disulfide-linked TMP (e.g., a double disulfide-linked TMP) comprises an amino acid sequence having at least 95%, at least 98%, at least 99%, or 100%, amino acid sequence identity to the following HLA-B*0702 (Y84C; A236C) amino acid sequence:

GSHSMRYFYTSVSRPGRGEPRFISVGYVDDTQFVRFDSDAASPREEPRAP
WIEQEGPEYWDRNTQIYKAQAQTDRESLRNLRGCYNQSEAGSHTLQSMYG
CDVGPDGRLLRGHDQYAYDGKDYIALNEDLRSWTAADTAAQITQRKWEAA
REAEQRRAYLEGECVEWLRRYLENGKDKLERADPPKTHVTHHPISDHEAT
LRCWALGFYPAEITLTWQRDGEDQTQDTELVETRPCGDRTFQKWAAVVVP
SGEEQRYTCHVQHEGLPKPLTLRWE (SEQ ID NO:186),

where amino acid 84 is a Cys and amino acid 236 is a Cys.

### Hla-b*0801 (y84c; A236c)

In some cases, the HLA-B heavy chain polypeptide present in a multiple disulfide-linked TMP (e.g., a double disulfide-linked TMP) comprises an amino acid sequence having at least 95%, at least 98%, at least 99%, or 100%, amino acid sequence identity to the following HLA-B*0801 (Y84C; A236C) amino acid sequence:

GSHSMRYFDTAMSRPGRGEPRFISVGYVDDTQFVRFDSDAASPREEPRAP
WIEQEGPEYWDRNTQIFKTNTQTDRESLRNLRGCYNQSEAGSHTLQSMYG
CDVGPDGRLLRGHNQYAYDGKDYIALNEDLRSWTAADTAAQITQRKWEAA
RVAEQDRAYLEGTCVEWLRRYLENGKDTLERADPPKTHVTHHPISDHEAT
LRCWALGFYPAEITLTWQRDGEDQTQDTELVETRPCGDRTFQKWAAVVVP
SGEEQRYTCHVQHEGLPKPLTLRWE (SEQ ID NO:187),

where amino acid 84 is a Cys and amino acid 236 is a Cys.

### Hla-b*1502 (y84c; A236c)

In some cases, the HLA-B heavy chain polypeptide present in a multiple disulfide-linked TMP (e.g., a double disulfide-linked TMP) comprises an amino acid sequence having at least 95%, at least 98%, at least 99%, or 100%, amino acid sequence identity to the following HLA-B*1502 (Y84C; A236C) amino acid sequence:

GSHSMRYFYTAMSRPGRGEPRFIAVGYVDDTQFVRFDSDAASPRMAPRAP
WIEQEGPEYWDRNTQISKTNTQTYRESLRNLRGCYNQSEAGSHIIQRMYG
CDVGPDGRLLRGYDQSAYDGKDYIALNEDLSSWTAADTAAQITQRKWEAA
REAEQLRAYLEGLCVEWLRRYLENGKETLQRADPPKTHVTHHPISDHEAT
LRCWALGFYPAEITLTWQRDGEDQTQDTELVETRPCGDRTFQKWAAVVVP
SGEEQRYTCHVQHEGLPKPLTLRWE (SEQ ID NO:188),

where amino acid 84 is a Cys and amino acid 236 is a Cys.

### Hla-b*3802 (y84c; A236c)

In some cases, the HLA-B heavy chain polypeptide present in a multiple disulfide-linked TMP (e.g., a double disulfide-linked TMP) comprises an amino acid sequence having at least 95%, at least 98%, at least 99%, or 100%, amino acid sequence identity to the following HLA-B*3802 (Y84C; A236C) amino acid sequence:

GSHSMRYFYTSVSRPGRGEPRFISVGYVDDTQFVRFDSDAASPREEPRAP
WIEQEGPEYWDRNTQICKTNTQTYRENLRTALRCYNQSEAGSHTLQRMYG
CDVGPDGRLLRGHNQFAYDGKDYIALNEDLSSWTAADTAAQITQRKWEAA
RVAEQLRTYLEGTCVEWLRRYLENGKETLQRADPPKTHVTHHPISDHEAT
LRCWALGFYPAEITLTWQRDGEDQTQDTELVETRPCGDRTFQKWAAVVVP
SGEEQRYTCHVQHEGLPKPLTLRWE (SEQ ID NO:189),

where amino acid 84 is a Cys and amino acid 236 is a Cys.

### Hla-b*4001 (y84c; A2346c)

In some cases, the HLA-B heavy chain polypeptide present in a multiple disulfide-linked TMP (e.g., a double disulfide-linked TMP) comprises an amino acid sequence having at least 95%, at least 98%, at least 99%, or 100%, amino acid sequence identity to the following HLA-B*4001 (Y84C; A236C) amino acid sequence:

GSHSMRYFHTAMSRPGRGEPRFITVGYVDDTLFVRFDSDATSPRKEPRAP
WIEQEGPEYWDRETQISKTNTQTYRESLRNLRGCYNQSEAGSHTLQRMYG
CDVGPDGRLLRGHNQYAYDGKDYIALNEDLRSWTAADTAAQISQRKLEAA
RVAEQLRAYLEGECVEWLRRYLENGKDKLERADPPKTHVTHHPISDHEAT
LRCWALGFYPAEITLTWQRDGEDQTQDTELVETRPCGDRTFQKWAAVVVP
SGEEQRYTCHVQHEGLPKPLTLRWE (SEQ ID NO:190)

where amino acid 84 is a Cys and amino acid 236 is a Cys.

### Hla-b*4601 (y84c; A236c)

In some cases, the HLA-B heavy chain polypeptide present in a multiple disulfide-linked TMP (e.g., a double disulfide-linked TMP) comprises an amino acid sequence having at least 95%, at least 98%, at least 99%, or 100%, amino acid sequence identity to the following HLA-B*4601 (Y84C; A236C) amino acid sequence:

GSHSMRYFYTAMSRPGRGEPRFIAVGYVDDTQFVRFDSDAASPRMAPRAP
WIEQEGPEYWDRETQKYKRQAQTDRVSLRNLRGCYNQSEAGSHTLQRMYG
CDVGPDGRLLRGHDQSAYDGKDYIALNEDLSSWTAADTAAQITQRKWEAA
REAEQWRAYLEGLCVEWLRRYLENGKETLQRADPPKTHVTHHPISDHEAT
LRCWALGFYPAEITLTWQRDGEDQTQDTELVETRPCGDRTFQKWAAVVVP
SGEEQRYTCHVQHEGLPKPLTLRWE (SEQ ID NO:191)

where amino acid 84 is a Cys and amino acid 236 is a Cys.

### Hla-b*5301 (y84c; A236c)

In some cases, the HLA-B heavy chain polypeptide present in a multiple disulfide-linked TMP (e.g., a double disulfide-linked TMP) comprises an amino acid sequence having at least 95%, at least 98%, at least 99%, or 100%, amino acid sequence identity to the following HLA-B*5301 (Y84C; A236C) amino acid sequence:

GSHSMRYFYTAMSRPGRGEPRFIAVGYVDDTQFVRFDSDAASPRTEPRAP
WIEQEGPEYWDRNTQIFKTNTQTYRENLRIALRCYNQSEAGSHIIQRMYG
CDLGPDGRLLRGHDQSAYDGKDYIALNEDLSSWTAADTAAQITQRKWEAA
RVAEQLRAYLEGLCVEWLRRYLENGKETLQRADPPKTHVTHHPVSDHEAT
LRCWALGFYPAEITLTWQRDGEDQTQDTELVETRPCGDRTFQKWAAVVVP
SGEEQRYTCHVQHEGLPKPLTLRWE (SEQ ID NO:192)

where amino acid 84 is a Cys and amino acid 236 is a Cys.

### Hla-c

In some cases, a multiple disulfide-linked TMP (e.g., a double disulfide-linked TMP) comprises: a) a first polypeptide comprising: i) a SARS-CoV-2 peptide of at least 4 amino acids in length (e.g., a SARS-CoV-2 peptide of from 4 amino acids to about 25 amino acids in length) that is bound by a TCR when the peptide is complexed with MHC polypeptides); and ii) a first MHC polypeptide, where the first polypeptide comprises a peptide linker between the peptide and the first MHC polypeptide, where the peptide linker comprises a Cys residue, and where the first MHC polypeptide is a β2M polypeptide that comprises an amino acid substitution that introduces a Cys residue; and b) a second polypeptide comprising an HLA-C MHC Class I heavy chain comprising an amino acid sequence having at least 60%, at least 70%, at least 80%, at least 90%, at least 95%, at least 98%, at least 99%, or 100%, amino acid sequence identity to the following amino acid sequence:
CSHSMRYFDTAVSRPGRGEPRFISVGYVDDTQFVRFDSDAASPRGEPRAPWVEQEGPEYWDRE TQNYKRQAQADRVSLRNLRGCYNQSEDGSHTLQRMYGCDLGPDGRLLRGYDQSAYDGKDYI ALNEDLRSWTAADTAAQITQRKLEAARAAEQLRAYLEGTCVEWLRRYLENGKETLQRAEPPKT HVTHHPLSDHEATLRCWALGFYPAEITLTWQRDGEDQTQDTELVETRPCGDGTFQKWAAVVV PSGQEQRYTCHMQHEGLQEPLTLSWEP (SEQ ID NO:193), where amino acid 84 is a Cys and amino acid 236 is a Cys; and c) at least one MOD, where the first and/or the second polypeptide comprises the at least one MOD. In some cases, the peptide linker comprises the amino acid sequence GCGGS (SEQ ID NO:177). In some cases, the peptide linker comprises the amino acid sequence GCGGS(GGGGS)n (SEQ ID NO:171), where n is an integer from 1 to 10. In some cases, the β2M polypeptide comprises an R12C substitution. For example, the β2M polypeptide can comprises an amino acid sequence having at least 90%, at least 95%, at least 98%, at least 99%, or 100%, amino acid sequence identity to the following amino acid sequence:
IQRTPKIQVYSCHPAENGKSNFLNCYVSGFHPSDIEVDLLKNGERIEKVEHSDLSFSKDWSFYLL YYTEFTPTEKDEYACRVNHVTLSQPKIVKWDRDM (SEQ ID NO:167), where amino acid 12 is a Cys. The at least one MOD can be a cytokine (e.g., IL-2), a 4-1BBL polypeptide, an ICOS-L polypeptide, an OX-40L polypeptide, a CD80 polypeptide, a CD86 polypeptide, a PD-L1 polypeptide, a FasL polypeptide, or a PD-L2 polypeptide. In some cases, the at least one MOD is a reduced affinity variant, as described elsewhere herein. In some cases, the first or the second polypeptide comprises an Ig Fc polypeptide.

In some cases, a multiple disulfide-linked TMP (e.g., a double disulfide-linked TMP) comprises an HLA-C Class I heavy chain polypeptide. In some cases, the HLA-C heavy chain polypeptide present in a multiple disulfide-linked TMP of the present disclosure (e.g., a double disulfide-linked TMP) comprises an amino acid sequence having at least 95%, at least 98%, or at least 99%, amino acid sequence identity to the HLA-C*0102, HLA-C*0303, HLA-C*0304, HLA-C*0401, HLA-C*0602, HLA-C*0701, HLA-C*0702, HLA-C*0801, or HLA-C*1502 amino acid sequence depicted in FIG. 9A, where the HLA-C heavy chain polypeptide comprises Y84C and A236C substitutions.

### Hla-c*01:02 (y84c; A236c)

In some cases, the HLA-C heavy chain polypeptide present in a multiple disulfide-linked TMP (e.g., a double disulfide-linked TMP) comprises an amino acid sequence having at least 95%, at least 98%, at least 99%, or 100%, amino acid sequence identity to the following HLA-C*01:02 (Y84C; A236C) amino acid sequence:

CSHSMKYFFTSVSRPGRGEPRFISVGYVDDTQFVRFDSDAASPRGEPRAP
WVEQEGPEYWDRETQKYKRQAQTDRVSLRNLRGCYNQSEAGSHTLQWMCG
CDLGPDGRLLRGYDQYAYDGKDYIALNEDLRSWTAADTAAQITQRKWEAA
REAEQRRAYLEGTCVEWLRRYLENGKETLQRAEHPKTHVTHHPVSDHEAT
LRCWALGFYPAEITLTWQWDGEDQTQDTELVETRPCGDGTFQKWAAVMVP
SGEEQRYTCHVQHEGLPEPLTLRWEP (SEQ ID NO:194),

where amino acid 84 is a Cys and amino acid 236 is a Cys.

### Hla-c*0303 (y84c; A236c)

In some cases, the HLA-C heavy chain polypeptide present in a multiple disulfide-linked TMP (e.g., a double disulfide-linked TMP) comprises an amino acid sequence having at least 95%, at least 98%, at least 99%, or 100%, amino acid sequence identity to the following HLA-C*03:03 (Y84C; A236C) amino acid sequence:

GSHSMRYFYTAVSRPGRGEPHFIAVGYVDDTQFVRFDSDAASPRGEPRAP
WVEQEGPEYWDRETQKYKRQAQTDRVSLRNLRGCYNQSEARSHIIQRMYG
CDVGPDGRLLRGYDQYAYDGKDYIALNEDLRSWTAADTAAQITQRKWEAA
REAEQLRAYLEGLCVEWLRRYLKNGKETLQRAEHPKTHVTHHPVSDHEAT
LRCWALGFYPAEITLTWQWDGEDQTQDTELVETRPCGDGTFQKWAAVVVP
SGEEQRYTCHVQHEGLPEPLTLRWEP (SEQ ID NO:195),

where amino acid 84 is a Cys and amino acid 236 is a Cys.

### Hla-c*0304 (y84c; A236c)

In some cases, the HLA-C heavy chain polypeptide present in a multiple disulfide-linked TMP (e.g., a double disulfide-linked TMP) comprises an amino acid sequence having at least 95%, at least 98%, at least 99%, or 100%, amino acid sequence identity to the following HLA-C*03:04 (Y84C; A236C) amino acid sequence:

GSHSMRYFYTAVSRPGRGEPHFIAVGYVDDTQFVRFDSDAASPRGEPRAP
WVEQEGPEYWDRETQKYKRQAQTDRVSLRNLRGCYNQSEAGSHIIQRMYG
CDVGPDGRLLRGYDQYAYDGKDYIALNEDLRSWTAADTAAQITQRKWEAA
REAEQLRAYLEGLCVEWLRRYLKNGKETLQRAEHPKTHVTHHPVSDHEAT
LRCWALGFYPAEITLTWQWDGEDQTQDTELVETRPCGDGTFQKWAAVVVP
SGEEQRYTCHVQHEGLPEPLTLRWEP (SEQ ID NO:196),

where amino acid 84 is a Cys and amino acid 236 is a Cys.

### Hla-c*0401 (y84c; A236c)

In some cases, the HLA-C heavy chain polypeptide present in a multiple disulfide-linked TMP (e.g., a double disulfide-linked TMP) comprises an amino acid sequence having at least 95%, at least 98%, at least 99%, or 100%, amino acid sequence identity to the following HLA-C*04:01 (Y84C; A236C) amino acid sequence:

GSHSMRYFSTSVSWPGRGEPRFIAVGYVDDTQFVRFDSDAASPRGEPREP
WVEQEGPEYWDRETQKYKRQAQADRVNLRKLRGCYNQSEDGSHTLQRMFG
CDLGPDGRLLRGYNQFAYDGKDYIALNEDLRSWTAADTAAQITQRKWEAA
REAEQRRAYLEGTCVEWLRRYLENGKETLQRAEHPKTHVTHHPVSDHEAT
LRCWALGFYPAEITLTWQWDGEDQTQDTELVETRPCGDGTFQKWAAVVVP
SGEEQRYTCHVQHEGLPEPLTLRWKP (SEQ ID NO:197),

where amino acid 84 is a Cys and amino acid 236 is a Cys.

### Hla-c*0602 (y84c; A236c)

In some cases, the HLA-C heavy chain polypeptide present in a multiple disulfide-linked TMP (e.g., a double disulfide-linked TMP) comprises an amino acid sequence having at least 95%, at least 98%, at least 99%, or 100%, amino acid sequence identity to the following HLA-C*06:02 (Y84C; A236C) amino acid sequence:

CSHSMRYFDTAVSRPGRGEPRFISVGYVDDTQFVRFDSDAASPRGEPRAP
WVEQEGPEYWDRETQKYKRQAQADRVNLRKLRGCYNQSEDGSHTLQWMYG
CDLGPDGRLLRGYDQSAYDGKDYIALNEDLRSWTAADTAAQITQRKWEAA
REAEQWRAYLEGTCVEWLRRYLENGKETLQRAEHPKTHVTHHPVSDHEAT
LRCWALGFYPAEITLTWQRDGEDQTQDTELVETRPCGDGTFQKWAAVVVP
SGEEQRYTCHVQHEGLPEPLTLRWEP (SEQ ID NO:198),

where amino acid 84 is a Cys and amino acid 236 is a Cys.

### Hla-c*0701 (y84c; A236c)

In some cases, the HLA-C heavy chain polypeptide present in a multiple disulfide-linked TMP (e.g., a double disulfide-linked TMP) comprises an amino acid sequence having at least 95%, at least 98%, at least 99%, or 100%, amino acid sequence identity to the following HLA-C*07:01 (Y84C; A236C) amino acid sequence:

CSHSMRYFDTAVSRPGRGEPRFISVGYVDDTQFVRFDSDAASPRGEPRAP
WVEQEGPEYWDRETQNYKRQAQADRVSLRNLRGCYNQSEDGSHTLQRMYG
CDLGPDGRLLRGYDQSAYDGKDYIALNEDLRSWTAADTAAQITQRKLEAA
RAAEQLRAYLEGTCVEWLRRYLENGKETLQRAEPPKTHVTHHPLSDHEAT
LRCWALGFYPAEITLTWQRDGEDQTQDTELVETRPCGDGTFQKWAAVVVP
SGQEQRYTCHMQHEGLQEPLTLSWEP (SEQ ID NO:193),

where amino acid 84 is a Cys and amino acid 236 is a Cys.

### Hla-c*0702 (y84c; A236c)

In some cases, the HLA-C heavy chain polypeptide present in a multiple disulfide-linked TMP (e.g., a double disulfide-linked TMP) comprises an amino acid sequence having at least 95%, at least 98%, at least 99%, or 100%, amino acid sequence identity to the following HLA-C*07:02 (Y84C; A236C) amino acid sequence:

CSHSMRYFDTAVSRPGRGEPRFISVGYVDDTQFVRFDSDAASPRGEPRAP
WVEQEGPEYWDRETQKYKRQAQADRVSLRNLRGCYNQSEDGSHTLQRMSG
CDLGPDGRLLRGYDQSAYDGKDYIALNEDLRSWTAADTAAQITQRKLEAA
RAAEQLRAYLEGTCVEWLRRYLENGKETLQRAEPPKTHVTHHPLSDHEAT
LRCWALGFYPAEITLTWQRDGEDQTQDTELVETRPCGDGTFQKWAAVVVP
SGQEQRYTCHMQHEGLQEPLTLSWEP (SEQ ID NO:199),

where amino acid 84 is a Cys and amino acid 236 is a Cys.

### Hla-c*0801 (y84c; A236c)

In some cases, the HLA-C heavy chain polypeptide present in a multiple disulfide-linked TMP (e.g., a double disulfide-linked TMP) comprises an amino acid sequence having at least 95%, at least 98%, at least 99%, or 100%, amino acid sequence identity to the following HLA-C*08:01 (Y84C; A236C) amino acid sequence:

CSHSMRYFYTAVSRPGRGEPRFIAVGYVDDTQFVQFDSDAASPRGEPRAP
WVEQEGPEYWDRETQKYKRQAQTDRVSLRNLRGCYNQSEAGSHTLQRMYG
CDLGPDGRLLRGYNQFAYDGKDYIALNEDLRSWTAADTAAQITQRKWEAA
RTAEQLRAYLEGTCVEWLRRYLENGKKTLQRAEHPKTHVTHHPVSDHEAT
LRCWALGFYPAEITLTWQRDGEDQTQDTELVETRPCGDGTFQKWAAVVVP
SGEEQRYTCHVQHEGLPEPLTLRWGP (SEQ ID NO:200),

where amino acid 84 is a Cys and amino acid 236 is a Cys.

### Hla-c*1502 (y84c; A236c)

In some cases, the HLA-C heavy chain polypeptide present in a multiple disulfide-linked TMP (e.g., a double disulfide-linked TMP) comprises an amino acid sequence having at least 95%, at least 98%, at least 99%, or 100%, amino acid sequence identity to the following HLA-C*15:02 (Y84C; A236C) amino acid sequence:

CSHSMRYFYTAVSRPGRGEPHFIAVGYVDDTQFVRFDSDAASPRGEPRAP
WVEQEGPEYWDRETQNYKRQAQTDRVNLRKLRGCYNQSEAGSHIIQRMYG
CDLGPDGRLLRGHDQLAYDGKDYIALNEDLRSWTAADTAAQITQRKWEAA
REAEQLRAYLEGTCVEWLRRYLENGKETLQRAEHPKTHVTHHPVSDHEAT
LRCWALGFYPAEITLTWQRDGEDQTQDTELVETRPCGDGTFQKWAAVVVP
SGEEQRYTCHVQHEGLPEPLTLRWEP (SEQ ID NO:201),

where amino acid 84 is a Cys and amino acid 236 is a Cys.

### HLA-E and HLA-G

In some cases, a TMP comprises an HLA-E Class I heavy chain polypeptide. In some cases, the HLA-E heavy chain polypeptide present in a TMP comprises an amino acid sequence having at least 95%, at least 98%, or at least 99%, or 100%, amino acid sequence identity to any one of the amino acid sequences depicted in FIGS. 20A-20D.

In some cases, the HLA-E heavy chain polypeptide present in a TMP comprises an amino acid sequence having at least 95%, at least 98%, at least 99%, or 100%, amino acid sequence identity to one of the following sequences:
(i) the HLA-E*01:01 (Y84C; A236C) amino acid sequence shown in in FIG. 20B (SEQ ID NO:1086), where amino acid 84 is a Cys and amino acid 236 is a Cys;
(ii) the HLA-E*01:03 (Y84C; A236C) amino acid sequence shown in FIG. 20D (SEQ ID NO:1088), where amino acid 84 is a Cys and amino acid 236 is a Cys;

In some cases, a TMP comprises an HLA-G Class I heavy chain polypeptide. In some cases, the HLA-G heavy chain polypeptide present in a TMMP comprises an amino acid sequence having at least 95%, at least 98%, or at least 99%, or 100%, amino acid sequence identity to any one of the amino acid sequences depicted in FIGS. 21A-21D.

In some cases, the HLA-G heavy chain polypeptide present in a TMP comprises an amino acid sequence having at least 95%, at least 98%, at least 99%, or 100%, amino acid sequence identity to one of the following sequences:
(i) the HLA-G*01:01 (Y84C; A236C) amino acid sequence shown in in FIG. 21B (SEQ ID NO:1090), where amino acid 84 is a Cys and amino acid 236 is a Cys;
(ii) the HLA-G*01:04 (Y84C; A236C) amino acid sequence shown in FIG. 21D (SEQ ID NO:1092), where amino acid 84 is a Cys and amino acid 236 is a Cys.

### HLA/peptide Binding Assays

Whether a given peptide (e.g., a SARS-CoV-2 peptide that comprises a SARS-CoV-2 epitope) binds a class I HLA (comprising an HLA heavy chain and a β2M polypeptide), and, when bound to the HLA complex, can effectively present an epitope to a TCR, can be determined using any of a number of well-known methods. Assays include binding assays and T-cell activation assays. Cell-based binding assay

As one example, a cell-based peptide-induced stabilization assay can be used to determine peptide-HLA class I binding. In this assay, a peptide of interest is allowed to bind to a TAP-deficient cell, i.e., a cell that has defective transporter associated with antigen processing (TAP) machinery, and consequently, few surface class I molecules. Such cells include, e.g., the human T2 cell line (T2 (174 x CEM.T2; American Type Culture Collection (ATCC) No. CRL-1992). Henderson et al. (1992) Science 255:1264. Without efficient TAP-mediated transport of cytosolic peptides into the endoplasmic reticulum, assembled class I complexes are structurally unstable, and retained only transiently at the cell surface. However, when T2 cells are incubated with an exogenous peptide capable of binding class I, surface peptide-HLA class I complexes are stabilized and can be detected by flow cytometry with, e.g., a pan anti-class I monoclonal antibody. The stabilization and resultant increased life-span of peptide-HLA complexes on the cell surface by the addition of a peptide validates their identity. Analysis can be carried out using flow cytometry, e.g., where the pan-HLA class I antibody comprises a fluorescent label. Binding of the peptide to various allelic forms of HLA H chains can be tested by genetically modifying the T2 cells to express an allelic HLA H chain of interest.

The following is a non-limiting example of use of a T2 assay to assess peptide binding to HLA A*0201. T2 cells are washed in cell culture medium, and concentrated to 10⁶ cells/ml. Peptides of interest are prepared in cell culture medium and serially diluted providing concentrations of 200 µM, 100 µM, 20 µM and 2 µM. The cells are mixed 1:1 with each peptide dilution to give a final volume of 200 µL and final peptide concentrations of 100 µM, 50 µM, 10 µM and 1 µM. A HLA A*0201 binding peptide, GILGFVFTL, and a non-HLA A*0201-restricted peptide, HPVGEADYF (HLA-B*3501), are included as positive and negative controls, respectively. The cell/peptide mixtures are kept at 37° C. 5% CO₂ for ten minutes; then incubated at room temperature overnight. Cells are then incubated for 2 hours at 37° C. and stained with a fluorescently-labeled anti-human HLA antibody. The cells are washed twice with phosphate-buffered saline and analyzed using flow cytometry. The average mean fluorescence intensity (MFI) of the anti-HLA antibody staining is used to measure the strength of binding.

### Biochemical Binding Assay

HLA polypeptides (HLA heavy chain polypeptide complexed with β2M polypeptide) can be tested for binding to a peptide of interest in a cell-free in vitro assay system. For example, a labeled reference peptide (e.g., fluorescently labeled) is allowed to bind to HLA polypeptides (HLA heavy chain polypeptide complexed with β2M polypeptide), to form an HLA-reference peptide complex. The ability of a test peptide of interest to displace the labeled reference peptide from the HLA-reference peptide complex is tested. The relative binding affinity is calculated as the amount of test peptide needed to displace the bound reference peptide. See, e.g., van der Burg et al. (1995) Human Immunol. 44:189.

As another example, a peptide of interest can be incubated with an HLA molecule (HLA heavy chain complexed with a β2M polypeptide), and the stabilization of the HLA/peptide complex can be measured in an immunoassay format. The ability of a peptide of interest to stabilize an HLA molecule is compared to that of a control peptide presenting a known T-cell epitope. Detection of stabilization is based on the presence or absence of the native conformation of the HLA/peptide complex, detected using an anti-HLA antibody. See, e.g., Westrop et al. (2009) J. Immunol. Methods 341:76; Steinitz et al. (2012) Blood 119:4073; and U.S. Pat. No. 9,205,144.

### T-Cell Activation Assays

Whether a given peptide binds a class I HLA (comprising an HLA heavy chain and a β2M polypeptide), and, when bound to the HLA complex, can effectively present an epitope to a TCR, can be determined by assessing T-cell response to the peptide-HLA complex. T-cell responses that can be measured include, e.g., interferon-gamma (IFNγ) production, cytotoxic activity, and the like. ELISPOT assay

Suitable assays include, e.g., an enzyme linked immunospot (ELISPOT) assay. In this assay, production of IFNγ by CD8⁺ T cells is measured following with an antigen-presenting cell (APC) that presents a peptide of interest complexed with HLA class I. Antibody to IFNγ is immobilized on wells of a multi-well plate. APCs are added to the wells, and incubated for a period of time with a peptide of interest, such that the peptide binds HLA class I on the surface of the APCs. CD8⁺ T cells specific for the peptide are added to the wells, and the plate is incubated for about 24 hours. The wells are then washed, and any IFNγ bound to the immobilized anti-IFNγ antibody is detected using a detectably labeled anti-IFNγ antibody. A colorimetric assay can be used. For example, the detectably labeled anti-IFNγ antibody can be a biotin-labeled anti-IFNγ antibody, which can be detected using, e.g., streptavidin conjugated to alkaline phosphatase. A BCIP/NBT (5-bromo-4-chloro-3-indolyl phosphate/nitro blue tetrazolium) solution is added, to develop the assay. The presence of IFNγ-secreting T cells is identified by colored spots. Negative controls include APCs not contacted with the peptide. APCs expressing various HLA H chain alleles can be used to determine whether a peptide of interest effectively binds to a HLA class I molecule comprising a particular HLA H chain.

### Cytotoxicity Assays

Whether a given peptide binds to a particular HLA class I H chain and, when bound to a HLA class I complex comprising the H chain, can effectively present an epitope to a TCR, can also be determined using a cytotoxicity assay. A cytotoxicity assay involves incubation of a target cell with a cytotoxic CD8⁺ T cell. The target cell displays on its surface a peptide/HLA class I complex comprising a peptide of interest and an HLA class I molecule comprising an HLA H chain to be tested. The target cells can be radioactively labeled, e.g., with ⁵¹Cr. Whether the target cell effectively presents an epitope to a TCR on the cytotoxic CD8⁺ T cell, thereby inducing cytotoxic activity by the CD8⁺ T cell toward the target cell, is determined by measuring release of ⁵¹Cr from the lysed target cell. Specific cytotoxicity can be calculated as the amount of cytotoxic activity in the presence of the peptide minus the amount of cytotoxic activity in the absence of the peptide.

### Detection of Antigen-Specific T Cells With Peptide-HLA Tetramers

As another example, multimers (e.g., tetramers) of peptide-HLA complexes are generated with fluorescent or heavy metal tags. The multimers can then be used to identify and quantify specific T cells via flow cytometry (FACS) or mass cytometry (CyTOF). Detection of epitope-specific T cells provides direct evidence that the peptide-bound HLA molecule is capable of binding to a specific TCR on a subset of antigen-specific T cells. See, e.g., Klenerman et al. (2002) Nature Reviews Immunol. 2:263.

### Immunomodulatory Polypeptides (“MODs”)

In some cases, MOD present in a TMP of the present disclosure is a wild-type MOD. In other cases, a MOD present in a TMP of the present disclosure is a variant MOD that has reduced affinity for a co-MOD, compared to the affinity of a corresponding wild-type MOD for the co-MOD. Suitable MODs that exhibit reduced affinity for a co-MOD can have from 1 amino acid (aa) to 20 aa differences from a wild-type MOD. For example, in some cases, a variant MOD present in a TMP of the present disclosure differs in amino acid sequence by 1 aa, 2 aa, 3 aa, 4 aa, 5 aa, 6 aa, 7 aa, 8 aa, 9 aa, or 10 aa, from a corresponding wild-type MOD. As another example, in some cases, a variant MOD present in a TMP of the present disclosure differs in amino acid sequence by 11 aa, 12 aa, 13 aa, 14 aa, 15 aa, 16 aa, 17 aa, 18 aa, 19 aa, or 20 aa, from a corresponding wild-type MOD.

Exemplary pairs of MODs and their co-MODs include, but are not limited to those set out in Table 2, below:

TABLE 2

| | |
|---|---|
| Immunomodulatory Polypeptide (MOD) | Co-MOD |
| 4-1BBL | 4-1BB |
| PD-L1 | PD-1 |
| IL-2 | IL-2 receptor |
| CD80 | CD28 |
| CD86 | CD28 |
| OX40L (CD252) | OX40 (CD134) |
| Fas ligand | Fas |
| ICOS-L | ICOS |
| ICAM | LFA-1 |
| CD30L | CD30 |
| CD40 | CD40L |
| CD83 | CD83L |
| HVEM (CD270) | CD160 |
| JAG1 (CD339) | Notch |
| JAG1 | CD46 |
| CD80 | CTLA4 |
| CD86 | CTLA4 |
| CD70 | CD27 |
| TGFβ | TGFβ receptor |

In some cases, a MOD present in a TMP of the present disclosure binds to its cognate co-MOD with an affinity that it at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the affinity of a corresponding wild-type MOD for the cognate co-MOD. In cases where the co-MOD comprises multiple chains, e.g., IL-2 receptor (IL2R), which comprises three chains, i.e., the α, β and γ chains, the variant MOD can possess reduced affinity to one or more chains of the co-MOD. The combination of the reduced affinity of the MOD for its cognate co-MOD polypeptide, and the affinity of the SARS-CoV-2 peptide for a TCR, provides for enhanced selectivity of a TMP. Reduced-affinity MODs are described in Published PCT Application WO 2019/051091, published 14 Mar. 2019 (Applicant Cue Biopharma, Inc.). See paragraphs [00124]-[00352], the disclosure of which is expressly incorporated herein by reference.

Unless otherwise stated herein, the binding affinity of a TMP of the present disclosure for a co-MOD is determined using BLI as set forth in published PCT Application WO 2019/051091, published 14 Mar. 2019 (Applicant Cue Biopharma, Inc.). See paragraphs [0052]-[0054], the disclosure of which is expressly incorporated herein by reference.

In some cases, a TMP, when administered to an individual in need thereof, induces both an epitope-specific T cell response and an epitope non-specific T cell response. In other words, in some cases, a TMP, when administered to an individual in need thereof, induces an epitope-specific T cell response by modulating the activity of a first T cell that displays both: i) a TCR specific for the SARS-CoV-2 epitope present in the TMP; ii) a co-MOD that binds to the MOD present in the TMP; and induces an epitope non-specific T cell response by modulating the activity of a second T cell that displays: i) a TCR specific for an epitope other than the SARS-CoV-2 epitope present in the TMP; and ii) a co-MOD that binds to the MOD present in the TMP. The ratio of the epitope-specific T cell response to the epitope-non-specific T cell response is at least 2:1, at least 5:1, at least 10:1, at least 15:1, at least 20:1, at least 25:1, at least 50:1, or at least 100:1. The ratio of the epitope-specific T cell response to the epitope-non-specific T cell response is from about 2:1 to about 5:1, from about 5:1 to about 10:1, from about 10:1 to about 15:1, from about 15:1 to about 20:1, from about 20:1 to about 25:1, from about 25:1 to about 50:1, or from about 50:1 to about 100:1, or more than 100:1. “Modulating the activity” of a T cell can include one or more of: i) activating a cytotoxic (e.g., CD8⁺) T cell; ii) inducing cytotoxic activity of a cytotoxic (e.g., CD8⁺) T cell; iii) inducing production and release of a cytotoxin (e.g., a perforin, a granzyme, a granulysin) by a cytotoxic (e.g., CD8⁺) T cell; increase in the number of epitope-specific T cells, and the like. As one example, the ratio of the increase of the number of epitope-specific T cells to the increase in the number of epitope non-specific T cells can be readily determined by known methods.

As depicted schematically in FIG. 17, a MOD (i.e., one or more MOD) can be present in a TMP at any of a variety of positions. FIG. 17 depicts the position of two copies of a variant IL-2 polypeptide; however, the MOD can be any number of and any of a variety of MOD, as described herein. As depicted in FIG. 17, a MOD can be: 1) N-terminal to the MHC class I heavy chain; 2) C-terminal to the MHC class I heavy chain and N-terminal to the Ig Fc polypeptide; in other words, between the MHC class I heavy chain and the Ig Fc polypeptide; 3) C-terminal to the Ig Fc polypeptide; 4) N-terminal to the peptide epitope; or 5) C-terminal to the β2M polypeptide.

As depicted schematically in FIG. 18, a MOD (i.e., one or more MOD) can be present in a single-chain TMP at any of a variety of positions. FIG. 18 depicts the position of two copies of a variant IL-2 polypeptide; however, the MOD can be any number of and any of a variety of MOD, as described herein. As depicted in FIG. 18, an MOD can be: 1) C-terminal to the MHC class I heavy chain and N-terminal to the Ig Fc polypeptide; 2) C-terminal to the Ig Fc polypeptide; or 3) N-terminal to the peptide epitope.

### CD80 Variants

In some cases, a variant immunomodulatory polypeptide present in a TMP of the present disclosure is a variant CD80 polypeptide. Wild-type CD80 binds to CD28. Wild-type CD80 also binds to CD86.

A wild-type amino acid sequence of the ectodomain of human CD80 can be as follows:

VIHVTK EVKEVATLSC GHNVSVEELA QTRIYWQKEK KMVLTMMSGD
MNIWPEYKNR TIFDITNNLS IVILALRPSD EGTYECVVLK YEKDAF
KREH LAEVTLSVKADFPTPSISDF EIPTSNIRRI ICSTSGGFPE PH
LSWLENGE ELNAINTTVS QDPETELYAVSSKLDFNMTT NHSFMCLIK
Y GHLRVNQTFN WNTTKQEHFP DN (SEQ ID NO:4).

A wild-type CD28 amino acid sequence can be as follows: MLRLLLALNL FPSIQVTGNK ILVKQSPMLV AYDNAVNLSC KYSYNLFSRE FRASLHKGLD SAVEVCVVYG NYSQQLQVYS KTGFNCDGKL GNESVTFYLQ NLYVNQTDIY FCKIEVMYPP PYLDNEKSNG TIIHVKGKHL CPSPLFPGPS KPFWVLVVVG GVLACYSLLV TVAFIIFWVR SKRSRLLHSD YMNMTPRRPG PTRKHYQPYA PPRDFAAYRS (SEQ ID NO:5). In some cases, where a TMP of the present disclosure comprises a variant CD80 polypeptide, a “cognate co-immunomodulatory polypeptide” is a CD28 polypeptide comprising the amino acid sequence of SEQ ID NO:5.

A wild-type CD28 amino acid sequence can be as follows: MLRLLLALNL FPSIQVTGNK ILVKQSPMLV AYDNAVNLSW KHLCPSPLFP GPSKPFWVLV VVGGVLACYS LLVTVAFIIF WVRSKRSRLL HSDYMNMTPR RPGPTRKHYQ PYAPPRDFAA YRS (SEQ ID NO:6)

A wild-type CD28 amino acid sequence can be as follows: MLRLLLALNL FPSIQVTGKH LCPSPLFPGP SKPFWVLVVV GGVLACYSLL VTVAFIIFWV RSKRSRLLHS DYMNMTPRRP GPTRKHYQPY APPRDFAAYR S (SEQ ID NO:7).

In some cases, a variant CD80 polypeptide exhibits reduced binding affinity to CD28, compared to the binding affinity of a CD80 polypeptide comprising the amino acid sequence set forth in SEQ ID NO:4 for CD28. For example, in some cases, a variant CD80 polypeptide binds CD28 with a binding affinity that is at least 10%, at least 15%, at least 20%, at least 25%, at least 30%, at least 35%, at least 40%, at least 45%, at least 50% less, at least 55% less, at least 60% less, at least 65% less, at least 70% less, at least 75% less, at least 80% less, at least 85% less, at least 90% less, at least 95% less, or more than 95% less, than the binding affinity of a CD80 polypeptide comprising the amino acid sequence set forth in SEQ ID NO:4 for CD28 (e.g., a CD28 polypeptide comprising the amino acid sequence set forth in one of SEQ ID NO:5, 6, or 7).

Suitable CD80 variants are described in published PCT Application WO 2019/051091, published 14 Mar. 2019 (Applicant Cue Biopharma, Inc.). See paragraphs [00170]-[00196], the disclosure of which is expressly incorporated herein by reference.

### CD86 Variants

In some cases, a variant immunomodulatory polypeptide present in a TMP of the present disclosure is a variant CD86 polypeptide. Suitable CD86 variants are described in published PCT Application WO 2019/051091, published 14 Mar. 2019 (Applicant Cue Biopharma, Inc.). See paragraphs [00197]-[00228], the disclosure of which is expressly incorporated herein by reference.

### 4-1BBL Variants

In some cases, a variant immunomodulatory polypeptide present in a TMP of the present disclosure is a variant 4-1BBL polypeptide. Wild-type 4-1BBL binds to 4-1BB (CD137).

A wild-type 4-1BBL amino acid sequence can be as follows: MEYASDASLD PEAPWPPAPR ARACRVLPWA LVAGLLLLLL LAAACAVFLA CPWAVSGARA SPGSAASPRL REGPELSPDD PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:10).

In some cases, a variant 4-1BBL polypeptide is a variant of the tumor necrosis factor (TNF) homology domain (THD) of human 4-1BBL.

A wild-type amino acid sequence of the THD of human 4-1BBL can be, e.g., one of SEQ ID NOs:11-13, as follows:

PAGLLDLRQG MFAQLVAQNV LLIDGPLSWY SDPGLAGVSL TGGLSY
KEDTKELVVAKAGV YYVFFQLELR RVVAGEGSGS VSLALHLQPL RS
AAGAAALA LTVDLPPASSEARNSAFGFQ GRLLHLSAGQ RLGVHLHTE
A RARHAWQLTQ GATVLGLFRV TPEIPAGLPSPRSE (SEQ ID NO:
11).

D PAGLLDLRQG MFAQLVAQNV LLIDGPLSWY SDPGLAGVSL TGGL
SYKEDTKELVVAKAGV YYVFFQLELR RVVAGEGSGS VSLALHLQPL
RSAAGAAALA LTVDLPPASSEARNSAFGFQ GRLLHLSAGQ RLGVHLH
TEA RARHAWQLTQ GATVLGLFRV TPEIPAGLPSPRSE (SEQ ID N
O:12).

D PAGLLDLRQG MFAQLVAQNV LLIDGPLSWY SDPGLAGVSL TGGL
SYKEDTKELVVAKAGV YYVFFQLELR RVVAGEGSGS VSLALHLQPL
RSAAGAAALA LTVDLPPASSEARNSAFGFQ GRLLHLSAGQ RLGVHLH
TEA RARHAWQLTQ GATVLGLFRV TPEIPA (SEQ IDNO:13).

A wild-type 4-1BB amino acid sequence can be as follows: MGNSCYNIVA TLLLVLNFER TRSLQDPCSN CPAGTFCDNN RNQICSPCPP NSFSSAGGQR TCDICRQCKG VFRTRKECSS TSNAECDCTP GFHCLGAGCS MCEQDCKQGQ ELTKKGCKDC CFGTFNDQKR GICRPWTNCS LDGKSVLVNG TKERDVVCGP SPADLSPGAS SVTPPAPARE PGHSPQIISF FLALTSTALL FLLFFLTLRF SVVKRGRKKL LYIFKQPFMR PVQTTQEEDG CSCRFPEEEE GGCEL (SEQ ID NO:14). In some cases, where a TMP of the present disclosure comprises a variant 4-1BBL polypeptide, a “cognate co-immunomodulatory polypeptide” is a 4-1BB polypeptide comprising the amino acid sequence of SEQ ID NO:14.

In some cases, a variant 4-1BBL polypeptide exhibits reduced binding affinity to 4-1BB, compared to the binding affinity of a 4-1BBL polypeptide comprising the amino acid sequence set forth in one of SEQ ID NOs: 10-13. For example, in some cases, a variant 4-1BBL polypeptide of the present disclosure binds 4-1BB with a binding affinity that is at least 10% less, at least 15% less, at least 20% less, at least 25%, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of a 4-1BBL polypeptide comprising the amino acid sequence set forth in one of SEQ ID NOs:10-13 for a 4-1BB polypeptide (e.g., a 4-1BB polypeptide comprising the amino acid sequence set forth in SEQ ID NO:14), when assayed under the same conditions.

Suitable 4-1BBL variants are described in published PCT Application WO 2019/051091, published 14 Mar. 2019 (Applicant Cue Biopharma, Inc.). See paragraphs [00229]-[00324], the disclosure of which is expressly incorporated herein by reference.

### IL-2 Variants

In some cases, a variant immunomodulatory polypeptide present in a TMP of the present disclosure is a variant IL-2 polypeptide. Wild-type IL-2 binds to IL-2 receptor (IL-2R), i.e., a heterotrimeric polypeptide comprising IL-2Ra, IL-2Rβ, and IL-2Rγ.

A wild-type IL-2 amino acid sequence can be as follows: APTSSSTKKT QLQLEHLLLD LQMILNGINN YKNPKLTRML TFKFYMPKKA TELKHLQCLEEELKPLEEVL NLAQSKNFHL RPRDLISNIN VIVLELKGSE TTFMCEYADE TATIVEFLNRWITFC-QSIIS TLT (SEQ ID NO:15).

Wild-type IL2 binds to an IL2 receptor (IL2R) on the surface of a cell. An IL2 receptor is in some cases a heterotrimeric polypeptide comprising an alpha chain (IL-2Rα; also referred to as CD25), a beta chain (IL-2Rβ; also referred to as CD122: and a gamma chain (IL-2Rγ; also referred to as CD132). Amino acid sequences of human IL-2Rα, IL2Rβ, and IL-2Rγ can be as follows.

Human IL-2Rα: ELCDDDPPE IPHATFKAMA YKEGTMLNCE CKRGFRRIKS GSLYMLCTGN SSHSSWDNQC QCTSSATRNT TKQVTPQPEE QKERKTTEMQ SPMQPVDQAS LPGHCREPPP WENEATERIY HFVVGQMVYY QCVQGYRALH RGPAESVCKM THGKTRWTQP QLICTGEMET SQFPGEEKPQ ASPEGRPESE TSCLVTTTDF QIQTEMAATM ETSIFTTEYQ VAVAGCVFLL ISVLLLSGLT WQRRQRKSRR TI (SEQ ID NO:16).

Human IL-2Rβ: VNG TSQFTCFYNS RANISCVWSQ DGALQDTSCQ VHAWPDRRRW NQTCELLPVS QASWACNLIL GAPDSQKLTT VDIVTLRVLC REGVRWRVMA IQDFKPFENL RLMAPISLQV VHVETHRCNI SWEISQASHY FERHLEFEAR TLSPGHTWEE APLLTLKQKQ EWICLETLTP DTQYEFQVRV KPLQGEFTTW SPWSQPLAFR TKPAALGKDT IPWLGHLLVG LSGAFGFIIL VYLLINCRNT GPWLKKVLKC NTPDPSKFFS QLSSEHGGDV QKWLSSPFPS SSFSPGGLAP EISPLEVLER DKVTQLLLQQ DKVPEPASLS SNHSLTSCFT NQGYFFFHLP DALEIEACQV YFTYDPYSEE DPDEGVAGAP TGSSPQPLQP LSGEDDAYCT FPSRDDLLLF SPSLLGGPSP PSTAPGGSGA GEERMPPSLQ ERVPRDWDPQ PLGPPTPGVP DLVDFQPPPE LVLREAGEEV PDAGPREGVS FPWSRPPGQG EFRALNARLP LNTDAYLSLQ ELQGQDPTHL V (SEQ ID NO:17).

Human IL-2Rγ: LNTTILTP NGNEDTTADF FLTTMPTDSL SVSTLPLPEV QCFVFNVEYM NCTWNSSSEP QPTNLTLHYW YKNSDNDKVQ KCSHYLFSEE ITSGCQLQKK EIHLYQTFVV QLQDPREPRR QATQMLKLQN LVIPWAPENL TLHKLSESQL ELNWNNRFLN HCLEHLVQYR TDWDHSWTEQ SVDYRHKFSL PSVDGQKRYT FRVRSRFNPL CGSAQHWSEW SHPIHWGSNT SKENPFLFAL EAVVISVGSM GLIISLLCVY FWLERTMPRI PTLKNLEDLV TEYHGNFSAW SGVSKGLAES LQPDYSERLC LVSEIPPKGG ALGEGPGASP CNQHSPYWAP PCYTLKPET (SEQ ID NO:18).

In some cases, where a TMP of the present disclosure comprises a variant IL-2 polypeptide, a “cognate co-immunomodulatory polypeptide” is an IL-2R comprising polypeptides comprising the amino acid sequences of SEQ ID NO:16, 17, and 18.

In some cases, a variant IL-2 polypeptide exhibits reduced binding affinity to IL-2R, compared to the binding affinity of a IL-2 polypeptide comprising the amino acid sequence set forth in SEQ ID NO:15. For example, in some cases, a variant IL-2 polypeptide binds IL-2R with a binding affinity that is at least 10% less, at least 15% less, at least 20% less, at least 25%, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of an IL-2 polypeptide comprising the amino acid sequence set forth in SEQ ID NO:15 for an IL-2R (e.g., an IL-2R comprising polypeptides comprising the amino acid sequence set forth in SEQ ID NOs:16-18), when assayed under the same conditions.

In some cases, an IL-2 variant MOD of this disclosure exhibits decreased binding to IL-2Rα, thereby minimizing or substantially reducing the activation of Tregs by the IL-2 variant. Alternatively, or additionally, in some cases, an IL-2 variant MOD of this disclosure exhibits decreased binding to IL-2Rβ and/or IL-2Rγ such that the IL-2 variant MOD exhibits an overall reduced affinity for IL-2R. In some cases, an IL-2 variant MOD of this disclosure exhibits both properties, i.e., it exhibits decreased or substantially no binding to IL-2Rα, and also exhibits decreased binding to IL-2Rβ and/or IL-2Rγ such that the IL-2 variant polypeptide exhibits an overall reduced affinity for IL-2R. For example, IL-2 variants having substitutions at H16 and F42 have shown decreased binding to IL-2Rα and IL-2Rβ. See, Quayle et al., Clin Cancer Res; 26(8) Apr. 15, 2020, which discloses that the binding affinity of an IL-2 polypeptide with H16A and F42A substitutions for human IL-2Rαand IL-2Rβ was decreased 110- and 3-fold, respectively, compared with wild-type IL2 binding, predominantly due to a faster off-rate for each of these interactions. TMPs comprising such variants, including variants that exhibit decreased binding to IL-2Rα and IL-2Rβ, have shown the ability to preferentially bind to and activate IL-2 receptors on T cells that contain the target TCR that is specific for the peptide epitope on the TMP, and are thus less likely to deliver IL-2 to non-target T cells, i.e., T cells that do not contain a TCR that specifically binds the peptide epitope on the TMP. That is, the binding of the IL-2 variant MOD to its costimulatory polypeptide on the T cell is substantially driven by the binding of the MHC-epitope moiety rather than by the binding of the IL-2.

Suitable IL-2 variants include a polypeptide that comprises an amino acid sequence having at least 90%, at least 95%, at least 98%, at least 99%, or 100%, amino acid sequence identity to any one of the following amino acid sequences:
APTSSSTKKT QLQLEHLLLD LQMILNGINN YKNPKLTRML TXKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:330), where X is any amino acid other than Phe. In some cases, X is Ala. In some cases, X is Met. In some cases, X is Pro. In some cases, X is Ser. In some cases, X is Thr. In some cases, X is Trp. In some cases, X is Tyr. In some cases, X is Val. In some cases, X is His;
APTSSSTKKT QLQLEHLLLX LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:331), where X is any amino acid other than Asp. In some cases, X is Ala;

APTSSSTKKT QLQLXHLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:332), where X is any amino acid other than Glu. In some cases, X is Ala.
APTSSSTKKT QLQLEXLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:333), where X is any amino acid other than His. In some cases, X is Ala. In some cases, X is Thr. In some cases, X is Asn. In some cases, X is Cys. In some cases, X is Gln. In some cases, X is Met. In some cases, X is Val. In some cases, X is Trp;
APTSSSTKKT QLQLEXLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:334), where X is any amino acid other than His. In some cases, X is Ala. In some cases, X is Arg. In some cases, X is Asn. In some cases, X is Asp. In some cases, X is Cys. In some cases, X is Glu. In some cases, X is Gln. In some cases, X is Gly. In some cases, X is Ile. I n some cases, X is Lys. In some cases, X is Leu. In some cases, X is Met. In some cases, X is Phe. In some cases, X is Pro. In some cases, X is Ser. In some cases, X is Thr. In some cases, X is Tyr. In some cases, X is Trp. In some cases, X is Val;
APTSSSTKKT QLQLEHLLLD LQMILNGINN YKNPKLTRML TFKFXMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:335), where X is any amino acid other than Tyr. In some cases, X is Ala;
APTSSSTKKT QLQLEHLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCXSIIS TLT (SEQ ID NO:336), where X is any amino acid other than Gln. In some cases, X is Ala;
APTSSSTKKT QLQLEX₁LLLD LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:337), where X₁ is any amino acid other than His, and where X₂ is any amino acid other than Phe. In some cases, X₁ is Ala. In some cases, X₂ is Ala. In some cases, X₁ is Ala; and X₂ is Ala. In some cases, X₁ is Thr; and X₂ is Ala;
APTSSSTKKT QLQLEHLLLX₁ LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:338), where X₁ is any amino acid other than Asp; and where X₂ is any amino acid other than Phe. In some cases, X₁ is Ala. In some cases, X₂ is Ala. In some cases, X₁ is Ala; and X₂ is Ala;
APTSSSTKKT QLQLX₁HLLLX₂ LQMILNGINN YKNPKLTRML TX₃KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:339), where X₁ is any amino acid other than Glu; where X₂ is any amino acid other than Asp; and where X₃ is any amino acid other than Phe. In some cases, X₁ is Ala. In some cases, X₂ is Ala. In some cases, X₃ is Ala. In some cases, X₁ is Ala; X₂ is Ala; and X₃ is Ala;
APTSSSTKKT QLQLEX₁LLLX₂ LQMILNGINN YKNPKLTRML TX₃KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:340), where X₁ is any amino acid other than His; where X₂ is any amino acid other than Asp; and where X₃ is any amino acid other than Phe. In some cases, X₁ is Ala. In some cases, X₂ is Ala. In some cases, X₃ is Ala. In some cases, X₁ is Ala; X₂ is Ala; and X₃ is Ala;
APTSSSTKKT QLQLEHLLLX₁ LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCX3SIIS TLT (SEQ ID NO:341), where X₁ is any amino acid other than Asp; where X₂ is any amino acid other than Phe; and where X₃ is any amino acid other than Gln. In some cases, X₁ is Ala. In some cases, X₂ is Ala. In some cases, X₃ is Ala. In some cases, X₁ is Ala; X₂ is Ala; and X₃ is Ala;
APTSSSTKKT QLQLEHLLLX₁ LQMILNGINN YKNPKLTRML TX₂KFX₃MPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:342), where X₁ is any amino acid other than Asp; where X₂ is any amino acid other than Phe; and where X₃ is any amino acid other than Tyr. In some cases, X₁ is Ala. In some cases, X₂ is Ala. In some cases, X₃ is Ala. In some cases, X₁ is Ala; X₂ is Ala; and X₃ is Ala;
APTSSSTKKT QLQLEX₁LLLX₂ LQMILNGINN YKNPKLTRML TX₃KFX₄MPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:343), where X₁ is any amino acid other than His; where X₂ is any amino acid other than Asp; where X₃ is any amino acid other than Phe; and where X₄ is any amino acid other than Tyr. In some cases, X₁ is Ala. In some cases, X₂ is Ala. In some cases, X₃ is Ala. In some cases, X₄ is Ala. In some cases, X₁ is Ala; X₂ is Ala; X₃ is Ala; and X₄ is Ala;
APTSSSTKKT QLQLEHLLLX₁ LQMILNGINN YKNPKLTRML TX₂KFX₃MPKKA TELKHLQCLE EELKPLEEVL NLAQSKNFHL RPRDLISNIN VIVLELKGSE TTFMCEYADE TATIVEFLNR WITFCX₄SIIS TLT (SEQ ID NO:344), where X₁ is any amino acid other than Asp; where X₂ is any amino acid other than Phe; where X₃ is any amino acid other than Tyr; and where X₄ is any amino acid other than Gln. In some cases, X₁ is Ala. In some cases, X₂ is Ala. In some cases, X₃ is Ala. In some cases, X₄ is Ala. In some cases, X₁ is Ala; X₂ is Ala; X₃ is Ala; and X₄ is Ala;
APTSSSTKKT QLQLEX₁LLLX₂ LQMILNGINN YKNPKLTRML TX₃KFX₄MPKKA TELKHLQCLE EELKPLEEVL NLAQSKNFHL RPRDLISNIN VIVLELKGSE TTFMCEYADE TATIVEFLNR WITFCX₅SIIS TLT (SEQ ID NO:345), where X₁ is any amino acid other than His; where X₂ is any amino acid other than Asp; where X₃ is any amino acid other than Phe; where X₄ is any amino acid other than Tyr; and where X₅ is any amino acid other than Gln. In some cases, X₁ is Ala. In some cases, X₂ is Ala. In some cases, X₃ is Ala. In some cases, X₄ is Ala. In some cases, X₅ is Ala. In some cases, X₁ is Ala; X₂ is Ala; X₃ is Ala; X₄ is Ala; X₅ is Ala; and
APTSSSTKKT QLQLEX₁LLLD LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCX₃SIIS TLT (SEQ ID NO:346), where X₁ is any amino acid other than His; where X₂ is any amino acid other than Phe; and where X₃ is any amino acid other than Gln. In some cases, X₁ is Ala. In some cases, X₂ is Ala. In some cases, X₃ is Ala. In some cases, X₁ is Ala; X₂ is Ala; and X₃ is Ala.

### Scaffold Polypeptides

A TMP of the present disclosure can comprise an Fc polypeptide, or can comprise another suitable scaffold polypeptide.

Suitable scaffold polypeptides include antibody-based scaffold polypeptides and non-antibody-based scaffolds. Non-antibody-based scaffolds include, e.g., albumin, an XTEN (extended recombinant) polypeptide, transferrin, an Fc receptor polypeptide, an elastin-like polypeptide (see, e.g., Hassouneh et al. (2012) Methods Enzymol. 502:215; e.g., a polypeptide comprising a pentapeptide repeat unit of (Val-Pro-Gly-X-Gly; SEQ ID NO:59), where X is any amino acid other than proline), an albumin-binding polypeptide, a silk-like polypeptide (see, e.g., Valluzzi et al. (2002) Philos Trans R Soc Lond B Biol Sci. 357:165), a silk-elastin-like polypeptide (SELP; see, e.g., Megeed et al. (2002) Adv Drug Deliv Rev. 54:1075), and the like. Suitable XTEN polypeptides include, e.g., those disclosed in WO 2009/023270, WO 2010/091122, WO 2007/103515, US 2010/0189682, and US 2009/0092582; see also Schellenberger et al. (2009) Nat Biotechnol. 27:1186). Suitable albumin polypeptides include, e.g., human serum albumin.

Suitable scaffold polypeptides will in some cases be a half-life extending polypeptides. Thus, in some cases, a suitable scaffold polypeptide increases the in vivo half-life (e.g., the serum half-life) of the TMP, compared to a control TMP lacking the scaffold polypeptide. For example, in some cases, a scaffold polypeptide increases the in vivo half-life (e.g., the serum half-life) of the TMP, compared to a control TMP lacking the scaffold polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold. As an example, in some cases, an Fc polypeptide increases the in vivo half-life (e.g., the serum half-life) of the TMP, compared to a control TMP lacking the Fc polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold.

### Fc Polypeptides

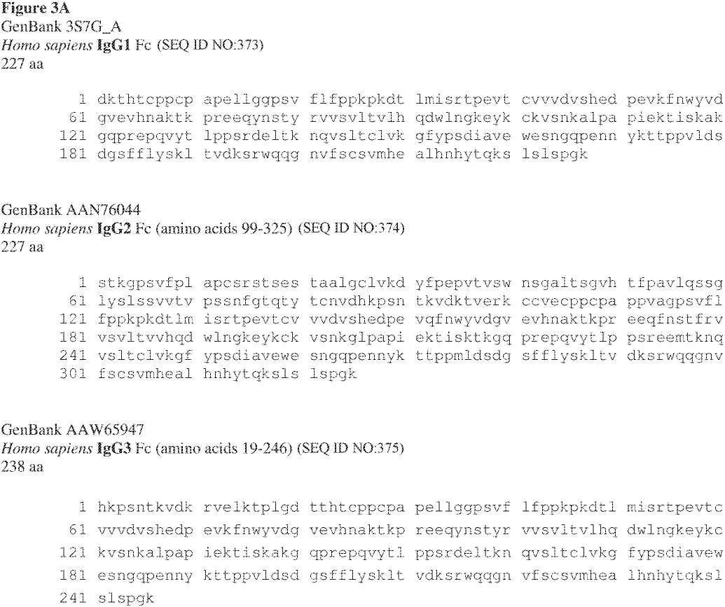

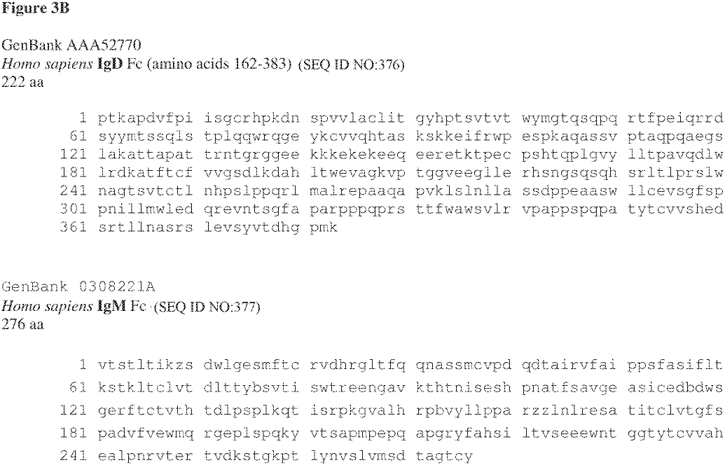

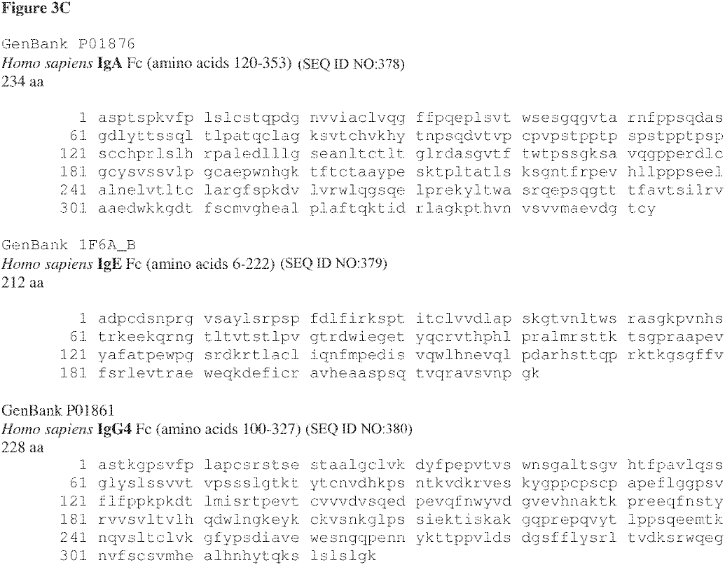

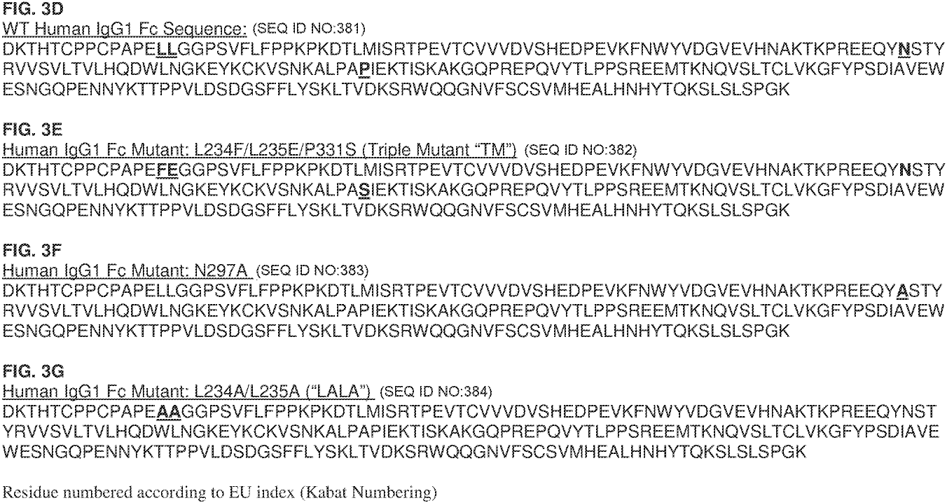

In some cases, the first and/or the second polypeptide chain of a TMP of the present disclosure comprises an Fc polypeptide. The Fc polypeptide of a TMP of the present disclosure can be a human IgG1 Fc, a human IgG2 Fc, a human IgG3 Fc, a human IgG4 Fc, etc. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to an amino acid sequence of an Fc region depicted in FIGS. 3A-3G. In some cases, the Fc region comprises an amino acid sequence having at least about 70%, at least about 75%, at least about 80%, at least about 85%, at least about 90%, at least about 95%, at least about 98%, at least about 99%, or 100%, amino acid sequence identity to the human IgG1 Fc polypeptide depicted in FIG. 3A. In some cases, the Fc region comprises an amino acid sequence having at least about 70%, at least about 75%, at least about 80%, at least about 85%, at least about 90%, at least about 95%, at least about 98%, at least about 99%, or 100%, amino acid sequence identity to the human IgG1 Fc polypeptide depicted in FIG. 3A; and comprises a substitution of N77; e.g., the Fc polypeptide comprises an N77A substitution. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG2 Fc polypeptide depicted in FIG. 3A; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 99-325 of the human IgG2 Fc polypeptide depicted in FIG. 3A. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG3 Fc polypeptide depicted in FIG. 3A; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 19-246 of the human IgG3 Fc polypeptide depicted in FIG. 3A. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgM Fc polypeptide depicted in FIG. 3B; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 1-276 to the human IgM Fc polypeptide depicted in FIG. 3B. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgA Fc polypeptide depicted in FIG. 3C; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 1-234 to the human IgA Fc polypeptide depicted in FIG. 3C.

In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG4 Fc polypeptide depicted in FIG. 3C. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 100 to 327 of the human IgG4 Fc polypeptide depicted in FIG. 3C.

In some cases, the IgG4 Fc polypeptide comprises the following amino acid sequence:

PPCPSCPAPEFLGGPSVFLFPPKPKDTLMISRTPEVTCVVVDVSQEDPEV
QFNWYVDGVEVHNAKTKPREEQFNSTYRVVSVLTVLHQDWLNGKEYKCKV
SNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVF
SCSVMHEALHNHYTQKSLSLSPG (SEQ ID NO:347).

In some cases, the Fc polypeptide present in a TMP comprises the amino acid sequence depicted in FIG. 3A (human IgG1 Fc). In some cases, the Fc polypeptide present in a TMP comprises the amino acid sequence depicted in FIG. 3A (human IgG1 Fc), except for a substitution of N297 (N77 of the amino acid sequence depicted in FIG. 3A) with an amino acid other than asparagine. In some cases, the Fc polypeptide present in a TMP comprises the amino acid sequence depicted in FIG. 3C (human IgG1 Fc comprising an N297A substitution, which is N77 of the amino acid sequence depicted in FIG. 3A). In some cases, the Fc polypeptide present in a TMP comprises the amino acid sequence depicted in FIG. 3A (human IgG1 Fc), except for a substitution of L234 (L14 of the amino acid sequence depicted in FIG. 3A) with an amino acid other than leucine. In some cases, the Fc polypeptide present in a TMP comprises the amino acid sequence depicted in FIG. 3A (human IgG1 Fc), except for a substitution of L235 (L15 of the amino acid sequence depicted in FIG. 3A) with an amino acid other than leucine.

The Ig Fc can be a variant of an Ig Fc polypeptide (e.g., a variant of human IgG1 Fc polypeptide) that has a substantially reduced ability to effect complement-dependent cytotoxicity (CDC) or antibody-dependent cell cytotoxicity (ADCC). See, e.g., the Ig Fc polypeptides described below comprising L234A and L235A substitutions.

In some cases, the Fc polypeptide present in a TMP comprises the amino acid sequence depicted in FIG. 3E. In some cases, the Fc polypeptide present in a TMP comprises the amino acid sequence depicted in FIG. 3F. In some cases, the Fc polypeptide present in a TMP comprises the amino acid sequence depicted in FIG. 5G (human IgG1 Fc comprising an L234A substitution and an L235A substitution, corresponding to positions 14 and 15 of the amino acid sequence depicted in FIG. 3G). In some cases, the Fc polypeptide present in a TMP comprises the amino acid sequence depicted in FIG. 3A (human IgG1 Fc), except for a substitution of P331 (P111 of the amino acid sequence depicted in FIG. 3A) with an amino acid other than proline; in some cases, the substitution is a P331S substitution. In some cases, the Fc polypeptide present in a TMP comprises the amino acid sequence depicted in FIG. 3A (human IgG1 Fc), except for substitutions at L234 and L235 (L14 and L15 of the amino acid sequence depicted in FIG. 3A) with amino acids other than leucine. In some cases, the Fc polypeptide present in a TMP comprises the amino acid sequence depicted in FIG. 3A (human IgG1 Fc), except for substitutions at L234 and L235 (L14 and L15 of the amino acid sequence depicted in FIG. 3A) with amino acids other than leucine, and a substitution of P331 (P111 of the amino acid sequence depicted in FIG. 3A) with an amino acid other than proline. In some cases, the Fc polypeptide present in a TMP comprises the amino acid sequence depicted in FIG. 3E (human IgG1 Fc comprising L234F, L235E, and P331S substitutions (corresponding to amino acid positions 14, 15, and 111 of the amino acid sequence depicted in FIG. 3E). In some cases, the Fc polypeptide present in a TMP is an IgG1 Fc polypeptide that comprises L234A and L235A substitutions (substitutions of L14 and L15 of the amino acid sequence depicted in FIG. 3A with Ala), as depicted in FIG. 3G.

### Linkers

A TMP of the present disclosure can include one or more linkers, where the one or more linkers are between one or more of: i) an MHC Class I polypeptide and an Ig Fc polypeptide, where such a linker is referred to herein as “L1”; ii) an immunomodulatory polypeptide and an MHC Class I polypeptide, where such a linker is referred to herein as “L2”; iii) a first immunomodulatory polypeptide and a second immunomodulatory polypeptide, where such a linker is referred to herein as “L3”; iv) a peptide antigen (“epitope”) and an MHC Class I polypeptide; v) an MHC Class I polypeptide and a dimerization polypeptide (e.g., a first or a second member of a dimerizing pair); and vi) a dimerization polypeptide (e.g., a first or a second member of a dimerizing pair) and an IgFc polypeptide.

Suitable linkers (also referred to as “spacers”) can be readily selected and can be of any of a number of suitable lengths, such as from 1 amino acid to 25 amino acids, from 3 amino acids to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids. A suitable linker can be 1, 2, 3, 4, 5, 6, 7, 8, 9, 10, 11, 12, 13, 14, 15, 16, 17, 18, 19, 20, 21, 22, 23, 24, or 25 amino acids in length. In some cases, a linker has a length of from 25 amino acids to 50 amino acids, e.g., from 25 to 30, from 30 to 35, from 35 to 40, from 40 to 45, or from 45 to 50 amino acids in length.

Exemplary linkers include glycine polymers (G)ₙ, glycine-serine polymers (including, for example, (GS)ₙ, (GSGGS)ₙ (SEQ ID NO:348), and (GGGS)ₙ (SEQ ID NO:349), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, Rev. Computational Chem. 11173-142 (1992)). Exemplary linkers can comprise amino acid sequences including, but not limited to, GGSG (SEQ ID NO:350), GGSGG (SEQ ID NO:351), GSGSG (SEQ ID NO:352), GSGGG (SEQ ID NO:353), GGGSG (SEQ ID NO:354), GSSSG (SEQ ID NO:355), and the like. Exemplary linkers can include, e.g., Gly(Ser₄)n (SEQ ID NO:356), where n is 1, 2, 3, 4, 5, 6, 7, 8, 9, or 10. In some cases, a linker comprises the amino acid sequence (GSSSS)n (SEQ ID NO:356), where n is 4. In some cases, a linker comprises the amino acid sequence (GSSSS)n (SEQ ID NO:356), where n is 5.

Exemplary linkers are (GGGGS)n (SEQ ID NO:357) linkers, where n is 1, 2, 3, 4, 5, 6, 7, 8, 9, or 10. In some cases, a linker comprises the amino acid sequence (GGGGS)n (SEQ ID NO:357), where n is 1; in some cases (GGGGS)n (SEQ ID NO:357), where n is 2; in some cases, (GGGGS)n (SEQ ID NO:357), where n is 3; in some cases (GGGGS)n (SEQ ID NO:357), where n is 4; in some cases (GGGGS)n (SEQ ID NO:357), where n is 5; in some cases (GGGGS)n (SEQ ID NO:357), where n is 6; in some cases (GGGGS)n (SEQ ID NO:357), where n is 7; in some cases (GGGGS)n (SEQ ID NO:357), where n is 8; in some cases (GGGGS)n (SEQ ID NO:357), where n is 9; in some case (GGGGS)n (SEQ ID NO:357), where n is 10. In some cases, a linker comprises the amino acid sequence AAAGG (SEQ ID NO:358).

In some cases, a linker polypeptide, present in a first polypeptide of a TMP of the present disclosure, includes a cysteine residue that can form a disulfide bond with a cysteine residue present in a second polypeptide of a TMP of the present disclosure. In some cases, for example, a suitable linker comprises the amino acid sequence GCGGSGGGGSGGGGS (SEQ ID NO:173). As another example, a suitable linker can comprise the amino acid sequence GCGGS(GGGGS)n (SEQ ID NO:171), where n is 1, 2, 3, 4, 5, 6, 7, 8, or 9. For example, in some cases, the linker comprises the amino acid sequence GCGGSGGGGSGGGGSGGGGS (SEQ ID NO:172). As another example, the linker comprises the amino acid sequence GCGGSGGGGSGGGGS (SEQ ID NO:173).

### Dimerized TMPs

A TMP of the present disclosure can be dimerized; i.e., the present disclosure provides a multimeric polypeptide comprising a dimer of a TMP of the present disclosure. Thus, the present disclosure provides a protein (a dimerized TMP) comprising: A) a first heterodimer comprising: a) a first polypeptide comprising: i) a SARS-CoV-2 peptide; and ii) a first MHC polypeptide; and b) a second polypeptide comprising: i) a second MHC polypeptide, wherein the first heterodimer comprises one or more MODs; and B) a second heterodimer comprising: a) a first polypeptide comprising: i) a SARS-CoV-2 peptide; and ii) a first MHC polypeptide; and b) a second polypeptide comprising: i) a second MHC polypeptide, wherein the second heterodimer comprises one or more MODs, and wherein the first heterodimer and the second heterodimer are covalently linked to one another. The covalent linkage can be a disulfide bond between an Ig Fc polypeptide in the first heterodimer and an Ig Fc polypeptide in the second heterodimer; thus, e.g., the Ig Fc polypeptides in the first heterodimer and the second heterodimer can be linked to one another by one or more disulfide bonds. In some cases, the two TMPs are identical to one another in amino acid sequence. In some cases, the first heterodimer and the second heterodimer are covalently linked to one another via a C-terminal region of the second polypeptide of the first heterodimer and a C-terminal region of the second polypeptide of the second heterodimer. In some cases, first heterodimer and the second heterodimer are covalently linked to one another via the C-terminal amino acid of the second polypeptide of the first heterodimer and the C-terminal region of the second polypeptide of the second heterodimer; for example, in some cases, the C-terminal amino acid of the second polypeptide of the first heterodimer and the C-terminal region of the second polypeptide of the second heterodimer are linked to one another, either directly or via a linker. The linker can be a peptide linker. The peptide linker can have a length of from 1 amino acid to 200 amino acids (e.g., from 1 amino acid (aa) to 5 aa, from 5 aa to 10 aa, from 10 aa to 25 aa, from 25 aa to 50 aa, from 50 aa to 100 aa, from 100 aa to 150 aa, or from 150 aa to 200 aa). In some cases, the peptide epitope of the first heterodimer and the peptide epitope of the second heterodimer comprise the same amino acid sequence. In some cases, the first MHC polypeptide of the first and the second heterodimer is an MHC Class Iβ2-microglobulin, and wherein the second MHC polypeptide of the first and the second heterodimer is an MHC Class I heavy chain. In some cases, the MOD of the first heterodimer and the MOD of the second heterodimer comprise the same amino acid sequence. In some cases, the MOD of the first heterodimer and the MOD of the second heterodimer are variant MODs that comprise from 1 to 10 amino acid substitutions compared to a corresponding parental wild-type MOD, and wherein the from 1 to 10 amino acid substitutions result in reduced affinity binding of the variant MOD to a cognate co-immunomodulatory polypeptide. In some cases, the MOD of the first heterodimer and the MOD of the second heterodimer are each independently selected from the group consisting of IL-2, 4-1BBL, PD-L1, CD80, CD86, ICOS-L, OX-40L, FasL, JAG1 (CD339), TGFβ, CD70, and ICAM. Examples, of suitable MHC polypeptides, immunomodulatory polypeptides, and peptide epitopes are described herein.

In some cases, a single-chain TMP of the present disclosure is dimerized. Thus, the present disclosure provides a protein comprising: a) a first single-chain TMP of the present disclosure; and b) a second single-chain TMP of the present disclosure, where the first and second single-chain TMPs are covalently linked to one another. The covalent linkage can be a disulfide bond between an Ig Fc polypeptide in the first single-chain TMP and an Ig Fc polypeptide in the second single-chain TMP.

### Additional Polypeptides

A polypeptide chain of a TMP of the present disclosure can include one or more polypeptides in addition to those described above. Suitable additional polypeptides include epitope tags and affinity domains. The one or more additional polypeptide can be included at the N-terminus of a polypeptide chain of a TMP, at the C-terminus of a polypeptide chain of a TMP, or internally within a polypeptide chain of a TMP.

### Epitope Tag

Suitable epitope tags include, but are not limited to, hemagglutinin (HA; e.g., YPYDVPDYA (SEQ ID NO:359); FLAG (e.g., DYKDDDDK (SEQ ID NO:360); c-myc (e.g., EQKLISEEDL; SEQ ID NO:361), and the like.

### Affinity Domain

Affinity domains include peptide sequences that can interact with a binding partner, e.g., such as one immobilized on a solid support, useful for identification or purification. DNA sequences encoding multiple consecutive single amino acids, such as histidine, when fused to the expressed protein, may be used for one-step purification of the recombinant protein by high affinity binding to a resin column, such as nickel sepharose. Exemplary affinity domains include His5 (HHHHH) (SEQ ID NO:362), HisX6 (HHHHHH) (SEQ ID NO:363), C-myc (EQKLISEEDL) (SEQ ID NO:361), Flag (DYKDDDDK) (SEQ ID NO:360), StrepTag (WSHPQFEK) (SEQ ID NO:364), hemagglutinin, e.g., HA Tag (YPYDVPDYA) (SEQ ID NO:359), glutathione-S-transferase (GST), thioredoxin, cellulose binding domain, RYIRS (SEQ ID NO:365), Phe-His-His-Thr (SEQ ID NO:366), chitin binding domain, S-peptide, T7 peptide, SH2 domain, C-end RNA tag, WEAAAREACCRECCARA (SEQ ID NO:367), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, S100 proteins, parvalbumin, calbindin D9K, calbindin D28K, and calretinin, inteins, biotin, streptavidin, MyoD, Id, leucine zipper sequences, and maltose binding protein.

### Drug Conjugates

A polypeptide chain of a TMP of the present disclosure can comprise a small molecule drug linked (e.g., covalently attached) to the polypeptide chain. For example, where a TMP comprises an Fc polypeptide, the Fc polypeptide can comprise a covalently linked small molecule drug. A polypeptide chain of a TMP can comprise a cytotoxic agent linked (e.g., covalently attached) to the polypeptide chain. For example, where a TMP comprises an Fc polypeptide, the Fc polypeptide can comprise a covalently linked cytotoxic agent. Cytotoxic agents include prodrugs.

### Nucleic Acids

The present disclosure provides a nucleic acid comprising a nucleotide sequence encoding a TMP of the present disclosure. In some cases, the nucleic acid comprises a nucleotide sequence encoding a single-chain TMP.

The present disclosure provides nucleic acids comprising nucleotide sequences encoding a TMP of the present disclosure. In some cases, the individual polypeptide chains of a TMP (e.g., a “split-chain” TMP) of the present disclosure are encoded in separate nucleic acids. In some cases, all polypeptide chains of a TMP of the present disclosure are encoded in a single nucleic acid. In some cases, a first nucleic acid comprises a nucleotide sequence encoding a first polypeptide of a TMP of the present disclosure; and a second nucleic acid comprises a nucleotide sequence encoding a second polypeptide of a TMP of the present disclosure. In some cases, single nucleic acid comprises a nucleotide sequence encoding a first polypeptide of a TMP of the present disclosure and a second polypeptide of a TMP of the present disclosure.

### Separate Nucleic Acids Encoding Individual Polypeptide Chains of a Multimeric Polypeptide

The present disclosure provides nucleic acids comprising nucleotide sequences encoding a TMP of the present disclosure. As noted above, in some cases, the individual polypeptide chains of a TMP are encoded in separate nucleic acids. In some cases, nucleotide sequences encoding the separate polypeptide chains of a TMP are operably linked to transcriptional control elements, e.g., promoters, such as promoters that are functional in a eukaryotic cell, where the promoter can be a constitutive promoter or an inducible promoter.

The present disclosure provides a first nucleic acid and a second nucleic acid, where the first nucleic acid comprises a nucleotide sequence encoding a first polypeptide of a TMP, where the first polypeptide comprises, in order from N-terminus to C-terminus: a) an epitope (e.g., a T-cell epitope); b) a first MHC polypeptide; and c) a MOD (e.g., a reduced-affinity variant, as described above); and where the second nucleic acid comprises a nucleotide sequence encoding a second polypeptide of a TMP of the present disclosure, where the second polypeptide comprises, in order from N-terminus to C-terminus: a) a second MHC polypeptide; and b) an Ig Fc polypeptide. Suitable T-cell epitopes, MHC polypeptides, immunomodulatory polypeptides, and Ig Fc polypeptides, are described above. In some cases, the nucleotide sequences encoding the first and the second polypeptides are operably linked to transcriptional control elements. In some cases, the transcriptional control element is a promoter that is functional in a eukaryotic cell. In some cases, the nucleic acids are present in separate expression vectors.

The present disclosure provides a first nucleic acid and a second nucleic acid, where the first nucleic acid comprises a nucleotide sequence encoding a first polypeptide of a TMP, where the first polypeptide comprises, in order from N-terminus to C-terminus: a) an epitope (e.g., a T-cell epitope); and b) a first MHC polypeptide; and where the second nucleic acid comprises a nucleotide sequence encoding a second polypeptide of a TMP of the present disclosure, where the second polypeptide comprises, in order from N-terminus to C-terminus: a) a MOD (e.g., a reduced-affinity variant as described above); b) a second MHC polypeptide; and c) an Ig Fc polypeptide. Suitable T-cell epitopes, MHC polypeptides, MODs, and Ig Fc polypeptides, are described above. In some cases, the nucleotide sequences encoding the first and the second polypeptides are operably linked to transcriptional control elements. In some cases, the transcriptional control element is a promoter that is functional in a eukaryotic cell. In some cases, the nucleic acids are present in separate expression vectors.

### Nucleic Acid Encoding Two or More Polypeptides Present in a Multimeric Polypeptide

The present disclosure provides a nucleic acid comprising nucleotide sequences encoding at least the first polypeptide and the second polypeptide of a TMP of the present disclosure. In some cases, where a TMP includes a first, second, and third polypeptide, the nucleic acid includes a nucleotide sequence encoding the first, second, and third polypeptides. In some cases, the nucleotide sequences encoding the first polypeptide and the second polypeptide of a TMP includes a proteolytically cleavable linker interposed between the nucleotide sequence encoding the first polypeptide and the nucleotide sequence encoding the second polypeptide. In some cases, the nucleotide sequences encoding the first polypeptide and the second polypeptide of a TMP includes an internal ribosome entry site (IRES) interposed between the nucleotide sequence encoding the first polypeptide and the nucleotide sequence encoding the second polypeptide. In some cases, the nucleotide sequences encoding the first polypeptide and the second polypeptide of a TMP includes a ribosome skipping signal (or cis-acting hydrolase element, CHYSEL) interposed between the nucleotide sequence encoding the first polypeptide and the nucleotide sequence encoding the second polypeptide. Examples of nucleic acids are described below, where a proteolytically cleavable linker is provided between nucleotide sequences encoding the first polypeptide and the second polypeptide of a TMP; in any of these embodiments, an IRES or a ribosome skipping signal can be used in place of the nucleotide sequence encoding the proteolytically cleavable linker.

In some cases, a first nucleic acid (e.g., a recombinant expression vector, an mRNA, a viral RNA, etc.) comprises a nucleotide sequence encoding a first polypeptide chain of a TMP of the present disclosure; and a second nucleic acid (e.g., a recombinant expression vector, an mRNA, a viral RNA, etc.) comprises a nucleotide sequence encoding a second polypeptide chain of a TMP. In some cases, the nucleotide sequence encoding the first polypeptide, and the second nucleotide sequence encoding the second polypeptide, are each operably linked to transcriptional control elements, e.g., promoters, such as promoters that are functional in a eukaryotic cell, where the promoter can be a constitutive promoter or an inducible promoter.

The present disclosure provides a nucleic acid comprising a nucleotide sequence encoding a recombinant polypeptide, where the recombinant polypeptide comprises, in order from N-terminus to C-terminus: a) an epitope (e.g., a T-cell epitope); b) a first MHC polypeptide; c) a MOD (e.g., a reduced-affinity variant as described above); d) a proteolytically cleavable linker; e) a second MHC polypeptide; and f) Ig Fc polypeptide. The present disclosure provides a nucleic acid comprising a nucleotide sequence encoding a recombinant polypeptide, where the recombinant polypeptide comprises, in order from N-terminus to C-terminus: a) a first leader peptide; b) the epitope; c) the first MHC polypeptide; d) the MOD (e.g., a reduced-affinity variant as described above); e) the proteolytically cleavable linker; f) a second leader peptide; g) the second MHC polypeptide; and h) the Ig Fc polypeptide. The present disclosure provides a nucleic acid comprising a nucleotide sequence encoding a recombinant polypeptide, where the recombinant polypeptide comprises, in order from N-terminus to C-terminus: a) an epitope; b) a first MHC polypeptide; c) a proteolytically cleavable linker; d) a MOD (e.g., a reduced-affinity variant as described above); e) a second MHC polypeptide; and f) an Ig Fc polypeptide. In some cases, the first leader peptide and the second leader peptide are a β2-M leader peptide. In some cases, the nucleotide sequence is operably linked to a transcriptional control element. In some cases, the transcriptional control element is a promoter that is functional in a eukaryotic cell.

Suitable MHC polypeptides are described above. In some cases, the first MHC polypeptide is a β2-microglobulin polypeptide; and wherein the second MHC polypeptide is an MHC class I heavy chain polypeptide. In some cases, the β2-microglobulin polypeptide comprises an amino acid sequence having at least 85% amino acid sequence identity to a β2M amino acid sequence depicted in FIG. 4. In some cases, the MHC class I heavy chain polypeptide is an HLA-A, HLA-B, HLA-C, HLA-E, HLA-F, HLA-G, HLA-K, or HLA-L heavy chain.

Suitable Fc polypeptides are described above. In some cases, the Ig Fc polypeptide is an IgG1 Fc polypeptide, an IgG2 Fc polypeptide, an IgG3 Fc polypeptide, an IgG4 Fc polypeptide, an IgA Fc polypeptide, or an IgM Fc polypeptide. In some cases, the Ig Fc polypeptide comprises an amino acid sequence having at least 85% amino acid sequence identity to an amino acid sequence depicted in FIGS. 3A-3G.

Suitable MODs are described above.

Suitable proteolytically cleavable linkers are described above. In some cases, the proteolytically cleavable linker comprises an amino acid sequence selected from: a) LEVLFQGP (SEQ ID NO:368); b) ENLYTQS (SEQ ID NO:369); c) DDDDK (SEQ ID NO:370); d) LVPR (SEQ ID NO:371); and e) GSGATNFSLLKQAGDVEENPGP (SEQ ID NO:372).

In some cases, a linker between the epitope and the first MHC polypeptide comprises a first Cys residue, and the second MHC polypeptide comprises an amino acid substitution to provide a second Cys residue, such that the first and the second Cys residues provide for a disulfide linkage between the linker and the second MHC polypeptide. In some cases, first MHC polypeptide comprises an amino acid substitution to provide a first Cys residue, and the second MHC polypeptide comprises an amino acid substitution to provide a second Cys residue, such that the first Cys residue and the second Cys residue provide for a disulfide linkage between the first MHC polypeptide and the second MHC polypeptide.

### Recombinant Expression Vectors

The present disclosure provides recombinant expression vectors comprising nucleic acids of the present disclosure. In some cases, the recombinant expression vector is a non-viral vector. In some cases, the recombinant expression vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, a non-integrating viral vector, etc.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

Numerous suitable expression vectors are known to those of skill in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other vector may be used so long as it is compatible with the host cell.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

In some cases, a nucleotide sequence encoding a DNA-targeting RNA and/or a site-directed modifying polypeptide is operably linked to a control element, e.g., a transcriptional control element, such as a promoter. The transcriptional control element may be functional in either a eukaryotic cell, e.g., a mammalian cell; or a prokaryotic cell (e.g., bacterial or archaeal cell). In some cases, a nucleotide sequence encoding a DNA-targeting RNA and/or a site-directed modifying polypeptide is operably linked to multiple control elements that allow expression of the nucleotide sequence encoding a DNA-targeting RNA and/or a site-directed modifying polypeptide in both prokaryotic and eukaryotic cells.

Non-limiting examples of suitable eukaryotic promoters (promoters functional in a eukaryotic cell) include those from cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

### Genetically Modified Host Cells

The present disclosure provides a genetically modified host cell, where the host cell is genetically modified with a nucleic acid of the present disclosure.

Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

In some cases, the host cell is a mammalian cell that has been genetically modified such that it does not synthesize endogenous MHC β2-M.

In some cases, the host cell is a mammalian cell that has been genetically modified such that it does not synthesize endogenous MHC Class I heavy chain. In some cases, the host cell is a mammalian cell that has been genetically modified such that it does not synthesize endogenous MHC β2-M and such that it does not synthesize endogenous MHC Class I heavy chain.

### Compositions

The present disclosure provides compositions, including pharmaceutical compositions, comprising a TMP (synTac) of the present disclosure. The present disclosure provides compositions, including pharmaceutical compositions, comprising a nucleic acid or a recombinant expression vector of the present disclosure.

### Compositions Comprising a TMP

A composition of the present disclosure can comprise, in addition to a TMP of the present disclosure, one or more additional components that provide desirable properties such as stability, solubility, etc., e.g., salts, solubilizing agents; surfactants, protease inhibitors, etc., a variety of which are known in the art and need not be discussed in detail herein. Pharmaceutically acceptable excipients for biologics have been amply described in a variety of patents and other publications, including, for example, “Remington: The Science and Practice of Pharmacy”, 19^{th} Ed. (1995), or latest edition, Mack Publishing Co; A. Gennaro (2000) “Remington: The Science and Practice of Pharmacy”, 20th edition, Lippincott, Williams, & Wilkins; Pharmaceutical Dosage Forms and Drug Delivery Systems (1999) H.C. Ansel et al., eds 7^{th} ed., Lippincott, Williams, & Wilkins; and Handbook of Pharmaceutical Excipients (2000) A.H. Kibbe et al., eds., 3^{rd} ed. Amer. Pharmaceutical Assoc.

A pharmaceutical composition can comprise a TMP, and a pharmaceutically acceptable excipient. In some cases, a subject pharmaceutical composition will be suitable for administration to a subject, e.g., will be sterile. For example, in some cases, a subject pharmaceutical composition will be suitable for administration to a human subject, e.g., where the composition is sterile and is free of detectable pyrogens and/or other toxins or such detectable pyrogens and/or other toxins are below permissible limits.

Where a TMP of the present disclosure is administered as an injectable (e.g. subcutaneously, intraperitoneally, intramuscularly, and/or intravenously) directly into a tissue, a formulation can be provided as a ready-to-use dosage form, or as non-aqueous form (e.g. a reconstitutable storage-stable powder) or aqueous form, such as liquid composed of pharmaceutically acceptable carriers and excipients. The protein-containing formulations may also be provided so as to enhance serum half-life of the TMP following administration. For example, the TMP may be provided in a liposome formulation, prepared as a colloid, or other conventional techniques for extending serum half-life. A variety of methods are available for preparing liposomes, as described in, e.g., Szoka et al. 1980 Ann. Rev. Biophys. Bioeng. 9:467, U.S. Pat. Nos. 4,235,871, 4,501,728 and 4,837,028. The preparations may also be provided in controlled release or slow-release forms.

The concentration of a TMP in a formulation can vary widely (e.g., from less than about 0.1%, usually at or at least about 2% to as much as 20% to 50% or more by weight) and will usually be selected primarily based on fluid volumes, viscosities, and patient-based factors in accordance with the particular mode of administration selected and the patient’s needs.

The present disclosure provides a container comprising a composition of the present disclosure, e.g., a liquid composition. The container can be, e.g., a syringe, an ampoule, and the like. In some cases, the container is sterile. In some cases, both the container and the composition are sterile.

The present disclosure provides compositions, including pharmaceutical compositions, comprising a TMP of the present disclosure. A composition can comprise: a) a TMP; and b) a pharmaceutically acceptable excipient.

In some cases, a TMP is present in a liquid composition. Thus, the present disclosure provides compositions (e.g., liquid compositions, including pharmaceutical compositions) comprising a TMP. In some cases, a composition of the present disclosure comprises: a) a TMP; and b) saline (e.g., 0.9% NaCl). In some cases, the composition is sterile. Thus, the present disclosure provides a composition comprising: a) a TMP of the present disclosure; and b) saline (e.g., 0.9% NaCl), where the composition is sterile and is free of detectable pyrogens and/or other toxins and/or such detectable pyrogens and/or other toxins are below permissible limits.

### Compositions Comprising a Nucleic Acid or a Recombinant Expression Vector

The present disclosure provides compositions, e.g., pharmaceutical compositions, comprising a nucleic acid or a recombinant expression vector of the present disclosure. Pharmaceutically acceptable compositions suitable for delivering such nucleic acids and recombinant expression vectors are known in the art and are not discussed in detail herein.

### Methods of Modulating T Cell Activity

The present disclosure provides a method of selectively modulating the activity of an epitope-specific T cell (e.g., a T cell specific for a SARS-CoV-2 epitope, the method comprising contacting the T cell with a TMP of the present disclosure, where contacting the T cell with a TMP of the present disclosure selectively modulates the activity of the epitope-specific T cell. In some cases, the contacting occurs in vitro. In some cases, the contacting occurs in vivo. In some cases, the contacting occurs ex vivo.

In some cases, e.g., where the target T cell is a CD8⁺ T cell, the TMP comprises Class I MHC polypeptides (e.g., β2-microglobulin and Class I MHC heavy chain).

Where a TMP of the present disclosure includes a MOD that is an activating polypeptide, contacting the T cell with the TMP activates the epitope-specific T cell. In some instances, the epitope-specific T cell is a T cell that is specific for an epitope present on a virus-infected cell (e.g., a cell infected with SARS-CoV-2), and contacting the epitope-specific T cell with the TMP increases cytotoxic activity of the T cell toward the virus-infected cell. In some instances, the epitope-specific T cell is a T cell that is specific for an epitope present on a virus-infected cell (e.g., a cell infected with SARS-CoV-2), and contacting the epitope-specific T cell with the TMP increases the number of the epitope-specific T cells.

The present disclosure provides a method of modulating an immune response in an individual, the method comprising administering to the individual an effective amount of a TMP of the present disclosure. Administering the TMP induces an epitope-specific T cell response (e.g., a virus epitope-specific) and an epitope-non-specific T cell response, where the ratio of the epitope-specific T cell response to the epitope-non-specific T cell response is at least 2:1. In some cases, the ratio of the epitope-specific T cell response to the epitope-non-specific T cell response is at least 5:1. In some cases, the ratio of the epitope-specific T cell response to the epitope-non-specific T cell response is at least 10:1. In some cases, the ratio of the epitope-specific T cell response to the epitope-non-specific T cell response is at least 25:1. In some cases, the ratio of the epitope-specific T cell response to the epitope-non-specific T cell response is at least 50:1. In some cases, the ratio of the epitope-specific T cell response to the epitope-non-specific T cell response is at least 100:1. In some cases, the individual is a human. In some cases, the modulating increases a cytotoxic T-cell response to a virus-infected cell, e.g., a virus-infected cell expressing an antigen that displays the same epitope displayed by a SARS-CoV-2 peptide epitope present in the TMP. In some cases, the administering is intravenous, subcutaneous, intramuscular, systemic, intralymphatic, pulmonary, distal to a treatment site, local, or at or near a treatment site.

The present disclosure provides a method of delivering a costimulatory (i.e., immunomodulatory) polypeptide selectively to target T cell, the method comprising contacting a mixed population of T cells with a TMP of the present disclosure, where the mixed population of T cells comprises the target T cell and non-target T cells, where the target T cell is specific for the epitope present within the TMP (e.g., where the target T cell is specific for the epitope present within the TMP), and where the contacting step delivers the one or more costimulatory polypeptides (immunomodulatory polypeptides) present within the TMP to the target T cell. In some cases, the population of T cells is in vitro. In some cases, the population of T cells is in vivo in an individual. In some cases, the method comprises administering the TMP to the individual. In some case, the T cell is a cytotoxic T cell. In some cases, the mixed population of T cells is an in vitro population of mixed T cells obtained from an individual, and the contacting step results in activation and/or proliferation of the target T cell, generating a population of activated and/or proliferated target T cells; in some of these instances, the method further comprises administering the population of activated and/or proliferated target T cells to the individual.

The present disclosure provides a method of detecting, in a mixed population of T cells obtained from an individual, the presence of a target T cell that binds an epitope of interest (e.g., a SARS-CoV-2 epitope), the method comprising: a) contacting in vitro the mixed population of T cells with a TMP of the present disclosure, wherein the TMP comprises the epitope of interest (e.g., the virus epitope); and b) detecting activation and/or proliferation of T cells in response to said contacting, wherein activated and/or proliferated T cells indicates the presence of the target T cell.

### Treatment Methods

The present disclosure provides a method of treatment of an individual, the method comprising administering to the individual an amount of a TMP of the present disclosure, or one or more nucleic acids encoding the TMP, effective to treat the individual. Also provided is a TMP of the present disclosure for use in a method of treatment of the human or non-human animal body. In some cases, a treatment method of the present disclosure comprises administering to an individual in need thereof one or more recombinant expression vectors comprising nucleotide sequences encoding a TMP of the present disclosure. In some cases, a treatment method of the present disclosure comprises administering to an individual in need thereof one or more mRNA molecules comprising nucleotide sequences encoding a TMP of the present disclosure. In some cases, a treatment method of the present disclosure comprises administering to an individual in need thereof a TMP of the present disclosure. Conditions that can be treated include Betacoronavirus infections, such as SARS-CoV-2 infections.

In some cases, because the TMPs of this disclosure also are able to prime naïve T cells, the pharmaceutical compositions of this disclosure also may be used to treat persons who are not infected but at risk of infection, i.e., the pharmaceutical compositions can be injected to cause a human or non-human to prime and activate epitope specific T cells and/or develop memory T cells that will be therapeutically useful in the event of a SARS-CoV-2 infection. Accordingly, in some cases, an effective amount of a pharmaceutical composition comprising a TMP is an amount that, when administered in one or more doses to individuals in a population who do not have an infection (e.g., a SARS-CoV-2 infection) and are at risk of infection, and/or to individuals who are at greater risk of severe illness from infection (e.g., a SARS-CoV-2 infection) than the general population, causes a human or non-human to prime and activate epitope specific T cells and/or develop memory T cells that will be therapeutically useful in the event of a SARS-CoV-2 infection.

In some cases, a TMP of the present disclosure, when administered to an individual in need thereof, induces both an epitope-specific T cell response and an epitope non-specific T cell response. In other words, in some cases, a TMP of the present disclosure, when administered to an individual in need thereof, induces an epitope-specific T cell response by modulating the activity of a first T cell that displays both: i) a TCR specific for the epitope present in the TMP; ii) a co-MOD that binds to the MOD present in the TMP; and induces an epitope non-specific T cell response by modulating the activity of a second T cell that displays: i) a TCR specific for an epitope other than the epitope present in the TMP; and ii) a co-MOD that binds to the MOD present in the TMP. The ratio of the epitope-specific T cell response to the epitope-non-specific T cell response, when measured as the ratio of the increase of the number of epitope-specific T cells to the increase in the number of epitope non-specific T cells (as discussed above), is at least 2:1, at least 5:1, at least 10:1, at least 15:1, at least 20:1, at least 25:1, at least 50:1, or at least 100:1. The ratio of the epitope-specific T cell response to the epitope-non-specific T cell response is from about 2:1 to about 5:1, from about 5:1 to about 10:1, from about 10:1 to about 15:1, from about 15:1 to about 20:1, from about 20:1 to about 25:1, from about 25:1 to about 50:1, or from about 50:1 to about 100:1, or more than 100:1. “Modulating the activity” of a T cell can include one or more of: i) activating a cytotoxic (e.g., CD8⁺) T cell; ii) inducing cytotoxic activity of a cytotoxic (e.g., CD8⁺) T cell; iii) inducing production and release of a cytotoxin (e.g., a perforin; a granzyme; a granulysin) by a cytotoxic (e.g., CD8⁺) T cell; and iv) increasing the number of epitope-specific T cells.

The present disclosure provides a method of selectively modulating the activity of an epitope-specific T cell in an individual, the method comprising administering to the individual an effective amount of a TMP of the present disclosure, or one or more nucleic acids (e.g., expression vectors; mRNA; etc.) comprising nucleotide sequences encoding the TMP, where the TMP selectively modulates the activity of the epitope-specific T cell in the individual. Selectively modulating the activity of an epitope-specific T cell can treat a disease or disorder in the individual. Thus, the present disclosure provides a treatment method comprising administering to an individual in need thereof an effective amount of a TMP of the present disclosure.

In some cases, the MOD is an activating polypeptide, and the TMP activates the epitope-specific T cell. In some cases, the TMP increases the activity of a T cell specific for the SARS-CoV-2 epitope. In some cases, the MOD is an activating polypeptide, and the TMP activates an epitope-specific T-cell (e.g., a T-cell specific for a SARS-CoV-2 epitope). In some cases, the T cells are T-helper cells (CD4⁺ cells), cytotoxic T-cells (CD8⁺ cells), or NK-T-cells. In some cases, the epitope is a SARS-CoV-2 epitope, and the TMP increases the activity of a T-cell specific for a virus-infected cell expressing the SARS-CoV-2 epitope (e.g., T-helper cells (CD4⁺ cells), cytotoxic T-cells (CD8⁺ cells), and/or NK-T-cells). Activation of CD4⁺ T cells can include increasing proliferation of CD4⁺ T cells and/or inducing or enhancing release cytokines by CD4⁺ T cells. Activation of NK-T-cells and/or CD8⁺ cells can include: increasing proliferation of NK-T-cells and/or CD8⁺ cells; and/or inducing release of cytokines such as interferon γ by NK-T-cells and/or CD8⁺ cells.

A TMP of the present disclosure can be administered to an individual in need thereof to treat a SARS-CoV-2 infection in the individual, where a virus-infected cell expresses the SARS-CoV-2 peptide present in the TMP. The present disclosure provides a method of treating a SARS-CoV-2 infection in an individual, the method comprising administering to the individual an effective amount of a TMP of the present disclosure, or one or more nucleic acids (e.g., expression vectors; mRNA; etc.) comprising nucleotide sequences encoding the TMP, where the TMP comprises a T-cell epitope that is a SARS-CoV-2 epitope, and where the TMP comprises a stimulatory immunomodulatory polypeptide. In some cases, an “effective amount” of a TMP of the present disclosure is an amount that, when administered in one or more doses to an individual in need thereof, reduces the number of SARS-CoV-2-infected cells in the individual. For example, in some cases, an “effective amount” of a TMP of the present disclosure is an amount that, when administered in one or more doses to an individual in need thereof, reduces the number of SARS-CoV-2-infected cells in the individual by at least 10%, at least 15%, at least 20%, at least 25%, at least 30%, at least 40%, at least 50%, at least 60%, at least 70%, at least 80%, at least 90%, or at least 95%, compared to the number of SARS-CoV-2-infected cells in the individual before administration of the TMP, or in the absence of administration with the TMP. In some cases, an “effective amount” of a TMP of the present disclosure is an amount that, when administered in one or more doses to an individual in need thereof, reduces the number of SARS-CoV-2-infected cells in the individual to undetectable levels.

In some cases, an “effective amount” of a TMP of the present disclosure is an amount that, when administered in one or more doses to an individual in need thereof, reduces the viral load of SARS-CoV-2 in the individual. For example, in some cases, an “effective amount” of a TMP of the present disclosure is an amount that, when administered in one or more doses to an individual in need thereof (an individual infected with SARS-CoV2), reduces the SARS-CoV-2 viral load in the individual by at least 10%, at least 15%, at least 20%, at least 25%, at least 30%, at least 40%, at least 50%, at least 60%, at least 70%, at least 80%, at least 90%, or at least 95%, compared to the SARS-CoV-2 viral load in the individual before administration of the TMP, or in the absence of administration with the TMP. In some cases, an “effective amount” of a TMP of the present disclosure is an amount that, when administered in one or more doses to an individual in need thereof (an individual having a SARS-CoV-2 infection), reduces the number of genome copies of SARS-CoV-2 in the individual. For example, in some cases, an “effective amount” of a TMP of the present disclosure is an amount that, when administered in one or more doses to an individual in need thereof (an individual having a SARS-CoV-2 infection), reduces the number of genome copies of SARS-CoV-2 in the individual by at least 10%, at least 15%, at least 20%, at least 25%, at least 30%, at least 40%, at least 50%, at least 60%, at least 70%, at least 80%, at least 90%, or at least 95%, compared to the number of genome copies of SARS-CoV-2 in the individual before administration of the TMP, or in the absence of administration with the TMP.

In some cases, administration of a TMP of the present disclosure to an individual infected with SARS-CoV-2 ameliorates one or more symptoms of SARS-CoV-2 infection in the individual, where symptoms include fever, cough, and/or respiratory distress or conditions such as pneumonia and difficulty breathing.

As noted above, in some cases, in carrying out a subject treatment method, a TMP of the present disclosure is administered to an individual in need thereof, as the TMP per se. In other instances, in carrying out a subject treatment method, one or more nucleic acids comprising nucleotide sequences encoding a TMP of the present disclosure is/are administering to an individual in need thereof. Thus, in other instances, one or more nucleic acids of the present disclosure, e.g., one or more recombinant expression vectors of the present disclosure, is/are administered to an individual in need thereof.

### Formulations

Suitable formulations are described above, where suitable formulations include a pharmaceutically acceptable excipient. In some cases, a suitable formulation comprises: a) a TMP of the present disclosure; and b) a pharmaceutically acceptable excipient. In some cases, a suitable formulation comprises: a) a nucleic acid comprising a nucleotide sequence encoding a TMP of the present disclosure; and b) a pharmaceutically acceptable excipient; in some instances, the nucleic acid is an mRNA. In some cases, a suitable formulation comprises: a) a first nucleic acid comprising a nucleotide sequence encoding the first polypeptide of a TMP of the present disclosure; b) a second nucleic acid comprising a nucleotide sequence encoding the second polypeptide of a TMP of the present disclosure; and c) a pharmaceutically acceptable excipient. In some cases, a suitable formulation comprises: a) a recombinant expression vector comprising a nucleotide sequence encoding a TMP of the present disclosure; and b) a pharmaceutically acceptable excipient. In some cases, a suitable formulation comprises: a) a first recombinant expression vector comprising a nucleotide sequence encoding the first polypeptide of a TMP of the present disclosure; b) a second recombinant expression vector comprising a nucleotide sequence encoding the second polypeptide of a TMP of the present disclosure; and c) a pharmaceutically acceptable excipient.

Suitable pharmaceutically acceptable excipients are described above.

### Dosages

A suitable dosage can be determined by an attending physician or other qualified medical personnel, based on various clinical factors. As is well known in the medical arts, dosages for any one patient depend upon many factors, including the patient’s size, body surface area, age, the particular polypeptide or nucleic acid to be administered, sex of the patient, time, and route of administration, general health, and other drugs being administered concurrently. A TMP of the present disclosure can be administered in an amount of from about 0.1 mg/kg body weight to 20 mg/kg body weight or more, e.g., from about 0.1 mg/kg body weight to about 1 mg/kg body weight, from about 1 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, or greater than about 20 mg/kg body weight.

Persons of ordinary skill in the art can easily estimate repetition rates for dosing based on measured residence times and concentrations of the administered agent in bodily fluids or tissues. Following successful treatment, it may be desirable to have the patient undergo maintenance therapy to prevent the recurrence of the disease state, wherein a TMP of the present disclosure is administered in maintenance doses, ranging from about 0.1 mg/kg body weight to 20 mg/kg body weight or more, e.g., from about 0.1 mg/kg body weight to about 1 mg/kg body weight, from about 1 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, or greater than about 20 mg/kg body weight.

Those of skill will readily appreciate that dose levels can vary as a function of the specific TMP, the severity of the symptoms and the susceptibility of the subject to side effects. Preferred dosages for a given compound are readily determinable by those of skill in the art by a variety of means.

In some cases, multiple doses of a TMP of the present disclosure, a nucleic acid of the present disclosure, or a recombinant expression vector of the present disclosure are administered. The frequency of administration of a TMP of the present disclosure, a nucleic acid of the present disclosure, or a recombinant expression vector of the present disclosure can vary depending on any of a variety of factors, e.g., severity of the symptoms, etc. For example, in some cases, a TMP of the present disclosure, a nucleic acid of the present disclosure, or a recombinant expression vector of the present disclosure is administered once every year, once every 2-6 months, once per month, once every three weeks, or more frequently. When used prophylactically to cause priming and/or expansion of epitope-specific T cells and/or induce T cell memory, the administration can comprise an initial dose followed by one or more subsequent doses that are administered one, two, three, or four weeks, or longer, after the prior dose.

### Routes of Administration

An active agent (a TMP of the present disclosure, a nucleic acid of the present disclosure, or a recombinant expression vector of the present disclosure) is administered to an individual using any available method and route suitable for drug delivery, including in vivo and ex vivo methods, as well as systemic and localized routes of administration.

Conventional and pharmaceutically acceptable routes of administration include intramuscular, intralymphatic, intratracheal, intracranial, subcutaneous, intradermal, topical application, intravenous, intraarterial, rectal, nasal, oral, and other enteral and parenteral routes of administration. Suitable routes of administration include pulmonary administration such as in a nebulized or other aerosolized form. Routes of administration may be combined, if desired, or adjusted depending upon the TMP and/or the desired effect. A TMP of the present disclosure, or a nucleic acid or recombinant expression vector of the present disclosure, can be administered in a single dose or in multiple doses.

In some cases, a TMP of the present disclosure, a nucleic acid of the present disclosure, or a recombinant expression vector of the present disclosure is administered intravenously. In some cases, a TMP of the present disclosure is administered intravenously. In some cases, a TMP of the present disclosure is administered intramuscularly. In some cases, a TMP is administered subcutaneously.

### Combination Therapy

A TMP of the present disclosure can be administered to an individual in need thereof in combination with one or more additional therapeutic agents. The TMP of the present disclosure and the one or more additional therapeutic agents can be administered at substantially the same time or at different times.

The administration of the TMP and the one or more additional therapeutic agents can be substantially simultaneous, e.g., the TMP can be administered to an individual within about 1 minute to about 24 hours (e.g., within about 1 minute, within about 5 minutes, within about 15 minutes, within about 30 minutes, within about 1 hour, within about 4 hours, within about 8 hours, within about 12 hours, or within about 24 hours) of administration of the one or more additional therapeutic agents. In some cases, a TMP of the present disclosure is administered to an individual who is undergoing treatment with one or more additional therapeutic agents (other than the TMP. The administration of the TMP and the one or more additional therapeutic agents can occur at different times and/or at different frequencies.

As another example, a treatment method of the present disclosure can comprise coadministration of a TMP of the present disclosure and one or more additional therapeutic agents. By “coadministration” is meant that both a TMP of the present disclosure and one or more additional therapeutic agents are administered to an individual, although not necessarily at the same time, in order to achieve a therapeutic effect that is the result of having administered both the TMP and the one or more additional therapeutic agents. The administration of the TMP and the one or more additional therapeutic agents can be substantially simultaneous, e.g., the TMP can be administered to an individual within about 1 minute to about 24 hours (e.g., within about 1 minute, within about 5 minutes, within about 15 minutes, within about 30 minutes, within about 1 hour, within about 4 hours, within about 8 hours, within about 12 hours, or within about 24 hours) of administration of the one or more additional therapeutic agents. In some cases, TMP is administered to an individual who is undergoing treatment with one or more additional therapeutic agents. The administration of the TMP and the one or more additional therapeutic agents can occur at different times and/or at different frequencies.

In some cases, the additional therapeutic agent is an inhibitor of an RNA-dependent RNA polymerase. In some cases, the inhibitor is remdesivir.

### Subjects Suitable for Treatment

Subjects suitable for treatment with a method of the present disclosure include individuals who have an infection with a Betacoronavirus, or who are at risk of incurring a with a Betacoronavirus infection (e.g., a SARS-CoV-2 infection)_and/or are at greater risk of severe illness from infection (e.g., e.g., a SARS-CoV-2 infection) than the general population. Subjects suitable for treatment with a method of the present disclosure include individuals who have an infection with SARS-CoV-2. Subjects suitable for treatment with a method of the present disclosure include individuals who are at greater risk of severe illness from infection (e.g., e.g., a SARS-CoV-2 infection) than the general population. Individuals who are at greater risk of severe illness from an infection such as a SARS-CoV-2 infection than the general population include individuals having one or more underlying medical conditions selected from the group consisting of chronic kidney disease, COPD (chronic obstructive pulmonary disease), Down Syndrome, heart conditions, such as heart failure, coronary artery disease, or cardiomyopathies, an immunocompromised state (weakened immune system) from solid organ transplant, obesity (body mass index [BMI] of 30 kg/m² or higher but < 40 kg/m²), severe obesity (BMI ≥ 40 kg/m²), pregnancy, sickle cell disease, a history of smoking, Type 2 diabetes mellitus, asthma (moderate-to-severe), cerebrovascular disease (affects blood vessels and blood supply to the brain), cystic fibrosis, hypertension or high blood pressure, an immunocompromised state (weakened immune system) from blood or bone marrow transplant, immune deficiencies, human immunodeficiency virus (HIV) infection, use of corticosteroids, use of other immune weakening medicines, neurologic conditions such as dementia, liver disease, overweight (BMI > 25 kg/m², but < 30 kg/m²), pulmonary fibrosis (having damaged or scarred lung tissues), thalassemia or other blood disorders, and Type 1 diabetes mellitus.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A T cell modulatory polypeptide (TMP) comprising:
at least one heterodimer comprising:
   a) a first polypeptide comprising:
      i) a SARS-CoV-2 peptide, wherein the SARS-CoV-2 peptide has a length of from about 4 amino acids to about 25 amino acids; and
      ii) first major histocompatibility complex (MHC) polypeptide;
   b) a second polypeptide comprising a second MHC polypeptide, and
   c) one or more immunomodulatory polypeptides (“MODs”),
      wherein the first and/or the second polypeptide comprises the immunomodulatory polypeptide,
      optionally, wherein the multimeric polypeptide comprises an immunoglobulin (Ig) Fc polypeptide or a non-Ig scaffold.

Aspect 2. A TMP of aspect 1, wherein at least one of the one or more MODs is a variant MOD that binds to its cognate co-immunomodulatory polypeptide (“co-MOD and exhibits reduced affinity to a cognate co-MOD compared to the affinity of a corresponding wild-type MOD for the cognate co-MOD.

Aspect 3. A TMP of aspect 2, wherein the variant MOD binds the cognate co-MOD with an affinity that is at least 10% less than the affinity of the wild type MOD for the cognate co-MOD.

Aspect 4. A TMP of any one of aspects 1-3, wherein the first or the second polypeptide comprises an immunoglobulin (Ig) Fc polypeptide.

Aspect 5. A TMP of aspect 4, wherein the Ig Fc polypeptide is an IgG1 Fc polypeptide.

Aspect 6. A TMP of aspect 5, wherein IgG1 Fc polypeptide comprises one or more amino acid substitutions selected from N297A, L234A, L235A, L234F, L235E, and P331S.

Aspect 7. A TMP of any one of aspects 1-6, wherein
a1) the first polypeptide comprises, in order from N-terminus to C-terminus:
   i) the SARS-CoV-2 peptide; and
   ii) the first MHC polypeptide; and
b1) the second polypeptide comprises, in order from N-terminus to C-terminus:
   i) the one or more MODs;
   ii) the second MHC polypeptide; and
   iii) an Ig Fc polypeptide; or
a2) the first polypeptide comprises, in order from N-terminus to C-terminus:
   i) the SARS-CoV-2 peptide; and
   ii) the first MHC polypeptide; and
b2) the second polypeptide comprises, in order from N-terminus to C-terminus:
   i) the second MHC polypeptide;
   ii) the one or more MODs; and
   iii) an Ig Fc polypeptide; or
a3) the first polypeptide comprises, in order from N-terminus to C-terminus:
   i) the SARS-CoV-2 peptide; and
   ii) the first MHC polypeptide; and
b3) the second polypeptide comprises, in order from N-terminus to C-terminus:
   i) the second MHC polypeptide;
   ii) an Ig Fc polypeptide; and
   iii) the one or more MODs; or
a4) the first polypeptide comprises, in order from N-terminus to C-terminus:
   i) the at least one MOD;
   ii) the SARS-CoV-2 peptide;
   ii) the first MHC polypeptide; and
b4) the second polypeptide comprises, in order from N-terminus to C-terminus:
   i) the second MHC polypeptide; and
   ii) the Ig Fc polypeptide; or
a5) the first polypeptide comprises, in order from N-terminus to C-terminus:
   i) the SARS-CoV-2 peptide;
   ii) the first MHC polypeptide; and
   iii) the one or more MODs; and
b5) the second polypeptide comprises, in order from N-terminus to C-terminus:
   i) the second MHC polypeptide; and
   ii) an immunoglobulin (Ig) Fc polypeptide.

Aspect 8. A TMP of any one of aspects 1-7, wherein the first polypeptide comprises a peptide linker between the SARS-CoV-2 peptide and the first MHC polypeptide and/or wherein the second polypeptide comprises a peptide linker between the immunomodulatory polypeptide and the second MHC polypeptide.

Aspect 9. A TMP of aspect 8, wherein the peptide linker comprises the amino acid sequence (GGGGS)n (SEQ ID NO:357), where n is an integer from 1 to 10.

Aspect 10. A TMP of any one of aspects 1-9, wherein the first MHC polypeptide is a β2-microglobulin polypeptide; and wherein the second MHC polypeptide is an MHC class I heavy chain polypeptide.

Aspect 11. A TMP of any one of aspects 1-10, wherein the one or more MODs is selected from the group consisting of a cytokine, a 4-1BBL polypeptide, a CD80 polypeptide, a CD86 polypeptide, an ICOS-L polypeptide, an OX-40L polypeptide, a PD-L1 polypeptide, a FasL polypeptide, a PD-L2 polypeptide, and combinations thereof.

Aspect 12. A TMP of any one of aspects 1-11, wherein the one or more MODs is an IL-2 polypeptide.

Aspect 13. A TMP of any one of aspects 1-12, wherein the multimeric polypeptide comprises at least two MODs, and wherein at least two of the MODs are the same.

Aspect 14. A TMP of aspect 13, wherein the 2 or more MODs are in tandem.

Aspect 15. A TMP of any one of aspects 1-14, wherein the first polypeptide and the second polypeptide are covalently linked to one another.

Aspect 16. A TMP of aspect 15, wherein the covalent linkage is via a disulfide bond.

Aspect 17. A TMP of aspect 16, wherein the β2M polypeptide and the MHC heavy chain polypeptide are joined by a disulfide bond that joins a Cys residue in the β2M polypeptide and a Cys residue in the MHC heavy chain polypeptide.

Aspect 18. A TMP of aspect 17, wherein a Cys at amino acid residue 12 of the β2M polypeptide is disulfide bonded to a Cys at amino acid residue 236 of the MHC heavy chain polypeptide.

Aspect 19. A TMP of aspect 17, wherein the first polypeptide chain comprises a linker between the SARS-CoV-2 peptide and the β2M polypeptide, and wherein the disulfide bond links a Cys present in the linker with a Cys of the MHC heavy chain polypeptide.

Aspect 20. A TMP of aspect 17, wherein the first polypeptide chain comprises a linker between the peptide epitope and the β2M polypeptide, and wherein the disulfide bond links a Cys substituted for Gly2 in the linker with a Cys substituted for Tyr84 of the MHC heavy chain polypeptide.

Aspect 21. A TMP of any one of aspects 1-14, wherein the first and the second polypeptides are covalently linked to one another via at least 2 disulfide bonds.

Aspect 22. The TMP of aspect 21, wherein:
a) a first disulfide bond is between: i) a Cys present in a linker between the peptide epitope and the first MHC class I polypeptide, wherein the first MHC class I polypeptide is a β2M polypeptide; and ii) a Cys residue introduced via a Y84C substitution in the second MHC class I polypeptide, wherein the second MHC class I polypeptide is an MHC Class I heavy chain polypeptide; and
b) a second disulfide bond is between: i) a Cys residue introduced into the β2M polypeptide via an R12C substitution; and ii) a Cys residue introduced into the MHC Class I heavy chain polypeptide via an A236C substitution.

Aspect 23. A TMP of aspect 22, wherein the linker comprises the amino acid sequence GCGGS (SEQ ID NO:177).

Aspect 24. A TMP of aspect 22, wherein the linker comprises the amino acid sequence GCGGS(GGGGS)n (SEQ ID NO:171), where n is an integer from 1 to 10.

Aspect 25. A TMP of any one of aspects 1-18, wherein the MHC class I heavy chain polypeptide comprises an intrachain disulfide bond between a Cys residue introduced via a Y84C substitution and a Cys residue introduced via an A139C substitution in the MHC class I heavy chain polypeptide.

Aspect 26. A TMP of any one of aspects 1-25, wherein the SARS-CoV-2 peptide has a length of from about 8 amino acids to about 12 amino acids.

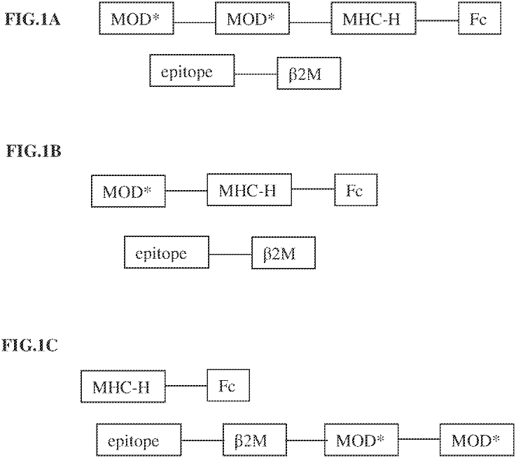

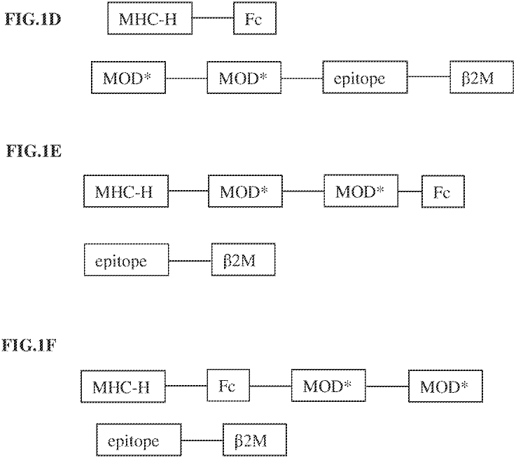

Aspect 27. A TMP of any one of aspects 1-26, wherein the first or the second MHC polypeptide comprises:
a) an amino acid sequence having at least 95% amino acid sequence identity to the HLA-A*0101, HLA-A*0201, HLA-A*0201, HLA-A*1101, HLA-A*2301, HLA-A*2402, HLA-A*2407, HLA-A*3303, or HLA-A*3401 amino acid sequence depicted in FIG. 7A; or
b) an amino acid sequence having at least 95% amino acid sequence identity to the HLA-B*0702, HLA-B*0801, HLA-B*1502, HLA-B*3802, HLA-B*4001, HLA-B*4601, or HLA-B*5301 amino acid sequence depicted in FIG. 8A; or
c) an amino acid sequence having at least 95% amino acid sequence identity to the HLA-C*0102, HLA-C*0303, HLA-C*0304, HLA-C*0401, HLA-C*0602, HLA-C*0701, HLA-C*0702, HLA-C*0801, or HLA-C*1502 depicted in FIG. 9A; or
d) an amino acid sequence having at least 95% amino acid sequence identity to the HLA-E polypeptide depicted in any one of FIGS. 20A-20D; or
e) an amino acid sequence having at least 95% amino acid sequence identity to the HLA-E polypeptide depicted in any one of FIGS. 21A-1D.

Aspect 28. A TMP of any one of aspects 1-26, wherein the first MHC polypeptide is a β2M polypeptide, and wherein the second MHC polypeptide comprises an amino acid sequence having at least 95% amino acid sequence identity to an HLA-A*0201 polypeptide.

Aspect 29. A TMP of any one of aspects 1-26, wherein the first MHC polypeptide is a β2M polypeptide, and wherein the second MHC polypeptide is an HLA-A*1101 polypeptide.

Aspect 30. A TMP of any one of aspects 1-26, wherein the first MHC polypeptide is a β2M polypeptide, and wherein the second MHC polypeptide comprises an amino acid sequence having at least 95% amino acid sequence identity to an HLA-A*3303 polypeptide.

Aspect 31. A TMP of any one of aspects 1-26, wherein the first MHC polypeptide is a β2M polypeptide, and wherein the second MHC polypeptide comprises an amino acid sequence having at least 95% amino acid sequence identity to an HLA-A*2401 polypeptide.

Aspect 32. A TMP of any one of aspects 1-31, wherein the immunomodulatory polypeptide is a variant IL-2 polypeptide comprising: i) an H16A substitution and an F42A substitution; or ii) an H16T substitution and an F42A substitution.

Aspect 33. A TMP of any one of aspects 1-32, wherein the SARS-CoV-2 peptide comprises from 4 to 25 contiguous amino acids of any one of the amino acid sequences depicted in FIGS. 13A-13J.

Aspect 34. A TMP of any one of aspects 1-32, wherein the SARS-CoV-2 peptide comprises from 4 to 25 contiguous amino acids of a SARS-CoV-2 surface glycoprotein.

Aspect 35. A TMP of aspect 34, wherein the SARS-CoV-2 peptide is a surface glycoprotein peptide depicted in FIG. 14 or FIG. 15.

Aspect 36. A TMP of any one of aspects 1-32, wherein the SARS-CoV-2 peptide comprises from 4 to 25 contiguous amino acids of a SARS-CoV-2 membrane protein.

Aspect 37. A TMP of aspect 36, wherein the SARS-CoV-2 peptide is a membrane protein peptide depicted in FIG. 14.

Aspect 38. A TMP of aspect 36, wherein the SARS-CoV-2 peptide is a membrane protein peptide depicted in FIG. 15.

Aspect 39. A TMP of any one of aspects 1-32, wherein the SARS-CoV-2 peptide comprises from 4 to 25 contiguous amino acids of a SARS-CoV-2 nucleocapsid phosphoprotein.

Aspect 40. A TMP of aspect 39, wherein the wherein the SARS-CoV-2 peptide is a nucleocapsid phosphoprotein peptide depicted in FIGS. 14 or 15.

Aspect 41. A TMP of any one of aspects 1-40, wherein the first MHC polypeptide is a β2M polypeptide, and wherein the second MHC polypeptide is selected from an HLA-A*01:01 polypeptide, an HLA-A*02:01 polypeptide an HLA-A*03:01 polypeptide, an HLA-A*11:01 polypeptide, an HLA-A*23:01 polypeptide, and an HLA-A*24:02 polypeptide.

Aspect 42. A TMP of any one of aspects 1-40, wherein the first MHC polypeptide is a β2M polypeptide, and wherein the second MHC polypeptide is selected from an HLA-B*07:02 polypeptide, an HLA-B*08:01 polypeptide, an HLA-B*35:01 polypeptide, an HLA-B*40:01 polypeptide, an HLA-B*44:02 polypeptide, and an HLA-B*44:03 polypeptide.

Aspect 43. A protein comprising a first and a second heterodimer according to any one of aspects 1-42.

wherein the first and second heterodimers are covalently bound by one or more disulfide bonds between the Ig Fc polypeptides of the first and second heterodimers.

Aspect 44. A nucleic acid comprising a nucleotide sequence encoding a first or second polypeptide according to any one of aspects 1-42.

Aspect 45. An expression vector comprising the nucleic acid of aspect 44.

Aspect 46. A method of selectively modulating the activity of T cell specific for a peptide epitope, the method comprising contacting the T cell with a TMP according to any one of aspects 1-42 or a protein of aspect 43, wherein said contacting selectively modulates the activity of the epitope-specific T cell.

Aspect 47. A method of treating a SARS-CoV-2 infection in a patient infected with SARS-CoV-2, the method comprising administering to the patient an effective amount of a pharmaceutical composition comprising TMP according to any one of aspects 1-42 or a protein of aspect 43.

Aspect 48. The method of aspect 47, said administering is pulmonary.

Aspect 49. The method of aspect 47, wherein said administering is intramuscular.

Aspect 50. The method of aspect 47, wherein said administering is intravenous.

Aspect 51. The method of any one of aspects 47-50, further comprising administering an inhibitor of an RNA-dependent RNA polymerase.

Aspect 52. The method of aspect 51, wherein the inhibitor is remdesivir.

Aspect 53. The method of any one of aspects 47-50, further comprising administering chloroquine or hydroxycholoroquine.

Aspect 54. The method of any one of aspects 47-50, further comprising administering a second TMP, wherein the second TMP:
a) has the same peptide epitope as the TMP of any one of aspects 1-42 or protein of aspect 43;
b) has a different peptide epitope from the TMP of any one of aspects 1-42 or protein of aspect 43;
c) has the same MOD as the TMP of any one of aspects 1-42 or protein of aspect 43; or
d) has a different MOD from the TMP of any one of aspects 1-42 or protein of aspect 43.

Aspect 55. A method of modulating an immune response in an individual, the method comprising administering to the individual an effective amount of the TMP of any one of aspects 1-42 or protein of aspect 43,
wherein said administering induces an epitope-specific T cell response and an epitope-non-specific T cell response,
wherein the ratio of the epitope-specific T cell response to the epitope-non-specific T cell response is at least 2:1.

Aspect 56. A method of delivering an immunomodulatory polypeptide (“MOD”) selectively to a target T cell, the method comprising contacting a mixed population of T cells with a TMP of any one of aspects 1-42 or protein of aspect 43, wherein the mixed population of T cells comprises the target T cell and non-target T cells, wherein the target T cell is specific for the SARS-CoV-2 epitope present within the TMP, and wherein said contacting delivers the one or more MODs present within the TMP to the target T cell.

Aspect 57. A method of detecting, in a mixed population of T cells obtained from an individual, the presence of a target T cell that binds a SARS-CoV-2 peptide, the method comprising:
a) contacting in vitro the mixed population of T cells with the TMP of any one of aspects 1-42 or protein of aspect 43, wherein the TMP comprises the peptide epitope; and
b) detecting activation and/or proliferation of T cells in response to said contacting, wherein activated and/or proliferated T cells indicates the presence of the target T cell.

Aspect 58. A T-cell modulatory polypeptide comprising:
a) a Betacoronavirus SARS-CoV-2-associated peptide, wherein the SARS-CoV-2 peptide has a length of from about 4 amino acids to about 25 amino acids;
b) a first major histocompatibility complex (MHC) polypeptide;
c) a second MHC polypeptide, and
d) one or more immunomodulatory polypeptides (“MODs”),
optionally, wherein the T-cell modulatory polypeptide comprises an immunoglobulin (Ig) Fc polypeptide or a non-Ig scaffold.

Aspect 59. A T-cell modulatory polypeptide of aspect 58, wherein at least one of the one or more immunomodulatory domains is a variant MOD that binds to its cognate co-immunomodulatory polypeptide (“co-MOD”) and exhibits reduced affinity to a cognate co-MOD compared to the affinity of a corresponding wild-type MOD for the cognate co-MOD.

Aspect 60. A T-cell modulatory polypeptide of aspect 59, wherein the variant MOD binds the cognate co-MOD with an affinity that is at least 10% less than the affinity of the wild type MOD for the cognate co-MOD.

Aspect 61. A T-cell modulatory polypeptide of any one of aspects 58-60, wherein the T-cell modulatory polypeptide comprises an immunoglobulin (Ig) Fc polypeptide.

Aspect 62. A T-cell modulatory polypeptide of aspect 61, wherein the Ig Fc polypeptide is an IgG1 Fc polypeptide.

Aspect 63. A T-cell modulatory polypeptide of aspect 62, wherein IgG1 Fc polypeptide comprises one or more amino acid substitutions selected from N297A, L234A, L235A, L234F, L235E, and P331S.

Aspect 64. A T-cell modulatory polypeptide of any one of aspects 58-63, wherein
a) the T-cell modulatory polypeptide comprises, in order from N-terminus to C-terminus:
   i) the SARS-CoV-2 peptide; and
   ii) the first MHC polypeptide;
   iii) the second MHC polypeptide;
   iv) the one or more MODs; and
   v) the Ig Fc polypeptide; or
b) the T-cell modulatory polypeptide comprises, in order from N-terminus to C-terminus:
   i) the SARS-CoV-2 peptide; and
   ii) the first MHC polypeptide;
   iii) the second MHC polypeptide;
   iv) the Ig Fc polypeptide; and
   v) the one or more MODs; or
c) the T-cell modulatory polypeptide comprises, in order from N-terminus to C-terminus:
   i) the one or more MODs;
   ii) the SARS-CoV-2 peptide;
   iii) the first MHC polypeptide;
   iv) the second MHC polypeptide; and
   v) the Ig Fc polypeptide.

Aspect 65. A T-cell modulatory polypeptide of any one of aspects 58-64, wherein the T-cell modulatory polypeptide comprises a peptide linker between the SARS-CoV-2 peptide and the first MHC polypeptide and/or wherein the T-cell modulatory polypeptide comprises a peptide linker between the MOD and the second MHC polypeptide.

Aspect 66. A T-cell modulatory polypeptide of aspect 65, wherein the peptide linker comprises the amino acid sequence (GGGGS)n (SEQ ID NO:357), where n is an integer from 1 to 10.

Aspect 67. A T-cell modulatory polypeptide of any one of aspects 58-66, wherein the first MHC polypeptide is a β2-microglobulin polypeptide; and wherein the second MHC polypeptide is an MHC class I heavy chain polypeptide.

Aspect 68. A T-cell modulatory polypeptide of any one of aspects 58-67, wherein the at least one MOD is selected from the group consisting of a cytokine, a 4-1BBL polypeptide, a CD80 polypeptide; a CD86 polypeptide, an ICOS-L polypeptide, an OX-40L polypeptide, a PD-L1 polypeptide, a FasL polypeptide, a PD-L2 polypeptide, and combinations thereof.

Aspect 69. A T-cell modulatory polypeptide of any one of aspects 58-68, wherein the at least one MOD is an IL-2 polypeptide.

Aspect 70. A T-cell modulatory polypeptide of any one of aspects 58-69, wherein the T-cell modulatory polypeptide comprises at least two MODs, and wherein at least two of the MODs are the same.

Aspect 71. A T-cell modulatory polypeptide of aspect 70, wherein the 2 or more MODs are in tandem.

Aspect 72. A T-cell modulatory polypeptide of any one of aspects 67-71, wherein the MHC class I heavy chain polypeptide comprises an intrachain disulfide bond between a Cys residue introduced via a Y84C substitution and a Cys residue introduced via an A139C substitution in the MHC class I heavy chain polypeptide.

Aspect 73. A T-cell modulatory polypeptide of any one of aspects 58-72, wherein the SARS-CoV-2 peptide is a peptide of from about 8 amino acids to about 12 amino acids in length.

Aspect 74. A T-cell modulatory polypeptide of any one of aspects 58-73, wherein the first or the second MHC polypeptide comprises:
a) an amino acid sequence having at least 95% amino acid sequence identity to the HLA-A*0101, HLA-A*0201, HLA-A*0201, HLA-A*1101, HLA-A*2301, HLA-A*2402, HLA-A*2407, HLA-A*3303, or HLA-A*3401 amino acid sequence depicted in FIG. 7A; or
b) an amino acid sequence having at least 95% amino acid sequence identity to the HLA-B*0702, HLA-B*0801, HLA-B*1502, HLA-B*3802, HLA-B*4001, HLA-B*4601, or HLA-B*5301 amino acid sequence depicted in FIG. 8A; or
c) an amino acid sequence having at least 95% amino acid sequence identity to the HLA-C*0102, HLA-C*0303, HLA-C*0304, HLA-C*0401, HLA-C*0602, HLA-C*0701, HLA-C*0702, HLA-C*0801, or HLA-C*1502 depicted in FIG. 9A; or
d) an amino acid sequence having at least 95% amino acid sequence identity to the HLA-E polypeptide depicted in any one of FIGS. 20A-20D; or
e) an amino acid sequence having at least 95% amino acid sequence identity to the HLA-E polypeptide depicted in any one of FIGS. 21A-1D.

Aspect 75. A T-cell modulatory polypeptide of any one of aspects 58-74, wherein the first MHC polypeptide is a β2M polypeptide, and wherein the second MHC polypeptide comprises an amino acid sequence having at least 95% amino acid sequence identity to an HLA-A*0201 polypeptide.

Aspect 76. A T-cell modulatory polypeptide of any one of aspects 58-74, wherein the first MHC polypeptide is a β2M polypeptide, and wherein the second MHC polypeptide is an HLA-A*1101 polypeptide.

Aspect 77. A T-cell modulatory polypeptide of any one of aspects 58-74, wherein the first MHC polypeptide is a β2M polypeptide, and wherein the second MHC polypeptide comprises an amino acid sequence having at least 95% amino acid sequence identity to an HLA-A*3303 polypeptide.

Aspect 78. A T-cell modulatory polypeptide of any one of aspects 58-74, wherein the first MHC polypeptide is a β2M polypeptide, and wherein the second MHC polypeptide comprises an amino acid sequence having at least 95% amino acid sequence identity to an HLA-A*2401 polypeptide.

Aspect 79. A T-cell modulatory polypeptide of any one of aspects 58-78, wherein the immunomodulatory polypeptide is a variant IL-2 polypeptide comprising: i) an H16A substitution and an F42A substitution; or ii) an H16T substitution and an F42A substitution.

Aspect 80. A T-cell modulatory polypeptide of any one of aspects 58-79, wherein the SARS-CoV-2 peptide is a peptide of any one of the amino acid sequences depicted in FIGS. 13A-13J.

Aspect 81. A T-cell modulatory polypeptide of any one of aspects 58-79, wherein the SARS-CoV-2 peptide comprises from 4 to 25 contiguous amino acids of a SARS-CoV-2 surface glycoprotein.

Aspect 82. A T-cell modulatory polypeptide of aspect 81, wherein the SARS-CoV-2 peptide is a surface glycoprotein peptide depicted in FIG. 14.

Aspect 83. A T-cell modulatory polypeptide of aspect 81, wherein the SARS-CoV-2 peptide is a surface glycoprotein peptide depicted in FIG. 15.

Aspect 84. A T-cell modulatory polypeptide of any one of aspects 58-79, wherein the SARS-CoV-2 peptide comprises from 4 to 25 contiguous amino acids of a SARS-CoV-2 membrane protein.

Aspect 85. A T-cell modulatory polypeptide of aspect 84, wherein the SARS-CoV-2 peptide is a membrane protein peptide depicted in FIG. 14.

Aspect 86. A T-cell modulatory polypeptide of aspect 84, wherein the SARS-CoV-2 peptide is a membrane protein peptide depicted in FIG. 15.

Aspect 87. A T-cell modulatory polypeptide of any one of aspects 58-79, wherein the SARS-CoV-2 peptide comprises from 4 to 25 contiguous amino acids of a SARS-CoV-2 nucleocapsid phosphoprotein.

Aspect 88. A T-cell modulatory polypeptide of aspect 87, wherein the wherein the SARS-CoV-2 peptide is a nucleocapsid phosphoprotein peptide depicted in FIG. 14.

Aspect 89. A T-cell modulatory polypeptide of aspect 87, wherein the wherein the SARS-CoV-2 peptide is a nucleocapsid phosphoprotein peptide depicted in FIG. 15.

Aspect 90. A T-cell modulatory polypeptide of any one of aspects 58-79, wherein the first MHC polypeptide is a β2M polypeptide, and wherein the second MHC polypeptide is selected from an HLA-A*01:01 polypeptide, an HLA-A*02:01 polypeptide an HLA-A*03:01 polypeptide, an HLA-A*11:01 polypeptide, an HLA-A*23:01 polypeptide, and an HLA-A*24:02 polypeptide.

Aspect 91. A T-cell modulatory polypeptide of any one of aspects 58-79, wherein the first MHC polypeptide is a β2M polypeptide, and wherein the second MHC polypeptide is selected from an HLA-B*07:02 polypeptide, an HLA-B*08:01 polypeptide, an HLA-B*35:01 polypeptide, an HLA-B*40:01 polypeptide, an HLA-B*44:02 polypeptide, and an HLA-B*44:03 polypeptide,.

Aspect 92. A T-cell modulatory polypeptide of any one of aspects 58-79, wherein the first MHC polypeptide is a β2M polypeptide, and wherein the second MHC polypeptide comprises an amino acid sequence having at least 95% amino acid sequence identity to an HLA-A*01:01 polypeptide.

Aspect 93. A T-cell modulatory polypeptide of any one of aspects 58-79, wherein the first MHC polypeptide is a β2M polypeptide, and wherein the second MHC polypeptide comprises an amino acid sequence having at least 95% amino acid sequence identity to an HLA-A*02:01 polypeptide.

Aspect 94. A T-cell modulatory polypeptide of any one of aspects 58-79, wherein the first MHC polypeptide is a β2M polypeptide, and wherein the second MHC polypeptide comprises an amino acid sequence having at least 95% amino acid sequence identity to an HLA-A*03:01 polypeptide.

Aspect 95. A protein comprising a first T-cell modulatory polypeptide according to any one of aspects 58-89 and a second T-cell modulatory polypeptide according to any one of aspects 58-89,

wherein the first and second T-cell modulatory polypeptides are covalently bound by one or more disulfide bonds between the Ig Fc polypeptides of the first and second T-cell modulatory polypeptides.

Aspect 96. A nucleic acid comprising a nucleotide sequence encoding a T-cell modulatory polypeptide according to any one of aspects 58-89.

Aspect 97. An expression vector comprising the nucleic acid of aspect 96.

Aspect 98. A method of selectively modulating the activity of T cell specific for a peptide epitope, the method comprising contacting the T cell with a T-cell modulatory polypeptide according to any one of aspects 58-89, or a protein according to aspect 90, wherein said contacting selectively modulates the activity of the epitope-specific T cell.

Aspect 99. A method of treating a SARS-CoV2 infection in a patient infected with SARS-CoV-2, the method comprising administering to the patient an effective amount of a pharmaceutical composition comprising a T-cell modulatory polypeptide according to any one of aspects 58-89, or a protein according to aspect 90.

Aspect 100. The method of aspect 99, wherein said administering is pulmonary.

Aspect 101. The method of aspect 99, wherein said administering is intramuscular.

Aspect 102. The method of aspect 99, wherein said administering is intravenous.

Aspect 103. The method of any one of aspects 99-102, further comprising administering inhibitor of an RNA-dependent RNA polymerase.

Aspect 104. The method of aspect 103, wherein the inhibitor is remdesivir.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1-104. (canceled)

105. A T cell modulatory polypeptide (TMP) comprising:
at least one heterodimer comprising:
a) a first polypeptide comprising:
i) a SARS-CoV-2 peptide, wherein the SARS-CoV-2 peptide has a length of from about 4 amino acids to about 25 amino acids;
ii) a β2-microglobulin (β2M) polypeptide; and
iii) a cysteine (Cys)-containing linker that links the SARS-CoV-2 peptide to the β2M polypeptide; and
b) a second polypeptide comprising:
i) an MHC class I heavy chain polypeptide;
ii) one or more immunomodulatory polypeptides (MODs); and
iii) an immunoglobulin (Ig) Fc polypeptide,
wherein the first polypeptide and the second polypeptide are covalently linked to one another via at least a first and second disulfide bond, wherein the first disulfide bond is formed between (i) the Cys residue in the Cys-containing linker between the SARS-CoV-2 peptide and the β2M polypeptide, and (ii) a Cys residue in the MHC class I heavy chain polypeptide; and the second disulfide bond is formed between a Cys residue in the β2M polypeptide and a Cys residue in the MHC class I heavy chain polypeptide.

106. A TMP of claim 105, wherein:
a1) the first polypeptide comprises, in order from N-terminus to C-terminus:
i) the SARS-CoV-2 peptide;
ii) the Cys-containing linker; and
ii) the β2M polypeptide; and
b1) the second polypeptide comprises, in order from N-terminus to C-terminus:
i) the one or more MODs;
ii) the MHC class I heavy chain polypeptide; and
iii) an Ig Fc polypeptide; or
a2) the first polypeptide comprises, in order from N-terminus to C-terminus:
i) the SARS-CoV-2 peptide;
ii) the Cys-containing linker; and
ii) the β2M polypeptide; and
b2) the second polypeptide comprises, in order from N-terminus to C-terminus:
i) the MHC class I heavy chain polypeptide;
ii) the one or more MODs; and
iii) an Ig Fc polypeptide; or
a3) the first polypeptide comprises, in order from N-terminus to C-terminus:
i) the SARS-CoV-2 peptide;
ii) the Cys-containing linker; and
ii) the β2M polypeptide; and
b3) the second polypeptide comprises, in order from N-terminus to C-terminus:
i) the MHC class I heavy chain polypeptide;
ii) an Ig Fc polypeptide; and
iii) the one or more MODs,
optionally, wherein TMP comprises one or more independently selected peptide linkers between any two of the components of the second polypeptide.

107. The TMP of claim 106, wherein:
a) a first disulfide bond is between: i) a Cys present in the Cys-containing linker between the peptide epitope and the β2M polypeptide, and ii) a Cys residue at position 84 in the MHC class I heavy chain polypeptide; and
b) a second disulfide bond is between: i) a Cys residue at position 12 in the β2M polypeptide, and ii) a Cys residue at position 236 in the MHC Class I heavy chain polypeptide.

108. A TMP of claim 106, wherein the one or more MODs is selected from the group consisting of a cytokine, a 4-1BBL polypeptide, a CD80 polypeptide, a CD86 polypeptide, an ICOS-L polypeptide, an OX-40L polypeptide, a PD-L1 polypeptide, a FasL polypeptide, a PD-L2 polypeptide, and combinations thereof.

109. A TMP of claim 106, wherein the one or more MODs is a variant IL-2 polypeptide comprising an amino acid other than histidine at position 16 and an amino acid other than phenylalanine at position 42.

110. A homodimer comprising a first and a second heterodimer according to claim 106, wherein the first and second heterodimers are covalently bound by one or more disulfide bonds between the Ig Fc polypeptides of the first and second heterodimers.

111. One or more nucleic acids comprising nucleotide sequences encoding the first and the second polypeptide according to claim 106.

112. A host cell genetically modified with the one or more nucleic acids of claim 111.

113. A method of making a TMP comprising culturing the host cell of claim 112 under conditions that produce the TMP.

114. A method of treating a SARS-CoV-2 infection in a patient infected with SARS-CoV-2, the method comprising administering to the patient an effective amount of a pharmaceutical composition comprising a homodimer according to claim 111.

115. A single-chain T-cell modulatory polypeptide (TMP) comprising:
a) a Betacoronavirus SARS-CoV-2-associated peptide, wherein the SARS-CoV-2 peptide has a length of from about 4 amino acids to about 25 amino acids;
b) a β2-microglobulin (β2M) polypeptide;
c) an MHC class I heavy chain polypeptide, and
d) one or more immunomodulatory polypeptides (MODs),
optionally, wherein the TMP comprises an immunoglobulin (Ig) Fc polypeptide or a non-Ig scaffold.

116. A single-chain TMP of claim 115, wherein
a) the TMP comprises, in order from N-terminus to C-terminus:
i) the SARS-CoV-2 peptide; and
ii) the β2M polypeptide;
iii) the MHC class I heavy chain polypeptide;
iv) the one or more MODs; and
v) the Ig Fc polypeptide; or
b) the TMP comprises, in order from N-terminus to C-terminus:
i) the SARS-CoV-2 peptide; and
ii) the β2M polypeptide;
iii) the MHC class I heavy chain polypeptide;
iv) the Ig Fc polypeptide; and
v) the one or more MODs; or
c) the TMP comprises, in order from N-terminus to C-terminus:
i) the one or more MODs;
ii) the SARS-CoV-2 peptide;
iii) the β2M polypeptide;
iv) the MHC class I heavy chain polypeptide; and
v) the Ig Fc polypeptide,
optionally, wherein TMP comprises one or more independently selected peptide linkers between any two of the components of the TMP.

117. A single-chain TMP of claim 116, wherein the TMP comprises, in order from N-terminus to C-terminus:
i) the SARS-CoV-2 peptide;
ii) the β2M polypeptide;
iii) the MHC class I heavy chain polypeptide;
iv) the Ig Fc polypeptide; and
v) the one or more MODs, and
wherein TMP may comprise one or more independently selected peptide linkers between any two of the components of the TMP.

118. A single-chain TMP of claim 115, wherein the one or more MODs is selected from the group consisting of a cytokine, a 4-1BBL polypeptide, a CD80 polypeptide, a CD86 polypeptide, an ICOS-L polypeptide, an OX-40L polypeptide, a PD-L1 polypeptide, a FasL polypeptide, a PD-L2 polypeptide, and combinations thereof.

119. A single-chain TMP of claim 117, wherein the one or more MODs is a variant IL-2 polypeptide comprising an amino acid other than histidine at position 16 and an amino acid other than phenylalanine at position 42.

120. A homodimer comprising a first and a second heterodimer according to claim 117, wherein the first and second heterodimers are covalently bound by one or more disulfide bonds between the Ig Fc polypeptides of the first and second heterodimers.

121. A nucleic acid comprising a nucleotide sequence encoding a single-chain TMP according to claim 117.

122. A host cell genetically modified with the one or more nucleic acids of claim 121.

123. A method of making a TMP comprising culturing the host cell of claim 122 under conditions that produce the TMMP.

124. A method of treating a SARS-CoV-2 infection in a patient infected with SARS-CoV-2, the method comprising administering to the patient an effective amount of a pharmaceutical composition comprising a homodimer according to claim 121.
